(19) United States
(12) Patent Application Publication
(10) Pub. No.: US 2004/0083501 A1
(43) Pub. Date: Apr. 29, 2004

(54) Plant genes that confer resistance to strains of Magnaporthe grisea having AVR CO39 cultivar specificity gene

(76) Inventors: Sally A. Leong, Avoca, WI (US); Rajinder S. Chauhan, Madison, WI (US); Timothy J. Durfee, Madison, WI (US); Mark L. Farman, Lexington, KY (US)

Correspondence Address: USDA-ARS-OFFICE OF TECHNOLOGY TRANSFER, NATIONAL CTR FOR AGRICULTURAL UTILIZATION RESEARCH, 1815 N. UNIVERSITY STREET, PEORIA, IL, 61604, US

(21) Appl. No.: 10/459,262

(22) Filed: Jun. 11, 2003

Related U.S. Application Data
(63) Continuation-in-part of application No. 10/415,058, filed on Oct. 3, 2003.
Application No. 10/415,058 is a 371 of international application No. PCT/US01/46331, filed on Oct. 19, 2001.
(60) Provisional application No. 60/303,897, filed on Jul. 9, 2001.
(60) Provisional application No. 60/242,313, filed on Oct. 20, 2000.

(51) Int. Cl. A01H 1/00; C12N 15/82

(52) U.S. Cl. 800/279

(57) ABSTRACT

Plant pathogen resistance gene (s) present in rice cultivar CO39 and other plant species is disclosed. The locus is referred to as Pi-CO39(t) and confers resistance to strains of the plant pathogen, Magnaporthe grisea (causal agent of rice blast and other plant diseases), having a corresponding AVR1 CO39 avirulence gene. Also disclosed are methods of using the resistance gene and its encoded products for improving resistance of plants to this pathogen.

This application claims priority under 35 U.S.C. §120 to U.S. application Ser. No. 10/415,058, filed on Apr. 11, 2003, which was a National Stage filing under 35 U.S.C. 371 of PCT/US01/46331, filed on Oct. 19, 2001 (corresponding to PCT WO 02/34927, published May 2, 2002), which in turn claimed priority under §119(e)(1) to U.S. Provisional Application No. 60/242,313, filed Oct. 20, 2000, and to U.S. Provisional Application No. 60/303,897, filed Jul. 9, 2001, the entireties all of which are incorporated by reference herein.

### BACKGROUND OF THE INVENTION

1Field of the Invention

This invention relates to the field of disease resistance in plants. In particular, the invention is drawn to novel resistance genes present in rice cultivar CO39 and other plant species, that confers resistance to strains of the rice blast pathogen, Magnaporthe grisea, having a corresponding avirulence gene. This invention further provides methods of using the resistance gene and its encoded products for improving resistance of plants to this pathogen.

2. Description of the Prior Art

Various patent and scientific publications are referred to throughout the specification to describe the state of the art to which this invention pertains. Full citations of such publications not appearing within the specification may be found at the end of the specification. Each of these publications is incorporated by reference herein in its entirety.

Rice is a major staple food for about two-thirds of the world's population. More than ninety percent of the world's rice is grown and consumed in developing countries. Rice blast disease, caused by the fungus Magnaporthe grisea, threatens rice crops worldwide. The disease can cause yield losses of ten to thirty percent in infested fields. Rice blast has been an ongoing problem in rice growing areas of the southern United States. It has now become a significant problem in rice growing areas of California, as well.

The “gene-for-gene” hypothesis has been advanced to explain the very specific disease resistance/susceptibility relationship that often exists between races of a plant pathogen and cultivars of its host species. The gene-for-gene hypothesis has been found applicable to many host-pathogen interactions, including that of the rice blast fungus, Magnaporthe grisea, and its host, Oryza sativa. To be able to understand and manipulate this host-pathogen relationship is of great practical interest as M. grisea is rapidly able to overcome new disease resistance in rice soon after their deployment. Moreover, M. grisea exists as a complex genus with many subspecific groups that are sometimes interfertile, but differ in their host range. How these different subspecific groups interrelate evolutionarily is of great concern to plant breeders since some of these alternate hosts are frequently found growing in close proximity to, or in rotation with rice, and M. grisea isolates infecting these alternate hosts can sometimes also infect rice.

Gene-for-gene resistance (also known as hypersensitive resistance (HR) or race-specific resistance) depends for its activation on specific recognition of the invading pathogen by the plant. Many individual plant genes have been identified that control gene-for-gene resistance. These genes are referred to as resistance (R) genes. The function of a particular R gene depends on the genotype of the pathogen. A pathogen gene is referred to as an Avr gene if its expression causes the pathogen to produce a signal that triggers a strong defense response in a plant having a corresponding R gene. This response is not observed in the absence of either the Avr gene in the pathogen or the corresponding R gene in the plant. It should be noted that a single plant may have many R genes, and a single pathogen may have many Avr genes. However, strong resistance occurs only when an Avr gene (which is usually a dominant allele) and its corresponding specific R gene (also usually a dominant allele) are matched in a host-pathogen interaction. In this instance, resistance generally occurs as activation of a HR response, in which the cells in the immediate vicinity of the infection undergo programmed necrosis in order to prevent the further advance of the pathogen into living plant tissue. Other features of the resistance response may also include synthesis of antimicrobial metabolites or pathogen-inhibiting enzymes, reinforcement of plant cell walls in the infected area, and induction of signal transduction pathways leading to systemic acquired resistance (SAR) in the plant.

The molecular basis of host-cultivar specificity and pathogenic variability in M. grisea has been partly elucidated with the identification, mapping and, in some instances, cloning of specific Avr genes from pathogenic isolates of M. grisea. For instance, AVR2-YAMO (more recently named Avr-Pita)(cultivar specificity) and PWL2 (host specificity) (Valent & Chumley, pp. 3.113-3.134 in Rice Blast Disease (R. Zeigler, S. A. Leong, P. Teng, eds.), Wallingford: CAB International, 1994) both function as classic avirulence genes by preventing infection of a specific cultivar or host. AVR2-YAMO encodes a 223-amino acid protein with homology to proteases, while PWL2 encodes a 145-amino acid polypeptide which is glycine-rich. Based on the predicted amino acid sequences of the proteins, both may be secreted (Sweigard et al., Plant Cell 7:1221-1233, 1995; Jia et al., EMBO J. 19:4004-4014, 2000).

Homologs of both AVR2-YAMO and PWL2 appear to be widely distributed in rice and in other grass-infecting isolates of M. grisea, thereby confirming that M. grisea isolates which do not infect rice still may carry host or cultivar specificity genes for rice. In some cases, homologs of AVR2-YAMO and PWL2 have been shown to be functional and to exhibit the same host or cultivar specificity as AVR2-YAMO or PWL2 (Kang et al., Molecular Plant-Microbe Interactions. 8:939-498, 1995; Orbach et al., Plant Cell.12:2019-2032, 2000; Jia et al., EMBO J 19:4004-4014, 2000).

As another example of a potentially useful Avr gene, the cultivar specificity gene AVR1-CO39, which determines avirulence on rice cultivar CO39, has been identified (Valent et al., Genetics 127:87-101, 1991) and mapped to a position on M. grisea chromosome 1 (Smith & Leong, Theor. Appl. Genet. 88:901-908, 1994). The avirulence gene in M. grisea (AVR1-CO39) has been cloned and sequenced (Farman & Leong, Genetics 150:1049-1058, 1998; see also PCT US99/04047 and commonly-owned co-pending U.S. application Ser. No. 09/257,585).

Indica rice cultivar CO39 was originally bred for blast resistance and agronomic value and has been lately used as a tester for blast pathogenicity assays as well as a recurrent parent for developing near-isogenic-lines. Genetic analysis of blast resistance in CO39 to M. grisea progeny, 6082, which carries AVR1-CO39, as well as to the Guy11 (AVR1-CO39) transformant has shown that resistance is controlled by a single dominant locus. The resistance phenotype is uniform and consistently of reaction type 1 and is inherited as a simple Mendelian trait among different segregating populations (F1/F2/F3/F4). The resistance gene(s), designated as Pi-CO39(t), has been mapped to the short arm of rice chromosome 11 (Chauhan et al., Mol. Genet. Genomics 267:603-612, 2002).

In addition to cloned cultivar and host specificity genes from M. grisea, the availability of the corresponding R genes from rice would provide useful tools for manipulating and augmenting resistance to this pathogen in the field. Accordingly, it is an object of the present invention to provide a new cloned rice R gene that confers resistance to strains of Magnaporthe grisea carrying the AVR1-CO39 avirulence gene. It is a further object of the present invention to provide methods for using the R gene and the corresponding fungal avirulence gene to confer or improve resistance of other cultivars and plant species to rice blast and other plant diseases.

### SUMMARY OF THE INVENTION

One aspect of the invention features an isolated nucleic acid segment from chromosome 11 of Indica rice cultivar CO39, which comprises one or more genes that confer resistance to strains of the rice blast pathogen, Magnaporthe grisea, that have the avirulence gene AVR1-CO39. The locus comprising the gene(s) co-segregates with one or more of the following markers: (1) RGA8 (GenBank Accession No. AF074889; Mago et al., Theor. Appl. Genet. 99:50-57, 1999); (2) RGA38 (GenBank Accession No. AF074895; Mago et al., Theor. Appl. Genet. 99:50-57, 1999); and (3) G320 (GenBank Accession No. RICG320A Fukuoka et al., DNA Res. 1 (6), 271-277 (1994). The gene (or, if more than one, the plurality of genes) is referred to herein as Pi-CO39(t). It should be noted that the term Pi-CO39(t) gene, if used in the singular herein, also refers to any plurality of genes associated with resistance to AVR1-CO39expressing strains of M. grisea.

According to another aspect of the invention, a transgenic plant comprising the Pi-CO39(t) resistance gene is provided. Expression of the gene in the transgenic plants confers a resistance response upon challenge with the gene product of AVR1-CO39 or microorganisms expressing the AVR1-CO39 gene. In a preferred embodiment, the plant is rice. In another embodiment, the plant is a monocot other than rice, which is susceptible to diseases caused by Magnaporthe. Such plants include, for example, turf grasses such as Lolium perenne. In another embodiment, the plant is a dicotyledenous species.

According to another aspect of the invention, a method of enhancing pathogen resistance in a plant is provided. The method comprises the following steps: (1) transforming the plant with the Pi-CO39(t) gene; and (2) pre-treating the transformed plant with either the AVR1-CO39 gene product or a non-pathogenic organism (e.g., an epiphytic bacterium or a non-pathogenic fungus) that expresses a portion of an AVR1-CO39 gene effective to trigger expression of a CO39-specific R gene in the plants. Triggering expression of the R gene in this manner will confer upon the plant increased resistance not only to Magnaporthe grisea, but also to other plant pathogens whose infective ability is reduced or prevented by the R gene product and its associated activity.

These and other features and advantages of the present invention will be described in greater detail in the description and examples set forth below.

### BRIEF DESCRIPTION OF THE DRAWINGS

FIG. 1. Schematic diagram showing a genetic map (upper portion of figure) linked with a physical map (lower portion of figure) of a region of rice chromosome 11-associated with Pi-CO39(t). The genetic map displays the resistance gene(s), Pi-CO39(t) with respect to co-segregating markers. Designations on the bottom of the genetic map of chromosome 11 of rice CO39 are names of genetic markers. Numbers on the top of the genetic map represent distance in centiMorgans (cM). The physical map is from Japonica rice variety, Nipponbare and Indica rice variety CO39. The physical map shows minimum tiling path of BAC clones from a Japonica rice variety, Nipponbare, and its relationship to a contig of BAC clones from Indica rice variety CO39.

FIG. 2. Structural organization of predicted genes in haplotypes of disease resistant (CO39) and susceptible (Nipponbare) rice genotypes at Pi-CO39(t) locus. NSL (Nipponbare serpin-like genes); CSL (CO39 serpin-like genes); NBR [Nipponbare NBS-LRR (nucleotide binding site and leucine-rich repeat) disease resistance-like genes]; COR(CO39 NBS-LRR disease resistance-like genes); note NBR2, NBR5 and COR2 are like a rice Pib-like gene; NBR3, NBR4 and COR3 and COR5 are rice Pi-ta-like genes; (N)KIN1, (N)KIN2 and (N)KIN3 are Nipponbare kinases, whereas (C)KIN1 is a CO39 kinase-like gene; COR6 is a rice Xa1-like gene; all other NBS-LRR genes in both the genotypes are similar to RPR1-like genes; and the inverted triangle indicate retroelement insertions. Genes, NBR17, NBR18, NSL3, NSL4, NSL5, and NSL6 are predicted in a Nipponbare BAC clone, 7H22 (Accession No. AC 119670) sequenced by The Institute for Genomic Research (http://www.tigr.org), while all other genes are predicted from Nipponbare BAC clones 44D14, 82N20, and 73N20 (Accession Nos. AC119071, AC119073, AC119072) sequenced by the Genome Center of Wisconsin (http://www.gcowlwisc.edu). RFLP markers, RGA38, RGA8, G320, and F5003 are tightly linked to disease resistance locus Pi-CO39(t) locus flanked by 73N20 CAPS1 and RPR1 gene in Nipponbare haplotype.

FIG. 3. CAPS analysis of a NBS-LRR gene, NBR2 and its orthologue COR2. Genomic DNAs from rice genotypes were amplified using gene specific promers, PCR products digested with 4 bp cutter, Taq1 and resolved in 1.5% Seakem agarose gel.

### DETAILED DESCRIPTION OF THE INVENTION

### I. Definitions

Various terms relating to the biological molecules of the present invention are used hereinabove and also throughout the specifications and claims.

The term “pathogen-inoculated” refers to the inoculation of a plant with a pathogen.

The term “disease defense response” refers to a change in metabolism, biosynthetic activity or gene expression that enhances the plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase. Such defense responses appear to be induced in plants by several signal transduction pathways involving secondary defense signaling molecules produced in plants. Agents that induce disease defense responses in plants include, but are not limited to: (I) microbial pathogens, such as fungi, bacteria and viruses; (2) microbial components and other defense response elicitors, such as proteins and protein fragments, small peptides, β-glucans, elicitins and harpins, cryptogein and oligosaccharides; and (3) secondary defense signaling molecules produced by the plant, such as salicylic acid, H₂O₂, nitric oxide, ethylene and jasmonates.

With reference to nucleic acids of the invention, the term “isolated nucleic acid” or “polynucleotide” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule.

With respect to RNA molecules of the invention the term “isolated nucleic acid” or “polynucleotide” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

With respect to antibodies of the invention, the term “immunologically specific” refers to antibodies that bind to one or more epitopes of a protein of interest, but which do not substantially recognize and bind other molecules in a sample containing a mixed population of antigenic biological molecules.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

When used herein in describing components of media or other experimental results, the term “about” means within a margin of commonly acceptable error for the determination being made, using standard methods. For plant transformation or tissue culture media in particular, persons skilled in the art would appreciate that the concentrations of various components initially added to culture media may change somewhat during use of the media, e.g., by evaporation of liquid from the medium or by condensation onto the medium. Moreover, it is understood that the precise concentrations of the macronutrients, vitamins and carbon sources are less critical to the efficacy of the media than are the micronutrient, hormone and antibiotic concentrations.

With respect to single stranded oligonucleotides and polynucleotides, the term “specifically hybridizing” refers to the association between two single-stranded nucleotide molecules of sufficiently complementary sequence to permit such hybridization under predetermined conditions generally used in the art (sometimes termed “substantially complementary”). In particular, the term refers to hybridization of an oligonucleotide or polynucleotide with a substantially complementary sequence contained within a single-stranded DNA or RNA molecule of the invention, to the substantial exclusion of hybridization of the oligonucleotide or polynucleotide with single-stranded nucleic acids of non-complementary sequence.

The term “substantially the same” refers to nucleic acid or amino acid sequences having sequence variation that do not materially affect the nature of the protein (i.e. the structure, stability characteristics and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

Nucleic acid sequences and amino acid sequences can be compared using computer programs that align the similar sequences of the nucleic or amino acids thus define the differences. In preferred methodologies, the Blastn and Blastp 2.0 programs provided by the National Center for Biotechnology Information (http://www.ncbi.nlm.nih.gov/blast/; Altschul et al., 1990, J. Mol. Biol. 215:403-410) using a gapped alignment with default parameters, may be used to determine the level of identity and similarity between nucleic acid sequences and amino acid sequences. However, equivalent alignments and similarity/identity assessments can be obtained through the use of any standard alignment software. For instance, the DNAstar system (Madison, Wis.) may be used to align sequence fragments of genomic or other DNA sequences. Alternatively, GCG Wisconsin Package version 9.1, available from the Genetics Computer Group in Madison, Wis., and the default parameters used (gap creation penalty=12, gap extension penalty=4) by that program may also be used to compare sequence identity and similarity.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined by Taylor (1986, J. Theor. Biol. 119:205). Polypeptides having sequences greater than 70% identical, preferably greater than 80%, and more preferably greater than 90% and most preferably greater than 95% identical to the polypeptides encoded by the nucleic acid sequences described herein are considered within the scope of the invention. When referring to nucleic acid molecules, “percent identical” refers to the percent of the nucleotides of the subject nucleic acid sequence that have been matched to identical nucleotides by a sequence analysis program. Polynucleotides having sequences greater than 60% identical, preferably greater than 70% identical, more preferably greater than 80% identical, and more preferably greater than 90% identical, and most preferably greater than 95%, 96%, 97%, 98%, and even 99% identical to the polynucleotides described herein are considered within the scope of the invention.

A “coding sequence” or “coding region” refers to a nucleic acid molecule having sequence information necessary to produce a gene product (RNA or protein), when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g. enhancers) in an expression vector.

Transcriptional and translational control sequences, sometimes referred to herein as “expression control” sequences or elements, or “expression regulating” sequences or elements, are DNA regulatory elements such as promoters, enhancers, ribosome binding sites, polyadenylation signals, terminators, and the like, that provide for the expression of a coding sequence in a host cell. The term “expression” is intended to include transcription of DNA and translation of the mRNA transcript.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene. These constructs may be administered to plants in a viral or plasmid vector. Other methods of delivery such as Agrobacterium T-DNA mediated transformation and transformation using the biolistic process are also contemplated to be within the scope of the present invention. In addition to specific methods described herein, the transforming DNA may be prepared according to standard protocols such as those set forth in “Current Protocols in Molecular Biology”, eds. Frederick M. Ausubel et al., John Wiley & Sons, 2001. In certain embodiments, such constructs are chimeric, i.e., the coding sequence is from a different source one or more of the regulatory sequences (e.g., coding sequence from rice and promoter from maize or Arabidopsis). However, non-chimeric DNA constructs also can be used. A plant species or cultivar may be transformed with a DNA construct (chimeric or non-chimeric) that encodes a polypeptide from a different plant species or cultivar, or a non-plant species. Alternatively, a plant species or cultivar may be transformed with a DNA construct (chimeric or non-chimeric) that encodes a polypeptide from the same plant species or cultivar. The term “transgene” is sometimes used to refer to the DNA construct within the transformed cell or plant.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a plant gene, the gene will usually be flanked by DNA that does not flank the plant genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and plant or animal cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

### II. Description

In accordance with the present invention, a novel rice resistance gene (or genes) has been identified and localized to a specific region on chromosome 11 of the rice genome. This gene is referred to herein as Pi-CO39(t), to denote its function as a gene in rice cultivar CO39 that confers resistance to strains of the plant pathogen, Magnaporthe grisea, that contain the cultivar specificity gene AVR1-CO39.

The genetic mapping of Pi-CO39(t) in rice line CO39 and identification of large insert clones linked to resistance are described in detail in Example 1. The resistance locus, Pi-CO39(t), was mapped between RZ141 (10.7 cM) and R2316 (3.2 cM), S2712 (1.2 cM), and 73N20CAPS-1 (0.2 cM) on one side (telomeric end) and RG211 (19.0 cM), RM202 (12.1 cM) and RG1094 (6.1 cM), and RPR1 (0.2 cM) on the centromeric end of the short arm of chromosome 11. Three additional markers, RGA8, RGA38 and G320, were found to cosegregate with the Pi-CO39(t) gene in 1200 F2 progenies which were phenotypically tested for resistance or susceptibility. These markers were used to isolate BAC clones from the resistant variety CO39 and the susceptible variety Nipponbare. BAC clones hybridizing to all co-segregating markers were obtained from the Nipponbare library; and a minimum tiling path of three overlapping BAC clones (73N20, 82N20 and 44D15) was laid out for sequencing. Screening of CO39 library with co-segregating marker RGA38 as well as BAC end probes from CO39 and Nipponbare BACs resulted in the identification of clones, 4A14, 59O11, 55P02, 27H09, 34N09, 52I18, 19I17, 19E06 1L23, 36K6, 4A14. Sequence analysis of the RGA8 and RGA38 homologs in CO39 yielded SEQ ID NO:6 and SEQ ID NO:7, respectively. These two sequences are about 97% and 84% identical, respectively, to RGA38 and RGA8 as reported by Mago et al. (GenBank Accession Nos. set forth above).

It is believed that part or all of the genomic region of rice containing Pi-CO39(t) resides on one or more of the CO39 BAC clones listed above. A contig of about 0.5 mb has been constructed in the relevant region of the Nipponbare library. Sequence of the relevant region of CO39 associated with Pi-CO39(t) locus on chromosome 11 are set forth herein as SEQ ID NOs:1-5, which when pieced together 3′ end of one SEQ ID NO to 5′ end of the next succeeding SEQ ID NO, yield one continuous polynucleotide sequence. A detailed analysis of the BAC clones and the genes predicted to be located on the clones is also set forth in the examples.

The following description sets forth the general procedures involved in practicing the present invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratory (1989) (hereinafter “Sambrook et al.”) or Ausubel et al. (eds.) Current Protocols in Molecular Biology, John Wiley & Sons (2001) (hereinafter “Ausubel et al.”) are used.

### A. Preparation of Pi-CO39(t) Nucleic Acid Molecules, Encoded Polypeptides and Transgenic Plants

### 1. Nucleic Acid Molecules

Pi-CO39(t) nucleic acid molecules of the invention may be prepared by two general methods: (1) they may be synthesized from appropriate nucleotide triphosphates, or (2) they may be isolated from biological sources. Both methods utilize protocols well known in the art.

The availability of the Pi-CO39(t) nucleotide sequence information enables preparation of an isolated nucleic acid molecule of the invention by oligonucleotide synthesis. Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC).

Pi-CO39(t) genes also may be isolated from appropriate biological sources using methods known in the art. In one embodiment, large insert clones have been isolated from BAC libraries of a resistant and susceptible rice cultivar. In an alternative embodiment, a cDNA clone comprising the open reading frame of the genomic Pi-CO39(t) locus may be isolated.

In accordance with the present invention, nucleic acids having the appropriate level sequence homology with part or all the coding and/or regulatory regions of Pi-CO39(t) may be identified by using hybridization and washing conditions of appropriate stringency. For example, hybridizations may be performed, according to the method of Sambrook et al., using a hybridization solution comprising: 5×SSC, 5× Denhardt's reagent, 1.0% SDS, 100 μg/ml denatured, fragmented salmon sperm DNA, 0.05% sodium pyrophosphate and up to 50% fornamide. Hybridization is carried out at 37-42° C. for at least six hours. Following hybridization, filters are washed as follows: (1) 5 minutes at room temperature (approx. 25° C.) in 2×SSC and 1% SDS; (2) 15 minutes at room temperature in 2×SSC and 0.1% SDS; (3) 30 minutes-1 hour at 37° C. in 2×SSC and 0.1% SDS; (4) 2 hours at 45-55° C. in 2×SSC and 0.1% SDS, changing the solution every 30 minutes. Alternatively, a modification of the Amasino hybridization protocol (Anal. Biochem. 152:304-307, 1986) is preferred for use in the present invention and is described in greater detail in Example 1.

One common formula for calculating the stringency conditions required to achieve hybridization between nucleic acid molecules of a specified sequence homology (Sambrook et al., 1989):

Tₘ=81.5° C.+16.6 Log[Na+]+0.41(% G+C)−0.63(% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. In a preferred embodiment, the hybridization is at 37° C. and the final wash is at 42° C., in a more preferred embodiment the hybridization is at 42° C. and the final wash is at 50° C., and in a most preferred embodiment the hybridization is at 42° C. and final wash is at 65° C., with the above hybridization and wash solutions. Conditions of high stringency include hybridization at 42° C. in the above hybridization solution and a final wash at 65° C. in 0.1×SSC and 0.1% SDS for 10 minutes.

Nucleic acids of the present invention may be maintained as DNA in any convenient cloning vector. In a preferred embodiment, clones are maintained in plasmid cloning/expression vector, such as pGEM®-T (Promega Biotech, Madison, Wis.) or pBluescript (Stratagene, La Jolla, Calif.), either of which is propagated in a suitable E. coli host cell.

Pi-CO39(t) nucleic acid molecules of the invention include cDNA, genomic DNA, RNA, and fragments thereof which may be single- or double-stranded. Thus, this invention provides a polynucleotide that is a source of oligonucleotides (sense or antisense strands of DNA or RNA) having sequences capable of hybridizing with at least one sequence of a nucleic acid molecule linked to M. grisea disease resistance. Such oligonucleotides are useful as probes for detecting Pi-CO39(t) genes or mRNA in test samples of plant tissue, e.g. by PCR amplification, or for the positive or negative regulation of expression of Pi-CO39(t) genes at or before translation of the mRNA into proteins.

### 2. Polypeptides

Polypeptides encoded by the Pi-CO39(t) gene may be prepared in a variety of ways, according to known methods. If produced in situ the polypeptides may be purified from appropriate sources, e.g., plant tissue.

Alternatively, the availability of nucleic acid molecules encoding the polypeptides will enable production of the proteins using in vitro expression methods known in the art. For example, a cDNA or gene may be cloned into an appropriate in vitro transcription vector, such a pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

According to a preferred embodiment, larger quantities of Pi-CO39(t)-encoded polypeptides may be produced by expression in a suitable procaryotic or eucaryotic system. For example, part or all of a Pi-CO39(t) gene or cDNA may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

The Pi-CO39(t) polypeptide(s) produced by gene expression in a recombinant procaryotic or eucyarotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein. Such methods are commonly used by skilled practitioners.

The present invention also provides antibodies capable of immunospecifically binding to Pi-CO39(t)-encoded polypeptides. Polyclonal or monoclonal antibodies are prepared according to standard methods. Monoclonal antibodies may be prepared according to general methods of Kohler and Milstein, following standard protocols. Recombinant monoclonal antibodies also may be prepared in accordance with standard methods, e.g., via phage display libraries of genes encoding human or animal antibodies or fragments, which may be panned with plant proteins. In a preferred embodiment, antibodies are prepared, which react immunospecifically with various epitopes of the Pi-CO39(t)-encoded polypeptides.

Polyclonal or monoclonal antibodies that immunospecifically interact with one or more of the polypeptides encoded by Pi-CO39(t) can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

### 3. Transgenic Plants

The present invention includes transgenic plants comprising one or more copies of the Pi-CO39(t) gene or genes. This is accomplished by transforming plant cells with a transgene that comprises part of all of a Pi-CO39(t) coding sequence, controlled by either native or recombinant regulatory sequences, as described below. Transgenic plants of any species are included in the invention. Preferred are monocots having susceptibility to pathogenic species of Magnaporthe; these include rice, wheat, barley, maize and other cereal crops and the cereal Setaria italica and Eleusine coracana, as well as turfgrasses such as Lolium perenne L. and Lolium multiflorium Lam.

Transgenic plants can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors or other plant viral vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994).

The method of transformation depends upon the plant to be transformed and the subcellular target of the transgene. Agrobacterium vectors are often used to transform dicot species. Agrobacterium binary vectors include, but are not limited to, BIN19 (Bevan, 1984) and derivatives thereof, the pBI vector series (Jefferson et al., 1987), and binary vectors pGA482 and pGA492 (An, 1986). For transformation of monocot species, biolistic bombardment with particles coated with transforming DNA and silicon fibers coated with transforming DNA are often useful for nuclear transformation. Alternatively, Agrobacterium “superbinary” vectors have been used successfully for the transformation of rice, maize and various other monocot species.

DNA constructs for transforming a selected plant comprise a coding sequence of interest operably linked to appropriate 5′ (e.g., promoters and translational regulatory sequences) and 3′ regulatory sequences (e.g., terminators). In a preferred embodiment, the Pi-CO39(t) gene under control of its own 5′ and 3′ regulatory elements is utilized.

In an alternative embodiment, the coding region of the gene is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine synthase (OCS) promoters. In preferred embodiments, a strong monocot promoter is used, for example, the maize ubiquitin promoter, the rice actin promoter or the rice tubulin promoter (Jeon et al., Plant Physiology 123:1005-1014, 2000).

Transgenic plants expressing Pi-CO39(t) coding sequences under an inducible promoter are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene) to name a few. The use of pathogen- and wound-inducible promoters is described in more detail below.

Tissue-specific and development-specific promoters are also contemplated for use in the present invention. Examples of these include, but are not limited to: the ribulose bisphosphate carboxylase (RuBisCo) small subunit gene promoters or chlorophyll and binding protein (CAB) gene promoters for expression in photosynthetic tissue; the various seed storage protein gene promoters for expression in seeds; and the root-specific glutamine synthetase gene promoters where expression in roots is desired.

The coding region is also operably linked to an appropriate 3′ regulatory sequence. In a preferred embodiment, the nopaline synthetase polyadenylation region (NOS) is used. Other useful 3′ regulatory regions include, but are not limited to the octopine (OCS) polyadenylation region.

Using an Agrobacterium binary vector system for transformation, the selected coding region, under control of appropriate regulatory elements, is linked to a nuclear drug resistance marker, such as kanamycin resistance. Other useful selectable marker systems include, but are not limited to: other genes that confer antibiotic or herbicide resistances (e.g., resistance to hygromycin or bialaphos) or herbicide resistance (e.g., resistance to sulfonylurea, phosphinothricin, or glyphosate).

Plants are transformed and thereafter screened for one or more properties, including the presence of Pi-CO39(t) protein, Pi-CO39(t) mRNA, or enhanced resistance to plant pathogens, in particular Magnaporthe grisea. It should be recognized that the amount of expression, as well as the tissue-specific pattern of expression of the transgenes in transformed plants can vary depending on the position of their insertion into the nuclear genome. Such positional effects are well known in the art. For this reason, several nuclear transformants should be regenerated and tested for expression of the transgene.

Transgenic plants that exhibit one or more of the aforementioned desirable phenotypes can be used for plant breeding, or directly in agricultural or horticultural applications. Plants containing one transgene may also be crossed with plants containing a complementary transgene in order to produce plants with enhanced or combined phenotypes.

### B. Uses of Pi-CO39(t) Nucleic Acids, Proteins and Transgenic Plants

The potential of recombinant genetic engineering methods to enhance disease resistance in agronomically important plants has received considerable attention in recent years. Protocols are currently available for the stable introduction of genes into plants (as described in detail above), as well as for augmentation of gene expression. The present invention provides nucleic acid molecules which, upon stable introduction into a recipient plant, can enhance the plant's ability to resist pathogen attack.

### 1. Pi-CO39(t) Nucleic Acids and Transgenic Plants

Pi-CO39(t) nucleic acids (genomic clones or cDNAs) may be used for a variety of purposes in accordance with the present invention. The DNA, RNA, or fragments thereof may be used as probes to detect the presence of and/or expression of Pi-CO39(t) genes. Methods in which Pi-CO39(t) nucleic acids may be utilized as probes for such assays include, but are not limited to: (1) in situ hybridization; (2) Southern hybridization; (3) northern hybridization; and (4) assorted amplification reactions such as polymerase chain reactions (PCR). The Pi-CO39(t) nucleic acids of the invention may also be utilized as probes to identify homologs from other rice cultivars and from other plant species. As described above, Pi-CO39(t) nucleic acids are also used to advantage to produce large quantities of substantially pure Pi-CO39(t) proteins, or selected portions thereof.

Of greater significance, however, is the use of Pi-CO39(t) nucleic acids to broaden the scope of resistance of rice cultivars and other plant species to a variety of M. grisea isolates, and even to plant pathogens other than M. grisea. For instance, in one embodiment of the invention, the Pi-CO39(t) coding region is operably linked to a heterologous promoter, preferably one that is either generally pathogen inducible (i.e. inducible upon challenge by a broad range of pathogens) or wound inducible. Such promoters include, but are not limited to:

a) promoters of genes encoding lipoxygenases (preferably from plants, most preferably from rice, e.g., Peng et al., J. Biol. Chem. 269:3755-3761, 1994; Peng et al., Abstract presented at the general meeting of the International Program on Rice Biotechnology, Malacca, Malaysia, Sep. 15-19, 1997);

b) promoters of genes encoding peroxidases (preferably from plants, most preferably from rice, e.g., Chittoor et al., Mol. Plant-Microbe Interactions 10:861-871, 1997);

c) promoters of genes encoding hydroxymethylglutaryl-CoA reductase (preferably from plants, most preferably from rice, e.g., Nelson et al., Plant Mol. Biol. 25:401-412, 1994);

d) promoters of genes encoding phenylalanine ammonia lyase (preferably from rice; e.g., Lamb et al., Abstract of the general meeting of the International Program on Rice Biotechnology, Malacca, Malaysia, Sep. 15-19, 1997);

e) promoters of genes encoding glutathione-S-transferase (preferably from plants, most preferably from rice, or alternatively, the PRP1 promoter from potato);

f) promoters from pollen-specific genes, such as corn Zmg13, which has been show to be expressed in rice transgenic pollen carrying the corn gene (Aldemita et al., Abstract of the general meeting of the International Program on Rice Biotechnology, Malacca, Malaysia, Sep. 15-19, 1997);

g) promoters from genes encoding chitinases (preferably from plants, most preferably from rice; e.g., Zhu & Lamb, Mol. Gen. Genet. 226:289-296, 1991);

h) promoters from genes induced early (within 5 hours post-inoculation) in the interaction of M. grisea and rice (e.g., Bhargava & Hamer; Abstract B-10, 8th International Congress Molecular Plant Microbe Interactions, Knoxville, Tenn. Jul. 14-19, 1996);

i) promoters from plant (preferably rice) viral genes, either contained on a bacterial plasmid or on a plant viral vector, as described by Hammond-Kosack et al., Mol. Plant-Microbe Interactions 8:181-185 (1994);

j) promoters from genes involved in the plant (preferably rice) respiratory burst (e.g., Groom et al., Plant J. 10(3):515-522, 1996); and

k) promoters from plant (preferably rice) anthocyanin pathway genes (e.g., Reddy, pp. 341-352 in Rice Genetics III, supra; Reddy et al., Abstract of the general meeting of the International Program on Rice Biotechnology, Malacca, Malaysia, Sep. 15-19, 1997).

The chimeric gene is then used to transform the plant of interest. Upon wounding or challenge with a plant pathogen, the resulting transgenic plants would be induced to produce the Pi-CO39(t) gene product, thereby triggering the R gene defense response. In this embodiment, care must be taken to avoid using a promoter that is induced by necrosis, since use of such a promoter could result in a self-perpetuating hypersensitive response that may be lethal to the plant (see, e.g., Kim et al., Proc. Natl. Acad. Sci. USA 91:10445-10449, 1994).

A preferred embodiment utilizes the Pi-CO39(t) gene controlled by its own regulatory sequences, rendering it either constitutively expressed or inducible by the product of the corresponding AVR1-CO39 avirulence gene that has been cloned. In this embodiment, the selected plant is transformed and a disease resistance response is generated by exposing the transformed plant to either or both of (1) the gene product of the AVR1-CO39 gene or (2) a suspension of non-pathogenic recombinant microorganisms (e.g., epiphytic or endophytic bacteria, or even a non-pathogenic strain of Magnaporthe) comprising the AVR1-CO39 gene. Upon pathogen attack, two levels of protection can occur in the transgenic plant: (1) the gene product produced by the recombinant epiphytes or endophytes triggers an interaction on the plant surface that prevents further penetration by the pathogen (e.g., the fungal conidia develop appresoria, but do not develop penetration pegs); or (2) the gene product produced by the recombinant epiphytes is carried into the plant tissue at the wound site, where it interacts with the corresponding R gene product and induces an internal disease defense response. Thus, this pre-treatment confers resistance to Magnaporthe isolates (and, presumably, other plant pathogens) which normally are virulent on those cultivars. These methods are described in detail in PCT US99/04047 and commonly-owned co-pending U.S. application Ser. No. 09/257,585, herein incorporated by reference.

In another embodiment, plants themselves can be co-transformed with Pi-CO39(t) and the fungal AVR1-CO39 gene. Co-expression of the genes results in an internal triggering mechanism to induce the resistance response.

It should be noted that constitutive production of the Pi-CO39(t) gene product may induce resistance without the aid of the AVR1-CO39 gene. Accordingly, it may not be necessary in all instances to use an inducible system.

### 2. Pi-CO39(t) Proteins and Antibodies

Purified gene products of Pi-CO39(t), or fragments thereof, may be used to produce polyclonal or monoclonal antibodies, which also may serve as sensitive detection reagents for the presence and accumulation of Pi-CO39(t) polypeptides. Polyclonal or monoclonal antibodies immunologically specific for Pi-CO39(t) polypeptides may be used in a variety of assays designed to detect and quantitate the proteins. Such assays include, but are not limited to: (1) flow cytometric analysis; (2) immunochemical localization of expressed proteins in cells or tissues; and (3) immunoblot analysis (e.g., dot blot, Western blot) of extracts from various cells and tissues. Additionally, as described above, antibodies can be used for purification of Pi-CO39(t) polypeptides (e.g., affinity column purification, immunoprecipitation).

The following specific examples are provided to illustrate embodiments of the invention. They are not intended to limit the scope of the invention in any way.

### EXAMPLE 1

### Genetic Mapping of Locus Comprising Blast Resistance Genes Pi-CO39(t) in Rice Line CO39 and Identification of Large Insert Clones Linked to Resistance

### Materials & Methods

M. grisea strains. Isolate ‘Guy11’—virulent on CO39 and 51583—originally collected from a diseased rice plant in French Guyana, was provided by J. L. Notteghem (Institute de Recherches Agronomiques Tropicales Montpellier Cedex, France). Progeny 6082, avirulent on CO39 and virulent on 51583, was produced by crossing isolate 2539 and Guy11 as described in Smith and Leong (Theor. Appl. Genet. 88:901-908, 1994). Guy11 transformant (G11XF18-1(0) A#6), carrying avirulence gene, AVR1-CO39 was produced by Farman and Leong Genetics 150:1049-1058, 1998). Fungal cultures were stored at −20° C. in 6-mm chromatography paper discs (Whatman) as described by Valent et al. (Genetics 127:87-101, 1991).

Seed germination, inoculation procedure and disease severity ratings. Seeds of rice genotypes CO39 and 51583 were procured from different sources as described in Smith and Leong (Theor. Appl. Genet. 88:901-908, 1994). Seeds were surface sterilized in 10% bleach and germinated on petri plates lined with moist blotting paper. Individual seedlings, usually 5-6 days after germination, were transplanted to disposable plastic square cubicles. The growth medium was Bacto professional planting mix (Michigan Peat Co., Houston, Tex.). Seedlings were flooded with water continuously. Seedlings were grown for 3-4 weeks in a growth chamber equipped with full spectrum white light GROW-LOX® bulbs (230 uE/m/sec) set for 16 h photoperiods. Day/night temperatures were 28° C./21° C., respectively, and percent relative humidity was 33%. Plants were inoculated at the three leaf stage, 20-25 days after transplanting.

Inoculum was prepared by growing each isolate on oatmeal agar plates under full spectrum white light bulbs (Sylvania GROW-LOX® 20W) (20-55 uE/m/sec) at 22° C. for 15-20 days. Spores were detached by gently rubbing the agar surface with a bent glass rod after adding 5 ml of 0.2% gelatin solution and sprayed on seedlings at a concentration of 10⁴ spores/ml. Plants were placed in a plastic bag and tied from the top. Bags were removed after 24 hours.

Seven days after inoculation, ratings for disease severity were recorded on the youngest leaf that was expanded at the time of inoculation. We based our judgements on the ability of affected tissue to support conidiation under high humidity conditions and thus complete the disease cycle (Valent et al., Genetics 127:87-101, 1991). To describe the interaction phenotype, a numerical system similar to that of Yu et al. (Theor. Appl. Genet. 93:859-863, 1987) has been adopted in this study: Type 0—no visible symptoms; Type 1—small dark brown, pin point-sized, non-sporulating lesions Type 2—dark brown, non-sporulating lesions 2-3 mm in length, Type 3—circular, sporulating lesions with the tan centers and dark brown margins; Type 4—large diamond-shaped, sporulating with tan centers and dark brown margins. Reaction phenotypes with lesion types 0, 1 and 2 were considered resistant while those producing reaction Types 3 and 4 were considered susceptible.

Inheritance experiments. Reciprocal crosses between CO39 (resistant) and 51583 (susceptible) were made. Each F₁ seedling was tested for disease reaction and grown to maturity to produce a F₂ population. Individual plant progenies derived from a single F₂ plant constituted a single F₃ family. Segregation data were tested for Mendelian inheritance using the Chi-square method. Twelve homozygous resistant F₃ lines and six homozygous susceptible F₃ lines were selected for preliminary mapping using bulked segregant analysis. Equal amounts of DNA from each line were combined to make-resistant and susceptible pools of DNA for bulked segregant analysis (Michelmore et al., Proc. Natl. Acad. Sci. U.S.A. 88:2236-2240, 1991).

Microsatellite analysis. Microsatellite primer pairs for 20 loci on rice chromosomes 4, 6, 11 and 12 were synthesized using an ABI DNA synthesizer according to the manufacturers instructions. Total genomic DNA (75-100 ng) of CO39 and 51583 as well as of pools from 10 resistant or 6 susceptible F₃ progenies was used as template for PCR amplification by initial incubation at 92° C. for 5 min followed by 35 cycles of denaturation at 92° C. for 1 min; annealing at 55° C. for 1 min and extension at 72° C. for 2 min and a final 4 min extension at 72° C. Polymorphisms between the PCR products from DNA of parents and the resistant or susceptible F₃ progeny were analysed by electrophoresing the PCR products on 40-cm-long 4.5% denaturing polyacrylamide gels (PAGE) run for 1.5 h at 75 constant watts and silver stained according to the manufacturer's instructions (Promega). The polymorphic and co-segregating marker, RM202, was tested with DNA of a large number of individual F₂ progenies. The PCR products for the RM202 marker were resolved in 3.0% MetaPhor® agarose (FMC Bioproducts) prepared in 0.5× TBE and run at 10.0 V/cm for 5 h.

Plant DNA extraction, restriction digestion, electrophoresis and Southern analysis. Plant DNA was prepared from fresh or frozen leaf tissue from individual plants according to the method of McCouch et al. (Theor. Appl. Genet. 76:815-829, 1988). Total genomic DNA of CO39 and 51583 was digested with several 6 bp restriction endonucleases to detect polymorphisms. The parental DNA as well as of individual F₂ progenies was digested with EcoR1, BamH1, EcoRV, Hind111, Dra1, Nae1 for mapping. Genomic and cDNA probes as well as microsatellite primer pair sequences from the high-density Cornell maps (Causse et al., Genetics 138:1251-1274, 1994; Chen et al., Theor. Appl. Genet. 95:553-567, 1997) and the Japanese Rice Genome Program (Harushima et al., Genetics 148:479-494, 1998) were selected. Electrophoresis and Southern hybridization analysis were done according to Farman & Leong (Genetics 150:1049-1058, 1998). Hybridization methods were modified from Amasino (Anal. Biochem. 152:304-307, 1986). The hybridization buffer was prepared according to the Amasino protocol, but without the PEG and NaCl and with reduced concentrations of NaHPO₄:0.125M NaHPO₄, 7% SDS, 50% formamide, 1.0 mM EDTA, pH 7.2. High stringency conditions were used (42° C., 16 h). Post hybridization washes were: 2×SSC+0.1% SDS and in 0.1×SSC+1.0% SDS at 42° C. for 15 min each and in 0.1×SSC+0.1% SDS at 65° C. for 20 min, respectively. The final washing conditions were of greater stringency than were the hybridization conditions, giving a T^{m} of 68° C. Thus, greater than 95% homology would be required to maintain a hybrid. None of the post hybridization phosphate-containing buffers described in Amasino (Anal. Biochem. 152:304-307, 1986) were employed.

Construction of Large insert DNA library. Indica rice variety CO39, which is a source of blast resistance locus in this study, was the plant material used for constructing a large insert DNA library.

The conventional binary cosmid vector pCLDO4541, designed for Agrobacterium-mediated plant transformation (Bent et al., Science 265:1856-1860, 1994), was selected as a cloning vector. The vector has a cos site and a polylinker from pBluescript SK/KS which can facilitate cloning of foreign DNA at five restriction sites (Cla1, Hind111, EcoR1, BamH1 and Xba1). PCLDO4541 has been used for stable cloning and maintenance of large DNA inserts without any rearrangements (Tao and Zhang, Nucleic Acids Res. 26:4901-4909; Wu et al., Genome 43:102-109, 2000). The preparation of vector DNA, restriction digestion, and dephosphorylation were done according to Zhang et al. (Mol. Breed. 2:11-24, 1996).

Isolation of high molecular weight DNA, partial digestion and size selection. Rice seedlings were grown for 2-3 weeks, etiolated for 24-36 hrs under complete darkness and 25-30 g of fresh leaf tissue was used for nuclei isolation as per the “nuclei isolation method” described by Zhang et al. (Plant J. 7:175-184, 1995). Fresh leaf tissue (20 g) was ground into fine powder using a mortar and pestle in liquid nitrogen and immediately transferred into ice-cold homogenization buffer (HB: 10 mM trizma base, 80 mM KCl, 1 mM spermidine, 1 mM spermine, pH 9.4, 0.5 M sucrose) plus 0.5% Triton®X-100 and 0.15% β-mercaptoethanol, mixed well and filtered through cheesecloth and Miracloth (Calbiochem®-Novabiochem™, La Jolla, Calif.) and centrifuged at 1800×g for 25 min. After washing in wash buffer the nuclear pellet was resuspended in 500 μl of HB and processed for microbead preparation according to Zhang et al. (Plant J. 7:175-184, 1995). Nuclei were embedded in low melting agarose microbeads. The microbeads were incubated in lysis buffer (0.5 M EDTA, pH 9.0, 1% sodium lauryl sarcosine, 0.1 mg/ml proteinase K) at 55° C. for 36 h, followed by treatment with TE buffer (10 mM Tris-HCl, 1 mM EDTA, pH 8.0) plus 0.1 mM phenylmethylsulfonyl fluoride (PMSF) for 1 h, three times. The mirobeads were finally kept in TE buffer. Partial digestion of high molecular weight DNA with BamH1 was performed in the beads according to Zhang et al. (Plant J. 7:175-184, 1995).

Size fractionation of partially digested DNA in microbeads. Partially digested DNA was size selected using a modification of the method of Osoegawa et al. (Genomics 52:1-8, 1998). Microbeads containing the partially digested DNA were applied to the central well of a 1% low melting temperature agarose (SeaPlaque® GTG® agarose) gel and the lambda concatamer size markers in the flanking wells. The fractionation was done in BIORAD CHEF-DR® III system. The DNA was separated in three different stages using a pulse direction of 120°. The first direction of the field allowed the DNA to migrate 1-1.5 cm from the wells toward the top edge of the gel by electrophoresis at 5.0 V/cm for 6 h with a pulse time of 15 s. In the 2nd step, the CHEF running conditions were the same but current was reversed in order to bring all the fragments remaining in the gel back toward the wells. Small DNA fragments (˜50-100 kb) which moved beyond the wells were excised and discarded. Fresh 1% low melting agarose solution was poured into the excised portion of the gel. New marker DNA was loaded into the flanking wells not previously used. The high molecular weight DNA was then resolved at 6V/cm for 16 h with an increasing pulse time of 0.1 s- to −40 s. Running buffer (0.5×TBE), CHEF chamber temperature (12° C.) and the angle (120°) were the same for all three steps. After electrophoresis, the flanking marker lanes along with peripheral portion of both the sides of high MW rice DNA lane were cut, stained with ethidium bromide, destained, washed in distilled water thoroughly, and aligned with the digested genomic DNA lane to mark the position of selected size ranges. Gel slices were cut at an interval of 0.5 cm to obtain gel slices containing DNA in the range of 150-500 kb.

Ligation, transformation, and storage of the library. Gel slices containing the size (200-300 kb) selected DNA were dialysed against TE buffer and the agarose digested with gelase (Epicentre® Technologies, Madison, Wis.) as reported by Zhang et al. (Mol. Breed. 2:11-24, 1996). Insert DNA, which was BamH1 digested, was ligated to dephosphorylated vector DNA in 1:3 ratio at 16° C. overnight. The ligated DNA was transformed into Electromax® DH10B™ E. coli cells (Gibco™ BRL, Grand Island, N.Y.) by electroporation using the Cell-Porator® and Voltage Booster system (Gibco™ BRL). The Cell-Porator® settings were the same as recommended by the manufacturer. Transformed cells were incubated at 37° C. for 1 h in 1 ml of SOC medium (2% Bacto tryptopane, 0.5% Bacto Yeast Extract, 10 mM NaCL, 2.5 mM KCL, 10 mMMgCl₂, 10 mM MgSO₄ and 20 mM glucose, pH 7.0), and then plated on LB plates containing X-gal (80 μg/ml), IPTG (0.55 mM), and tetracycline (15 μg/l). The plates were incubated at 37° C. for 18-20 hrs for blue/white color development. The white colonies were picked with toothpicks and transferred to 384-well microtitre dishes containing 70 μl LB cell freezing medium (36 mM K₂HPO₄, 13.2 mM KH₂PO₄, 1.7 mM sodium citrate, 0.4 mM MgSO₄, 6.8 mM (NH4)₂, 4.4% glycerol, LB 25 g/l). The microtitre dishes were incubated at 37° C. for 24 h. The library was replicated and stored at −80° C.

Arraying of clones on high density filters. Large DNA insert clones were arrayed onto high density filters using a 384-pin Biomek® 2000 Robotics Workstation (Beckman, Fullerton, Calif.). Each arrayed filter (7.5 cm×11 cm) contained duplicate copies of each clone's DNA in a 3×3 grid with no colony in the center (1,536 clones/filter). The library consisted of 23,040 clones arrayed on 15 filters. The filters were processed according to Zhang et al. (Mol. Breed. 2:11-24, 1996) and stored at 4° C. before probing.

Identification of clones linked to blast resistance locus (Pi-CO39(t)). The library was probed with a co-segregating RFLP marker RGA38 using the following conditions for probe preparation and hybridization.

BAC filter probing and hybridization. Probes were labeled using random hexamer Oligolabelling Kit (Pharmacia) except that 1 ng uncut lambda DNA was included along with the probe DNA and hybridized at 42° C. in 50% formamide, 7% SDS, 0.125 M Na₂HPO₄ (pH7.2) and 1 mM Na EDTA overnight. The BAC filters were washed three times for 20 min each in 2×SSC+0.1% SDS at 42° C. for 1st wash and in 0.5×SSC+0.1% SDS and in 0.1×SSC+0.1% SDS at 65° C. for 2nd and 3rd washes, respectively. The filters were exposed to Phosphor screen and scanned after 30 min-1 h exposure using a Packard Cyclone™ Storage system. Overnight exposures were also scanned to see the background hybridization of all colonies caused by hybridization of lambda to the vector to facilitate the determination of the clone address. Miniprep DNA from recombinant BAC clones was isolated using a modification of alkalines lysis method described in Zhang et al. (Mol. Breed. 2:11-24, 1996). Large scale isolation of BAC DNA was done according to the QIAGEN large-construct kit.

BAC DNA sequencing and assembly. BAC clones DNA was prepared using a QIAGEN Large-Construct Kit. Sequencing templates were prepared from random-sheared DNA generated by nebulization, size selected, ends repaired with T4 DNA polymerase (Boehringer Mannheim) and cloned into dephosphorylated EcoRV-digested pBluescript IISK-plasmid (Stratagene). Two different average sizes of 2-3 kb and 6-8 kb libraries were prepared for each BAC clone. Shot-gun clones were sequenced from both the directions using big dye terminator chemistry and run on ABI PRISM 3700 and 3730×1 capillary sequencers. Base calling and quality assessment were done using PHRED (Ewing and Green, Genome Res. 8:186-194, 1998), assembled by PHRAP and edited with CONSED (Gordon et al., Genome Res. 8:195-202, 1998). Shot-gun sequences were also independently assembled with SeqMan (DNASTAR). Contigs were extended and joined by primer walking on shot-gun clones as well as genomic DNAs of respective rice genotypes. Primers were designed with Autofinish (Gordon et al., Genome Res. 11:614-625, 2001) as well as Primer 3 (http://www.genome.wi.mit.edu/cgi-bin/primer/primer3_www.cgi).

Identification of genes in the sequenced region of CO39. Complete sequence of CO39 region and the orthologous region in Nipponbare genome cosegregating with the Pi-CO39(t) locus was used to predict genes using different gene prediction programmes such as GeneMark.hmm (http://opal.biology.gatech.edu/GeneMark/eukhmm.cgi), Genescan (http ://www.genes.mit.edu/GENSCAN.html), and FGENESH (http ://www. softberry. com/berry.phtml?topic=gfind&prg=FGENESH). Predicted protein sequences were checked in non-redundant databases using BLASTN, BLASTX, and BLASTP algorithms (http://www.ncbi.nlm.nih.gov) to identify the putative function. Presence of functional domains and motifs in the predicted orfs was detected by using SMART (http://smart.embl-heidelberg.de; Letunic et al., Nucl. Acids. Res. 30:242-244, 2002) and PSORT (http://psort.nibb.acjp/form.html). The identity and positions of transposable elements were determined by a combination of Repeat Masker (http://repeatmasker.genome.washington.edu/cgi-bin/RepeatMasker), SSRT (http://brie4.cshl.org/gramene/searches/ssrtool), FASTA, BLAST searches to the GenBank nonredundant database and TIGR rice repeat database (http://www.tigr.org). The MITEs were identified by blasting a 10 kb sliding window against BLASTn in the GenBank. Different modules (EditSeq, MegAlign and MapDraw) of DNASTAR (http://www.dnastar.com) were used for alignment and predicted phylogenetic distance analysis of sequences.

Expression analysis of NBS-LRR and Serpin genes. Leaf tissue was harvested from Nipponbare and CO39 plants from healthy as well as after different time points (0, 3, 6, 12, 18, 24, 36, 72, 96 h) of inoculation with Guy11 (AVR1-CO39). Total RNA was isolated using Ambion RNAqueous™—Midi kit and treated with DNase using Ambion DNA-free. Rice actin gene, Rac1 (Accession No. X15865) was included as a positive control to detect RNA quality as well as genomic DNA contamination in the RNA preps. First strand cDNA was synthesized using Ambion RETROscript® synthesis kit. For first-strand cDNA synthesis, 5 μg of total RNA was used and reverse transcribed using Ambion RETROscript® synthesis kit. The product of the first-strand synthesis reaction (3 μl) was then amplified using gene-specific primers designed from the NBS and LRR domains of NBS-LRR genes and the largest exons of serpin genes from both the Nipponbare and CO39 haplotypes. Amplification of NBS-LRR genes was done in ExTaq from TAKARA (www.takara-bio.co.jp), whereas that of serpin genes were amplified in LA Taq from TAKARA (www.takara-bio.co.jp), with GC buffer. The PCR products were resolved in 1.0 % Seakem agarose gel. Each primer sequence was checked against the complete sequences of Nipponbare and CO39 regions, respectively to make sure that it only anneals to the target NBS or LRR domain.

CAPS analysis. Cleaved amplified polymorphic (CAPS) sequence analysis of NBS-LRR genes predicted in CO39 and Nipponbare haplotypes was carried out by digesting the gene-specific PCR products with a range of 4 bp and 6 bp cutters. Several rice genotypes including from the pedigree of CO39 as well as popular US rice varieties were tested for resistance or susceptibility to M. grisea strains, Guy11 and Guy11 (AVR1-CO39). Rice genotypes are listed in Tables 6, 7). Genomic DNAs of these rice genotypes were extracted, PCR amplified using NBS-LRR gene-specific primers, digested with restriction enzymes polymorphic between 51583 and CO39. Digested PCR products were resolved in 1.5% Seakem agarose gel.

### Results

Genetic Analysis of resistance in CO39. Resistance phenotype of the F₁, individual F₂ progenies, and F₃ families was consistently of reaction Type 1 and the reaction phenotype of the susceptible seedlings was reaction Type 4 in all segregating populations. All the reciprocal F₁ tested (i.e. CO39×51583 or 51583×CO39) were resistant to M. grisea strain 6082, thereby, indicating that resistance is controlled by a dominant locus in the nuclear genome of CO39. Resistance to M. grisea progeny 6082 in three F₂ populations consisting of 604 F₂ progenies derived from different F₁ plants, segregated as a single dominant locus (Table 1).

Resistance to a Guy11 (AVR1-CO39) transformant in one F₂ population consisting of 235 F₂ progenies also segregated as a single dominant locus (Table 1). Inheritance of resistance was also confirmed in F₃ families derived from the F₂ populations used for mapping the resistance locus. Testing of 78 F₃ families against the Guy11 (AVR1-CO39) transformant and 59 F₃ families to M. grisea progeny 6082 also showed that resistance is controlled by a single dominant locus. The segregation ratio of F₃ families was: all resistant: segregating for resistance: all susceptible and fit into 1:2:1 ratio (Table 2). The disease resistance locus in CO39 was designated as Pi-CO39(t).

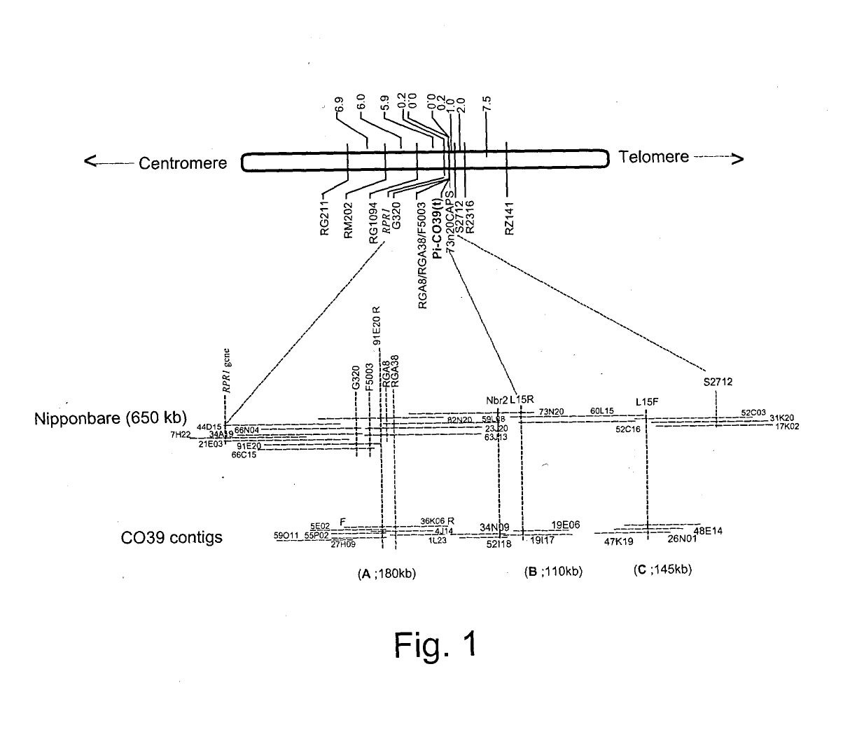

Mapping of resistance gene(s) Pi-CO39(t). Microsatellite loci were randomly selected from four nice chromosomes 4, 6, 11 and 12 that were previously shown to carry many disease resistance genes (McCouch et al., in Rice Blast Disease, Zeilger, R. S., Leong, S. A. and Teng, P. S., eds., pp. 167-186, 1994). Most of the test loci were found to be polymorphic between CO39 and 51583. Microsatellite locus, RM202, co-segregated in bulked segregant analysis of resistant and susceptible F₃ progenies, substantially as described by Chauhan et al. (Mol. Genet. Genomics, 267:603-612, 2002) herein incorporated by reference in its entirety. The resistance locus was fine mapped on chromosome 11 in two different F₂ populations consisting of 154 and 103 progenies, respectively. The program Mapmaker Version 2.0 (Lander et al., Genomics 1: 174-181, 1987) was used to determine association between molecular markers and the resistance locus using Kosambi Centimorgan function and LOD value of 3.0. The genetic map of resistance locus with respect to co-segregating markers is presented in FIG. 1. The resistance locus, Pi-CO39(t), was mapped between RZ141 (10.7 cM) and R2316 (3.2 cM), S2712 (1.2 cM), and 73N20CAPS-1 (0.2 cM) on one side (telomeric end) and RG211 (19.0 cM), RM202 (12.1 cM) and RG1094 (6.1 cM), and RPR1 (0.2 cM) on the centromeric end of the short arm of chromosome 11. Four markers, RGA8, RGA38, RGA CO39 and G320 perfectly co-segregated with Pi-CO39(t) among all the F₂ progenies tested. Primer sequences for amplifying RGA8, RGA38 and RGA CO39 are given in Table 3. RGA8 and RGA38 are resistance gene analogues mapped on chromosome 11 of rice (Mago et al., Theor. Appl. Genet. 99:5-57, 1999). These four markers have been tested on 1200 individual F₂ progenies. All the resistant F₂ progenies recombinant for different mapping markers were confirmed to be of the genotype RR or Rr. The frequency of recombination for different markers is given in Table 4.

Construction of a large DNA insert library of CO39. The large DNA insert library of the disease resistant genotype CO39 used in this study, was constructed from high molecular weight DNA isolated from nuclei in which more than 95% of the chloroplasts and mitochondria were removed during the preparation of nuclei. The DNA embedded in microbeads was partially digested with BamH1, size selected and ligated using a single size selection of 200-300 kb.

The library consists of 23,040 clones arrayed in 60 384-well microtitre dishes. About 65 random clones were selected, digested with Not 1 because Not 1 cuts out the cloned insert DNA. The Not 1 digested BAC DNAs were separated by pulse-field gel (PFG) electrophoresis (initial pulse time: 5 s; final pulse time: 15 s, 6V/cm, 11° C., 120°, 15 h) to determine the sizes of the cloned fragments. A lambda concatemer ladder from New England Biolabs® Inc. was used as a PFG marker. Insert size of recombinant clones ranged from 60-185 with an average insert size of 100 kb. All the 65 clones contained foreign DNA and one or more Not 1 site within the insert DNA. This library represented about 5× rice genome equivalents with a theoretical probability of 95% coverage of each gene. Contamination of chloroplast DNA in the library was less than 1% as determined by probing with a fragment of the chloroplast gene, rbcL.

Identification of large insert clones linked to blast resistance locus, Pi-CO39(t). The CO39 library was screened with co-segregating probe RGA38 as well as BAC end probes from CO39 (5E02F end probe) and Nipponbare (91E20R end probe) several positive clones were identified (shown in FIG. 1). The largest clones, 36K06 (100 kb), 5E02 (70 kb) and 34N09 were selected for shotgun sequencing. The CO39 DNA (7.68 kb) flanking the ends of 36K06 and 34N09 was amplified by PCR using primers from the clone ends and partial sequence of RGA8 from CO39. A total 231.0 kb DNA of CO39 was sequenced and constitutes the CO39 region linked to Pi-CO39(t). The coordinates of the aforementioned major clone sequences in CO39 are presented in Table 5, below.

Screening of BAC library from Japonica rice variety, Nipponbare. Rice variety, Nipponbare is the model rice genome being sequenced by the International Rice Genome Sequencing Project Consortium. Extensive resources are available for this genome project, including several large fragment libraries which have been end-sequenced and fingerprinted, a YAC physical map, and contig information obtained through overlapping fingerprinted clones. We screened a HindIII BAC library of Nipponbare consisting of 36,864 clones (http://www.genome.clemson.edu) with an average insert size of 128.5 kb markers with the three markers co-segregating with Pi-CO39(t). Several positive clones were identified including five clones hybridizing to all the three co-segregating markers, RGA8, RGA38 and G320. All five of the positive clones were confirmed to contain the expected restriction fragments associated with the markers by restriction digestion and probing with marker clones as well as PCR to identify the expected amplicon associated with the markers. The physical location of RGA8, RGA38 and G320 has been assigned by restriction and Southern hybridization analysis using a subset of representative BAC clones from the contig. A minimum tiling path of three BAC clones (OSJNBa0073N20, OSJNBa0082N20 and OSJNBa0044D15) was developed for sequencing. End sequences from the BAC clones in contig were used to walk in the CO39 library. Nipponbare is susceptible to the M. grisea strain 6082 and Guy11 (AVR1-CO39).

### EXAMPLE 2

### Sequence Information for CO39 Region and Selected Segments of BAC Clones Containing Co-Segregating Markers

The following sequence information relates to polynucleotides relevant the present invention.

CO39 Region Sequence (SEQ ID NOs:1-5): DNA sequence of CO39 region (231.0 kb) encompassing Pi-CO39(t) locus when these SEQ ID NOs are pieced together sequentially end-to-end, 3′ to 5′.

CO39.RGA8seq (SEQ ID NO:6): PCR product (360 bp) amplified from rice variety CO39 using primer sequences from the published RGA8 sequence. 84% identity to the published RGA8 sequence.

CO39.RGA38seq (SEQ ID NO:7): DNA fragment of 493 bp cloned from rice variety CO39 using primer sequences from the published RGA38 sequence. 97% identity to the published sequence.

### EXAMPLE 3

### Comparative DNA Sequence Analysis of Resistant and Susceptible Cultivars at the PiCO39(t) Locus

A total of 310 kb DNA from CO39 and 416 kb from Nipponbare was sequenced and annotated for putative genes using different gene prediction programs trained for grasses and Arabidopsis. All programs predicted almost similar numbers of open reading frames with different lengths. FGENESH (monocots) predictions were more accurate compared to GeneMark.hmm and Genscan, with few exceptions, based on the complete gene length when compared to known genes and the presence of signature motifs. Eight NBS-LRR genes were identified in the CO39 region. Gene predictions using FGENESH and BLASTp indicated the presence of several disease resistance (NBS-LRR)-like genes with highest similarity to RPR1, Xa1, Pib and Pi-ta. RPR1 (rice probenazole—responsive) is involved. in induced resistance to blast in rice (Sakamoto et al., Plant Mol. Biol. 40:847-855, 1999); Xa1 confers a high level of resistance in rice to race 1 of bacterial blight (Xanthomonas oryzae pv. oryzae) in Japan (Yoshimura et al., Proc. Natl. Acad. Sci. U.S.A. 95:163-1668, 1998); Pi-ta is a rice blast resistance gene (Bryan et al., Plant Cell. 12:2033-2045, 2000); Pib confers resistance to rice blast (Wang et al., Pant J. 19:55-64, 1999). All the predicted genes in CO39 and Nipponbare belong to non-TIR NBS-LRR subfamily of plant disease resistance genes suggested to be more abundant in monocot genomes (Meyers et al., Plant J. 20:17-332, 1999). The NBS-LRR gene clusters are flanked by clusters of Serpin genes in both the genomes. Serpins are serine/cysteine proteinase-inhibitors, whose biological function is not clearly defined in plants as yet with an exception of a Cucurbita serpin-1 (CmPS-1), which has been correlated with host-plant defense by reducing the survival and reproduction of an aphid (Yoo et al., J. Biol. Chem. 275:35122-35128, 2000). The Drosophila serpin gene, Spn43Ac has been implicated as a negative regulator of a Toll-mediated antifungal defense pathway (Levashina et al., Science 285:1917-1919, 1999).

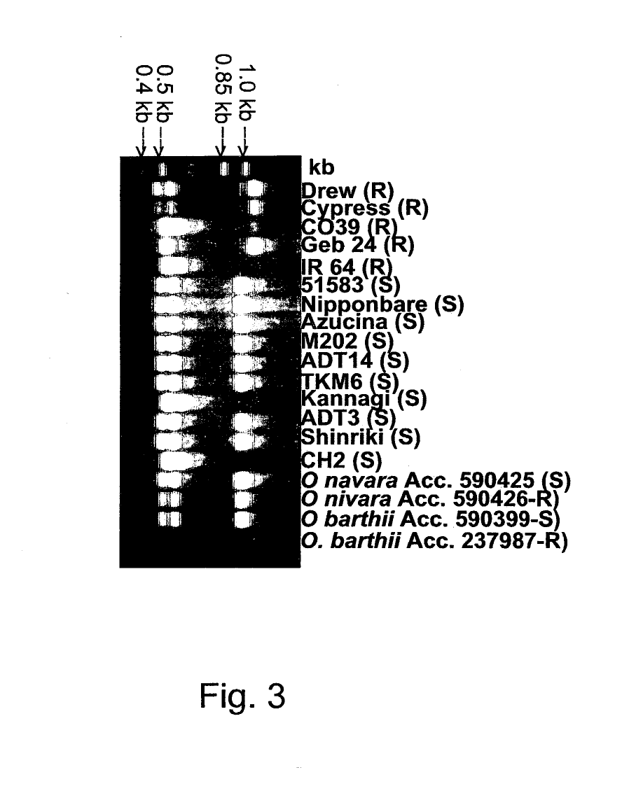

Analysis of Predicted disease resistance genes in rice varieties. Popular rice varieties were characterized for resistance (R) or susceptibility (S) to Guy11 and Guy11 (AVR1-CO39) strains of M. grisea differing for a single gene for avirulence, AVR1-CO39 (Table 6 & Table 7) and subsequently tested for the presence/absence of predicted genes by PCR as well as genomic DNA hybridization to find any functional correlations. CAPS analysis on NBS-LRR genes indicated that except for COR3, COR5, COR6 and COR8, the NBS-LRR genes at the Pi-CO39(t) locus are structurally similar between CO39 and many susceptible genotypes (FIG. 3). Genes COR5 and COR6 were not detected by Southern hybridization in the genomic DNAs of Drew and Cypress, which like CO39 are resistant to AVR1-CO39-containing strains of M. grisea. The CAPS haplotypes for different NBS-LRR genes were structurally more conserved across most of the susceptible genomes, whereas resistant genotypes showed diverse haplotype structures (FIG. 3), thereby indicating that structural rearrangements at the Pi-CO39(t) locus might have occurred during the evolution of resistance specificity to AVR1-CO39(t).

Expression analysis of NBS-LRR and Serpin genes. Expression analysis of predicted NBS-LRR as well as serpin genes was performed to study the expression kinetics in relation to the resistance response as well as to validate the exon-intron coordinates predicted by different gene prediction programs. All the NBS-LRR genes are constitutively expressed in CO39 and Nipponbare and no change in expression was observed in response to infection by virulent or avirulent strains of M. grisea except for RPR1, which showed induced expression in response to probenazole. Two serpin genes showed induced expression in response to M. grisea infection in CO39 and Nipponbare while the other serpin genes were constitutively expressed. Co-expression of serpin genes along with NBS-LRR genes during M. grisea infection in both resistant and susceptible genotypes indicates that the serpins might have as yet an unidentified role in regulating resistance response or proteoysis of host or/and pathogen factors involved in resistance or susceptibility.

### EXAMPLE 4

### Detailed Gene Predictions for CO39 Sequence Region

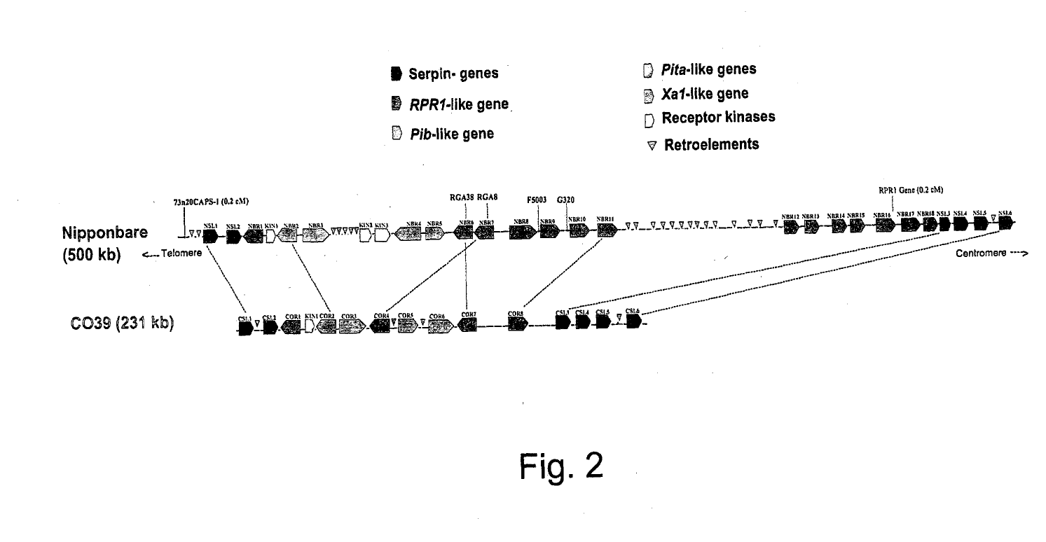

The coordinates of predicted genes in the CO39 sequence region are given in Table 8 and are referenced to the composite sequence represented by SEQ ID NOs:1-5 referred to in Example 2. The structural organization of predictive genes in haplotypes of disease resistant (CO39) and susceptible (Nipponbare) rice genotypes at Pi-CO39(t) locus is illustrated in FIG. 2. The NBS-LRR disease resistance-like genes have well-defined domains or small motifs (Table 9).

While certain of the preferred embodiments of the present invention have been described and specifically exemplified above, it is not intended that the invention be limited to such embodiments. Various modifications may be made thereto without departing from the scope and spirit of the present invention, as set forth in the following claims. All references disclosed herein or relied upon in whole or in part in the description of the invention are incorporated by reference.

TABLE 1
Segregation of resistance in F₁ and F₂ generations of
crosses between indica lines C039 (R) and 51583 (S)

| | M. grisea | No. Plants | | Expected | | |
|---|---|---|---|---|---|---|
| Cross | strain | R | S | ratio | X² | P value |
| F₁ (CO39X51583) | 6082 | 5 | 0 | all R | — | — |
| F₁ (CO39X51583) | Guy11 | 0 | 4 | all S | — | — |
| F₁ (51583XCO39) | 6082 | 4 | 0 | all R | — | — |
| F₁ (51583XCO39) | Guy11 | 0 | 3 | all S | — | — |
| F₂ Family 1 | 6082 | 205 | 59 | 3:1 | 0.99 | 0.5-0.3 |
| F₂ Family 2 | 6082 | 150 | 47 | 3:1 | 0.14 | 0.7-0.5 |
| F₂ Family 3 | 6082 | 109 | 34 | 3:1 | 0.11 | 0.7-0.5 |
| F₂ Family 4 | Guy11 | 180 | 55 | 3:1 | 0.32 | 0.7-0.5 |
| | (AVR1- | | | | | |
| | CO39) | | | | | |
| Total F₂ | | 644 | 195 | 3:1 | 1.38 | 0.3-0.2 |
| Progenies | | | | | | |

TABLE 2
Segregation of blast resistance in F₃ families of crosses between
indica rice lines CO39 (R) and 51583 (S)

| No. F₃ Families | M. grisea strain | R:Seg:S | 1:2:1 | P value |
|---|---|---|---|---|
| 78 | Guy11 (AVR1-CO39) | 16:44:18 | 1.38 | 0.5-0.6 |
| 59 | 6082 | 13:34:12 | 1.40 | 0.5-0.6 |

TABLE 3
PCR-based markers linked to PiCO39(t) locus

| Marker | Primers (5′→3′) | Size | Source |
|---|---|---|---|
| RGA8 amplicon | F:GGATGGTCGTGTCTCAAACC | 300 | CO39.rga8 (SEQ ID NO:2) |
| | R:AAGGCGACATGTTGAGGAAG | | |
| RGA38 clone | F:GGTGGGGAAGACGACATTAGTC | 500 | CO39.RGA38seq (SEQ ID NO:3) |
| | R:GGAGGCCATGACACCTATCCAC | | |
| RGA CO39 | F:CTTTCCATTGAGTCTTGAAGTCTTTGT | 2690 | CO39 Region (167.764-170.354) |
| | R:GGTAACTAACTTGAGGGAACTTCCAGA | | |

PCR conditions for all primers were: 3 min at 94° C., 30 cycles of 1 min at 94° C., 1 min at 62° C., 1 min at 72° C. and a final extension of 5 min at 72° C.

TABLE 4
Cross-over frequency among markers relative to Pi-CO39(t)

| Marker | Number of Crossovers |
|---|---|
| RM202 | 43 |
| RG1094 | 29 |
| RZ141 | 39 |
| RG211 | 62 |
| R2316 | 15 |
| RPR1 | 2 |
| 73N20CAPS-1 | 2 |
| RGA8 | 0 |
| RGA38 | 0 |
| G320 | 0 |

TABLE 5
Coordinates OF CO39 clone sequences in CO39 region sequence

| Clone | Sequence Coordinates (kb) |
|---|---|
| 34N09 | 1-79.339 |
| DNA Fragment between 34N09 and 36K06 | 79.340-87.015 |
| 36K06^{a} | 87.016-178.566 |
| 5E02^{a} | 155.211-231.028 |

^{a}There is a sequence overlap of 23.355 kb between clones 36K06 and 5E02

TABLE 6
Disease reaction of popular rice varieties and wild Oryza species
to Guy11 and Guy11 (AVR1-CO39) strains of Magnaporthe grisea

| | M. grisea | |
|---|---|---|
| Rice Genotype | Guy11 | Guy11 (AVR1-CO39) |
| CO39 (I) | S | R |
| Drew (J) | S | R |
| Cyress (J) | S | R |
| 51583 (I) | S | S |
| Nipponbare (J) | S | S |
| Azucina (J) | S | S |
| M202 (J) | S | S |
| M201 (J) | S | S |
| Crocoderi (J) | R | R |
| IR64 (I) | R | R |
| IR8 (I) | R | R |
| O. barthii (acc. 237987) | R | R |
| O. barthii (acc. 590399) | S | S |
| O. nivara (acc. 590426) | R | R |
| O. nivara (acc. 590425) | S | S |

TABLE 7
Disease reaction of CO39 pedigrees to Guy11 and
Guy11 (AVR1-CO39) strains of M. grisea

| | M. grisea | |
|---|---|---|
| Rice Genotype | Guy11 | Guy11 (AVR1-CO39) |
| CO39 | S | R |
| Geb24 | S | R |
| IR8 | R | R |
| Peta | R | R |
| Latisail | R | R |
| Taichung 65 | R | R |
| ADT14 | S | S |
| ADT3 | S | S |
| Kannagi | S | S |
| Shinriki | S | S |
| CH2 | S | S |
| TKM6 | S | S |

TABLE 8
Gene predictions for CO39 region*

| Gene | Predicted ORF | Coordinates |
|---|---|---|
| CSL1 | Serpin | 17274-21077 |
| CSL2 | Serpin | 51815-53833 |
| COR1 | NBS-LRR | 54358-57920 |
| COKIN | Kinase | 61573-65670 |
| COR2 | NBS-LRR | 66445-70578 |
| COR3 | NBS-LRR | 73918-79143 |
| COR4 | NBS-LRR | 82949-86779 |
| COR5 | NBS-LRR | 91967-96149 |
| COR6 | NBS-LRR | 124491-133102 |
| COR7 | NBS-LRR | 137162-140282 |
| COR8 | NBS-LRR | 168173-172768 |
| CSL3 | Serpin | 183628-186984 |
| CSL4 | Serpin | 187548-190361 |
| CSL5 | Serpin | 198235-202140 |
| Protease | Protease | 202538-208667 |
| CSL6 | Serpin | 223565-225700 |

*Non retroelement-associated

TABLE 9
Domain, subdomain predictions for CO39 genes

| | | | | | RNBS-D | | |
|---|---|---|---|---|---|---|---|
| | P-loop | Kinase-2a | Kinase-3a | GLPL | (Non-TIR) | LRR Domain | PSORT |
| Consensus | GVGKTT | LVVLDDWW | GSRIIITTRD | CGGLPLA | CFLVCALFPED | | |
| RPR1 | GMGGLGKT | ENFLIVLDDV | NFQASRIIITTR | CQGLPLAI | LRNCFLYCSL | LRR | cytoplasm |
| COR8 | GGLGKTT | SCLIVLDDVWD | PQASRIIITTR | CKGLPLA | QKNCFLYCSL | LRR | Plasma membrane |
| COR7 | GGLGKTT | KCLIVLDDVWD | NFQATRVIITTR | CHGLPLA | RNCFLYCSL | LRR | cytoplasm |
| Xa1 | GNGGIGKTT | KKFLIVLDDV | GNMIILTTRIQS | LKGNPLA | LQQCVSYCSLF | LRR | cytoplasm |
| COR6 | GIAGVGKT | KKFLLVLDDVW | GNMILVTTR | NGNPLA | LQQCFLYCS | LRR | cytoplasm |
| Pita | GSGGVGKTT | RYFIIEDLW | NNSCSRILTTEIE | KCGGLPLA | CLKACLLYLS | Rudimentary LRR | cytoplasm |
| COR5 | GAEGIGKT | RYFIVIDDLWA | GSRIITTTKVDE | KCGGSPLA | CLKTCLLYLS | Rudimentary LRR | Plasma membrane |
| Pib | GMGGLGKTT | KSCLIVLDD | TSRIIVTTRKENI | CDGLPLA | LKSCFLYL | LRR | cytoplasm |
| COR1 | GMGGMGKTT | KRYLVVFDDV | CRNGSRIVITTR | CGGLPLA | LRNCFLYCS | LRR | Cytoplasm |
| COR2 | GFCGCGKTA | NRYFIVIDDIQ | DKDIGSRIVVTTT | CDGQPLA | LKACLLYFG | LRR | Cytoplasm |
| COR3 | GPAGIGKTA | RRYLIIIDGLW | VNSFSRILITA | CGGLPLA | FKTCLLYL | LRR | Plasma membrane |
| COR4 | GMGGLGKTT | RKCLIVLDDVW | DQGSRVIITTRK | CQGLPLA | LRNCFLYCS | LRR | Cytoplasm |

RNBS-D (non-TIR) domain is predominantly present in monocot genomes
Pita, RPR1 and Pib: Rice blast resistance genes
Xa1: Rice bacterial leaf blight resistance gene

### References:

Amasino R. 1986. Acceleration of nucleic acid hybridization rate by polyethylene glycol. Anal. Biochem. 152:304-307.

Bent A. F., B. N. Kunkel, D. Dahlbeck, K. L. Brown, S. R. Schmidt, J. Giraudat, J. Leung and B. Staskawicz 1994. RPS2 of Arabidopsis thaliana: A leucine—rich repeat class of plant disease resistance genes. Science 265:1856-1860.

Bryan G. T., K. S. Wu, L. Farrall, Y. Jia, H. P. Hershey, S. A. McAdams, K. N. Faulk, G. K. Donaldson, R. Tarchini and B. Valent 2000. tA single amino acid difference distinguishes resistant and susceptible alleles of the rice blast resistance gene Pi-ta. Plant Cell 12:2033-2045.

Causse M., T. M. Fulton, Y. G. Cho, S. N. Ahn, K. Wu, J. Xio, J. Chunwongse, Z. Yu, P. C. Ronald, S. B. Harrington, G. A. Second, S. R. McCouch and S. D. Tanksley 1994. Saturated molecular map of the rice genome on an interspecific backcross population. Genetics 138:1251-1274.

Chauhan R. S., M. L. Farman, H. Zhang and S. A. Leong 2002. Genetic and Physical Mapping of a blast resistance locus Pi-CO39(t) that corresponds to the avirulence gene AVR1CO39 of Magnaporthe grisea. Mol. Genet. Genomics 267:603-612.

Chen X., S. Temnykh, Y. Xu, Y. G. Cho and S. R. McCouch 1997. Development of a microsatellite framework map providing genome-wide coverage in rice (Oryza sativa L.) Theor. Appl. Genet. 95:553-567.

Ewing B., and P. Green 1998. Base-calling of automated sequencer traces using PHRED: II. Error probabilities. Genome Res. 8:186-194.

Fukuoka S., T. Inoue, A. Miyao, L. Monna, H. S. Zhong, T. Sasaki, and Y. Minobe 1994. Mapping of sequence-tagged sites in rice by single strand conformation polymorphism. DNA Res. 1:271-277.

Farman M. L. and S. A. Leong 1998. Chromosome walking to the AVR1-CO39 avirulence gene of Magnaporthe grisea: discrepency between the physical and genetic maps. Genetics 150:1049-1058.

Gordon D., C. Desmarais and P. Green 2001. Automated finishing with Autofinish. Genome Res. 11:614-25.

Gordon, D., C. Abajian and P. Green 1998. CONSED: a graphical tool for sequencing finishing. Genome Res. 8:195-202.

Grant M R L Godiard, E. Straube, T. Ashfield, J. Lewald, A. Sattler, R. W. Innes and J. L. Dangl 1995. Structure of the Arabidopsis RPM1 gene enabling dual specificity disease resistance. Science 269:843-846.

Harushima Y., M. Yano, A. Shomura, M. Sato, T. Shimano, Y. Kuboki, T. Yamamoto, S. Y. Lin, B. A. Antonio, A. Parco, H. Kajiya, N. Huang, K. Yamamoto, Y. Nagamura, N. Kurata, G. S.

Khush and T. Sasaki 1998. A high-density rice genetic linkage map with 2275 markers using a single F2 population. Genetics 148:479-494.

Kiyosawa S. 1981. Gene analysis for blast resistance. Oryza 18:196-203.

Lander E. S., P. Green, J. Abrahamson, A. Barlow, M. J. Daly, S. E. Lincoln and L. Newburg 1987. MAPMAKER: an interactive computer package for constructing genetic linkage maps of experimental and natural populations. Genomics 1:174-181.

Letunic I., Leo Goodstadt1, Nicholas J. Dickens1, Tobias Doerks, Joerg Schultz, Richard Mott, Francesca Ciccarelli, Richard R. Copley, Chris P. Pontingl and Peer Bork 2002. Recent improvements to the SMART domain-based sequence annotation resource. Nucl. Acids Res. 30:242-244.

Levashina E. A., Langley E., Green C., Gubb D., Ashbumer M., Hoffmann J. A., Reichhart J. M. 1999. Constitutive activation of Toll-mediated antifungal defense in serpin-deficient Drosophila. Science 285:1917-1919.

Mago R., S. Nair and M. Mohan 1999. Resistance gene analogues from rice: cloning, sequencing and mapping. Theor. Appl. Genet. 99:50-57.

McCouch S. R., G. Kockert, Z. H. Yu, Z. Y. Wang, G. S. Khush, W. R. Caffman and S. D. Tanksley 1988. Molecular mapping of rice chromosomes. Theor. Appl. Genet. 76:815-829.

McCouch S. R., R. J. Nelson, J. Tohme and R. S. Zeigler 1994. Mapping of blast resistance genes in rice, pp. 167-186 in Rice Blast Disease edited by R. S. Zeigler, S. A. Leong and P. S. Teng. CAB International, Wallingford, UK (in association with the International Rice Research Institute, Philippines).

Meyers B. C., A. W. Dickerman, R. W. Michelmore, S. Sivaramakrishnan, B. W. Sobral and N. D. Young 1999. Plant disease resistance genes encode members of an ancient and diverse protein family within the nucleotide-binding superfamily. Plant J. 20:317-332.

Michelmore R., I. Paran and R. Kesseli 1991. Identification of markers linked to disease resistance genes by bulked segregant analysis: a rapid method to detect markers in specific genomic regions by using segregating populations. Proc. Natl. Acad. Sci. USA 88:2236-2240.

Osoegawa K., P. Y. Woon, B. Zhao, E. Frengen, M. Tateno, J. J. Catanese and P. J. de Jong 1998. An improved approach for construction of bacterial artificial chromosome libraries. Genomics 52:1-8.

Sakamoto K., Tada Y., Yokozeki Y., Akagi H., Hayashi N., Fujimura T. and Ichikawa N. 1999. Chemical induction of disease resistance in rice is correlated with the expression of a gene encoding a nucleotide binding site and leucine-rich repeats. Plant Mol. Biol. 40:847-855.

Smith J. R. and S. A. Leong 1994. Mapping of a Magnaporthe grisea locus affecting rice (Oryzae sativa) cultivar specificity. Theor. Appl, Genet. 88:901-908.

Tao Q. and H. B. Zhang 1998. Cloning and stable maintenance of DNA fragments over 300 kb in Escherichia coli with conventional plasmid-based vectors. Nucleic Acids Res. 26:4901-4909.

Valent B., L. Farrall and F. G. Chumley 1991. Magnaporthe grisea genes for pathogenicity and virulence identified through a series of backcrosses. Genetics 127:87-101.

Wang Z. X., M. Yano, U. Yamanouchi, M. Iwamoto, L. Monna, H. Hayasaka, Y. Katayose and T. Sasaki 1999. The Pi-b gene for rice blast resistance belongs to the nucleotide binding, leucine-rich repeat class of plant disease resistance genes. Plant J. 19:55-64.

Wu Y., L. Tulsieram, Q. Tao, H. B. Zhang and S. J. Rothstein 2000. A binary vector-based large insert library of Brassica napus and identification of clones linked to a fertility restorer locus of Ogura cytoplasmic male sterility (CMS). Genome 43:102-109.

Yoo B. C., K. Aoki, Y. Xiang, L. R. Campbell, R. J. Hull, B. Xoconostle-Cazares, J. Monzer, J. Y. Lee, D. E. Ullman and W. J. Lucas 2000. Characterization of cucurbita maxima phloem serpin-1 (CmPS-1). A developmentally regulated elastase inhibitor. J. Biol. Chem. 275:35122-35128.

Yoshimura S., U. Yamanouchi, Y. Katayos, S. Toki, Z. X. Wang, I. Kono, N. Kurata, M. Yano, N. Iwata and T. Sasaki 1998. Expression of Xa1, a bacterial blight-resistance gene in rice, is induced by bacterial inoculation. Proc. Natl. Acad. Sci. U.S.A. 9:1663-1668.

Yu Z. H., D. J. Mackill, J. M. Bonman, S. R. McCouch, E. Guiderdoni, J. L. Notteghem, and S. D. Tanksley 1996. Molecular mapping of genes for resistance to blast (Pyricularia grisea Sacc.). Theor. Appl. Genet. 93:859-863.

Zhang H., S. Choi, S. S. Woo, Z. Li and R. A. Wing 1996. Construction and characterization of two rice bacterial artificial chromosome libraries from the parents of a permanent recombinant inbred mapping population. Mol. Breed. 2:11-24.

Zhang H., X. Zhao, X. Ding, A. H. Paterson and R. A. Wing 1995. Preparation of megabase-size DNA from plant nuclei. Plant J. 7:175-184.

We claim:

1. A polynucleotide isolated from chromosome 11 of Indica rice cultivar CO39, flanked by genetic markers 73n20CAPS-1 and RPR1, which polynucleotide comprises one or more genes that confer resistance to strains of Magnaporthe grisea having avirulence gene AVR1-CO39.

2. The polynucleotide of claim 1, wherein the one or more genes co-segregates with a marker selected from the group consisting of RGA8, RGA38 and G320.

3. The polynucleotide of claim 2, part or all of which is contained on one or more BAC clones from rice cultivar CO39 selected from the group consisting of 5E02, 36K06 and 34N09.

4. The polynucleotide of claim 3, wherein the one or more genes is selected from the group consisting of serpin-like genes, NBS-LRR genes, rice Pib-like genes, rice Pi-ta-like genes, receptor kinases-encoding gene, rice Xa1-like genes, and protease-encoding genes.

5. The polynucleotide of claim 4, comprising one or more open reading frames selected from the group consisting of CSL1, CSL2, CSL3, CSL4, CSL5, CSL6, COR1, COR2, COR3, COR4, COR5, COR6, COR7, COR8, kinase-encoding and protease-encoding.

6. The polynucleotide of claim 1, wherein said polynucleotide is SEQ ID NOs:1-5 or a polynucleotide that is substantially the same thereas.

7. An isolated gene comprising an open reading frame selected from the group consisting of CSL1, CSL2, CSL3, CSL4, CSL5, CSL6, COR1, COR2, COR3, COR4, COR5, COR6, COR7, COR8, kinase-encoding and protease encoding.

8. A transgenic plant comprising the portion of the polynucleotide of claim 1 that confers the resistance.

9. The transgenic plant of claim 8, wherein said plant is a monocotyledonous species.

10. The transgenic plant of claim 9, wherein said plant is selected from the group consisting of rice, maize, wheat, barley, oat, rye, millet and turfgrass.

11. A method of enhancing pathogen resistance in a plant, the method comprising the steps of:
a. transforming the plant with a portion of the polynucleotide of claim 1 that confers the resistance; and
b. pre-treating the transformed plant with an agent selected from the group consisting of an AVR1-CO39 gene product and a non-pathogenic organism that expresses a portion of an AVR1-CO39 gene effective to trigger expression of a CO39-specific R gene in the plant; the pretreatment resulting in the enhancement of pathogen resistance in the plant.

12. A method of enhancing pathogen resistance in a plant, the method comprising the steps of:
a. transforming the plant with a DNA construct comprising a portion of the polynucleotide of claim 1 that confers the resistance, operably linked to an inducible promoter; and
b. exposing the plant to conditions that cause the inducible promoter to induce expression of the polynucleotide, resulting in the enhancement of pathogen resistance in the plant.

13. The method of claim 12, wherein the inducible promoter is wound-inducible or pathogen-inducible.

14. The method of claim 12, wherein the inducible promoter is chemically inducible.

15. The method of claim 12, wherein the inducible promoter is also tissue-specific.

16. The method of claim 12, wherein the inducible promoter is also organelle-specific.

17. The method of claim 12, wherein said portion of the polynucleotide of claim 1 is a fusion with a portion of a polynucleotide of AVR1-CO39 that confers host recognition by Pi-CO39(t).

18. A method of enhancing pathogen resistance in a plant, the method comprising transforming the plant with a DNA construct comprising a portion of the polynucleotide of claim 1 that confers the resistance, operatively linked to a promoter that constitutively expresses the gene in the plant, the constitutive expression resulting in the enhancement of pathogen resistance in the plant.

19. The method of claim 18, wherein said constitutive expression is in a specific organelle or compartment of said plant.

20. The method of claim 18, wherein said portion of the polynucleotide of claim 1 is a fusion with a portion of a polynucleotide of AVR1-CO39 that confers host recognition by Pi-CO39(t).


SEQUENCE LISTING (72 sequences)

SEQ ID NO: 1 - DNA, 49680 bp, Magnaporthe grisea
ggatccccaatcatgatccgaaaacggaaatttcatgccatccaatcataccatagcgagtctatcgaagcaaacaaaagtttcagatgctgctctggtgttaattgaggagttcaatcctggcaacggttttataggtatttctatagatatctgaatttcaatccttgaggcacgagctggttgtttggtgttgtgcagtggtatagagtactccctccgtcccaaaaaaaaaaagacaaactctaatttccgtgtccaacgtttgaccgtccatcttatttgaaaaaattatgaaaaaatttaaaaagacaagtcacgcataaaatattaatcatgttttatcatctaacaacaatgaaaatacgaatttaaaaaaaattccctgtaagacggaccgtcaaagttggacacggaaacctagggtttgcatttttttttacggagagagtagttcctatcatatgctgcgtttctttattattctaggacatttatttgcttgttttattcagatacccccgttttcacaaatatccaagatacaagccgtgttttagtccttttcaaaaaaaaattgaagtttgaaggtaactaaacacagcctatatattcaccatttctgtgtgaaacgatgaaggatttgactatgacggaaacaagtaaaatatcactactcattttgctagttgagagatatcataggttgaagttcctttccttttgtttttaggaaatagatccaatttctgagttttccttcaaatcaaggaaatgctgtaacctattcccagttattgttttcggtgtttcctagcacatttgttcaattttgcacttgttgttataacaactagggagatagcccgcgtattcatgcgggtcatgcatcgtaagcagcatttgagaatggatcatattaatggaatagcctctgcaatgttttgagagtatatttccttgacaaatattcatcgtccgtatcgctttgataatttctctaatctatattatatatatatttaatttaactgtataagcaattataaataatcttcatcatcatttaagacttagtttgtttataaaacatgttaaataaattaagtatagattcaaagaaagtcatttttttctttttttagttgaatgttttgggtccattggtgaaaacctaagacctagctaaactcgcttacctaggtaaactcgttgtttacggttttctttaaggaattggattttctttggactattcatattttatagttagttctattttcttagatagtgtatatctaatttagctacacgtgtttgcaaaaacaacatatgatgatgcaacctgtgttgttagatcatttcaaaattcttttatggcaattcaaatgacatgtaaaagggcaggggtattccacctcaataataaaatgtacgacagttcacttgtaatttttatttggatactactggttttatttagctatgcatgtttacggaatgatgtaaaatgacgtaatatatgcagtcaatttgtttgagctatttcgattatatttggatggtatgcaaagcgcatgggtcgaatgtggctgaatgcgatattttgtatatatgaaataagatgtgtaacttaaaagttatggttgttttaatatctaaaattattgattatttctcttatattttaatagctaaatatagggaggggacatgcaatggtgtgcgcatgtgatggggtgaggaggtggatgcgatttattttctaaccttttctaaataatgtaacttaaaaccaataatgataggtttcattattttcctcataatttctaagattatttatgatttttaggacataatacgtggcagcgtaagattttttttaaattagatctaatagttaaaaataattggtcatcgatttaattgaaatattttgtaattttaagacgtgacatatggtgacttaagtgtgatctaaaggctggttaattggttttattatgtagaatgatttaatatgacatattacatattttgatgttttaaattgtaatgttccattgtcatttagtttccatgcagagtattttaatttttgtatagtaaaatacagctataatggatctttaattatttaattaattgtaaataaaaaatagttcaaacaaattaaaaatcaaatataattttagatactataaatcgtttagctacgttgttttatattaggttgttagttaaaaaaaatagaagaggagaggaggggagtaagaagggagaggaccataggtatatgttcagcgggaatagggccggagattttttttttagtgatagatataattttagatctctgagaaaatatgtcaggtgttgtattttctaatataagctaaatgatgctaatcatatggtttaaaaatctaggtgagtttttctaaaaatgtatcaagtgaccaatagctagtctcttgccatataggatccaaagtaatacatttattgtacttgttaaatatagttaatttggaaattgaattataaactttttattgtaaataatcccatgacatgcacgaataaaaaaaatcagctctatgatttagacaatacagttgttttaaaatttttatcacaaaatatgttttctatatactaattgcttgaaaataggaggggaggtctccctcgtgcatcacgtgaggccagggtgtggtatatggtacgtaactacatacgtatacataatgagccttttcttttgatggctggaggtggggtgtgggccttttctttttcctctttagatcaatctgatggtagaaaatatggatcatcgaatattatcgaatattagtgaaaatcaacggttagatgttttgcttttttctctaatttgctagagtaacacgtggcaagttaagagcgtttctagaatgccacatggcgacttaagagctgctagccctgggccaacaaatcccacatgacgatgcaacaaatcgccccacgcgtggcgcgggagttttttttcgcgtacggataggatgaaagacaccccaatcgaacgatgaggagaacgtttgtagaaagtttaatggacttttagtatataatagatagatagatagatagatagatagatagatggtgtgaacatcatggaaaatgctgtgaaacaaaaggattacagagggactgactagggcagccctaatgggcgatcgatcgcccgccaatggggggtagcgatcagattcgctaccctctcccccctccctccctccacctgatttccttttttggcaccgtattactttcctattttagtaaatttatacacataaagtttatacacctcaagttcacacatctaaagtttagagacccaaagtttataagtcaaaagtttatatatccgattcaaatttgaatttgaattcaaatattttttatatatagtatttctatacatctaaagtttatacacctaaagttagtttatagacctaaagtttatacacctcaagtttacacatctaaagtttaaatacatgttacaaatttatatttgaattatatccgattcaaatttacctgtttcaaatttgaatttgaattatatccgattcaaatttgaatttgaatttaaatattttctatatatagtatttctatgcatctaaagtttatacacctaaagtttatagacccaaagtttatagacccaaagtttataagtcaaaagtttacatacccgattcaaatttgaatttgaattatatccgattcaaatttgaatttgaatttaaatattttctatatatagtatttctatacatctaaagtttatacacctaaaatttatagacccatagtttataagtcaaaaagtttacatacccgattcaaatttgaatttgaataattttttatatatagtatttctatacataattttttctaactttttgttttttttttaaatttttgtgtggtgtactgcagtaaggaaggagggagaagtgtatctagtagtatagggagggggcggacgggtgatcgctggacaggcgggggagcgatcacccgcccattagcatttctggactgactacgcaaccaacaaatcactgaaaaacaaaatttacagaccgagcgccttgggtaaactgattttgacggcaatccactcacaattgatcacaattgagagttgcataatataggctatgttcggttaattcccaagagaggaggattggagaggatttattccacacctgttgtggtgtggaattatttccccttatttcccctaaaacccctccaattcccttcaacctcctataaaccgaacaagatcttagaggtttggcccaataaattggtgccagcccaatccacatcatcttcaagttcaacacagtatcaggccctgtttcttttagcttgggattattataatctagattattgagtcagattactataagctagattgttataatctgtattaaaataagcggttagttgtttctttcctagattattagagcctagattattgggtttgcaggtctaaagagggagtggagtggcatggtgggtaattttttacgcaataatctagaaaaagctcacctaaatgagcttattagattatataagctgggctctagattataataagctattttaataagttgtctgtttctttcagtttactcccaataatttagattataataatcccaagctgaaagaaacacggccttatgcaagtggagtacttattagaaaaagtatgcaagtattttttttattatattttagacacattgaaatttatatatgttttcatttcaatgtatccgatgcaaaatttgtgaccagatgggattaataatcaattatttttccagtcaccagatcagcacactgacccagcctagatacatataagcacagtacaatatttttcgtttcgatggatgatgatagtactagatacgaaatgggatgccggccggccaatctcaacgctggcatgcaccgcgcgcggcgaggcattgtcgaagatgaggctgatgggcgcgctggcggcgtacgtgaacaccaccggcgaccccttcacgatatgcaggccgtcgttgacgaggcagagcccgtcgttgccggcggcgtggatcttgctccggtcgacgccagccgggcacgctgcacttgtgctctctccgtattttaatatatgacgccgttgactttttgacaaacatttgatctttcgtcttattcaaaaaattatgtaattataatttattttattgtgacttgatttatcatcaaatattttttaagcatgatataagtatttttatatttgcataaaaattttgaataagacgagtggtcaaacgttgattaaaaagtcaacggcgttatacattaaaatatggagggagtagtttattagtgccacttctgactactgatcaagcggaccatgttatctgtacccgataaaattaactaaatagtatgcatgcataatttagattttgcctaagcacttgccatgatcaaagttagcttcttccatttactgtaggaattctataacgatattgtactgtgattgagggagtactaacattgactccggtactcatgtaccacaaaacttcaagtgtaaaattaactcacacatcgtgaagtaaatatcatcaaattcagcatataatactatcgacacataaatttgtcttttttttctcaatgtacacagtttctaaaacacgatttccggtagaatgatgatataacataccagccacttgcactgtacttttagaaagaaaaaaaatgtgtagaatttaaagacagtggatagaaatccagtctctcaaaatggatatacacagccacgtagaacaatatatcttcactcatcaggggattgaattaaatccatcgccgtaatcacgaagatacaatcaaatcgctaattttgggataatctgatatatattgcccagatatacatatatgctcctccaaaattttttatatatagtgacattctctacactaattcttcacacaaaagagagtgaagcaaacgcatcaaatcactagctcaccaccatgacgaccaaggccaccacccttctcctcctcctcgccgccgccgccgccgcggcgctcctctgccacgtccacgtcgccgtcgccggcgaggacccggagccgtgcgacccgtcggacatcaccatcgcgacggtgaagaccgggcgggtcgtgggcggcctgccggagttccaggtgacaatcggcaacgagtgctcgtgcccggagggcgacgtcgtggtgtcctgcctcgacggcgtgcccgccggcgtcgaccggagcaagatccacaccgccggcagcgacggcctctgcctcgtcaacgacggcctgcagatcgtgaaggggtcgccggtggtgttcacgtacgccaccagcgcgccgatcagcctcgccttcgacaacgcctcgccgcggtgccagcgttgagctcggctagccgccggccgccggcatggcatcccatttcgtatcaccgtccatcgaaaccaaacccatcttcttaaatttaaatatgtaatctaggctgggtcagtgctgatctggtgaatggaaaaataattgattgttagttccctcttgtcacaaatatttgcaacggatacattgcaatgaaaacatataaatttcaatgcgtataaaaattttaaaatatactttgcatactttttcacttgcataatactatgttggacttgaagttgacgtggattgggccggcccatcaatttcttgggccaaacctctattggattttgaaactctcaattatgatcaattgtgagtggattgatatgttttgaaacggataaccgaaggctctcggtctgaaaattttgtttttgggtgatttgttggttgcgtagtcagtccctctgttaatccgtttgtttcacagcatttttccatagtgttcacaccattgtttataagaacaagtgcaaaattgaacaaagatgctaggaataccgaaacaattactgggaatagggtacaacatttcctagatttgaaggaaaactcagaaattggatctatttcgtgaaaacaaaaggaaaggaacttgaacctatgatatatctcaactattatctaaaaaaatgatatgtctcaactagcaaaatgagtacgtaatgatattttacttgttggcatcatagtcaaatccttcatgttttcacacggaaatgtcgaatatacgcagtgatatttgtgaaaacgggtaatgactgaataaagcaagcaaataaatgtgcaagagtcctacaataataaagaaacacatgcgataggaactatctatcactgcacaacaccaaacaaccagctcgtgcctcaaggattaagattcagacacctgtggagaaatgctttttactctcagttggtaacctcctataatcccggttttccaaccgggagtaagaatctgggactaaatattgttatctttagtcccggtttaaataactaggaataaagatcgatctttagttccggttagtgttaccaaccgggactaaatatttagtcccggtctaaagttttttttcttttttttttctattttgtatatctcactcgttttctcaaatcattcatagcaatatagttcatcccaaatcattcacaacaataacatagattgaatcacacattcaacacagatcaaatcattctttaaggaaaaaagaaaaaaaaatgcacgcaacagcgccggcctccaccgcctgccgccgcaccggccaccgcgccggccgccgccgggggcggccctctgtgtgccaccaccgccgccgcctcccctccaaaataaaacggggggggccgcccccccctccctcctccgcgcccggccgtcacgcgaggccgcctccgccgctgcccggccgccgccaaatcgtcccctccacgaggccgcctgagaatggagagaagaggggaggatgagatgaggaagagtaagagatgggatgaatgaggagagaaggaagatgaagagatggggtgaagaggaggaagagataagggaggaggagatagaacttattaccatgcgcctcctcctcctcgttctcacgcgcacgcctcagcctctccgcccatgccgatcttttttcaatagatctttagtcccggttgatgttaccaaccgggcctaaagataataaaggggtctgacactgcctaccttttttttaaccaggattaaaaatggtctttagtacaccaaccgggattaaagataaaaaagtagctctaaacttttgaacctggactaaaggttatctttaatctcggtttttattgtaaccaggattattgtagaatttgatcgacatatgtacccatatactacaaagtgataatatcctccattcctccaggcaacggtttatagattcttctatatcacgaatcttaatccttgaactcctcaattaacaccagagcagcgactcgtcacagtatgattggatatcatgaaatttcctttttttgcatcatgatagattgataatactagctttctccgtttcacaatataaaactttctaacattgctaacatttatttagatgttaataaatctagatatatgtatgtgtttagattcattaatatatatatgtatatgggcaatgctagaaagtcttgcattgtgaaacggggggagtagatgtaatcacaatttcagcatttaattaacttgctggtttttgcatttagcatgttaaatatcacaaattccgtttttccatcatgaacttatccatgtactttgctattaatgtttaatatatattgagtttaaaaaaagagagtagtatgttaaatattggtccacaagcacgctgctcagatcaacaccgtcgtagctgcgtgcaatcttttcttttggccatcagcaacaagcactaaatttggacgactaaattaaattgactaacgctacataatcacaattttagcttcgtccattaatttagagtgggtcgtcacagcttggcctaagaaattggtgggccaacctagcgctcatctcttaagtccgccatactgcctccatcttttagtaaaccatgcatgctgttgatttttcgatatgtttgactattcgtcttatttaaaaatttatcatcttcggtttcttaatgttttagatacgctatgcaaaacatatttttaactatattttttattataatatgtacaataaatattttttcttacttttattatagtattttgtaagataaatagttattgttagttaaagaattatggaactggatgaaataagtaattatcatttattttatttgtacgtttaagtatgatttatatcttatgaatttgcatcaatttttaaataaaatgaataatcaaacatgtgtccaaaatttaatagcgtcaactacaaaaaaccgagaggggactatgcaagtttttttttctttttattttgaaaaaaaaaacactgaaatgtagctactttcatgtgcccgatgcaagtttattttccattcaaaagatcagcccatccagacgaccagttacataaaaagaagagatcgtttcgatggaagatgatgcaaacgattaatggtacgcctgcagccaagctcaacgctggcaccgcggcgtggcgttgtcgaaggcgaggctgatgggcgcgctgggcggcgtacgtgaacacaaccggcgaccccttcacgatctgcaggccgtcgttgacgaggcagagcccgtcgctgccgccggcggcgtggatcttgctccggtcgacgccggcgggcacgccgtcggggcaggacagcacgacgtcgccctccgggcacgagcactcgttgccgatcgtcacctggaactccggcaggccgcccacgacccgcccggtcttcaccgtcgcgatggtgatgtccgacgggtcgcacggctccgggtcctcgccggcggcgacgcctacgtggatgtggcagaggagcgccgcggcggcggcggcgaggaggaggagaagggtggtggccttggtcgtcatggtcgtgacgatgatggcgttagataagcttgatggtagatggtttggttcaactctctgtggtgtgaggaaaattagtgtagatagtgtcactatttatagggatcatcagtttgtagtactatatgggtacataattaagggcaacatatcagattagctcaatttggtgatttgcatgtatctcttcgtgattacgaagatggacatgcatcctccattcctcctacgcgttttttttctctcaaaaatgtactgcaaatggtatatcagaaatcacgtgatttgatcgatcttttcccctccatgtatgactgtatgatgagtggaggaatggaggatgttatacgaagatggattctaatccctatagagagggataatttgtactatgtgaatagtactacaaatactgttaataatatgttgaatttgatattttccacaatctaaaatatcattttataatccgtacatatatataggtggtagaagctaactttcaccataatataattgttcacgttattaaagtaacagtcattatatacaaagttgtcatagtattatacttgtgcaactattttttgtttatcaaagtgatcgaggttacatcttactgactcttttagttgatattttactgactttattaaaatgcctaaaacatcgttttttgtgaatggagggaatgaatgagtaagctttattttttttttaaaaaaaaaagggaacatgaaatgagtacgcgcgatttcagtctgcatgcaaaaatgtgttaagtggcaggagagcaatgccacgtacgtgggccgaccgaggaataatgcatgctgatcaccttagttcatacattctatccttccaaagattttctcgaagatgatcattttagatcgtgatatatgatcgatccgtttcttaattactccctcgatccgtttatattatataagtccttaatcaaaattttaaagtttgatcaagtttttagcaaaatatagtaacattttcgtattcaatgtcaaatttagtggaattaatttgatactgtagaagttgctttttttttataaatttggacaaatttaaataattttgattaagaaaaaattcaaacaacttaaaatataaacggagagagcagctcacatatgtacttccctctgtcccaaaatgtgtggaacgttatcaaataactgcatcagtttcacgcatgcagtataacattacaaataaaattactttagataaacggaatctatttactccctctatgttccaaaatgatgggggagtaactattttaatgtcattaactgcagccaagttagttggtacaacatccgttttattataatttgttttgaatttttttcataattcaaatttctttatatttgaccaaatttatagaaaaatataagaaacaaaactgacaaaatatgttagttgtactcaaaattcaagggattttgctacatagttattttgggatgaaattagtatgtcttattgaactaataggagaatgataagagtcaacaagtcagatcaaatcgcactatcttagggtagtagcaatacgtcccgccaaacatccacataggaagaaaagcagtcgacgctcccctctccacaatgcactcatctttggttggacccaccacttttctttttaggccaaccccaccaccttttcttctcctccccctctctctctctctctcggaaaatctaataggcgatcgatcgcggtagcgaccaggtagcgatcgccacccctccaccctccctctatatttacttttattgtactgggatatatcataaggttgattttttttgacggttggaatatcttagaaataagttgtttaaagtttgattaaacaaaaagaaaagggagagagaatgcagagtcagacatgcatacgatgctccgccacccctagctccctgtacgcatgcaacgccacagagatgcggcaaagtgagcgacgtttgatggctagctttctccagaaaagtttggaggaaattatcgatccggcgcgttacgttagtgtgcatgtccattaaaatcctgacacttgataaatcgtgcaagttacatgctatgctaaggcgtgctacatgtttgtattatcttttttttttctcaaagtataactccaataaaacttcaaattgttttcttcttctaaattacttatctgaattgaaatccgattacatcattatattcgttacaattaactcttgctaataagatctcacatgattatatttttttataaacatatatcttcaatttataagttgtctgtgtggaggaagaggaacaagaggaagcaccaaaccgcaggaaagaaaaagaaaataatttggtgtcaatcaaaagacttaatttctaaaaataaggatggaaaaaaatcactcactcactacctagaaaggtacttaacattaatccaatttcaggttgaaatatatcatcaatctcccttcccaatgcaatcaagttgttaactagttagtagttacacatcgctggacactacccactctgaccgggtaagtagggaaaatatacggtggaaaccagctatgcagtgaaatcttgaaaactaccgttggataaaaaatggacgtctaagatttgttcacgtcattacgagtaaaaatactcaaggtaaaatctttacttatgagtaaatccgtgagtaaaatttttactcgtgatggcgtggacgaatgtcagacgtccatttttcgatccaaagatagtttctaagtttccacatgtggttttcaccacatatttttccgggtaagtggatcttataggaggcagcctttgtatgtaggggcgaattaaatttgtgctctctctacttcaaaaaaagaaatcaaattctaactataaaatatgtgttcgtccaaattcatagtcagcatttgtttttttagacggatggtgtaggtatttgaacccatagttttctccagacaatagaaatatgctatcaaacttagccaattacattaaaagttattgcactgaaagtgatcttaaaacccacataatcacaattatatatacttgatttacttcatatgaatgtagtctacgacgatatgttaatttcgatcctaatatatattgtagttagaccattactactattgcccatggttatatatttgaatgtccttgtaataataaactgctattgacttttaaaaattacgttggtaccatataatgtgtacgggcgccaaaaaaagaaagaggacaataagtgagataaaaggctcatcattttgattctaaacttcattaaattgacgaggttaaaagacaatttcgaatgacaaccacggaaggcgcaccctgccctcagcaatgcacctactcaaatatgtaagaaaaagttacacgtatagaaatttgttaaactccttagaaacgttgattagtcaaaattttgtaaggattttcgttggtgtcctaaaaattttggaagagttatttaaagttagcatcctccaaaatatgaaataccgaatgtttgtaattaatcataagttgtcacgggaggcgcaggcgcagtgtcgacgacaggaggagctgatccggccgctgcataggtcttatcggcggttgatccggctactggagatgtctcctttgcggaggttgatccggccgccgcagaggtcgccgtgtcggcggttgatccagccgccgcagaggtctccttgtcggcggttgatccggcccccgccgcagaggtatctgatcccgcggccttgtcccctggcagcttcgtcagaggatacaggtcctcgagcaccgcgtcggcgaaatccagcaagctccacaattgcacgaacagcgcgacgacgttgcgcttcttctcctccgtgcacgagccgtcctggcagcaggtgttgcagaggggcgggcggcgcgtgccggtggggccgcggagcagctgcgtgaggctggcgtggatggcgaggagcttctcgtcctcggggaccttgacggcgtcgtcgccggtgccctggggctccagggcgtcgttcacggcggcgaaggcgtggcggcagctgtcgaggcactggtccaccttgttcttgcactcgtccacggcggcggtgcagtccttggcgccgtccctcgccacgaaccaggcggtggcgatgcaaccccgggcgatgctcctggcgtccttcttccccgcgacgatgttggacggcatgttcttcaccagtcgctggcacgcctgcacgtccgtcgtcttggggcacatggcctccatgtccaagatgttggggtcggggatggcgccggcgttgggggtggcgctggcggcggagtcgtaggcatcggccgccgcgacgagaacggctaggaggaggaggaggaggagtgacggcattgcgggagaggcggccatgactgggcttagctagctttgtcgatggagatgaattaatggtgatcgatggctaaagaggccgtggttacgccgtttttatataggctaatcgcactgcgtttacgcctgcatggatctaattaataggagagcttcacaagtgtttggttcagtttggtacacttctattttctattttaagtcattattagcagcgaaagtatatccggctgtaaattaattaatgggtacacttagctaagaacaggtaatcgatatcccctcgtatatagtcctttccgattgaacatatatactagtaatcagcgtaaaaccaatttaaaggctatgtttgttagtagttgggaacttttgttttcctccgcacgtaaattgaagtagcttattagcatataagtaattaagtatcagctaaaaaattaaaaaggattaatatgtatttttacatcaactttcttacagaaaacttttacaaaagtgcactaaacagttcgagaaacgtaatcacagaaaccgaggaagcagaagttaggaagagatatgtttgggtttttagtagctcttcgttgaacagacaacatttgtgatatatatatggctacatttgtgttatcttgttcgatcataaaagaacttcatgccaagtaaaagggaaactcatcaggccatgcatccactactcggcctctccctctaacacctctctccttctccctctgctgttgctcaatctcttctacactcgccctctcttcctccctattttagggctcaccctcgcacgctctccagcgacaacacaccaccaccctgcagtctccagcaatggcaatgacaagaagaggagatgtaggaagaccaaatctacggcctgagctcagcgctatcacatttgtcaccaagcacaagaattgatgacaacagtgatgatgggaaagctagggtggaggccgctagatctaactaactccttgctgttctgagcatggtgtgacgtcggtgacaaatgtcactagaatagttatgtgaagtggtaaaaaaaagttcattttttataaaaaaaaattgttctgacagttgaccacctagtattactaatctacgaaaatggatctaataactcgagtggacgtccacccatttagtgcatgtcaactagatagttatgaaaaaaattgaaaaaatataaacaagactcgagctgttaaaatagtttcccaacatctatagtagttttcctaaaaagattaaaaaggacacgcgtatgctcatcccatccatggtcggtgtcgccacctggccagaccactcatcccctccaacattgtcatggaggacagaccaagtgctatcgagaggagatgaccagagatgccgccatcctcgtaccggccatgtgtacctgaaccatgctcgtcgtgccatcagctcgtcaccatccatctcgatgtcatcgtcgacatgcttgccatgcccgagcagctagctattagaaaaagtcgaggcaactagctgccgccatcaacacagccgctcgtcgtcgtcgaggccactccaccaaacacttaagcctgtgcctctcctttgttgaagttgcagcatcaagcaccatctgatccgctaacactaccttcctacttcctccaacctttgccaccttctacttcctccaccctttgccaccgacaccaccccaccatgaggtcagggtcatctggatctcgtggaccatgactattaggaggccaggcctggtcatggttagaaatttcggactgaaatttctgaaattccaatcatttcggacccctctgatatgatattattctggccaaaatttttatttttttaaatttttcatgatttaggtaatatttgttcaaatttaactaaattttgttcgaaatttcggtatatcggtgacctccgatcaaatcagctaaaccaaaaaagtagaaagaggaggtggaagcaagctttggatctagacaacctggtgacaaggacggcgagcttggttgggtgatctcggggcatcaacagcctcaaggaagaggtggaaagctccactatgggaacacaatatgtgcggtagcggtggagttggagcaagaggtgaggccatggcaaagagggcaagtgcgagagggtcattaagcagcagggtgcgagcaagagagaaggtgagcggttaggtagacggggaacaaagatacgtcatccgtgaagatagacagatagatcttatctgcaatgtgtcatatttacaatatatcactgatattgcaaccgtcacagacgtggggccaaaacgatgatcctacttttgtgatggttcatctcccacttcctccatatcttatcttagacagtataatatatcttttggggttatgaatgcctggttctgttgtaatgtgtaggtttgattgccccctcgtcatgaccaccagatcgagcatgagcgcgagcgaaggaattggaattggcaatggcgaggacagatagctcttgaccaactccacaaccaccgtgatatatgtttgtggagtaatgtttcatattaatctatcttgtcaatgtcttttatttttttaatgactatttaggtgactatttaggtggcataaaaaaagataatattccttccacggatgaaaacactttctccaactatagtacatatataaagttttttttacatataaagtacacttatatgttagaaaaatagaaggaaaaaattcaaataccccactaaactttagatgaaagtccgtctagcattctaaaatttgtaatatagattgtattgcaaagtttagggggttagatgtccggttttaaagtttagggtgttaggcggagcttaggagagtatttggactttttccaaaataaaatattcaatgagcccagaaactgaaaatatcgccggcatcctttttaggtaatcgtgcacgtcacaattcacaaacagaagtatacatgtagagttctactccgaatcaaatctaaacttcgaaacccctacaaattgacgcatcgctagctgcaacgccacctattataccggattaggttaacatctcgtgcagccagccagccaagaagtcgtcgatttgtcatggcggcggcgaaggaggacgacacggtcggcggcggcgaagcctgccggtccggccaggcggcgctggcggcgcgcctgctgaagagcctcgcggcgggcgcgtcgggcgcggcgggcggcaacctcatcttctccccgctgtccatccacgtcgccgtcgcgatgatgtcggccggcgccggcgggagcacgctcgccgagatccttgccgtggccggcgcgccgtcgcgcccggagctggaggcgttcgtccgcggcgtcgtcatggaccgcgtcctcgccgaccagtctcccgccggcggcccgtgcgtctccttcgcgtgcggctcgtggctcgacgcgtcgtactcgctcaagccgtcgtaccgcgacgccatcgtcggcacgtacaagggcgccgcctccaccgtcgacttcaagaatcacgtaaatcctctcgcaaatccacctcaaattcatcagatctactctctccgtttataaatatttaacgccgttgacttttctaaatatgtttaaccggtcgtcttattaaaaaatgtaagtaattattaattcttttcttatcatttgattcattgttagatatgcttttatgtatacatatagttttacatatttcatataagtttttgaataaaacgaacggtgaaacatgtgctaaaaaaatcaacagcgtcaaatatttagaaacggagggagtagctaattcatcaacttttgtttgatttgatatttgatgtgtggtggccattgatgatgcagccggtggaagcaaggaaggagatcaacgcgtgggtggcgagggcgacgaagaacctgatcacggaggtgatcaagccggagagccagagcgtggacacgaggcacgtcgtcggcaacgccatctacttcaagggcgagtggctcgccccgttcgacaagtccgacacggcggagcgcgagttccgccgcctcgacgggagctccgtcgaggtccccttcatgcagcggccggcggggagctaccaccacgtcgcctgccacgacggcttcagggtgctccgcctgccgtacaaggcgacgggcgacacctacaacctcaagctccgctacagcctcccgtcgttcgccatgctcgtcttcctccccgacgaccgcgacggtcggtctatcggacctgctcgacaggatcacctcctcgccggatcggagttcgtcgatgaccacctcccgccggagtgcgtccccgtgggcaggagttcgacgatgatctcgggttctcgctgtacgacgcactggtggagaaaccctttgtagtcccggttcgtaacccccctttagtcccggtttccaaaccgggagtaccaatccgggactaaagatcgatctgacctttcccgggtggcatgggaggaagagtagggaaaacaatttggtggagaccggatgtaaaccggcttctgtgcaatcctgaccccgatggttgccgcgtgtggacaatctcagaatcggacgtctagctcgttgcggcttcaagcgcaacgcaaatacggcagatgagcctcgctcgactcggctcccgggccgtcggcttgcaggctcagattcctctccagctctcctaccggttcctccgcagccgccgccaccggcggcaagcggcgcctttctcatctacagtctctctcgtcaccactccctttctcgaacatcgtccactcggcggtgatggcgagggcaaccggctcgctggccacaacaccacaatcgacgacaccggcccactgcggcaacaactccatgcatggttggtcctttcggcaagaaacttaattaccggtaaggagattgagattgtggatgatgacgcggaggatatcactactaggaaaagcattatcacaagcgggccaaaacggtcttcgcaggcggggcggccgtccgcctgtaagccaacggctgtgaagccgatcttgtgaagataggtagaaaatattgaaatttattaataatttgcccattcatagaaattcttgcatgtagatttttatacatggtgagaagcactgcccaagtactagagaattttcagaatttgacatccaatacgcaccgtcttcagcataagccatccaattcgcactactttcagaatacaccactaggacgcgaattttcttcgttttgggcactccgtccacggacgtcagttgttgtcgtcgcccgtccgtctgccgccgcccgcgtccccgactccccgtccatccgccgccgctgccgccgcgtccccgtcaatccgccggcgctgctgcgcgcgtccccatccgtcgagaaagggacgccccctcctcgggtcgccggtcgttgagggagcggaggtgtgacggtgcaagggagggtgagggtgcgtcgtgcccgcggtctccggtgacaggtgactctctctctctcgggtccatccgtcggcgccagcgccgctactcccctcccctcccctccgttcgccggcgctatcgacggatggggacgcgcgcggcggcgccggcggattgacggggatgcggtggcggcggcgctggcgatggaccgggacacgagcggcggcgccggcgatggacagggacgcgagcggcggcggcggcggcggcggatggacagggagtcggggacacgggcggcggcagacggacgggcgacgacgaggactgacgtccgtggacggagtggcccaaaacgaagaaaaatcgcgtcctagtggtgtattctgaaagtaatgcgaattggatggcttatgctgaagacagtgcgtattggatgtcaaattctaaaaattctccaagtactatgtgagctgctcatgtctccaaaaggattaactctatcagtactcagggcaaatctaatgttccgtgggtctttagcaaactctctatactgagcatcaaagtttatccattgacgagtatctgtcgggtgtcgaatcatcccatccttcttgcacccttcttcatgccagcgcattagttccacatctcgcgggtttgagtatatccgtttaatgcggtctttgacaggaagctttttctcggcaggatttttctctgtcttcccttagttatgtcagacggttctaggccagtattgctattcgatttgtaccggctagcaccgcatgttggacattcatctaaagaagcgtactctttcctgtacagtatgcagtgattaacgcacgcatgaatcttttccacactaagtgaaaggagacatatgcttcctcatattgttcttctcagcgtgtgccttagcaataccaataaatttagacacttcgtttctatatggagacaaaaacctcggccaatgatacatccattcccgattttccatatctctaaaaaaatgaacataaattatttgcagaattaataccaaatttgtagggtttattgaaaaaaacgtatcgctatttaaaactaattaactaaagattaattgtaaattagtaaaaaaacaaagtttatttcaatttttttcatacagacatgccaaattagtatattaggaaaatctaacaaaatcatatcatcggcatattacaaactaatataattattttttgactgcaccaaataaatagaaattaaaaattttattctctctctctatatgacaagatctatatctagatctagaactaggaggtggatgtgatagcaaaataaaaaaaaaaattgagaatttatgtttccacattaaaccactacaacaagaggcatcaagtattacacatacatctaacatcaacatatcaaaagaaattaaattaaaaaacaatgagcttttccttcacaattttgcatgcataaatccatcacgcggccgagggaggacggccgcggaaggaagaacgaaggaagagagggacagaggggggggaggaggaaagagggagaggggaggggagagaaagagggggaggggaaagaagaggagaggagagaggagaggagggggagaggaagaaagggggaaagaaaaagggggaagagagaagagaagggggggagagagaggagaggaagagagaagaagagggagaagagggagaagggaagaaaaaagaagaaagaaacctccccactcccccacgtcggccgtcgaaccccgccgctcccggccacgtcccgctccctctccccctttttaagcatcgccgttctcctctctcgctttttccccatttcgccgcactctcccgagctccccatcgcctccgcgccgtgcaaccacccgccgccgctccttcccgctctccggccgccgctagccgccgctaggctcgtcgccgtctcgcgccgtcgccgccgttagctttacggggtcgagatctaactcggccgccccttccccgtcgaggttcgccgccggagacccatccccgtcgtgtgccggcgagtagcgccatggccaccgccttcggccggcgctctcgtcctcccacgctctttcctctctcgctaacccctctccttttgttcgttaggaggctccggagctcgtcccgccgtcgccgtcatcccccgtcgctcgccgtcgtcggtcggtgtcggtgaggtcctcctcctcgttttcctctctctcccgcaccgcttagccgccgcgtcaccgcttagccgcgtagccgccgatctccgccgccggtcggtcgccgtcggtcgccgccttgcgccgctgccgcatgcgcgccgccgccgtgcgagcgtcgcaccgccggcgccatggtcgccgccgtctcgcgccgcgcccgcgccccccggccgcgaggtcccgccgccgtccgatctacccttgggtagatcggggccgtcggttcggccgccacgtggcgccgccgcccctcctttcccccgtgccgctgacccgtgggcccgccagccgccgcccctcctctctcctccccgtgccgctgaccggtgggtcccgcatgtcggcgccgcccccggtgatgacgtcagccctggaattaattgcgcaataatttatttaaggatttcttttatagtaataaacacagtgaatcttctaaaattcataactaattcatccgagctccgtttaatcccattcaagtctcagtaaatcaagaaaaatgtgtagaatccattaaaaatggttttgttctctgttccagtagtctcatagcctgtttgcaatgtttgctttgtatgtcgctagattccgatccctccgacgctccggtgtacttcgagatagtcaccgaagtccctccagcagcagagcaaggcaagtcatgcttgtcacttgaacatattgatcctatattgcaaatgctctattgtttttctttcaaatattgcattgttttataatatcactatgggatggatacctaatgttacagccatgcttttggatatcatgctcctttgttagcttgggttataacatggttagagttaggactaggaattgcttagccatgcttagttcaactagttcacgatggggaaaatcatactaatgtttagactgttggttctaacgataatgttggtttgacgccaccgctctggtgttggttaaataaaatgaaccacatggtgggctgtgggtgcatggttttgctggtcgcacccatggcagttaaggaccggttcgcgggatgccctggaagaatttatcgtacttaccacaagccagcgtgggcaacggctgggcttgtagtgtagctttcctctagtcgacgcatccaggcaagggtgggcgtgatggagcggggcgggccctgcgacggcttctgtcgcttccggattcacctaggcacgagaggggactgcccgtcgcccgctggggacgggggtgaaacctgaggtgtggtgtgcttggctagagggggttatgcgaagggtcctgtcacggcctctttccggtatgtcgtggtggcatgtcggcgcacggaaacgtgtcgtggggctgtgtcttgtgggtacagttgtacacctctgatcagagtaaaactattcgaatagccgtgcccgcggttatgggcggtcaaccagattcaccgtgattagtctcaccttagtgtaatcctttaatttggttagtttaggtggatggttgggcctgtcgcaacgtggtgtagcgttggacagtggatgattaatactgcttaattacttatacaactgtctaaagctttaaatctctgtttataaatgctcgctttatgcaaatgaaccactttagctcttctgtgatgattccctgcatcatacctctattccggtatgacttgctgagtacagtgggtagtactcagtcttgctctacttttcccaacccccagaggtcgagttcgggtcagatgaaggtttctccgaggagtaggcttcgtccgtgccgtcgaggatgcctgtggagtggtgttcccgctgcagtttagtcaaggcttagctccagtttcttttgttttccgctgcatttatgtaagactttataatgtttgtaagacgtggatctgtatgtcaacattgtcgtttgtgtaccccggctggtcctggacggggattttaatgcacattctgcttggaattctattcgggaatttctgggcgtgacaagttggtatcagagccgaccttgaccgcaggacaagccaaatggaaaaacctaagagccctctgaatccttttcattgcatatgttctttgcaattggatttgttccgggaaaattgggatctgttcttgtggttcaagggaaaaatcttggtcgctcgtttaagtgagatttttcaaaacgagtcagtctagggttttagtaaaattattgggatttgttcgtcagtcagttgaggtttgaggggtgaaatgtattaggtcctagctcaggaatttgggttgccaggtatttatgctttgttcgttattattattatgtatctaatttctcattattttatcgtcatgtgagtctgtcttatttattcgctctcatgcaaaatgatctatgcataaaattttgctcttatttgattcccttatttgagtcttttatcgctttatttaaatttggatttgagcatgcattgatcctactcctttgtcttctctagatggccggtcacccacccaaccaccgcggagaaggcatgatcgactttgtggcggagctggcacgcatgacactggtggtgggctaccccgacgcgccggagtacacgaccattccaccgctcagtggcgagcttcctcaccgtgtccgcttggaggtccatggttacgtggggacttgcctcgctaacatggcggtcgaggcatcaggaggcacggcagatcacgcttgtcaggaggcagcctacctgatgatggccagactccgggagcgccacaactatatcttccacgacacggcgtaccgctaccaccccagtcgagcccacggtgatggcgtcagctccttccgtccgacagctggcgagaacgacaccactttcggtcacatgtgtgctgtgatgagaggtctggacaggatgcactcggatctgcacaaggcgaccaaggccttgaatgatgggaagctcatgaggatcgtcgctctgaaggacgagatagcgcgtctgaagaaggagaacgctcagctgaagggtctcccagcgccaggaggagtccggatccgcactacgccccgcaagactacgaaggcacctgtgcgcatccagctcgcgcccaggaacccacctccatccgcagcttccgcagcacctccagttccgcccgcagctccggcagttcctccagctccagctccagctccagctccagctccagctcccgcgttcgttctctccttcgctccagcatcagccgccagaggtccagccagtggcaccggagggtggttgtccgctactcccagtggcagccgcgatctcagcagcagcagctccagtggctcgcagtcaggcagcggcgagacgtatctgccaggctccaggagtcgctcagagggacctggtgacgagcaggaggacgctttcgactccaccgaccgcaggagccgttccgagcattaggctcgtttttcccactagtttaccgactgtctagcttttgttcgttagtttgtgtgtttaggttgtttgtttgggtcagtcgacggtcgatatcatgtgcctatggtatggctgtcttaataaggattggctgcttgctgttttaagtgttttaattcagatcagaacaattgcagtttaaattccttggtaataaagcttagttcctgagcatctgtttttcttcttctccccgtctgctcttccctccagatggtctacaccaggactggcaaccgcacgacaggcgagggcagcaacggtgaagaacgcgttgatggtatgcaccccaacggcgatcccggcaacggtccgccaccacttcccgagaatccgacacttgcccaagttatggcgcatcagactcagatgatggcagccatgatgcagcagatgcagcaacagcaccagcagatgcaccagaggatgatgcagtacgctgaacagcagcaccagcagtttggtccccctccccctcagtccaagctgcctgagttcttacgtgtcaggccacccaccttctccagcaccaccaaccccatggaggcaaatgactggctccatgcaatagagaagaagctgaatctcctgcagtgcaatgatcaggagaaggtcgctttcgccacacaccagcttcagggtcctgcgtcagcttggtgggacaaccacatggccacccgcccaccgggtactgaagtcacgtgggcagagttctgccgtagcttcaggaaggcacaggtgccagatggggtcgtggcacaaaagaagagggaatttcgcgcactccatcagggcaacaggacggtgacagagtacctccacgagttcaatcgcctcgcgcgatatgcaccagaggatgtccgcaccgacgccgagaagcaggagaagttcatggcaggcctggatgacgaactgaccaaccaactaatatccggggactacgccgactttgagaggctagtcgacaaggcaattcgtcaagaggaccaacgcaacaagatggataggaagaggaaggccgctcagttcagggcacaccagggaagtcaccagaggccgcgcttcactcccggacagcagggtggacctaccaccatgatcgtccgtcagcaccgcccgttcaaccctagcaacttccaccagggcaccagtggcagtcagaaccaccatggaggtcagcccaaccgcagtgcagctccacgcccaccgacggcaccggcacagtcaggccagcccgcgcaagccaagaaggagacgggagcaaaaccagggtcctgcttcaactgtggcgagcttggccacttcgcggacaagtgcccgaagcccaggcgtgccgggccaaggtttatccaggctcgtgtcaaccacgcatctgcggaggaggcacaagcagcaccagaggtcgtactgggtacgtttcctgttaactcaatccctgcaacagtactttttgattctggtgcaacgcattcgttcatctcaaagaaatttgtcgggatgcatgggttaataagagaggaacttagcacaccgatgcgggttcataccccagggaatagctccacttcggttcaattcagcccttctataacaatagaaatccaaagatcaccatttctagccaacctcatccttctcgactccaaaggcctagatgtcatccttgggatggattggctgaccaagtttaagggagtcatcgattgtgcgaatcgcacagtcaccttaaccaacgagaagggagaaaccgtggtgtacaaatccctagtctcgccaaggaaaggggtctcattgaatcagattgagacggaaatcccaggggacaccgtagagaagaactcgagaaaattggatgatatacccatagtctgcgagtatccggaagtgttccccgaagacctcactaccatgccacccaagagggagattgaattccggattgacttggcaccgggaaccgctccaatctataagagaccgtacagaatggcggccaacgaattagcggaggtcaagaagcaagttgatgaacagctgcagaaagggtacatacgaccaagcacttcgccttggggcgctccggtcatttttgtggagaaaaaggataagacgaagaggatgtgcgtcgattatcgcgcacttaacgaggtcacaatcaagaacaaatatcctttgccgcgaatcgacgatttatttgatcaactgaaaggagctaaagtgttttctaagatagacctgcgatcaggctaccaccagttgagaatccgggaagaggatattccgaagacggccttcaccactcggtacgggctatatgagtgcacggttatgtccttcggacttaccaacgcacccgcattcttcatgaatctgatgaacaaagtgttcatggaatttctggacaagtttgttgtggtgttcatcgacgacattttgatctactcaaagtccgaggaagaacatgaacaacatctccgtctagtactcgagaagttgaaggagcaccagttatacgccaagttcagcaagtgtgacttctggttgacggaagtcaaatttctgggccatgtcattacagctcagggtgtggcagttgatccatcgaacgtggagtcagttaccaagtggaccccaccgaagaccgtgtcgcagattcgaagttttctaggactggctggttactaccgccgattcatcgaaaacttctccagaatcgcccgacccatgactcagttgcttaagaaagatgagaagtttaaatggacagctgaatgcgataagagctttgaggagcttaagaagaaattagtatccgcaccggttttgatcctgcccgaccaaatgaaagacttccaagtctattgtgatgcatcccgtcacggacttggatgtgtcctaatgcaagagggcagagtggttgcatatgcctcgcggcagttgcgtccacacgaaggaaattacccaactcatgacttggaattggcagcggtggttcatgctttgaagatctggaggcactatctgatcggcaaccgctgcgaagtatatacggaccataagagtctgaaatacatcttcacccaaccggacctaaaccttcgacaacgaagatggttggagctaatcaaagactatgacatgagtattcactaccacccgggtaaagccaatgtggtggcagacgctttaagccggaaaagttactgcactgccctatgcatcgagggcatgtgtgaggaattgcggcaggagtttgaacatctcaacatgggaattgttgaacacgggtttgtagccgctctggaggcacggcctacgctcgtggatcaagtcagagcagctcaagtaaacgacccggaaatagcagaactgaaaaagaacatgagggttggaaaagctcgagatttccacgaggatgagcacggcacaatctggatgggagaaagattgtgtgtacccgacgacaaggagttgaaggatctgatactcaccgaagctcatcagacccagtactccattcacccaggcagcaccaaaatgtatcaagatctcaaagaaaagttttggtgggtcagcatgaggagagaaatagctgaattcgtcgcactctgcgatgtttgccaacgagtgaaggcagagcaccaaagaccagcaggtttactccaacctcttcaaatcccggaatggaaatgggaggagattggaatggactttataacaggactgccccggacatcatcgggacacgattcaatctgggtagtcgtggatcggctcactaaggttgctcacttcataccagtacacaccacctatgcgggaaagagattggcagagttgtacctcgcaaggatcatgtgtctgcatggggtacccaagaggatagtatccgaccgaggtagtcagttcacctcaaagttttggcagaaactgcaggaagaattggggacccgcttaaatttcagcacggcctatcacccgcaaactgatggtcagaccgagcgagtcaatcaaatcttggaggatatgctgagagcctgcgctcttgattttggtggagcttgggacaaaagcttgccatatgcggaattctcgtacaacaacagctaccaggccagtctacagatggcaccgttcgaagcgttatacggccggaagtgccgcacacctctcttctgggatcagacaggcgaacgtcaactgttcggaacagaagtattagcggaagcagaggagaaagtcagaatcatcagggaaaggttgaggattgcccagtctcgacagaagagctacgcggataaccgtcgaagggagctcacttttgaggcaggagattatgtgtatcttcgtgtcactccgctccggggagtacaccgttttcagacgaaaggtaagttggcaccacgcttcgtgggaccatacaaaattttggaacgaaggggagaagttgcttaccagctagaacttccatccaacatgatcggcatccatgacgtgttccacgtttcccaactaaagaagtgcttaagagtacctgaggaacaggctgattcggaacacatcgatatccaggaggacctgacctatgtggagaagccggttcgcatacttgataccagcgaaaggaggaccagaaacaaggtcaccaggttttgccgagttcagtggagtcatcattcggaagaggaagcaacctgggaaagggaagatgagttgaaagccgcccatccgcacctcttcaccagctcctccgaatctcggggccgagattccgtttaaggggggtaggtttgtcacaccccgaagttttcctccctagccaaattttaattttataagtgaccctaagaaaatgctggagaaatcaggagaaaaccctaagaaaaaaatgcaaataataatcggaattgacatgtggaatttttcttgagttctacgtgtcgaaattcattaacaggatttttagtggaattttcagagctcgagaaataattgtaaccaattaaaaatcaccaaagtgcaatttttaattccagggaaatccctttttcttttttctttttctttttcccctctttttcctcctttttgcgaaatgggccgtcggcctatttcccttttacttcccccccctcctccttgctgggccggccgcaggccgaggcccagcccagcagccgccccgcccgcctcctctcggggtcgccgacaggtggggcccacctgtcgggcccgtctcccacctcccgccgatcccggcgcgccgccgcgccgaaaccgccgccgcgccctcgtctccgcctctcccgggccacgtcggccacgtccgcgcgtgcgccgagtcgtccgcgccttttcccctctccctcccgttcgcgcccgcgcccgagatggccgggattcgaaaatcgaatcccgcctctctctcctccccactcccccacgtcggccgtcgaaccccgccgctcccggccacgtcccgctccctctccccctttttaagcatcgccgttctcctctctcgctttttccccatttcgccgcactctcccgagctccccatcgcctccgcgccgtgcaaccacccgccgccgctccttcccgctctccggccgccgctagccgccgctaggctcgtcgccgtctcgcgccgtcgccgccgttagctttacggggtcgagatctaactcggccgccccttccccgtcgaggttcgccgccggagacccatccccgtcgtgtgccggcgagtagcgccatggccgccgccttcggccagcgctctcgtcctcccacgctctttcctctctcgctaacccctctccttttgttcgttaggaggctccggagctcgtcccgccgtcgccgtcatcccccgtcgctcgccgtcgtcggtcggtgtcggtgaggtcctcctcctcgttttcctctctctcccgcaccgcttagccgccgcgtcgccgcttagccgcgtagccgccgatctccgccgccggtcggtcgccgtcggtcgccgccttgcgccgctgccgcatgcgcgccgccgccgtgcgagcgtcgcgccgccggcgccatggtcgccgccgtctcgcgccgcgcccgcgccccccggccgcgaggtcccgccgccgtccgatctacctaaactatccaaattaaaggattacactaaggtgagactaatcacggtgaatctggttgaccgcccataaccgcgggcacggctattcgaatagttttactctgatcagaggtgtacaactgtacccacaagacacagccccacgacacgtttccgtgcgccgacatgccaccacgacataccggaaagaggccgtgacaggacccttcgcataaccccctctagccaagcacaccacacctcaggtttcacccccgtccccagcgggcgacgggcagtcccctctcgtgcctaggtgaatccggaagcgacagaggccgtcgcagggcccgccccgctccatcacgcccacccttgcctggatgcgtcgactagagggaagctacactacaagcccagccgttgcccacgctggcttgtggtaagtacgataagttcttccagggcatcccgcgaaccggtccttaactgccatgggtgcgaccagcaaaaccatgcacccacagcccaccatgtggttcattttaattaaccaacaccagagcggtggtattaaaccaacattatctcataaacttatagtctaggttttaaatgagattccccattgtgtgctagttgaactaagcatggctaagcaattcctagtccaaagtctaatcatgttataacccacgctaacaaaggagaatgatatccaaaagcatggctataacaaaaggtaaccatcccatagtgatattataaaacaatgcagtatttgaaaggaaacaatagagcatttgcaatataggagcaatatgttcaagtgactagcatgacttgccttgctctgctgctggggggacttcggcgactatctcgaagtacaccggagcgtcggagggatcggaatctagcgacatacaaagcaaacattgcaaacaggctataagactactgaaacagagaacaaaaccatttttaatggattctacacatttttcttgatttactgagacttgaatggacttaaacggagctcggatgaattagttatgaattttagaagattcactgtgtttattactataaaagaaatccttaaataaattattgcgcaataaattccagggctgacgtcagcgcggggcggcgcggcgccgacatgcgggacccaccggtcagcggcacggggaggagagaggaggggtggcggctcgcgggcccaccggtcagcggcacggggaggaggatggggcggcggcgccacgtggcggccggatcgacggccccgatctacccaagggtagatcggacggcggcggcggctcgcggccggggcgcgcgggcgcggcgcgagacggcggcgaccatggcgccggcggcgcggcgctcacacggcggcgcgcgcatgcagcagcggcgcaaggcgacgatcgacggcgaccgaccggcggcggagagcggcggctagaggaggagcggcggctacgcggctaagcggtgacgcggcggctaagcggtgcgggagagagggaaaacgaggagggggacctcaccgacaccgatcgacgacggcgggcgacggaggatgacggcgacggcgggacgagctccggagtgccctaaggaacaaaaggagaggggttagagggagagaggagagtgcgagagagcgagagcgccggccgaaggcggcggccatggcgccactcaccggcacacgacggggacggctctccggcgacggacctcgacgaaagaggggtggacggggtagatcttgaccccgcgaacctaacggcggcgtcggcgcaagacggcggcgagcctagcggcggctagcggcggccggaggaaagggatgacaggtggggtccacgggtcccacctgtcggagggagagagagaggggagaacgagagagggaggcaaagcagacttcgcggggaagtggggaaacgggccgacggcccaagaggagagggaggaggcgcgcggcctgcgggaggggaaggaggggttgggccgaaaatggcccaagggggatggagggagattttatttgttttctttttttttatttgattatatgaactttgtattgttatagttatttcttgagccccgaaaattcacggaaaattccggagagtactttagggcacaaagaatattacaaaatattcctggccatgacttttaaaggaaatttcaaattccctcaatatttcacttgattaaattgctttaacttttatttaatttctagaaaatgcattattaaatgatttttaatcccgaacgaaaatcggggcgttacacggatggacagggactcggggacacgggcggcggcagacggacgggcgacgacgaggactgacgtccgtggacggagtggcccaaaatgaagaaaaatcgcgtcctagtggtgtattctgaaagtaatgcgaattggatggcttatgctgaagacagtgcgtattggatgtcaaattctaaaaattctccaagtactatgtgagctgctcatgtctccaaaaggattaactccatcagtactcagggcaaatctaatgttccgtgggtctttagcaaactctctatactgagcatcaaagtttatccattgacgagtatctgtcgggtgtcgaatcatcccatccttcttgcgcccttcttcatgccagcgcattagttccacatctcgcgggtttgagtatatccgtttaatgcggtctttgacaggaagctttttctcggcgggatttttctctgtcttcccttagttgtgtcagacggttctaggccagtattgctattcgatttgtaccggctagcaccgcatgttggacattcatctaaagaagcgtactctttcctgtacagtatgcagtgattaacgcacgcatgaatcttttccacactaagtgaaaggagacatatgagtttcttcgcttgatatgtgctggatggtagtgagctaggcctcgggagcattttctgcagaagttctaacggactgtcgaaactggtatcagaccatccatgtctcgcctttaacctcataagttcgagaactgaccgtagtgttgtgaactcactgtcacagtccttcgactcatcatacaaaacttccttaactgccttctgtaaagcttcaaagttatttaaacctctagccgaccccatcagcacctcgggttctgcatgacgtaacatttcctctaggtcgattttgtcctcaacagaaccatcgtcatgcaccatgtcttcaacttgtgttagaacaacattttcaggcccgtccacttgttcttcgccatgacgtgtccatatttcctacttgtccataaatccacgagttactaagtactcgttcattttaaaagcatcgtcgaagtcccaggctatttaattcttacagtcagcacatggacaaattatcttcctcatattgttcttctcagcgtgtgccttagcaataccaataaatttagacacttcgtttctatatggagacaaaaacctcggccaatgatacatccattcccgattttccatatctctaaaaaaatgaacataaattatttgcacaattaataccaaatttgtagggtttattgaaaaaaacgtatcgctatttaaaactaattaactaaaggttaattgtaaattagtaaaaaaacaaagtttatttcaatttttttcatacagacatgccaaattagtatattaggaaaatctaacaaaatcatatcatcggcatattacaaactaatataattattttttgactgcaccaaataaatagaaattaaaaattttattctctctctctatatgacaagatctatatctagatctagaactaggaggtggatgtgatagcaaaataaaaaaaaattgagaatttatgtttccacattaaaccactacaacaagaggcatcaagtattacacatacatctaacatcaacatatcaaaagaaattaaattaaaaaacaaggagcttttccttcacaaattgcatgcataaatccatcacaaattgaggtctaaaagcttaaaaaattgcatacctagtgtggaaatgagtagatctaaacaccgtagaaaaatccatgcggcgcgacgtcttcgtacgtcctcagccagggcggtcgccctcagccactctctatcgccgccatgcggacgacggacgagagaggctgcatcggcgacagtggctgctgctgagaagagatgtcgatgtgatctgagccggtgagccgacgggcccagagccatcgagcgagtcgcgactcctccatcctgtttgcgctgcgcttggagccatgcacagcgagacgttcgatcctaaggttgtccacgcggcaaccatcggggtcggggctgcacgggagccggtttacacccggtctccaccaattttttttccgaagaggagtgtatagactatagagaggaaagtgatttgcttcctccgtttcacaatgtaagactttctagtattacccatattcatatagatgctaatgaatctagacattatatagattcagtagcatcatatctatatgaatatggacaatgctagaaagtcttaccttgtgaaacggagggagtatttattatcacttcatggaaattatctttgattttgcatttcacttcaaacttcaaaggtggcacgatgcatcaacatgtggcatacaggattcaaaatttcggattttggaaaattttggttctatcggaccccccccccccccccgatatgatattattttggccgaaagttttatttttttcatgaatttggtaatattttattcaaatttttaaaaaaaattcatataattcggaccgaaattttcaccctacaaatgaccaaaccgaaatattaaaccctggtggcatatcgttacataaaacatgcaaactcaacatatgtaaataataataatattcagttcaaacagtacgtgttagtttggtcgaattcaaatatggattgatatatcttataatgggtctattatattaaaaaaaaaagagttgttttttaactttttggttgacaacgttccctagaaagaaaaaattccacctccgcctatatagatcgagtgttatagaccatattgcatatgagaagaggaaaacaaattcctacaaaaatgtttctgcgttttttttagataaaatgtttctgcatgttctagacacacggttgttgcttaatttagtgcttagttatagtaaagaatagcttctctacttattagatgaaaacagatatataagttgagaattgaaatttacaggtgatgaatatgcatggttttttttttttttttggaaactatgcatggttattcagattgcagagcttataggtgaagtcgtaaatatgttgcattgatgacaaacttttttgggcaactgaaacatcttttgccacagccttggcgaatacttggaccaaattcttcaatttttgtttgctgctttgccacaaaattttagttaacggctatggttgattgaaaaatttcatccaatattttgtttaggaaacattaggcacatcagagtatcaacttagaccttgttcgattaactccttacaaacattgctgtagtcaaaattttgtaaggattttttattggtgtagtaaaaattttggaagagttatttaaagttcgtatagcatcctaaaaaatattaagtaccgaatgtttgtaattaaacataagttgtcggcggggccgcaggcgcagtgtcgacgacaggagctgatccggctgccgaagaggtctccttctcggcggctgatccggccgctggataggtgttcttatcggcggttgatccggccaccgcagaggtctccttgtcggcggttgattcggctgctggagaggtctccttatcggcggttgatccggctgccgcagaggtcgccgtgtcggcggttgattcagccgctggagaggtctccttatcagcggttgatccggctgctgcagaggtcgccgtgtcggcgattggttcggccgctggagaggtctccttatcggcagttgatccggctgccgcagaggttgccgtgtcggcggttggttcggccgctagagaggtctccttatcggcagttgatccggctgccgcagaggttgccgtgtcggcggttgatttggcggctggagaggtctccttatcggcagttggtccggctgccgcagaggttgccgtgtcggcggttgattcggccactggagaggtctccttatcggcagttgattcggccgccgggtaggcgtttttgtcggcggttgatccggctgccgcagaggtcgccgtgtcgacggttgattcggccgctggagaggtctccttatcgacagttgattcggccgccggagcggtctccttgccttcggttgatccttttgccgcataggctaccttatcggcggttgatccggctgtcgtcgtggccttggtccccgtcgtcttcgtcaaaggatacaaatcgtcgagcaccgcatcggtgaaatccagaaagtcccacatgcgcgagaacaccgagacgacgttgagcttcttggcgtccttgcacgagccgtcgtcgcagcacgtcttgcagagaggcgggcggctcacgccgccggcggggccacggagcagctgcgagaagctggcgtggatggcgaggagccattcgtcctcggggaccttgacggtgttgacgccggcgccggagggcttgagggcgtcggtcacggcggtgctagcctggacgcagctgtcgaggcacttgtcgggcttcttgcactcctgcacggcggcgatggcgtccttggtgaactgctccgccgagaacccgacgctggagatgcacagcttggcgaggcccttggcgtctttcgtctctgcaaactcgggcggcaggaccttcaccagttgctggcacgccttctcgtccgtcgtcttggggcacacggcctccttgttgaagatgttggggtcagggacggcgcccgcggttgatccggccgctgcagaggtctccttgtcggccgccgcagcggtctccttgccagcggttgatcctgttgccgcataggcctccttatcggcggcgccggcatcggctgccgcgacgaggacagcatggaggaggaggaggaggagtgtcgccattgcgggagaggcggccatgactgggctagctatagctttgtcgatcgatcgagatgattttgctgggatgaattaagaggccgtggttacgcgttattatataggctaattgaatagcgtttttcgcgggtatggagtggtatttggcaggagagcttcacaagtgtttggttcagtttggtcgtcagtagcctccgttcattcagagtacgtgtttgctcctctcttgttttctattttaagtcattattagttctatttttagcgagtagcgaaagtggccgtaaattaattaatgggtatgcttaccacagaataggtaatcgatatcccctcatagttcttgccagttgaacccaaataatcaagcaccagttaactttttttttctttgctcaatagcttattaggacgtaattaattaattattagctaaaaacttaaaaatggattaatatgtatttttacaacaacatttctttagaacatttttaaaagcacactatttaataatttgagaagtatatgctcaagaaaaacgagcggaaggtgggaagagggaggttagaatatagtcttaacttactgccttgttatatttgggtttttttttacacatttgtgaaatttcatggtagctttttttaatgggttaattagattcatgccattataaatttgtcagttttgaaatataccgttagcattcaactatttgtaaccataccattataatttcgtggttatttggtttatgccactacctactcctttattgtattttctaaactttttggacaaaattaccccatcttcttgaacctctcctctctactgttcatcttccccatcactgcatctgacaaggtcatttggcacaacggcctgcgcagtgctgtctgacgctccgaattctaacggcagatgcgcacggctcgacccaatcagggagcaacagggaggcatcgccagcgatgggatttgagtctttgtcttcgtcgtggtcttcaagcagcaaactagccccagcaagctcattcgttgatcccttcatcttcaagcaagcagcccgtggcaaccagcagccacatattgagcctccatcaggtcttcggcgcccagtcgccgccgtcgtcgtccacaacggcaagccgctccgtccgcagcggctctagatccaggtctttgcatcgtaagcaccgccgctcgtcgtcgtgggtccccttgccgccggtgcagcacaccgctatttttggcctcctgtcgagctagcagaagcatcaaacgtccttgccatctatcttgaatcgatctagagcagcagcagcatgagcagagaaagatgacaggccggatgagggaggagagggaggtagaagatgagggtattttcgtccaatgcatgtaaaatacatatcataatggtatcactgagattacaaataaacaacaatggcatcgttacaaatacgcaaataataatggcatattttcaaaaccagcaaatttttaatggtacgtatctaattaaccctttttttaaaagcaaaggcatgatgttattctgttaataaaaaatgtttattaactaattacttcgtgtcaagtaaaaagaaaattcatcccgccatccacaactctgcctctccgtctaacctctctcacctagtatccttctccctactagttagcgtgcacgtgcaaggcacgtcgacgtatattgaatagaataaatcgaaacaaatacatccatcgcttaaatcaattttgaccatacagttcatacttctagggataactcaatgcgattagtgcatcaaatgcaaattatgtgcaaaagaagagtttttttgggcatgtcatattgagcacaaacatcctgtaaaaagcacacaacattatgttctaattaatttttcttcaaataagaatcttattgggtaaatataactcatagaacaataatttacagtgtttccttaaacttattgaacccatcaggtaaaataaataaaatatctgtccacaatgcataagtaatcttgctcctataagtatgagctcatagctccatagacaacatggcaatcattgaagtagatatgaaggtaagatgatcttttggcaggtcatcatgcattaacatcttccagaaaaagacataaaatctgtcttcttagactatatcagcaaaggcacacagcttccactttgtaccttgtttttttacataatgatctagactgacaattttatgatactaccagtctcgtgtccaagtttgtcataatatgtccatgcacaataccacatcccactggaaatacataattacactcaaatcaaattataactgtatatatcattagctggtgagcttacctggagaaaagaacagtatcgtgtcactttcctggtgagcttagctggtgaacattaaattatgcaaacaatctatgttgttctcagaaacaagaactgaccgagttccgagtgacagaaactgcaaggatgaagccaaccaattatgcaacaaatggtggcacgagcgataaatttcataagtaataggtgaaaaaaggtaccatgaagaaagttatttaataaaatgaatgcacttttcaataagtagttagacaatgtggcattctgagaggacaatttcagattccccaggctgatgcacccaaaaccaacaaatctagccatggtgggtgtactggcttgtagcacccgtatttttattttcatttatttaaaatcttgtcatttaaaaatttggagttaatttggttcttaattgttttttgccaaatagatctcaaatcctctcttagaattgcctttaagatcccatgaaaccctaggttaaactcaaattcaaatccctcccaaaattctccccaaaattcaaactctacaaatctccggaaaattgtgaaagctcactgttcgaattgggcctccttctctcttctctctcccaggcccaactcccctccatccctcctctcttcagctccccgagcaggccggcccaactcgggccactactgtttatctccttcctcaggccagccagctcacgcacgccaagtgttcgacgaaatgtctcactgagtcgctgcactgtttctccatcgtgaccgagccgcattgcaccgagccactctcccgtgcgctgcgcgtcgtgccaacatccacgccgcacctcccccgcaccaaactgacgcccgcgccgcgccaaagccgcgtcgtgccaagcccgaccgcgctccggtttctctccaggagacacggagccacgccgccgcatgcgtcatcccctgtcccgtcgccgtccatccgatgtcgcgggcatcacctcgccgtcccgtcgcccgtcgtgtcgacgccaacggataacggccgccacctcgtcctccatcaccctagccctaggccgagtcaccctcggtctacccttatccgttccgtttgcttcgtgaggaaggagcttagcctccgagctgcgtctctctctctttctctcgcgtcttgtcgccacagccgcatgacgccgctcccgtcgcctcacgccgtcaccttggccatcgcgtcgccgcctcacatccgcggacgcgcgtccctttgccgtcgcctcgtcgtcttgtcgccatctcctcctgggaagccgccgaaccgcgtctctccctctatctcgcgtcgaatcgcccccgccgcatgatgtcgcccatgtcacgtcgctctgagagccctaccccttgaatcctggccgtagccgtcgtcggccgcgcgtcacccatgttccgccgccgttcccgctctccgcgcgtcgtctcgcatcgcgtcgcgccgtcgccgtcgccatcgcgtcgctgcagtcgcatcgccgcgttccgtcgctgtgacctcgctgacgccacgccaccgcagcgccgtcgcatcgacatcccctccgtccgggaatccggccgaaacagctagccggctacccccggttctcccgcgtctagctgccagcaagcgccgcaccgccccaacccgcgccgcctataaaaggagagctgagccccatctcctctccacaccatcaccatctctctcctctctctttctctctcattttgctctctgtgccgccgcctagagccgccgtgccctgccgtcgagtgtcgccgtcgccggtggtgcctcgccggagcttcgccggaagtcgccaccgccttccccggccgccggtcgcctcgccgcatcccgccgccgcagcgcgtcgtcgtgctgcacccgggccgagtcgagcaggcgagaggaggaagaagacctcctccaagccactgacaagccgggcccgcgccttctttcctccattggccgctgacaggtgggccccctccctttgctgttagtctagcggaatattcccttgccgtccaataaataaattccagaaaatgcttctattctttgaaaatccgtagaaaatcaaccgtaactccgattgactccgttcaagttcctaaatttttctaaaattgagatctacatagtagcaccattgtttctctggtttgagcttgttttattgccttttgtttattagctttgttttcgcgttaagagcccgagttcgaagaagccccggacgccgctttcgaaggagaagaccccaaccctgtgcaaggcaagcccctttgatcacattgcccctatattttcaatgatatgttgtttgcaaagatgcattgtttagatcacacgattaacatgtttaggcctcggattgtttatacaaccgacggaccaatctagtagtcgcacttatattacataggttgatgtttccataactttgatagctctacaaatgtttgcgccggtatcgttacacgattaccgaggtagtttcgcttagccatgcttacgttgttgccgtgtgggaaacttgggttattccaaatacatgtttaaaaatccggtacgttgtaccgaggagttgtgaaataaaatgtgttgtgaaagtggtgatgatgcggagtcatgcctttgtggtttggctacgtattgtcgttaaggaccgattcctttgggaaatccatccgaacacgttacagtgcgaccacacgggtattatgggatgcctctggttaagtaaattgttatcacatgggagttggtgtgccaatggttgtggaatgccgacgatgaggagaagaagtgttacacatttatttgcccatcatgggtgtctttttgcctctattgtggcgtggtaccagtaggagtaggaggcatcctcggtatttcctgagtggatgacagctgttattgcctaatactcggctatggatcttagcccaactaggtggcatgcgattttcgtgggttagctttggaaaggccttatcacgaacttccggtaccggccagtaccggtgtaagtttcgtgtcgtgtgggtaaagcgtgcaacctctgcagagattaatgaactgttcgaacagccgtgcccacggtcaagggcgagtgtaaggtgtttctcatagcgtagatttgtttgacaaaggtttgtggaaaagtggttactgagaagggtaacgttaccagagtccgcctgtgtggcagacagctaacctgggtggttaggaacgggtctgtgtgacctggggtatcgtccgacactgaggtgttgttagggccatgatgatggcgatgttgtgctgggataaccaggtgttttgtgtggattagtgttgttgtggaaagtgagaaggaaccaagaggaaacaaaatatttgttgttcgttaaccgtgacaagttgttgcaaagcaacttgtggaatgggtcgaggcgtgagcctcctcctgacaactaaaacttgactcactcactaggagttttgtgaaataaatcattttgccagaaaggcagctttcgcaaataaaaccgtagcctccttgttaaaccgacatgtcatccttttccccaacttgctgagtacgttaaagtactcacccttgcccttataataatatatagagtttgaagatgaagatgtcgacccgaccgccgaggagtatctccaggagcaagccgagtatgaagacttctagaagtttcgtgcctagccccaagcctcgcctgtgggataagttagatgcctaggttcttccctagtgcatggttgcttagtcgcttagtgctccgcgctgttcgcttctgcgccgtcgcttttatggttttctgtaatgttgtgttactcatctatgggcctgtccatgatagcaacttatgtaaggactactacccagtgatgtaataaagcagtttactttgattccacatttattctgtgtgtaccagctagtgtatcctaggactggtactatatcacaggtaatgcataacaccccggtgtgatttagctgtgttaatggctaatgaatgtcggggcgttacaatcatccatgatatcatcaagcagaagaaagaaatcttgaagctacccagactttctctatacatcaagcttttatgtacaatcagctcattgctgaaaaaagagagagaatagatcaatcaagaacagagaaatcaaacaaaatgatcctttagtagccaagctgcaaaacatatgaatgaaaaatttttataatcacaatcacagtcaaaatttacaaactacctttattgaaacatataatctgtttataagaaatccacgtgaatatatatatatatataagcaaaagtaacgaaataatcactacataattattttaactatagaaaggaatgaaaagggaccaaagcaaaggtcaacttgttagcctcattgtacttaatcagctccgtgccgaagggtggttttctgatgtcctcttaagtgcagagcatctgcagaaacaaattgtgcatcgtgatcatgccaatgcatgtttgaatgtttagctacagagcaatgatagtgtacgactccaaaaggtaccattgtggcgttgaactgtccttccatcctcacaaatccagtctggtatggccagaacagtttgtgctcttcaccctacaaaattcagaccatcattttaaacaaacaactacatcctaaatttatgaattattgatcaaatgaaaatcaacaagttgatatatttaactgaaaagttagtttactctggaccaagactgtacaattcaaattaattaagcagagagcgacagatcatagctagctagtcaaggatcgatgtgacgaatgtttaactcaccgaagaatcgatctccacattattgcttatatcgtgtggcaatggtcgcggcagtagcagcggcggcggagtcggcgacggcggcactggtggcggcgaagtcggcagtgacggtagtggtggcggcggcgaagtcggcagagacggtgacggtggcgccagcggagtggatggcgattcatccatcgtggctggggcagtttgttttggtgtagtcgcggctaggggagtaatcgactgcgaggtagtctctgtgtatgtattgtggcggcggtgaggcggtcctgcgggcggggcggcgtggggatggcgcggcgcgcggtccggcgggcggcgcggcgtggggatggcacggcgcggcggtctggcgggcggtgcgccgtgggttaggtccggcgggagtgcgcgttaatttctttcgcagcgtttccttttttttcttttgttgggtgcgcgcggtgcagttttttttcttagggataacaggtgggttccacgcgatagtggatctgaaggattgcaggattggggatgaactcgtactttttatattagtatagatgcgcgcggtgcggtttttttcttagggatgacaggtgggtcccacgcgatagtggatctgaaggattgcaggattggagatgaactcgtactttttatattagtatagatgttgttgctcaatcttctgcactccccctcccttcctccctattttaggactctcacactcctctccagcgacaacactgttacagccactgttcaccatacggcagcagacaatctgatcagacggaagccgcgttcatcccgtgtgcgtcagcatagcgagcaaaattgcttggcccgaataggttgaatttccaactgtatccggtgcatctatataagcctggatgaagagaggagaggggtatgaaaactgtaagtgttaaatatgtgatccgaattttgggtccacaatatattcggattagctatctaagatgactctactttatagaattagttttctattaggactcttagatttctcctatatatcccttgtaagttgcatggggagggtgcgacagcccaatgtatcccctgcgtttgtatccaattcctctatagcgatcattgtcggtcggcgtccgtggttttttcttgcaagggtttttccacgtaaaattcgtttctccgttgtgcatgtattttcataacaagtggtatcagagcattggaaattgatctactagctcctggtgaccggcatattttcccgatcgatttttgatcttatcagcgacgctgctgtgttcatccgatcaggagatcgatctactacccggctgcagtcgcgttcatccttctgccggatcaacctgtcggcggagtccatctgtgcggcgttaatctgcatcgttgtgccgcgcacgtccccaaggaaaaggtttggcagcagctgcgcagccatcaatccgccgagaagacgcaacaaagcggcagcagcgagaagaagcagcacacgcgcgcccgcgcaagtcaggcagccagccatcgtcctgttcaagtgaattcaatttcacgtttagtatttgcttcacgcacacgtcattcataccaggagattttcagatcttgctatgcgcacacaatcaagtctaccaggaggttttatgcggagatcaaatacttcgttttatgcacctagagtgtttcgcatttctgctaaggtttgacaaattgtaccagcagattcaggattttattcccaaggcgagtttgaagtttgatctaccgctcctggattgagatgcaatctcgtcatggcaagtgaagatgcaagttggcgcatgtttcaaggaggataagtcgtgcacctgagattaaaaaaaaaagtacattcaagtcgtcgagagggagcctcgctcatgtctagatcacaaggtgatcaaattgtccggcgaattttgttaccattttacccaacataccaggaggttatcaggggatctaagtacttcgttttgtgcacaagaatttttcacgtttctgtttggattccagaaattaatgccagcagattcaggattttattcccatggcgagtttgaagtttgtgcatcagtttcgcgtctctgctgggttccacaatgtcatactagcagatttaggatttgttcccaatggcgagtttgaagtatgatctacctcttctagattaagggtgttatggattatattccaagtgactatttggcgcacattgattttatcatcatgaggttgtttggagattgaagattttttatgctacaagggaaattcgtatcaaggtggagattgttaaatatgtgatccgaattttgggcccacaatatattcagattagttatctaataggactctactttataggattttataggattagtttcctattaggactcctagatttctcctatatatatcccttgtaagttgcctggggagggtgagacagcccaatgtatcccttgcgttagtatccaattcctccatagttatcattgccggtcggcgcccgtggttttttcccgcaagggtttttccacgtaaaattcaattcgtgtctctgttgtgcatctattttcataacagtaagagcaattctacggtccttgaggaggtaccaaaaatttagtgtaaaattgctcaactaaactacttcccaagaggtttagtacataggtgtaatattgtgtaaaattgctcataatacctaggtaccaagagataccaaatttacaatagaaaaaatggtagctcatggtacctcctcaaggacaataaaattgctcaactagactacttcccaaaactcaatacatctacctcttctccaaggcactatactctgttcttcgtgttcttctttatcctgccaatttcccctccaatctctctctctctagatctcccctacatggaaattatctttgattttgcatgtttttcaaaggtcataaaaaaattgacacgatgcatcaacatgtggcatacagctggcacatatatgatatgccagtatttttgacatgcaaactcgaatttccaacatatgtgaaaaattaataattttgagtccaaacaatacgtgtaatttttgttatttttttgttatttttttccattttgtat

SEQ ID NO: 2 - DNA, 49600 bp, Magnaporthe grisea
  misc_feature (1)..(49600): Continuation of Sequence ID 1, representing bases 49,681 through 99,280
aggttgaattcaagcatggattgatatatcatatatgggtctattatatcaaaaaaagttataagtttttcttgacatgtacaaaacgagtaaacgttcccttgagagaaaaaattccacctccacctatatagaccatattgcgtatcagaagaggaaaacaaattcatacaaaaatgtttatgcgtgtttttttagataaaatgtttctgcatgttctggacacacggttgctgcttcatttagtgcttagttataaagaatagcttctcttcttattagatgtaaaaggatatataagttgagaattgaaatctacaggtaatgaatatggttattcaatttgcagagcttatacatgaagtcgtaaatatgttgcattgatgccaaacttttttggcaattgaaacatctttcacgacagcgttgacgaatacttggaccaaattcttaaaaaaagaaaaattgttgccacactttgctatgccacaaaattttagttaacggctatgcatggttgattgaaaatttccatccaatattttttgtttaggtttagttcattaggtacatcgtcagagtatcaacttagaccttgttcgactaacttctacgagaagagaaaaggattggagaggatcaatctcctccaattaactgtgtgttggcgctggtccgcttagcagcggtcggtccgtgctaatcttctcctggttctgtgtgttggcgatgtcggtgtgtgggtgttggtataattttttttgtttttcctggttacaaccctccaaggttataatcttgtagttttttcctgctctatcaataaaacttcgcaccgtcttgtgtgagtcgttaaaaaaaaattctcctccaattaactatcaattctcttgggagagagattaaccaaccaaagtcttgaatgaggaatcatctgttgttagattggcttgagatggtgtttaaaaggctatgatttatccagagcacaactaatatacaagatgtactttgacacaagggataatgcatataggaccgtgaaaaagctaatacggtatatatttcagtacaaagtgataaatatttagtacaaaattgctcatgtacagacattagtacccttgatcacgtcaattgaatttgagcatgtgccttgcttaggcccggtttagatggcaaaattttttgttttgagatgtcacattggatataagggcacacatttgaagtattaaattttgtctaaaaacaaaacaaattacatatttcgcttggaaactgcgagacaaatttattaagcctaattaatctgtcattagcaaatatttactgtagcaacacattgtccaatcatggtgtaattaggtttaaaagattcgtctcacaatttacacgcaaactgtgtaattgattttttttccacatttaatactccatacatgtgtccaaacatttgatgtgatgggtgaaaagtttttgttttgggaactaaacaagaccttatattggtaaatttatgtgcacttctggtttataagttagatatattattctaactctatttttccatgtgtctttcttctattgatgaattttgaatatgcacttttggtttatccatactaccttaaaagcggagttgtcctcctttcatcccctcctcccatcgaagtaaccacctagatctaatgctttattcttaatcacccttctaacccattactaattatatggcttaagccattgcaacacttaaaccccgtatacgtcaacaaaccacgtgtccattaagatgaaattatgaaacagtatcaaaatccgctacaacatttcatccatatatagtgaaacattacgagtacactagctgaaacattattggtggaaaaaaaaaagaaacaaatccccctaataggacgtttccgttatatccaaacagttgttacaaatggcgctagtggtggggacacgtagtgggggtggcgcgtgtgggaggcgaggtggaggcgagcatgggtggcgcccgattttttttcaccgcatcgggcgcccgattttgagtattttcgaagtaacactttacccatacaactatacaagcatgtaaaagagcaatccaattttcacttaagtataacaagcgtctgttcttttccctaccgcactactactttgcaatattgccgggctttatacaaagaaaatttccgcatggccggcggttttgtaggttttctagtttgaatcgattttctttactactgtgtttagttcagcgtaaagtttagattttggttgaaattgaagatgatgtgactgaaaagttatgtgtatgacaggttgatatgatagaaaagtactgaagtttggatccaaactttggatctaaacacagcctatattactctcataaaaaatagatagatacgaattcgatctgccgacttttaaacctcccttcctcttttcccgcgatcgacgaggcagcagttcgaatggatgacggcgaggcggcgcgacgtcgtcgggccatctccggcggcctgacggcgctggccgtccgcctcgccgaccgcctcggcgccgcgagccccggccgcaacctggccttctccccgctctccgtctatgcggcgctctccctcgccgccgccggcgcggccggcggcaccctcgacgagatcctcgccgtgctcggcgccgcgtcgcgcgacgacctggcggcgttcgtcggccgcacggccgagacggcgctcgccgacaggtgcccggagtccgtcgggccgcgcgtcgtgttcgcgtccggcgtctggtgcgacgccgcgcgcccgttcaagcccgcctaccgcgccgccgtcgccgccgagtacaacgccgaggccaccgtcgtcgacttcaagaacaaggttggtatatactccatgaacaatccatgatcgatcaatgtgttcttgattttggtttctgcgaaggcagaggaagcgaggaagcagatcaacgcgtgggcgtggcgggcgacggggaagctcatcgccgacgtcctaccgccgagatccgtcggcccggagaccgccgtcgtgctcggcaacgccatctacttcaagggcaagtgggatcgtcccttcaatgagagcgacacggagaggaagccattctaccgccacggcgtcgccgctgccgacgtgccgtacatgtcgagccgttcctaccagcgcgtcgccgtccacgacggcttcaaggtgctcaagctccgctaccgatcgccgcgactcctccgcgacaagcgcaagcgcggcggcggcgacgtcggcggcgagttcacgcggtacgccatggccatcttcctccccgacgcgcgcgacggcctgcgcggcctcgtggagaggatggcgtcgcgggcgggattcctgcacgagcacatgccggcggcgtggccggtgcccgtcggcgagttcagggtgcccaagttcaaggtgtcatgcggcggcagcgtcgtcggcgccctcgagcagctgggcctccggctgccgttctcgccggagctcgccgacctgtccgacatggtggaggacgacggctccggctcgccgttgttcgtcggcgacgtccagcacaaggcggtgatcgaggtgaacgaggaaggaaccgtggctgccgccgccaccatgacacggatgcctccttccggtgtgccgccgccgccggtggacttcgtcgccgaccatccgttcgcctacttcatcgtggaggagatgtcaagtgcggtcgtcttcgccggacacatcgtcgacccgtcgatggaatgaaatatcttggaaagattcatgtaaaatttgtcgaattttaagtcatgttttacattattctcgtcagttctgcagacttgcaaagtgaaggatacgcttccaaacgacaaacagatgaacaatgatggataatatctctcatacatgtaatatgcattttttctttttccaaatttgatttataatggagttatagcccgatcagagagagaagtagataccataaatatacagacaattgcaatggaagttctgcataccagaaattttacagtatcacaacacaccagattacaacatgcctagaaacgatatagaaatgatgatccttgcactatttgcaaataaagctgggtcaggatgtgccatttcttgcaagaaaaccagcttcccaacaaccagaaaatctgaaaaactagaataaacttccggatggtaaaataccgttgttccaggacttattaacaggattaagcattatgcaattacagaattacagtatccacaaaattatagaaatctgagaaaacctgcaatcacggaatcgccagttgtgcagcaaaaagcattgtattcttcagaaccatgcaggactagactatactgaattagtatcactctacacgaagctcagtaacatgcttcatcttcatgtgcagttggattagtcctcctcttcgccggacacatcgtcgacccgtcgatggaatgaaatatcttggaaagattcatgtaaaatttgtcgaattttaagtcatgttttacattattctcgtcagttctgcagacttgcaaagtgaaggatacgcttccaaacgacaaacagatgaacaatgatggataatatctctcatacatgtaatatgcattttttctttttccaaatttgatttataatggagttatagcccgatcagagagagaagtagataccataaatatacagacaattgcaatggaagttctgcataccagaaattttacagtatcacaacacaccagattacaacatgcctagaaacgatatagaaatgatgatccttgcactatttgcaaataaagctgggtcaggatgtgccatttcttgcaagaaaaccagcttcccaacaaccagaaaatctgaaaaactagaataaacttccggatggtaaaataccgttgttccaggacttattaacaggattaagcattatgcaattacagaattacagtatccacaaaattatagaaatctgagaaaacctgcaatcacggaatcgccagttgtgcagcatgcagcactgtattcttcagaaccatgcaggactagactatactgaattagtatcactctacacgaagctcagtaacatgcttcatcttctcgtgcatttggttcatcttccattcagctttgaagccattgtgcaagcctagaagggtgagtttcttaagggagctaaggcattcaatgccttcagggacgctcttcagatttgacagtgacacaatgtataaaccttcgatacatggaagggcatcatcttcaattttcagaacattgacatcagacatgttcctcaagacaagcgtcttcagcagtggaaaggactttgcaggaagaaccaaagtttgtgcactattgctatgaaccttgttaagactcaaataagtcaggtttggcacgcacgacgcgagtgcttcaaatggatcgttctcaaggtgacacccacttagagctagatactttaggtttcctccatagtcctgaaacatcgggcaatcaagtgtcctctcagcagtgcatcctctgataattagcttttggagcattgtggatgtcggtatgagattttcaatgttaagcttctctttttcatcacttgcacacagaagcaagctataaagaagcggcatcttagacaaggaagcaaacagctctgtgcagtgctcatattttatattgtcaatccagaggtttcttaattggatcattttctcgagctgcccccccaaatccttgctagcttctacagtctcaagtgtctgcagttcttctaaccttgacaacccttttggtgcttccactccacagaaatatcggaattccatccttgtctcatcacttaatctgtcagccagtaggtgtcttaacttgtagagcttcacaatcccaccaggtaatttttctatcttggttgacttagcattaagagtttgaagattcacgagcttctctatagagtccggtagacattttactgctgtgttccttaggccaatatacctcaagttaaaaagatcccctatagatggtgggacttcatcgattactgagtcttggagctcaaggacagtaagatgcttagattcagataacattgatggtacaaagtccgtaaatgattcaagtgcgatcatggttcgaagatgtgggaattttatctttgatacatcatgttttgtctgcttccatctacatgctgacaggcgacgcacatttgtgttcatctgcaccatttcactaaaattgtttgcggtaccaaacaactctgctttggagatagaaagagccaactctctcaaaatgtcatgcatcctgcatattctaaccctgccaagctcatcattatgcacaagttgaagcatgtttcgatgaaccagctccatgagatatccctctgccacctcctccagagtgctgtccccattttttaccacaaaaccttctgcaatccatagcttcacaagaacctcccttgataaaaggtaatcttctgggaacatgctgcagtacaaaaaacaatttctgagctctccaggcaagtcataatagctcaaagttagaactcctcggacctgatcatccttctctagctcacatggaagttgatcataaatctgcttccacacacactctaattgcatccttgcagacaacaagctacctattgatgcaattgcaagtggaaggcccccacatttctttacaatctcaacagctatcttctgcaactctgatgagcacttcgccagtggaaagagcttacatttatttacaatattagtagctagatcgtgacgctcagatggcaacttttccagcaataactcatcacatttctttacaatatcgtgagctagatccttaagttgcgagtaattcaccagtggcaagcccttgcatttctttgcaatatcagtagctagctcctttagctccgatgggcactctgtcagggatgaatctttgcttttcttagcaatgtctgtggctagttcctgcagctttaatgggtactcaaggtcattgctattaggaaatgcctttttacagaacagaaggagtgcatccttcaattctaacggattaagtttgagctggcaacccttgtgagctagagcagccacatcccctctccttgtcgtgatcacaatgcgacttccattcctgcaatccacaaacacgtcatgaattccatcataagctcgacggtcccatacatcatcaaatacaaccaaatatctcttttgtgtcagtgcttcctgcaactctctcttcaattgtgaaacacccaaattgtcaatatcatttgaagcttgctgcctctggttgacaagaccatcagccattgcgagtttgcaggtacgaatctccaataatagctttcttaccaaagcctccacagtaaatgcttgagagacagtgagccacacattaatgtcaaattcctccttatgatgttcatatacattaatgaccaatgtagtctttcccatgccacccataccccacagagatatcaccccgagagctggaattttgggaccgagtaattgctttaagtgttgcctgtagttgtccatcccaacaagatcctcatctttcataagttggggaaaattgtacagaggaaatcgctgttcagagacggtgacttgagtaggaataagctgactgaccgggtgcacccacatgtccttcaactttgaaacatgctcaatatccttctctatttgaattaactcatcagcaattccattaaaaacaacagcatagtgcgatcccttaacaatcttattcaggaacttatctttcttgaattgaatagaatggtagaaaaagctgtccaatacatcctcaacacggtacgccagcattcgcacctctgcgatccatcccttgaggagctcatcgctgaggtaagaggcacccattttccgtataaagaagttcatcatcaaaaattgcctccgtatccgttgcacagtagctgagagctccattagattagaaattttggatttagcaaccatggtcgcttcaattgctgcataagaaccaatctttgagagagcaagtagtgcagcttccgccatctatgctgaaaagataaacaagcaagtgtcttggagattacatagctgaaaagataaacaagcaagtgtcttgcagattacataatgtggtatgctgactggtatatatgttgaaacattaagcgagaaaaacaaatttgcacaaaacttcttccaaatgtaatattgtcatggttttttcctcaactctgtgcgtctgcactatcgattaatgtacacatacaagaaatgaactcatcatacaatacaagtctagctgtctaggcaattaagttttaatgctgtttagcaatgattcttatgagaagttgaggacgatatgaattgactgaattgctgtagcaatggtgtttctttggatgcacaaagagaccggtcatactacaccaagctatatctttgtcataagcctcaggaatatcaaaagacaagcagctctcacaaaacagaggtgacagaaggtagatctgcctcaggacgtaatacagtcagagcagactttatcaaaatgcggtgtcgcaaagaaagactagagaacaagcaaaattccctccccttttttttgtcctgctgtgctcaggtcggcaattcgtcgagcaggacgcgtgcgagcccagcaagcacgcagggacgtgaatccggctaccggcatctgcgtggtggttgccccctactgccgttccaagacccgtaggcaggcgcggccatgcgggagcgaagtagcatgggcgaggccaacgctgcgcggcggcacacagagaaaggggattttttttccctacagtttcatctccatttcagcagaaatggagacgagcagctcaatcaagcacacggcgcgcaatataagtgacagattgtcagattaatccaaccaaaacaaatcaacaaaaaacattgatggctgagcaaccaaaaatatcaagcattcgtcgcgcccgtacgtacctggcaacgatgcagagcgagcctctccaatggcggcttctccctcccgcgctccggtttgacctcgcttgggtcctcgcgaacaagctgatactactgtagtagtactaactactcctcacgcggtcgcgcccttccttctcttgccgcgctccataaacaaattaaacaaatccgacctcgtcgcctcgatgacttggttggttgacttcccttgttaactgattactgcggctaccacggagcaggagcgcgcgaggttgggttcgccattgacgcggactccacatcgcaatccgatccgtcgctgtagaagaagaagaagagaaaaccatggcggggcggggtgtgggggtgtgtgtggtccgcggcgtgtgagcttcgttgtgctgcccagtctgaaaccaactgttgcaacttgcaacgtctcgcttcatagagcaaagaaacctttgacctgtcagctagcaactctactcaacagcctacctacatggcctgtttggcacagcttcagctctagctctatccctcctggagcgggagctcagccaaacagtttcagctccactaaaactgggagtagtgttgggtggagctctctaacaaaataaactagagttgtggagctgggtttaggcaactccacaactccacttcagacccaactcctggagctaaatttaagagttggagctgtaccaaacaagcccataaatgcatacttttatatagttggtaaaaatttttggggaatgctacgtaccggaatctcgtccggacgagactcttgcttatttaaagaggagcgatacctgaccatacacgttctgagtagcagcagcagtagcctgtgcaggacacggtgccgctccagcacgcacgcatgcatggccgacgtgggtggcagcgctgcggcgcccacgcccgcaccgtcaacgcactgccccgcgatcgcgagctcctcgcggggggcgctgactgcggcgcccacgtccgcaccatcgacacactgccccacggccgcgagcccctcgccggtggcgccggcgccggcgcaggggaggaggagcccagcgcggacgcggcggcagatgaggaagtagagcgtgaaggtgctaccctcgtcagggcgagcacgacgagcatgtacttggctagctcgtcgaggcagcggaggacatggccgccggcgacaagctccgacatggcgtgaacgatggaggatgcggctgttggtgacgagctccggcatgggacgtggatgacaaaggacgcggccgtcggcgacgagctctgacgtggcgtggacggtggaggatgcggccgtcggcgacgagtccggtgtggggcatggatggcggaggacaaagccaccggcgacgaactccgatgtggcgcggatggtggaggatgtggccgccgacgacgagctccggcgtgggacgtggatggtgggaccatcggcgacgacgctttggtgctcggctcctgatatctggtaggtatcatctgatacctcgcaaatatcaccttctacgtggtaagaatcacatgatatctgataggtatcatctgatacctcacaagtatcacctaatacgtgatagaatcgcatgatacctgataggtatcacccgataccggttaggtattaggacctgatacctcgcaagtatcaccctctacgtcgtaagaatcgcatgatacctgataggtaccatctgatacctcacaagtatcacctgatacgtggtagaatcacatgatacctgataggtatcatctgataccggttatgtatcaggacctgatacctcacaagtatcaccatctacgtggtaagaatcacatgatacctgatagatatcatctgatacctcgcaagtatcatctgatatgtggtagaaacgcatgatacatgttaggtatcaaacctgatacctagccggtatcatcccgttcgaaaacgggattccgttgtatagcagcccccaaaattttttgccccatatatcacatcggatatacatacgggtacacatttgaagtattaaacgtagtctaataacaaaacaaattacagatttcaccagaaaactgcgagacgaatttattaagcctaattaatctatcattagtaaatgtttattgtagcaacatattgttaaatcattgcgcaattaggcttaaaagattcgtctcgtaattttctagcgaactatgtaattgaatggtttcttttgtctacattaaatactccatgcatatgtctaaatattcgttgtgactgcgttaaaaattctgtttggtaactaaacagtcctttagtactactccatttgatcatatgtttgataagatttgtcaaacttttacaacttaaaacatcaatactacatccatcctagaatatagcaatctagggtaggtctcatcccatcctagtttgttatatcctgggatggagggagtaggttcttaggtaaattacagaatggaccacctaatttttttaccaaagttttaatttggaattcgtcttaacctatcttttcactgtagaccaccatttttttttccttatgtttcattttgcaccccgttcttcttctctaagtctaacatgtcatggttggctcacaactaaatggtaagaagaccattcgcttatacgtgtgaggaagttgtctcaataagtagtgtcatgatatcatgaatttactagattatataaatctagtatataattatgatcaaagttgtattttaaaaactataaaaaaatcaaacattgtgtatttatgaacaaagggataagtttatttagttcatcatactatttacaatcaaaatttcaaaaatttatacaacaaaataggctccacatttcagttttttgggtacaaaaaatgacatgccacctttttttattcattaacgcacatggaacatggtggaggatgtggccgccggatggtggaggatgtggccgccggcgacgagctccggcgtgggacgtggacggtgggaccaccggcgacgacgctttggtgctcggctcctgatatctggtaggtatcatctgatacctcgcaaatatcaccttctacgtggtaagaatcgcatgatatctgataggtatcatctgatacctcacaagtatcacctgatacgtggtagaatcgcatgatacctaataggtatcacccgataccggttaggtatcaggacctgatacctcgcaagtatcaccctctacgtcgtaagaatcgcatgatacctgataggtaccatctgatacctcacaagtatcaccatctacgaggtaagaatcacatgatacctgatagatatcatctgatacctcgcaagtatcatctgatacgtggtagaaacacatgatacatgttaggtatcaaacctgatacctaaccggtatcatcccgttcgaaaacgggattccgttgcatagcagcccccaaaattttttgccctatatcacatcggatatacatacgggtacacatttgaagtattaaacgtagtctaataacaaaacaaattacagatttcaccaggaaactgcgagacgaatttattaagcctaattaatctatcattagtaaatatttattgtagcaacatattgttaaatcatggcgcaattaggcttaaaagattcgtctcataattttctagcgaactatgtaattgaatggtttcttttgtctacattaaatactccatgcatatgtctaaacattcgttgtgactgcgttaaaaattttgtttggtaactaaacaggcctttagtactactccatttgatcatatgtttgataagatttagtcaaacttttacaacttaaaacattaatactacatccatcctagaatatagcaatctagggtaggtctcatcccatcctaggatggagggagtaggttcttaggtaaattacagaatggaccacctaatttttttaccgaagttttaatttggaattcgtcttaacctatcttttcactgtagaccaccattttttttccttatgtttcattttgcaccccgttcttcttctctaagtctaacatgtcatggttggctcacaactaaatggtaagaagaccattcgcttatacgtgtgaggaagttgtctcaataagtagtgtcatgatatcatgaatttactagattatataaatctagtatataattatgatcaaagttgtattttaaaaactataaaaaaatcaaacattgtgtatttatgaacaaagggataagtttatttagttcatcatactatttacaatcaaaatttcaaaaatttatacaacaaaataggctccacatttcagttttttgggtacaaaaaataacatgccacctttttttattcattacataaatatcaccaggcatatcataatcacaatttcacatccaacttatattggagtcaagtcaactaatgccaagttgtactgctgatgcttcatttcagctcctgcttaccaagtatttatccccactctcttccctggagaagaaatccgttcatctcctctgccctgctgcatcctttggttgttgcgtgagcagcagctagcacctctgcagctgcaaagcttctccaggttagtgcattcagatcaaaagctgccttatgttcaaaggctaataaacattacaccgttctttacttctttaggtatgtccgtctttgtgtttttttttaggacagctcatcctgactcagtatgtgagaaacccttatttatttttatttgaatttgaaggcttgttagtttgaatgacagtaatatgaattatgagacaccagttagcaacaatatatcacaaaagccatactgactactcaatactagtgcattcaaaagttgtttctgttcaatatatgtgagactcttatatttatctgaattgttgtagtatgaactacgaaacaccagttagcaacaatatcagaaaagccatactgactactgatgcatgaagggtacttagaagcagctttcatataatactgagaaatcatatatttctatgtctcaacaattataatttttgtctccgttttcttagctttgcgttattttgcttgtgactctatttctcttttattggattagtgctcaataatcagaaatggaaataccaacaaccgagctaccatcacaatttctgagggaaatcactgatgagttctctgatgagcgaaaagttggtgaaggtgcatttggaatagtgtataaggtaagttttttgtagttttattctcttcattttcaaaagttcatagaaaagatgtcaatttggtatgagggactcaacagggaatgcttaaagatgggacagaaattgctgtgaagaagcttcgggaaacatcaccgattcatgacaatcaatttaaaaatgaggttgggagtcttatgaaggtcaatcacagaaatatagtcaagttaattggctactgttatgaaatacaaaagaaggtggtggagcataatggaaaatatattcttacagaggcagtagaaaaactgctgtgctatgaatatatatctaatggaagccttgacaaacatcttttcggtatgataatgcaataactaaatatttccagtcattttccctgtttgcaatgtttacaagatttagatattagcatgatgtaatctactctaggttctactgaagtatacaataagtcaaaaaggtaagaagatcaagctctcactaggttgtgggctcatgaaaattccttgtattttttggcaattagtaagtaatacgattaatcctaatgttatttgttttccataattacttcttcaggtgaatcaagtcgacttgattggcacacacgcttcaatataattaagggggtttgtgagggtttgcatttcctacacaaaggatcagaaagacctattattcatttggatattaaacctggtaacatactgttggatgacaatatggtgccaaaaattgctgattttggtctatcaaggctcttgggtgaagaacaaaccagagtttgcacacaaaatgtcatgggagcaatgtaagtcaccaaacacattccaatgtcaattgttttgtagttttcctacaaggtaaatttcatttgcattagtcacataatcctcaaactattgatgctgatattatacagaggatacatggcaccggaatatttatatcgaggtgaaatctccactcaatcagatatatacagtttaggtcttctaattatcgagatcacaacaggggagaaaaacttccccaatagagaggacatcattgctaagaacttcattgagaatgtaaggcaaaaataatgactgtaccagttttataacaaatttactgttttcatactaaggagttctttcagaataatgttatatataagttcgtgtgctgtcgttgtttataggtacgccgaaattggaccaatatggtgcatatatcactgaagtacccattgttagacacaaattgcctccagcaggtaaagacatgcattgaaattggactcagctgtgttcagaccaaccggaaggacaggccttctataggggaaattgtcaatatgctcagttgaggagcaccatactcactggtagacaaggtatggctataattcaagctaaaaatgtataattgcattgacattgttctggttctaacatcctatttaaaataccagatctctccctttccaacatggtcgtcagtttgggagaggcactggagcaaccgagatgtatgagccttatcgttgatgcttcttagctacaagcaataagatcctatgaaagagattctcatctcgaaataaaatgttcttacagattccatcttgaactacattccatctccttttatacttgtaatcatgatataacaaaatattggagaaaacgctgtttccactctccaatcatgtgtttgacgcagacaattataagttttatttaattccactttgatgtacaacagggaactaaactctcgttgctataacgctccgcaccacatgcttcgttttttaacaaattttgtatttgtgttcacgtgcaatgttgtcatttttttttgaaccaaacaaagtgcaaaatcattccatttgtcaaaccaaataaactcaaaaaacatgtcattccatccatcaaagcatcatcccatactaggccattccatcatgaatggcactgcactcctcaaaccaaaaccatcataaatactagcttaggaagtcgtacattcgtgtgactcgaatcatcctatagtgtgctactaatagtaaagtttaaaagaagttgtgttgttttaagaaacatatttgtcttgtagataatttaaaggaagtaacgtaaaaaagtgtgactgttatgcggctttttatgtgaaacgttataatgtatcaattgtaatagtcatgcatgctaaaatttaatgggataaatgattctataagttaaatttaaacttatttaaggatatagcttagaatttttccttgaaaacaagcatgcaagcctttttctctatttgttggttacatagtacaagtacagatgatcgatgataggggagacaataatgcaatttattgagaaatcaaactttgcaaggtgttcaatgtaaatatgagagaatgtatgggtactttggcaaattctcactgatcatccgagtgcactgtccttcccgagacatcaggagaaaccatgtaattctggttatttccaacaatccttcatcgtacggctatgattcattctaatgttgtttttgtttgcttttcaatcggacggtcatcatgcaccacatgacatggctctttctgtttccatggtcagattgtcttcacttggtaagatactcaggacaaacaatcgacttaaaaataaaggattatcaaacgaccgaattccctatcaggtggattctatagacaacatattatgagggcagccagttagaaccaggattttatacaacttgtagctctgccaaacaggacggagacaaaacatgtactaggtatgccacatcactacacataatgcagacagaacaagtagctgttcccatgaatccccctgatttcatcaagggaggtaacagggagtgtaggaaatcaaaggtattttgatgacgatgattgtgtgaaggacaagtaaacacaaaatgcacaatcagaaagcaacaggtccaaataactgaccgggaagccacgtgaagattctagatccttgtcatgtacaagaaggcaccatgccattcggcagctcactcacagcaggagacatctgaaagccccatattgttgaaagaataatgcagttgatcttttgagcttgattggtctgcaaagttgttttgtttgttcagcacagaagaggattccaatccatggtcaagttgcattttgttgttcacttgttctggtccctctggtgcaatagaatattctgtagtctcacttttgactgcctccattacactatcaacgtctgtatgttcactttctgctgtatcaacagatttgagaagcaatacttttggcctattcggatgctccttagcagcctttacccattcctgtcttgtagctggagtgactccagaatgaagagttacctcctgaagatgtttaaaacattcaatttggatgtcagaaaggccatgtagatctttgcatagcagctgaagtgtgacaagaaatggcagagctccttcttcaattactgggaatgttggatattgtagcacaatacaaaggcgtagcagcctggggaacccctgaacttttatgatgaacttctcgagttcatcggcaaccagtttgagatactgcaagtagctcaaattactcaaggcttcaaggcctgttgtaaatttagtagatgaaaggcaaagctccttgagaccacgaagtgatgtaacaaactgaggcaattgggggaagttgccatgtaatttcaacgagctgaggtagcagggttcttttaaggaatttatcgcatcttcagagcatccactgaaatgaagcgataaagaacgggtaccaatatttgcttccttttcatccagaatgaactgttgaatggcctccctaagatcagtccaatcggtgctacctgcagacgacgtacaccaaatcttcaactttctcaacttattcatgtacctcataagatgcagaaacccttcgcttccgttgctagcaaatcctgctagcgtctgcaagttgctttttcctttcaagagaaactcttgcacttcagtcttctgcttgactttatctgagagcttaaattttccaagcagatgaattaggcatggcaacccgaagacttctaccggcattatctttactttcgatctccttacatcgagtgcctccaaatctttcaatttggcaatatcctttggaagtttggagatattacccccaaggctcagatatttcagaagcaccagattgcatatttctttgagatgatcatccttcaaatcatcacatttttcaagatctaaaactcgcagcaattgatacttgctgaagtctaatacggttgtacctgcctccccaaagactgccagagaccgtacaagtgataaatcaataccgttgaagttatcaccattcacaacagtgtcaccatggagagaaagccgacgaacatatttgggcacgaacttgtcatcacagaaaaaggtgacaaagttctgagagattgacatatgcgatatgaattcacgcatcataccgtacgtttggcatgtcttgactttatcattattgcttacattgatgggctcaatgatgttccggtccattagcacattgaaagcagccgtggaatctatgttactcgatgaagacagtggttctacaaatccctcggccaaccatcgcctcagcagacttttcctcctgattggatgatcagatgggaacatgccaaaatatagtaagcaggccttgagagcatggccaggtagactggtgtagttgcggaccagcactcgccacatcctttcaagtgttttatcattctctaggtgataatgtagccggttgcagagatcttcacagttaggccccattggccagccattagcttgcaggaattcacctatggtaacaagagcaagtggctggccatcacatttcttcaaaattgctgctgaatctggctgcttgtaatgtgaatacctctctgggcaagctttcttggacaacaattgctttgagcagttcttatcaagcctgctcattttgtgcaaatacccattcgcagaacagcaggcattagctactgactggatggtcgtcgtgaccactattctgctgccaatatccttatcagtcggaaaagccgatttgatgcttttccactgatcttctgtttgaatgtcatcgataacaatgaagtacctgcaaggcgaggcagatcaagaaagaataattgtaaacatccccctctggagccacagtaatcaacaaattgaaatgaataatttgtacaatttcatacggtagagcaatttagaagcagtgaattgttttttccatttttattctactacagtaaatttgaaaaagatatcgcgcatgaaattgtatctgttcatgattcattaggtagttgtctgtagtacttgctattgaaagtgtcagtcgcactgaagttgtacaaaagaacaggcgagttgactaaaatgttgacctcaaattcatgtgaaaaatggatcaggcaatttaagcagaaagttgaggattttcacctgttcttctccaactgctgtctgatatctacacacagctgaccagcatcggagtccccatggaaagaacggccgtcggcgtcaagcctgcggagcaattcgccgagcacctcccgtgcactccggtgcccggcgcaaacccacgcgtgcctctcgaatctcccgccgccggtctctcggttgtacgcctccgcggcgagggcggtcttccccagcccgcagaacccgacgatggcgatcaccttgagctgctccggctgccgctccgccagctgctccaggagctcctcccggtccgcgtcgacgccgaccaggtccgcctcgtggatgcgcggatctgaggcagatgagcacgacggccatggctcatccacctgcgccgcagccgatgagtagacctgaccgtgttgaccgggcgccgccgccgtgtacctctgcttccgctggttcgcctccgccgccatcctcttcagccgctgcagctgctgggcaagctgcaggtttctctggagcttcttgggggcctggacggcgcggcggaccggtgactgctgctggtccctggccgcgcggtacaggacgccgtcgatgcagtcctcgatgccgtgggcgagctcgcgtaggtcctccatgcatagcgtctgtacggcggcggcggcggggtggtcgagggagaggtcgtcgtcgatggcgccgacgatcatgcggagctccttgatgaggaaatcgatgtcgcccttgacgcccttgtggagcttatgcttatcgtcgacgagggagaagagcctcggcaggatggccttgatgaatccgctcaaaagcgcggcctccatcttcgaccttcaaccagctacgatccgctccgattcgattgatcacttgctcatggagagctcgggaaggggatagcagactacgaagtattttttaataagacgaaatgtcaaacgttgaaaaagtcaacggtgttatatattaaaatatggagagagtacttcaattaaaccatatatttattaatactccctcaatacttcaattaaaccatatattgattaatactccctccatattttaatgtatgacgccgttgacttttttacaacgtttgatcattcgtcttatttaaaaaatttatgtaattatcatttattttattgtgacttgattcatcattaaatattctttaagcatgacataaatattttcatatttgtacacaaatttaaatgaatggtcaaatattgattgaaaaatcaatagcatcatacattaaatacggagggagcagagatcaaaactagaaagttcacatcagatgaacttcttccagggcaactgagaaggaggatcggttaatgttggcgggttagggccacgtgttagtactaaaatgacagaaaactctcaatagggaacttccagaaaatttcaggagaaaaaaaatgcaagaaaaaaagaagggtgaatagtcaaagttgtcctccaaattttatctaaggctcagtttagaccttaatattttaatctatccgtatcagtcctttaatttttcattttagtttaaacttattcttgaaccaacaactctcttgataaatgacacttatacccccattattcacatatataagagaaaattgcatgcttgattttttttatagataatgcacagtaaattatgtaaaccgaaaataaattatatactagaaaatgtataggatagcaattgattaagcttcatgtgcacatgttaaaataatgagaagtacatctgcaatgttgtttattcatcatttttttctcttttctagtatatagttgtatacaaattaagggcaaaagggatattttataatagaaatattgactagagatagccatatggcataataaatgggcccaaggatgatttcaaaccaaaacaaatatttaaaggagctatgtggacaaaataaaacgtgaaggaccaaactgagcctttggtaaaacttgaaggactaaattagatattcactaaaaaagaactcccgagagaacattcagaaaattcaacgagaaaaaaaaacgcaaaaaggaactaaagggttgaaaaccagaaaattcgaggaaagaaggaaaggcaagaaaaatgaagaaaattatattaagttagtcattcacaatgaacgaaacaagggaatttttctcggtgttgcagctttttgaaaaaacatataatgtattttttctagcatgattgaaaatcttcgaatacttaaaatatagggtaaatttcacagaacctataagttcttttactaaactatccaaaactataaatttaaaatgttgtatcataaaactacatatttaacatcaaattatcacaaaactacaaatctatggtcaagtatcacaaaactatatatttaatattgaagttatcacaaaactacagatttttaagtttaaatcctaacaccatttgttatgttaaaattataaacattatagttttatgataatgttgttacaaaatatgtagttttgttactgtccatcttaaatgtgcactttatgacaaatttgattttaaatttatagttttgtgatacacagagttaaatatatagtttcatgatagtttggttatagtatatatatagttttatgaaatttactctaaaatatcctgcatgcttaataggaaaataccctttaaaatatcacatgatttttttcagatagaaaactttataaaatccctatattagaaaagttgcaaacaatagagggttataattaacgaacgtgccaactagcatgccttctagttaagatatgagaaaattccttctatgcctctgaaattttagccgatcccttctatgtctttgaatattcttcacttacttgtatgcctctaaattttgatttggatctcttccatacccctgctgtcaattgaccgttagttgaccgtttaataggtatagaaaagtcagttttacccttcaatacatgaggaatatgtagaaatttgtaaactatattgttgcacatatataaatttgttgtgtgattataatatacgtaagtttgttgtacatagaaattttaataaatattaaaaagtatgtgtagcattttctttaaatgataaacatagttttggaaatcattcctatatattatatatgacatgaaaaaaatgttgaactaccataaaaaatgctacatatactcttttgataagtggataaatattagttttatgtcattatgtaaaaacaaactttgccacgaataatggcttgtacaacaaaatcatgaatataatagtcttatataacaaaattatacgcttccaacaataaagttggtctgcaatttccagatttccttcatgtgtaagggtaaaatggttattttcataggaattaaacattcaactaacggaattagtatgaaagggatcaaaacaaaaattcaggggcatacaagggagtggacaaaactcggggacgtaaaagggattggctaaattttcagggatatagaagaaattttctcttaagatatagacaacctatcacgattcgagtcactcgattacttgtggaccttacaatatgtggactacaagtaatcaatcatggcatggatcatcacctcccccagataaaccgaaaagaagggaaagacaaagttggaccgattccattgttgaccataggtacccatccatctttttttttttcatttcttagagaaaaagttaaaacaaactatgatactataactgaacttatttaacttaatgtatcacaaaattatagatttaactcaatgtatcatataactatagaacattaaatttatcataaaattaaacatttaagataaaatatcataaaaatacccacttagaaataaaattatcatagaattataggtttgaaaccaatttaatcacaaaactatcatgtttatgactcaacgtaacagtgatgataatgattcaaactataaaatctgtagttttataataaatttaatattaaatatgtagttttataattacttggtattaaatatgtagttttgtaataaatttgatgttaaatatatagttttgtgatatactccctccgtattttaatgtatgacgccgttgattttttgacaaacgttggatctttcgtcttattaaaaaaattatgtaattataatttattttattgtgacttgatttgtcatcaaatattctttaagcatgctataagtatttttatatttgtataaaattttggaataagacgagtggtcaaacgttggttaaaaagtcaacggcgtcgtacactaaaatatagagggaataattttttaaatatcttgttttgttatagttaggtcaacgtcagtgaattttactcggaacgaaaagaaaaggagaaacaaaccaatccgtaccgattttccccaatttagctgcactgctctctccccgacttctgaatcataatgtagagagagtgatgagttcttcttcccttggcgccatggatgctccggcgagcttttccctaggtgccatgggccctctcctcaggaagctcgattcgcttctggtggctcccgaaatccggctgcctaagccattgaaggagggaatcgagctgctcaaggaagatctagaagagataggcgtctccctggtggagcactccgtggtcgattcaccaacccacaaggcaagattctggatggatgaggtgcgtgacctctcctaccacatcgaggactgcatcgacaccatgttttcgatgcgctccggcggcgacgacggcaagcccagatccgagcgccgccacaaggttggccgtgcgaagatcgacggtttctccaagaagcccaagccgtgcacaaggatggctaggatcgcggagctcagggctctggtgcgggaggcgagcgagcggctcgaaaggtaccagcttggtgacgtctgcggctcgagctctcccgttgtgttcaccgcggatggtcgggctcgacctctccaccatggggtttctgctaatcttgttggcgtcgatgagttcaagacgaagctgaacaggtggctcagcgacgaggaggggccgcacctgaaggttgctgccattgttggacctgcagggattggtaagacggcacttgctacagagttgtaccgtgaccatagatggcaattcgagtgccgagctttcgtccgggcgtcgcgaaagcctgacatgcagcggcttcttgggggtattctctctcaagttcagcgtcgccaacgatcgtctgatgcctacgctgacagcacggtgcagagcctcattgataatctccgggagcatctccaagataggaggtatttctttgccacacaagtatgctgcttctgcacgcacttataatcgttctttttcaccaaagcacgcaataaatcatcagtagcatgttaaaaggagatttgtgtaaggcaacttatttctcgagtactggtagatatacagcaccagacaacttagtttgttaatgacaacattgagtcattgaccgtgatttttatttgtgtgaatgaaacaagcaattattaaatgaaaactcctccaatgacacaactagtggattatcctgcgcaattttgcgcagagctagataattaaaatagaattggattcatttgatacgcagttcttaaattctatcttctgtaggtacaattgtattttctacataatagatttaaaaggtagaattggatttcatttacaactcttatttatgtttctattcatacaattgtattttctatacaaccatatcgatcaatctacaccgttataatatgggctcgatatgatctaacggttcaaatctttttcttgttctccgattaatgtgggaatttctggcctagcttccacaacaaatgtagtaatgtagtgggttcttttcgagctatctttaaaataataatatagtaggtagatagatttcagtatctaattcttttaacaattggagagtaaaacacaatagcctcttaatgaatattgcatgtctgtaactgatgatggaatagtcatgtgctttggcttgcagtttacttttatagtattcctttttatgcttgctacttcttttagaatagcatcataaaagtaactatatatgaaataattttcaggtaccttattataattgatggtttgtgggagacggcagtatggaatattgctaacagtgcttttccagatgttaacagtttcagtagaatattaataactgcggatatcgaacaggtagctctggaatgctgcggttacaagtatgattatattatgaggatggaacctcttggcagccttgattccaaaaaagttttcttcaataaagtttttggctccgaagatcaatgtcctcctgagttgaaggaggtgttaaacacaatcctagaaaaatgtggtggtttgccacttgcaatcatcagcatagcaggtcttctaggaagccaaccggaaaatccagtgctatgggactatgtaacgaaatatctatgttccagtttgggtacaaatcctactttgaaagacgtggtgaaagaaacgctaaaccttagctacaacagtcttcctcatccgttcaaaacatgtctgctatatcttggtatgtatcctgatgggcacataatgttgaaggctgatttgatgaagcaatggtcagctgaaggttttgtctctgccaatgaagcaaaagacacagaagaaattgtagataaatactttgatgagcttgtcaacaggggaattctagaacctgttgagattaacaagaatggcaaggtgttgtcctgtaccctgcaccacgctgtacatgaccttgtcatgcccaaattcaacgatgacaaatttactatgtcagtagattactctcagactattacaggaccttctaccatggttcgtcgtttgtcccttcatttcagcagtaccagatacgcgacaaaacctgcaggaataatattgtcacgagttcgatcacttgccttttttggcctcctcaattgtatgccttgtattggggaatttaagttactgcgagttctaatcctagaattttggggcagtcatggtgagcaaaggagtttaaacctaataccagtatgtagattatttcagctgagatatttgaaaacttcaggtgatgtagttgttcagttaccagctcagataagtgggctacagtacttggaaacactggaaatagacgcaagagtgtcagctgttccatttgatcttgttcatctcccgaacctactgcatctccaacttcaagatgagacaaaattacctgatgggattggctgcatgagatctttgcgtacactacagtactttgaccttggtaacaactctgtagacaacttacggggtcttggagagctgacaaacctgcaggatcttcatctcagctattctgcaccgtcatctaatgagggtttgatgataaatctgaatgctataacctcttcacttagcagacttagtaacctcaaatctctgattctatcaccaggtgctataagcatggttattttctttgatatatcaagcatcatatctgtggttcctgtatttcttcagagacttgagctgttgccacccatctgcatcttttgcagacttcccaagtcgattggacaacttcacaaactctgcattttgaaagtttccgttagagaacttctgacaactgatattgataacctcacaggattaccttccctcacagttctctcattatatgctcagacagctccagagggaaggtttatcttcaaagacgggactctcccagttctcaagtatttcaaatttggatgtggtgaactctgccttgcttttatggctggagcaatgcccaatctccagaggctcaaattagttttcaacatccgtaaaagtgagaagtaccgccatacgctttttgggattgagcaccttgtaagcctccaggatattgccacacgaattggggttgacaccagcaccggcgaatctgatagaagggctgcagagtcagcattcaaggaaactgtcaacaagcatccaaggtgtcttaggtccagcttgcagtgggtggtttccactgaggaagaaagtcatcctttggagaaacaacatcataagagagaaaaaggctcatcagctggacatggggttttagagaaagaatcagtggaagatagcgagaagaacactgatagagtgcaaacattattgtcaccccaactgtcaaacatggagagtgtggtggaaagtgcattaacggggcaacgaactaaggtgagttcctcaagctgagccgaaaattgatctttatcattttttgaaggacaaacaagactagaccctagtagataggaaatgacgcaaataactgtaaggttacagtacaaattaaagttggatttgcaatcgtacttccacttttaaggtttgttgtaagtacctttcttcattgaatcggtatctcttaaataacatctctttaagatgggagaaaaccttcagtacatgtacactcataaagtcttaattcatttagaaaattgaacatggacatgaataaagcatgcttgtccctttggtaaacatatgttgatgagttctttagcaatctgatgtcctattttgaatcttaataatgactaatatattgttttgcttgttgatgcagatagttgttaaggtgccatgcggaaaatcccgagcaaaagccatggcgctggctgcgtcagtgaacggtaaatctgcgcaaatatattcttccggttgtgcagtcctgatcagtactcctactagtttatgcctttatgcttgcaggggtgaacagcgtggagataacgggggaggacaaagaccagctggtggtggtcggctgtggcattgacgctgttcgcctggcggctctcctgcgcaagaaatatggcctcgccgagatcttggttgtggagtcagttaagaaagagaagacacagctggctggagagaaaggtgcatcggtacctgattacaataagcaccctacctataatgcaccggagttggactatgtagagtactatccactacaaatgacggaagatcctaaaggttgctcaatcatgtgaaaactatgtgatattacgtgtttctctgtgtgatgaataattagctacattagctgaagttctgagtgtcagtggtttacccttaggttgactggcaccattagtgggatggtagtcatcggtgcaggatttcatccatcagtgttccagttccaatgactataaatatagctatgtgcatccttaactattttacactagtaatttgaccgtgctaatgctacgatgacaattaatattgcaactgaaacgaccaaatgacatcaacactatgtttaggcgatggtgcatacaacgagatagttataaggttcaaaataaaaggagtgataacgagagttactaaaaaaaatgtttatcaacatgtaccccacgatattatctgggaacaagctttgctaaaaagtagtacatgtattgcttggcaatgtatctcgtgcacatacatatcccaatgcactcatacactcactcactatcagccctaggatctcaatcggatggactggatccaagagagagaattttgcaaaaaacctcccgcttcttcatcggtaatttccgaataacccacgaagcttcctttattctattgcatctaggctcctgtcgttcctcccccaaatcgtacagcaaaccgactcctccttctccttgtacgcctgtaccactatctcctctcctgcgttgttgaccagatgtgtgcggccagcaagctgacgacggttgaggaggtctcacaatgggaatagcaggcttcatggggcgaagtcatcggccatggctagcaacccagatgcatggaccttaaccctccattgactgtgcaagcagatatctcctctgacagggatttgactgcaggaggtattttgattcatcttctacgggcactctccggtgagcatggttaccaaggagactcaagccgtcatgtagtcctgccgcacggagacgaagacaaggggccattttgtttgataccaaaaacatacagtcccaatttgttggaaaattggatagtacatgtgttagtttgtaatgagaccaaatatttagtagtgcctctattctatttggtcatattatagaagcttcttcactgtactataataatttgattttatattggtaacaatccaaatgttattgtagtgtcaaagtttacctatattttgtcactaccaatgaaccaaacaagcccaaggtttgcagtggcctagtcagtagcggggagctaaggcccggctagcatgggctgcccgggctccacctcatttaacagctgcacacttaattaatctcctacttaatctgtcggatggttggccaaatcagcggggcaaactagcgaagtatggtttaacatcatcgtacatgtgtgcagtctgcagcttagagaccaaagagaggcagtacacagccatattgttccatcgctttaatttgtagcagtggtttttctctccctaaatcacaagttagcaaccactactattaaatttgtcgtctatattaagtcataactgacatgtgtaggtgtatggattaatgtatactactttgtaattagtcttacccagggtctagaaattttctagatccgccaccgggcctagtgaaagggcatgtaacagacctgactagcacctcaaggttctgagttcaaatctccataggagcggatttcagattaggttatttgagggctaagttctctgttcaatagactaagtttctactttaaaaaggctgcatatatccggttggatgtagagaccgaataaacacacccttctctaaaataaataaataaataaataacaagcccaaggcaaagtttcttaagatgatttgggggagaagcagcaggagcctagatgcaatagaatagaggaagctcatggggttatctgtgaaattacggtgaagaagcgggcggttttctgcaaagttatatctcttggttctattccatccgattaaggatgtgtttggatggagggatatgggtggataggacatggcggcccattttttttgtgtttggttcctggtcggaggtgggatagagtggcccggaggagaatattctccccagatgccggatgggaggatccggccgatttggccggatgcagcggcccgagcattcgggcgggcagggcgagcgacagggtgggtcgcgtcacccctctcaccatcgacaacaacgatggcggcgaccggtgagaggaggcgcggcggcgactggcgagagaggaggcgcggggagaagaggacgtgccaccgccgccgccgccaaccggatctggaggcgtggcagcggccagtgggaggaggcacggcggcggccggcgagagaggaggcgtggggggaagaggacgcgccgccaccgccgccgccgaccggatctggaggcgtggcagtggccggtgagaggaggcgtggcggcggccggcgagagaggaggcacgacggcgaccggcgagagaggaggtgcggggggaagaggacgcgccgccgccgccgacgacgatcggatctggaggcgcggatgtggctggtgagaggaggcgcggcggcggccggagagagaggaggcgcggggggaagaggacgccccgccgccgccgccgccaacgacaacggggtggcagctggcgagagcacatcggaggagttttttttttcttttttccggttttctctcctttcccctcttagtatttctttgtgattttttatttctatttataattttttttgtttgtagtagattgatatttttgtaggtttggagaatttgaggtggtacactcaccattacaaatgaaaaggtgattgtaaccactcatccatcctattcctccaaccaaacaaaaaaaatagatcgccatatccactttcatctctataaccaaacaaaaaactggatcgccatatccagcaaaatatggaccgccatatcccattcaaccttgaccgggaaccaaacacatcctaagtggctgatagtgagtgagtgagtgcatgagtgcattagtttgtatatgtgcatagaatacgttgccgtattgctcatctcaaaatacacaaaataaatatataaatttagctcgacttaagccatccatttacttgatgtggtcctggttatctggacaaggctgcaaaacaaccaaaatgtattcactttagttattatttttatttacacaaaaatttttagcaatttcttaacatgatgataaactactgtcataagcaaggtagacccgcttggatgactaacaaatggacctatgaactgaaattgaagctcagttgatacatctgtaattaaagtcagttgcttgatcagtgttttcatctgtatacaaataaacgaaggaaaacagtcaatatatgtaaatttcgacaaaaaaaaaatagcatgccttttgtcatatcacttacaacttatatgaatatgactaaagtttttatatatcattagaaatatatagctgcattcaacattttgaacatcatatattgcaattttctaaaccaatgcgtcaccaaatatggaagacgtttgtggaatatgcagacaagccacgtgttttcatctatgtgtagaaataaactaaggaaaaccattagttttcacggtcgaacccacgtcacaagtagaaaataaaaatacaatgttgtactatattttacattttacatttataaaaggtggtaaatgttcactagtacaaaacctttcatctgtgacggcctttaacatgtctgaaattagtgggccgttacggatgagtcatctgtgacgggccaaaaaattgtccgattgagatatggtcgcacaaccagttagatgggcataaaactacgacccatcactgataggtgtcatcggcccggccgaatcctgggctttccatggaaaaaacaaatggagacaacatacagcattattacaccacaattgcaaatgcaatatggtataatgtggacacactgcatcaaaagccaatcttagagcaaacaacagcacggactgaacttattgtagcaaacgaactagtcatggagctcaattaaaacaggagtaaatccctattatcagacaaaaacaaggaatcactgggaaaggcagcctaaacaggacatgtttgttatatgaaataaatctgaaggcaaatggggcaacacagaaattacaggacattcaatgatccaggaatgtttgtataacaatttaaagcatgacatgtaaccaaagaaaactttaccgaccatatatatgcaaattcaacaaattactggaaagaaccaaatctgtgatgtacttgcacatagggaaactttatgcaacatctatatacggacgtctgcaacatgcatcatcttctggtgcatcccattaccattccattgaattttgaagtctttgtgcaggctcgtcagccagagcttcttcagggagctgagggactcaatgccttgagggaccttattcagcttctgcagggacacaatgtacaagccttcaatgcatggaagggcgccatccatgatctttatctggttgacatcaggcatcttctgcaatacaagcgtctttaggtaggggaacgcctctgcatcaagaactaaagtttctgcactgtgcatgttgttcagttttaggtaagtcaggtttgacaagtgtgatgcgagcatccccagtggatcttcaccaagatgacaccaacttagagctaaatatttgagatatttactatggctacgaaatatcgggtagtctagtgtgcccttagcacattgccctctgataattaacctgtggagttctgtagacctgggcttgagtgcctcggaagcaaagtggctcattcttatcctttgcagaaagaagcaagctggaaagaagtggcatatttgacagtgccgcaaaaatacttgcacaatcagtagaacttatgttgtcaatccacaatcttcgtagttgcatcagtttcttcaactgctcggccaaacctttgctagcttccacggtctcaagagtctgtagttcatttaggttggatagatctttaggtgcttgcattcctatgaaataccggaactccgactgcttttcatcagcatatctatcagctaacaggtgccttagcttctttattttagtgattcctcgtggtagcttctctatttttgtttgcttaatgtctagagtgtagaggttcgacaacttctcaacagaatcagggagtgacttgaccttcgtgcgccgtaaaccaatgtaacgcagattaaacaaattccctattgatgatggcacttgagtgatttctgaatcttgaagctcgagaacagtaaggtagcttgagtgagacaaaactgaagacagcatatcaatggaagatgaaaatgctccaagtgatactatggttcgaagacgtagaagtttgagatttggtgtagtactgtctttccatcggtaagttgagagacgacgaacatccttatcaatctctaccattgtgccaaaatcatttgcagagccaaacttctcctctttagcagcagaaagggccaggtctcgcataatgtcatgcattccacaagtattcaccctgccaagctcatcatactctgtaacttgaagcatattcctgtatatcaattccatgagatttccctcagctactgcctctggtgtgttgttctctttcctcaggacaaagccttctgcaatccacagatgcacaaggctctcacgggagagcgggtagtcttccgggaataggctgcagtacaaaaagcagtttcttaggtctcctgacaggtcatggtagctcatatttaaaattgcttggacatgattgttctttgacaactcgcttctaagttgattgtatgcttgattccaaacgtaatgtgaccgtgaccttgaagacaagaggcagcctattgacacaattgctagtggaaggccctgacacctctcaactatagatttggcaactttcacgagttccgtgggacactcatggtccttaatgttataaaatgcccttctgcagaatagatcaaagccatgaatttcactcaatggctggagatcaaggtgacatgttgaggaagcaagagctgccacatgattcttccgtgttgtgattattacgcgacttccttggtcattctggaatgcatcacgtatttgaaagtatgcttcttgatcccagacatcatcaagtacgatcaagcattttctaactttgagcatccgctttatttcctctttcaaatcatacacatccattttgtcaatgctacttgacagtggtggttctgtgtaaccaactttcctaagcagcttccttaatagagcatccacagtgtaggtttgagacacaaccatccatgcatgagcagagaagttgatcttttcacgttcatacacatttgtgaccagggtggttttccccagtccgcccatacctgacactgttatcaccttactgtccagctcgtcggtgtacaaccattcagtcagcagtctcctgttgtcttcaatccccacaagatcttcatctttcacaagttctgggaagctgtcccgggacctctgtctttccatctcagtgagcgggtcagaaacaagctgggatggatgcaaccactgatccttgagttctataacttgtttgatttccttctctatcttgactacctcattagcaatttcagtgaaaactgaaacataatgtgatgctttgatgaagtattttttaaggaaccattcttccgccatttgcacagagtaatacgagtatttgtccatgacatcctcaacacggtaggccaccttcctcacctccccaatccaacccttgacgacttcatcagtgaggtatgtcgtgcctatctgcaatataacgttgttcatggttgtcaattgtttcctcatttgctcaatcttctcatccagatcctttagattattaaccttttcagacagtttggcaatcaattctttagcgatttcatttgctaaagcatcaccgatcttcgttagagcaaggaggaccgcctctgccatttgtgctgaacaaaataaacatattatcatgttatggacatacaaatatcacccaaagtttttggcctcagttttactttgatgttgaaaattggaaacacttttctattgtagaccatagtcagactatgataataatgacactactggatgaggctcatgatgtataacttagcaattaaatcagtaacacgggccatgaatcaaggttagatctaatctatattaatgcatttacttggaacaacctagcaacatcaaaaacaaaactgctcatgtttcagtcacgtcatacctagatcggaagataccttttcaatttttttttaaaagaactacagtaggtaagtagagagaaaccggttaattaagataaagctccaatttattgagggattaaatgataattcttggagggtatctttttaattaaaaataataagtaaaaaaactgccgacagggaatcaaacttgggtggtgaggttcaattcccaaactccagttaaaaaatttgagagaaagtgtgagttctttcattagattgtatctatatctaacatattatgctgttgggaagtgcttgggtcagtaaaagaaacattattatacacaagcgatgtaatagcaattcaaaatctgcttgtttacatcaatttgagtatcgatttactgtttagtacttaactataatataaccctttgcttgtctgagacattaattttgtggaacgaatgaatttgttgtggttcacaacaaaataataaatgaatcagcaaggtactgaatgataaaactagtattagataagcatgtaatatttcttggaacatggttgcatttataaaataaaaggaaaagtatattttacttccctaaattatttggctgaagcagttaatcctaagcaattttaagttcgctctattccctgaactactaaaactggctagcttaactctctcgttgctttgtcgtttttgtttgtccttacacaagatgtgttttaaactgaaattgtgtgagatgatatgctcgctcagagtcaaatttcagaacaaatttcccaatattttcggcccggcccggatccaaacagtagggcactaaagtttttagtccagagactaattttagtccatagactaaacgatccaaacaaggtctaactcatcttatatttaacgtgtgagctattttaggacttgtttgtggagcttatagcacatgcaacttttcccaaaatcaaaagctcgcttttagtacagattctgagaagttaaagttgtagaatccagaaaataaactagataaagtacctctctggatgaagtacctctctgataaagccagctttgagaagtttatcgagttcttctcgtatggcctgttttcggtctggtgcaaatcgccgcagtctttgctttactggcttggcatcgggtcgcaccatgagtttgtgctcgatcacctccctggggacccccggcatgtcggacggctgccaagcgaacacgtcagcattgtcgcggaggaaggtgatgagcgcgaattcctatttctcacttagtgatgcctcgatcttgacggtcttgtcggggttggcactagagagtggaacaatcttgatcgcgccgtctggcttcggcgtcttgtttgtcttggccactttcttgggttgctcagttgtagcgggtgggctaggcgtgtgctcgaccatgtcgaggttctgcttgtcgcactgtaccgctagcttggcgttcccttgaatactgattgtttcttttggtcccggtatcttgagcacttgatatgcgtagtgagatgcggccatgaacttcgcaagtgcagttctcccgatgatggcgttatatgctatatcgaattcagcgacatcaaaggtgatctactccgttcggaagttgttcgcttggccaaaagtcacaggcaacgtaattttgcccaatggtttggacgaggattggggagtaattccatggaagggttgatcggtgggtgtcaactcgcttcgtgggattcccatcgcgtccaaagtgctggcgaagagaaggttgattgagctgccaccgtcgatgagaacccgcgcgaccttgatattccgaatagtgggttcgaccacgatcggatagcgtcctgggatgacggcagtattgggatggtcttcttctgaaaatttaatcttctgttcagaccacttcatcttgggtgtagccccctgtgatgtcgaacagacttcgcgttcgaccttcttgtattctcgttttgaggagtacgctgtggagcctccgaaaatgtgagagacgtggaggttagagtctggatatgctgaatccgattcctgagcagccgcctccgcatccttctcgatcactcgtactcgcttgcctttttcaagcgccatgtgcttctcgagcgactttttgaaaacaaggcaatcttccatagaatgtctgttcatcttgtgtatggggcaccatgctttctttgcgttgctatcttgtgggtctgggcgcttgggtgggttcgcgtattctgctgcgagaacttccgcctgagctttccttttcccactcttgcgatttttcttcttgcttgactcgggtgcgttcatggccagtttcttttctccccccgtcttcggtttgtcgttcttgcgtctcaacgcatcgtctacgtgggcgcatcgctcaacgatctcgaatagtctccgagtagttgtgatgcgtcttgtcgccaactcttgggtagtatagcgatctcggacaccggacttgaaggcgcgaattacagaagcatcggtgatgtcggggattgtatttctgcactcgttgaagcgccgaacatattccctcaaggattcacccgagttctgtgtcaacgcatgtaggtcgtcttctatcgcgtgacgcttgtaagttccttgaaagttggcgacgaactactgccacaggtctgcccacgaagaaatcgagtagggcgggagatgcattagccatgaacgtgcagagccttttaatgcggttggcagataatttgccaacgcgttgtcgtctgctccagcagcgtagagtaccgtggagtagacttgaaggaattcttctgggtcagtactcccatcgtacttctctattgctccgggtcggaatctctcaggccatcggacatcacggagggaacggccggaagccctacacccagcgctgggggcggtggcttgtcggcgatcgtgtgtctttcgtggatgtctgtctgacgacgaagatgaagaagatgatgacgatggatcgcttggatccggtgtgggattacgaggtcgtcggctcggttctcgagaatcctgtcgtcgtcctccgttgttgtcccggcgccgatctctgtcgttctcgtcgttatggcggtgtcctcgaccagtatcaccttgcatccgcctttcgcgatcatcatgatcgtggcggttgtggcgattagcacggtcttggtcttcgttcgagcggccctctcgttcctcgcgatgtcgtgaagagacgcgatgtcgggagcgattcccgtcgtcttgcgtgcgtcgtgcctctcggcggccattgagatggtcgcggagatcaacggtgctgcgaggtggaggggtagcttgaaggggtgactcctgacgttcgggattttcgccatcgacatcgcctcttggtggctgttcctggtgtgctgcggcggcggcctcttcgaacgcattgctgaggttggttaccgactcccgtagacgttccatccactgtgcgaggtcatcatttaggacgggatcgtaaggggtctccctcaatattgcgttgatggtcctgatgtgctgggccagtgtagtcacttgattttgcgcttccgcattagcatgtgctgatgtgctggtcccgtcgatagccaatacctcgcgtggcgtactcgttggcgatgagccatagtcctcgagtaaatgcaggcctgatggttcgtgggaaccaatgttgatttcgctaacgaatcgatctgagatcagcatttcttgttccttgtcttcctctctaaggggtatgcctgactgatggaccttaggagatgacgtctttgtggcgctgagaaagaccctgcagcttgagaggtcgtgattctttgtcttgtgaataggacagtagacatcacgagttggagagatacgctgaattccacgctctttgcaagcgagaatttcggcgtggacgtcgaggaaaacccggcaagcttgcaaagtgtgtttcctggttttgtggataggacaccaggaccttgtcgcttcggaagttattctcgtcggctcgtggttctcgtcgtaacgacaaggtttggccgcagtagaattcttctcgagtttggatgtagtcgatgttccgttctccgcttcggcgggaggatccttcgacatgggatcgtcttgggctgtagccactgcgttctcgtttctgattattgtctgagatcggcatcgtggtgaaaaccgggaagacgatttcgccgacttgagtccacaccagcattgttgagcagggattccctggttgaggttgacgctgatgctgttgtcaacggagctggatgacgtaatagtagaaccttgagtaccaagtgcccctacctggcgcgccactgtcgacgggtgatacccgtagaccagatatagagggtattggggtacgttggtacaaggatctacgtaatacgacatcgaacaaacagaaaacgaagatttatactggttcaggccccttaacaggtaatagccctaatccagttgatatgggattatatgatggaaatcacaggttacaaaaggggaacgatggaactcgatgaaaccatcgagaacgtaatcgagtaggttcgactagctatcccgacgatttggctcctgcaggctccgacttcgtaggctgtggtggttgtgttggatctgagattcgatccctcgggtcctcccaggggtcccttttatatcgcaggtcaggcggtttccaagtcgaactcggagacatcggaccctacacgatacattgacgacccagtcctgtccgagttgaactcttattctctgcaacctccgtgaaggatttccttagtgtatacggagaacgtccgtatatgcgcgggcatgccatatcgatatgtgacgtgtatcgaagggtagagggtatatctaacccgtaaccctaacaaatacttaattaagcacgcactaatggaccactccattatccgtgcatactgttcgtgttgggaatgggctggaacgaacacagccctataatatactaaaacatagtagtagcctttttaaaaagtatgataggagatttaattttaaaatttaaataaaaaaatgctgagacagggttgtgccacttgagtataaacaaaatctgttgtatgggtgcctggtgggcatgcgtgggggtatcggagctgttgggattggacatgtaaaggtcggagcgaggaacaggatatattgttgttcttcgttgcccttaaaacgaagaagagagaaatcactctccaaataactcctgtcgtacctttgcagtactgccgtgtctcccctaacatatcataatctcatggttagctgcggttttcttttcgattaatgatgttcctttttctttccaatgaattacctcctatttcatgattttcgtccatatgaggttctggctgatagcagctcatgatgtgttcccttttgatcaactgcaggcagaaacaagaatggaagctccgattagcgcttcgctgggtgccatgggctcccttctacagaagttgaaggagctcgatagcccgactatccatcaactcatatctgagcttagcgtcatacaagatcgtttggagaagctttcaaatgtgcgagatccatccctcacggtgatttactggatgaaggatgtgcgcgagctttgctacgacatggaggactgcatcgacttgttggtcaacgctgatgctggtgccaagatgggttgggatgacaccatctcaggattcttgactcttgtcaaggaggcgaatgagcgatggagcatctataatcttgatgctgatgctggatcaagtcccacaaacatggttgtacatcatcatcttccaactgtgttcagcaaggttgttgaccctgttggcatggatggcccgagaaacaagctgcgtggttggatggctgaagacgaagatgaaaatctcaaggtgttatgcattctcggtgctgaaggaatcggtaagaccaagcttgccgaactactatggcgtgaacttgaaggaaaattcgagtgccgggcttttgtgcagacggccaaaaagcctgatataaggatgattctgaggaatatactctctcaagttcggccgctccaaccagtcgtcccttgtgctgtgaccaacctcatcgacgatgtcaagaatcatctaaaaaacaggaggtttgcaaatcttctttttgttttttagaacaaaaatccattgtcatgatatttttcttggatattttctcttttgcttgacaaaacacgtctgatacagaccaaattatttgtcaggtactttattgtaattgatgatttatgggctccatccgtatgggatgttgttagacatgcttttcctgtgaataattgtgggagcagaataataacaaccacaaaagttgatgaagttgctctggcttgttgcgtttataaccctcagtacatattcaagatgaaagctcttagtgtcgacgagtcgaagaagttgctaatcagtagaacttttggctctggagagcatcccggtcaattccacaaagtcacacaagagattgcaagaaaatgtggtggctcaccactagcaattatcatcatgggcagtcttctagcaatccaacatgagacagtacagaacttggaagatagattgaagtactggcaatatgtagaaaatttcttgtgcaataatttgagggtaaatgctacttctgatgagatactgaagcaagtactgaacctttgctacaacagtcttccttgttgtttgaagacatgtttgctatatcttagtgtatatccggataactacatacttttgaaggaagatttggtgaatcaatggatagcccaagattttgtctgctcgtcaacacgggaaaatgccatggaagttgccatgagttatttcaataagcttgtcaatctgggactcatccaatgtagggatatcaagtataaatctgacatattgacctatgcagttcaccatattgtacatgaatttatcacacgcaaggcccaggaagagaattttgtcactgcaattgattattctcaaacgacagtaagacttacttacaaggttcatcgattgtccctacacttcggcagtgcaacatatgctactacaccggttaatattgggctatcagaaattcggtcttttatatttattgggctatttaactgcatgccttcaattaaagagtttaagcttcttcgtgttgcatgtcttgatttttcgtgtgaagatggaaacatgatagacctcacagaaatttatcaactggttctattgagatatatgcaggtcaattgtagcgttacagtgaaactgccagatcagatgcagagttttaaacacttgcagacactggaaataaatgcaaacgacgcagttgttccatcgaatgttcttcatctcagtttcttaccactccagttccaaggtatgccaaatcaaccaggagttactgatggcatcaagaatcacagtatggatcctagtacctcacacacgactgctagcctccatgattcaacgagcgcatcctctcctcccgttcaaacactggagctgttgccatctgagtgcatcttttcagaaattcccgagtggattgctcattttcgagaactccgtattttgaaaattgtgatcagggaattcctggatagcgacatggatatcctgacagaattgggtacccttgcagctctctcactgtatgtccaggaacccactgcaaaaggcatcgtgttcaaaaaaggtacattttctgctctgatattctttgaattcaggtgtcataaactgcgaatagagtttcaggaaggggcaatgcccaatcttcagaggctgtatctagttttcgatgcccatagaggtgagggatatggtattgcgcttttgggcgtggagcacctattaaaccttacggatatcagtgcaagaattggaattgccactggtgctatcgaatctgaccggggagctgccgaatctgtttgcaagaatgccattggcaagcatccgtacggtcctaccttcaaagttcttggaaaagtcagtcaggttcaggaagctaatcatctggacagacaaaaacagagctctgacgaagatggttcttcaagcaaacgataccgaatagagatagacatacgccaaacaacaaagagagaactagaagatacaaggaacctttccgatagcaggtgtggattgttctcataaataggaagccatcctatttagtttttttatgatatttctttaccatcttatttagggatttttcacaataataattgtttaccatatgaatgctttaaaagggagatgcactctgtgaaagcgtgtccgggagaaaggtgttctccgtatgattggttaaccaaacaacaccgaactctaaagatggaaccagaggatgagcgtaaccttgacgatagcaggtatgggttgtttatacttgccgatagtataataagtatataacaagcgtttatgtcacccacaaacacatgagagtttatccttaaaatactagtcaaaggataaacatcctcaaacctcacgaaatttatgttggagcaaatttcacataacttcaggttttgaggaatccatgatgcaaatccctagattttataatttgtttcataaaacctcaagtttgggggcaaaatatttcattaaacctcacctaaaaatttatatcatcaatttaactaatatatttatctataacaattgtaaaaaatatatatgagacgtttttaagtacatagatgattttcaacattttgcgatcgtagtagtttgatcatttatacgtatatttaacaaattattatatcaatattagttaggattatttattttttttagtgaaaaactaggaaggtagcccacgcatctgcgcgggcatcttatttaaaccatcataatttgtgttattaggattgtgaaatgttatgattttgcctctcggtgaaaaaaaattaaaggttcctttgtaacacatgattgaaaaaaaaagaatagaaaatgtacaaaaatatgataggaatacatgtgtaaaatagagtattgaaaagaacggatgttttaatggagtgtagaaaatcatgggaattgaaaatttattaacccacctagatcaaaatatcatctatcaatgcataaatcataaccaaacaaactttccattagcagcttgattgcctacatgctatgtgaaggaataaaacatgcatggacatcttggttttattttattgccgtagggatatacgacgacttttacttaagatgttatttgataatagtttaaatatatgatatttgtcctaaaaatgtttttaagtagttatgatactttctagattgtatagatttataaaccaccaagattataatttatagctcactctatgggcactttgttaatatcgttcaaaattatactactatatctttttcttttgaatatgagtgtttgatattttcaaaattaaatgttgagcgttagttattctaacaatcattgacatatttaattaggcagtaaaatattaaaataattatattctatatgatgactgagtccggcatcataacattatgcgtgcattataactcgtcacatgttctaagtattttcttagttataaagatataaaaaatatatacttcaaactgataatttgtctattttgatatgagtgcgcaataaagcacatttgagcatcgtaaccattcatttatattaaaaggttgaaactgtacctccattatttataagaaagacagtgattaatgtaaacttattccttaaaaatctacttactactcccatttctaagtatagccatgattttccgtgatcaactttaaccgtccgtcttttaaaaaacttaaaaaaaattaaaaacttaagttacatataaagtaactattcatgtcttattgtctcataataataaaaatactaattataaaaaaaatccaaataagatgaacgatcaacattgggcatgaaattttatggttgcacttaaaatgggacgggggagtacgaaacaacttatgtgtcattttttttacttttaatctaattgagttgtatataagatatatgctttgtacatatggatgttggagagggaggagtgattatgtacgggagggagtgggaggtggaagggatttgtgttttgaaggattgatttaatggttagaaataatgggtccaccgatttaagttagaacggatagctaagtatttttcttttttcttataatttttatatgattttttctaatttattaaagttccatgtagcggtttaggagcattctgcatgatgtttaatggacttaaatttttttgtgtgtttcttgcatggtataggtagaaaatgattttttttccttcagaattgctacgacatatttgtggaaaccaaaggataatacaatgatgaatcaataaggaattcaaattgcatactgttttttctatcctataatcggaagcttatagcttgatttatttttacttttaaaccctaaatgtagctttgttttattttattttttttggatttcagaatgaaaaaaaaaacagacatagagggttgtgaggccaacgtgtctacgtacttacatacggtgcttatgtgcacgtgtatttagtgatttttagcgataaaattaactgttaggttttcatgatagatctaatggtttctaacggtgtatttatttatttttattttagaaagaaaaagaaaagaaaaagagggacatgaggccagctggttacttagatacgtacgtgctgtgcttgtatatatgcacgtgtccgtgggattgatggagataaatttaattgatcgtttttaaaaatagatctaatggttgacaacaatgggtccatcgggaatagattaaaaaaatcagatgttttgccttcttttgatttttttagaaataattatagcgccacgtagagacttaggagcatttgtagggttgctatgtggcggcttagtagcgtttgtagtaagtttaatggacttttattatataatagatagatagataatagatggataatagatagataacataacatcaattatatacatgtgggtcccatgtgctggtgctatgatgtattttggatcatagacatctttttaagtaaaggaatacaatagtatcacaaattggatataaatttaaaataatttaaaagatggaatgtttaaagagctacgtatatttgcgtaaatttgtaaattgtatgaaataacgtcgatttttctttggaacggtactacgttcttttttttctttatttttctctatcttataagtacatgcacgtggtcttctttttctttttcttgcagcaaacaattaatggtttagcttttttttttcttttagtcgtacgtacgtgaggaaggtatttatcttttaggcaacagttatatatttctcctgtgaaaggaaggtattattttttaataacagatgcaatctaatggtctaaaatatcgggaccaccaatttaagtgaaaatcaacggtgagattatatagtgccatgtggcgatctaggagagtttgtaggattgccacgtggcggcttaagagcatttgtaggaagtttaatggaattttagtatataataaaagatttgggggggggggagaagataaatgagacaagcggaatgtgtactttgagttaacatgaatttgtgtacaaataatcatatgtaaaaatgaaatacgaggattttacataaatatggggctttttgaaacattttgccctaaaacatgaggttcctggaagcaaatcacaataactggtttttgtgttttggaagccccaatacctttattcgatttaaacaccgaggtagggaaaaaagtttacacccatcagtccaccacacaacaaaagcatttctcaattacaacaaacatgttatctgacacacttcctggaccttataagccttgtagttttcttattctccgactatacctataataataaattaaattttaattgtgtttcatgtgcaaactgaaacttatggagaaattttatgattttcaaagatgaacctcaaagtaccattttataacgtaaaatttttgctccctcttagtacttcatgtaccaggaggtatctaaattttataatgcaaaatctcatatatatcttcaggtgtactcaaggtccaaccgtaaaatcactcttatatgtatctacctctctttctcgtctcgtacattcatttctttatggtctacgttggtttgtacacaggacattccaagcaaccgatcctccaaagtcttcccaagaccgcaagttaggtatgtctattgcatctccaactccgaccacattatatcgaatgctagaacttctacttgttttacgtagaactatctacaatttcattgcaatttacacatggatatatttgttttattccaaatataatcgtacctcctgtagtactctaatttgtgtgggatcagtctgagtactgttacaaaatgaaaccaaatatttatttgctttcttattaatatgatagctttctagctaggtcacgaaacgaacttggaggattatttctttatcatgttttaataataactaggaaggtagattatcaaaaaaagaaaaacaaaactaggaaggtagcccacgtattcgcgcgggcatgtattgtagattgcgtttaaaaacttatatagatggaatatcaaccggatgacacctgtcatgtttcaaaagcagtatttctccataaattttcttcgcttaggtaatttttttttaaactatttgtttgcttttgaaacatgtaatatttaagacatccctataaatagttatatatgatttcttcagtatagagttagatctagtctgtttataaaacttgtctaactgaaaagcaagtaaggtgttttgtcttatttaa

SEQ ID NO: 3 - DNA, 49600 bp, Magnaporthe grisea
  misc_feature (1)..(49600): Continuation of Sequence ID 1, representing bases 99,281 through 148,880
ttgaatgtttagtggattttttaacacattctaaagaactgttatcattaacaaatatgttttatttgtagcatcaagttagtcatccttttaattggttcagattgacgcaagctgttgcatacttacataacaaagatatgtcgtagacctcgcttcctcgtgatatgcgtgatatatatatatatatatatatatttaaaagaaaatacctaattaacataagtccaaaaatttaaatttaatagtaacaaatataacattttaactaccttatatatgcaataatatgcgaagaaactcataattctatgatacagaatagatataaggccattaatatctaagcatttaaaaactgagaaaattccttccatatccttgaattttagccaatcccttatgtgcccctaaattttctgcactcccttgtatacctctgaattttgatttggatcccttccgtacccttgccattaattgaccgtttaataggtataaaaaagtcagttttacccttcaatatgtgaggaatatgtagaaatctgtaaactatattgttggaagtataaaatttgttgtgtgattatcatattcataactttgtcgtacaaaaaaattaatatatattaataaagtatgtgtagcattttttaaagataaatctagttttggaaagcattccaatataatatatacaacaggagaaaatagagtaaatttcacaaaactacagatactatgactaaattatcacaaaactacatatttaactcagtgtatcacaaaactacacatttaacaccaaatttatcacaaaactacacatttaagatgaagtgtcacggaactacatgtttagtaactaaattatcataaaactacaggtttagaaccaatttaatcataaattaataatgtttatagccaaaagttgtagttttgtgataagtttaatattaaatatgcatttttgtgatacttaacctaaaatctgcagttttgtgataaatttggtgctaaatgtgtagttttgtgatacaactccttaaatgtgtagttttgtgataatttagtcaaaatacctgtagttttgtgaaatttactcgagaaaatatgttgaactaccaaaaaattgctacatatgctcttttaataagtaaataaatattatttttatgtcattatgtaaaaataaactttgccatgaataataccttgtacaataaagccatgaatataatagtcttatataacaaaattatacacttccgacaatacaactagtctacaactttcagattcccttcctgtctaagggcaaaaaggtccttttcattggaattaaacattcaaccaacggtaaactaacggaatgggtacgaaagggatcaaaaccaaaattcaggggcatacaagggagtggacaaaactcgggggcgtagaagggattggctaaactttcaggggtatagaaggaattttctcaaaattgtaaaacgtaacatgcaatggtcggtccaatttaaggaaagagtaagaactaattcttttacaagcttattagattttattcaaatagtccaagttagtccttttatcattttacttataccatgatagatctaatttaattattgtgcaattatatacaacgataaaatctatataattctttttcataatttcataattgtttgaatgtcacgcaaaagtcagaaaatataacctcaaagtgacatatcaaaagttaaatatagtgaatatggctctttaaatataaaatagtcacaaacagaacaatagtagtcatataatttagatgttatagttgcttactttatagcattacttttgttttctaactaatatatttaggaatattactcatagttatattcattttacttttatgatttttttgactggacattttacttttatgattgttgtcgagggaagacccccgatcgcggggagtcgcgtgacccccctttcgtgagttcggccgagggcagagactcgtctctccgaagtcgcgtgttcgtccctccgatccccggttagatagcacacaattcgattacccaggttcaggccactctggagcgtaataccctactcctgtgtttgggattatcagtgaactcttacaaggttccccttgctttcccctccaaaagccccccctttttgtgggcaaggtcctccttttatagcttaaggggataccacatgcgccgtctctaccctccccctccatcggagggaggggagggggccctgctcgctcctgccaccgggtttcaacccgtgccttttgctggaagctaagcatcggttggtccttgagtggggcccagcaccgtctcccggctgacacggggggtagccctgtcattccctcataaatgagtgaggacttgcggcagccctgcatcgaacgacgtccggatgtgacggggcgtacctaccgccactccgcggagacagaggcgacgtgggggcacgaccacccgttcaatcggccgtgaccggcaatagactggtcacagaccggtcgcatccgactgacaggggtcggtcgggcacgccgcacgtctgccctcgccgcattgaatgcagcaagggacggttggggcgctgcacatttatggccgcccagcctggactcccctctcacacttccccccgagggagggcctcgggctaaaacgacgggagagcccgaggggggtgacgtcacccccgaggggatgtcgtcgacccccgaggctctccccatcgccttagtgactcacgaggtatgtgggtgggaccagctcgcagagccacgtggctggaaagggaattagctctcctttactccacatacccccgggcccatatctccgacagtagcccccggcgccatgtcagacgcgggcttcctgggatcggtctgacatggtgcctcacgacctccctagctggggttatgggcccggcggggagtttaaaggagtattgccgagcggccagattagcgggccagccatagcggcggttcggtcgcctctgatcgcgctaaagacgaggatgcttgggacgcgataatcaggggatcggttagaccacgcctcgattacctcgcccgctattcctacggcggttgtggtgacggttcactcagcttgcgcttatagccgttacgcattccacctcgagtgcgccggtgataggcgcactaaaggtgggacgcggatacgacgggcgacgtggtgaacgggcgcgggaaatggaaagacaactgcggacatgggggcaattatgagggcgggaaacgaggaggcgagcgcggatgaggcgagaattaaaagggagtggaggcagggacctcccctccttcctttcgctaaccgccgactcgtcctctcgcctctcgaaccctaacactctcaccactccacgcagctttgcgcgccacgccttctgtcactgccccgccggccatcatggctcaagacaccggcgacacagtgctcccttcttcccgcatctcggcagacagacatcttagcctcccccgcaaaattatgccggacggcgccgtcgtgagggcgggggagtcacggccacggccgcgatatccggagcggtccgtgcacctcttgtccttcgccatggcaggattgattccgcctttctcccgatttttccacgaggttcttgatttctacgaaatccatgccttgcatctcgcgcccaacgccgtgatgacactgagcatctttgcgcacctgtgcgagatgttcatcagggtgcgaccgacgatgcggttgttccaggccttcttcgtcccgcagctgctatagggtgcggtggtgggcggatgttacttccagccccgaccaggcacggcggggcagtacatcgagagccacctccgcaagaaaagggaggactggaagaaagactggttctacactgcgctgccggatcacccacgacttcgagttcccgccggtcctcccgagagatccgccgcatggctggcggcttcggagctgggcgaggaatatgacgcggtgtgggatcgtctcaggggcctgcgaagcttgggcctcacaggggcaatggtcttcggcgactactttcaccgccgtatcgcccctctccaggaacgatcccgtggcgcgtgggagtataccggccccaacgaccccatgcgcacgcatgcgggccagcgctgggactggggcgaggaggacgccaagatggtgattcggcgggtgctgggcctggactccgccgagcaaacactgatcccggatgggatcctccccctctgctgcgaccgcgaccgggagagcatcttggccgtgatgtcggtcgtcggcgccggcaggggccggcctggccgtgggggcgccagcggtagcgccgccagcgccggtggtggcggcgcgtccgtgggcgggaggcggccaagcggtccaggcggtggtagcgactcacgggcgcccggctcgggccgcaaacccagagacgacccggcaagtgacccgaaggggaagcggaaggcgaccgaatcttggccgccatctcccccgcgtggaggaggcgccgagcgcgcgacagaccttccaccggcgggtcacaagcgacctgccgggcccgaggccggtcggaagaagaaacggctccggaagatagggcaaacggagccgtgccgagggagcttcatcgagcccccaaagtggaccttcaactgtcctccccgcaggtacaaacgctggcgctcacttcggtgctcttacctagctcggcctcttgttccctcccctttatttttcttttgttaacgcgatctgggttcccttgtagcgatattccttctcaggggacctcggggtcggacccctcacggaggacgaaatccgcacaaccggagcggtccgggcctggccaatccaagaccacagggtcttcgcaacctcgcggtggggtccacaggacacgtactgggcccgatcccgagcgacccttagccgggactcgacctaccgtgggcggcggtggcgtcgacgcccctcccgggggcggctccgagactggggtccatgcggagcgccacccaggatcggttggtgcggacggtcgtgagcccgaggttgaggtcgaggctgggggcgtgtcacgcccggaatttctatccaaaattccaaacgcttacatgtgtgttaagccctcgtccaggaatcaaccgaggcacacaatgacaatattgataatagagtacaaataattaactattaaatactcgcaaattaaaatattacagaggtagatagttcctctcaaagaaaataaaacattctacgcagcggaaagataaaactaaaacaaacgacggaacagcgacggcaactccactccacaggcaacttaaccagaataagctcaattctcaagatcatcaccttcactgaagtactcctcctctgatgaatgaatattgcaagaatgagcatatgacatactcaacaagccacacagcaaatatgcaagtgcacaggataacaaaggatggcataatatagctcatttgcagaaaacagcatttaataaatatttaagagaataataaaacagttatataattaatcaatattaaatcaacaccatacaacacacccgtgttgcataggcccaaccacatttgatcaaccaaccccggctgaacaaattaaccctccaaaccaggaattaaccattccaaaccaggaattaaattaattatcacattattaccattaatgagtagtgtgagactaatcacgaaaaacattgctcaactcacccataaccgcgggtacggctattcgaatagattaaactctgatcagaggtgtaccactgtacccacaagacacaaccccacatcatgttaccatgtgcctcaataccaccacggtatctcggaaaggaattgtgacattatccctcacataacacaattcaccacagtgcaccgtacctggatcataatcaccccctctacaaccagaggcatggactccccagcgacccccatggacttctcgccacttctcagtctggcgcactttaatgaactatgctatacaaaagataaagccgttgcccacgctggcttgtggttggcacgataaatgtatcacaacagtagctcgcgaaccggtccttaattgtcatgagcacaaccatcaaaaccatatgctcacaacccaccttagccaggttttagttatcaattaattattatcacacaattgaccatcgtgagctaccattaaaataaccacaattattaacataaaataatgaagtagtttaccattaacccttttagttagctaatgtttctaagcatggctaagcaaacctagatatatagcatttagctgaaccaatccaatatataaggttcatgctaatcaaattataacccataggaaaataaagtcaacttcggccataattaactgggaaaggttcaccacccgatgacattcgaaaataatgcataagttgaaataaaataatagctttaaacaggatcaacatgctcaaagggttgtttgggatctgtgtgacttgccttgggcttccacaaacgcttcggaaccttcctcaacggaaacgctctcctccggaacgtcggaaactaaagcaaataaacaaaatcaacaaaacagtacaaaaacaagcataaacagtacatgtggatatttttaacatgtagatcttgattttagaagaatttagaaacttgaaccacctaaaacggatctacaggttatttagttatgatttttcaaagttttaatctatttaaaaatcgagaaaaagggaaaaaggaaaaggatgacgtcagccctggcagaggtgacgtctgccctagggttgacgtcagcctgacgtcagcgacggcggccggctcgggacgacgagctcgccggcgctacggacctcggcgacgagattggagggcaccaacgcgtagagggcgacgagaggaactcaacggtacccaccccgacacgagacgatgaacgacggcggccggcgacgaagaagaaggcggcggcggctcgggtcgacggcgtcgggggtgctccggtggtcggcgacgcaaacggaggggcggacggccttctcctcctcacggcgaagctaatggcggtggcggcgaccggcggcgacgacggagacggcggcgcggcaatgccgaaggacggccggcgacggcgacggctaaaagcacgcggcgacggcgctaggaggcacgggaggactcaggaaagggggcaaacgagagaggagatcacggggaacatttatatgagctcaaatcgaagagatcgaaccgcaaacggatggaatcgaatcgggaagaattcggacttatttttggagataaactcgaaaccgaatccaaatctcatccaaaactcaaccatttgctttgattcttgggggaaaaggaagaggagaccgagaggaacaaaacccctcaatcaatttgaagagatcgcatcggatttggccggaatcgatgcgggaaaaacggctagggttcggcgcgggcgcggcggcggcgggaggttgaagacgacctgacaggtgagccccacctgtcagcctccgggtgagaggggcgcgcgggcgcgggcgcggcttgggccgagctgggccgagcgcggggaaagagagagaagagaggagagatgggccggattcggcccaaggaccaagaaggggactttttgaacttttttgaataaaacaatttgtaaaatgttgtttcaattattaaaaatacttcccttgctcaaataattcccagaaaaatctagaaaatagaggagcaagcatagtatttaataaaattttatttagctcatttttatgttgaaaattttcctagaaaattaaggtttagcttgtaggcttttaatttaatttctaaaaattccaattaaacaacaatttataaatttaggatttttgggatgtgacagggcggccctgagcccgaggccgaggccgaatccacccgaggactggaagcgggggacgaaaagaacagccgctcccccttgcgtttggggcgccctcggggctggcctcgcagaaggggggccgagagcagcccccgtctcgtggggggtccagcagcactccgtcgtcccgggggtgacccgtgggcttttcttccccatccccggcgagcatggagccacttctgcaggtgctcgccgccgccgactccactgtccgggaggggctcaatacccaagttcaagccctggcggaagagcggacgaccctggatgccgaatgggctcagcttgtggcggaccgcgcgcgagtcgacgaggggcgccgtgccgtggaggacatggtgagggtggggcgcaaaaaacactaagcccacctagccaaaatctaggcgcgcgaggaggtgctggactccgtcatgcgggagacggaggaggagcggcaggcggcgctgatcgcctcgagcgccctggacgaagcgctgggcgatatccgccttcagtacgaggcccatgccgaggacctagcggagagggtcaggaatgcgcgtggcaccctcgacgcagccgctgcccacgagcggcgagcagcggaggccgacgcctcgttgcaggcccggactgcggcactcgaggctgaacgcaaggccttggacgatcgtgcccgctccgtgcaggagtttgaagccacgatccgtcggcggattgaggtccttgatcggagccagcgcgaacaaaacgcgcgcggccaggaacaggcacagcgggcccaggagctgggggagcaagcacgcctgctggatcagcgggagtccaccttggccacccacgagagaacggcggcggaaacggaggcctcccttcaccttcgtgaagaggccgcggctgagcatgatcggatcactcttgccgcggaagcttccgcgggccgccgcgcggaagagctacggctgcgggaggaggtgtgccaggaacgggacgccgcactcgccgagcgcgaagccgaggtaaaccgccgcgaggtagccttgcgccggcttggcaagcagcttgcgaagcgcgaggaggctgctgctaggcgcgaggcccggcatctggagagcgctcgcgccgagcgcgcggcgataacggcgagggcttctgaactggaggcccgggagaagcacctggcagccggcgggccgcccggcggcgcggggttggcgagctcgctcgccaaggctcagagtacccttgccgacctggggcgcctggtgcaagatcaggctagggagattgcggccctccgcctcaccaacgaactcgggcccgggcagctctccgacgccgtcgctcggctggagcgcgcggcgcgccgagtcggcgttcccgtgcgccagcatagccaacttccgcccacacaaccagcgctcgcgttccggttggacgggctagcggcgggcctggagagactggaagaggaggtcggcgagaccataaagtcctcgtcagcttctttggcacgggccgcggtggagctggttctcgcaagccaccaagcgcgcgaccccgacttcatgccgtggcgcgcacttgaagatttttccctgggaactgaagcgagggcccgggagcaagttcgggaggcagccgacgcgattgtctcgggcttcgagggatcggccccccgcttcacctccgggctggcctcggacgaagaaagcgggagcgggggcgacgatagcgacgaagactgggacgacattgtcagcggcgccggcctcggagggccgggcaccccgtgactatccgcgccttcaattccctcttgttgtgcgcccgtagcgggcacctcttgtatttaggccgtcgtgccatcctgccttgttttgcaattgaaacttccttagctccttttcccctttcgtttcttctattcattgctccttccactgaatgtctttgaaacagagagaacaacaaaaaaaacaggggaaaaggggataatcagtccgtgatcgagatgccgctttgaccccatcccccccctcgggcttgttctagcgagatggtgaatccgcgtttggtccagggcacctaagtagtgatgggcaaacccactgctccgagggagccgcctagcgggactggccagaccgaggccgcgtcgaccgggggtcggggacggccgtgtgtaggcccttccgatcctccggggttcggcgcaaccctgggcacggcccgcgttcgtgctccctttcggggtttccaggccgcctcacagcacttaggattcagagcaaacgcagattaccccttaaaccaggacaagccagaggaagggaaaaaaacggtctttgggtaaaagtacataagaattcattgcttgtaagaggaaaaaaggatacacagtggtagcccccagccaggcgcacacaaaccgaggggttgtgcgggggctggcccgagctccaagaaagcaaaaaagtctcgatattatggatagaagcgacggaggtgttcgatattccaggggttaggcaattctgtgccgtcctccgtggttaaccgaacggagcctggccagggcacgccgatcactttgtatgggccttcccacattggcgagagcttgctcagccctaggcgcgactgaacgcggcgtaggacgaggtcgccaacttgcagtgatcgggcccggacgttacgctgatggtagcgtcgcaggctctgctggtagcgggcggcacgcaaggccgctcgccttctccgttcctcaagataatcgagatcatcgcggcgaaggtcatcctggttggtctcgctgtacagtgtgacgcgaggcgatcccagggagagctcggagggtaggaccgcttcggcgccgtacaccagaaaaaacggcgtttcaccggtggcgcggcttggggtggtccgatttgcccacaacacggcgggcaactcctcgagccatgaatccccgtgcctcttcaagacgttgtatgtcttggtttttaggcctttgaggatttcggcattggctcgctcgacttggccgttgctcttggggtgggcgggcgaggcaaagcataacttgatgcccatgtcgtcacagtaatcgccgaacagctcactggtgaactgggtgccgttgtctgtgataatgcggttgggcactccgaaccgagaggtgatgcccctgatgaatttgagggcagaatgcttgtcgatcttgacgacagggtaagcctcgggccacttggtgaacttgtcgatggcgacgtacaggtgctgatacccgcctcgagccgctttgaaaggtccaaggatgtctagccccagaccgcaaaaggccaagagagggggatgacctgcaaggcttgggccggctggtggttctgcttggcgtggaactggcatgctttacaccgctggacccattcttgggcatcttgtaatgcagtcggccaataaaagccttgccggaaggctttcccgactaaagttcgtgaggcagaatgggccccgcactcgccctgatgggtgtcggctatgagctcgatgccctgctctcgagaaacacacttcaagagtatcccgttggcggcacgccggtagagggtcccttctacgaggacgtaccgcttagagatgcgcgcaagcttttcagcttctgcgtggtcctcaggaagagtattgccgtcgagatacgcccggatatcagtcatccatgcgacttggcgaggggggtcagggtcgaccccctcgggccccggggggcacggaggagggtcggggtcgaccccctcaggctccgaggggcgcgggtcggcaggagtcggggcggagggcatgtcgttggagcccctcgaggggttcggtcgtgctgatggttggacaagcctttcttcaaaggtccccgggggaagtggggctcgcaccgacgcaacacgcgacagctcatcagcgagcgtgttgtcgcgccggggcacatgccggagctcgattccgtcgaagtggcattccatgcgccgtacttcgcggacatatgcatccatttgagggtcggtgcattgatactctttggagacttgatttacgaccagctagaagtcaccttggaccaggagacggtggatgcccattccggctgcagccctcaatcctacgaggagcccttcgtattctgccatgttgttcgtggctcggaagtcgagccgaacgacgtatttgaggacgtctccgcttggcgaggttagtgtgactccagctcccgcgccttgaagggtgagggagccgtcgaaatgcataatccagtgtccgccacgaatgtcggcgtttgggtcctctttctcttcgggagaggaagggggtttggacggaacagggtcgtccactggggcccattccacgatgaagtcagccaagacttgacttttgatcgcgtgccgcggttcgaagtgcaggtcgaactcagccagctcgatggcccattttaccacccgtccggtgccctctcggttgtgcaagatttggccgagagggtacgatgttaccacggaaacccgatgtgcttggaagtaatggcgcaacttcctcgaggccatcaatatggcgtaaagcattttctgggcctgcgggtaccgggttttcgcatcccgaagcgcctcgctgacaaagtagacgggccgctgcaccgttcggcggggccggttcgtgttgtcaggggcggatgatccggggcgcgcctgctcgggggccctcgtgggattccggattatcttgggatcacagggctcggggcctcctgccaaagcctcgccttctcctccgggggcctcggggtcctcccgggggctgggggctttgggggacgaggggccatcacgagtcggagggacctcccgctcggtctcctcgcgttcaactactagggcgggcactcaccacgtggggggtcgcagccaagtagatgtagcaatggctcctccggccccggggccaccaggatagggggagaagtgaggtaagctttcaactgattgagggcttgctcagcttcctccgtccatacaaacggcccggggcgcttgaggagcttgaataggggcagcactctctcgcccagtcttgatataaatcgactcagggcggccatgcatccagcgacacattgcacatccctgagcttgctggggggcgcatcctctctatcgcgcgtattttctcgggatttgcttctatgcctcgggaagagactagaaaaccaagaagcttgcccgccggcacgccgaacacacacttatcggggttcagctttacgcgcgtggacctaagactatcgaaagtctcggccaggtcctgcagcaatgtgtcctggcggcgcgtttttaccactaggtcatcgacatacgcctctacattgcgccctatctgattacttaaagtaattcgagtcatgcgctgaaaggtaggaccggcattttttaacccaaaaggcatggttgtataacaaaaggtgcctacaggagtaatgaaggcagttttttcttcatcttccctagccatgcgaatctaatggtacccagagtatgcatctagaaaacacaaaaggacgcaccccgcagtggagtcgactatctgatctatacgtggtagagggaagggatctttagggcatgccttattgaggtctgtgtagtcgatgcacatccgcagcttgccgttggccttcgggacgaccaccgggttcgccagccactccggatgaatcacctcgtggatgaaatcagcctccaggagccgcgccacctcctccctgatgaaggcttgtcgctccggggcttgacgccgcactttttgccggactggtcgcgcatccagtcgcacggcaaggcggtgctcaatcacctccctggggaccccgggcatatccgacggcttccatgcgaaaacgtcagaattcgcccgcaggaaggagacgagcgcgccttcctatttggcgtccaaggttccgccaatcttagcggttttggacggatcatcgccaaggggaatctccttgacgggaacctcgtcggccaaggtgaggcgcttcttgggggcgcgggacgcgggggcctccggggcctgcggccccgtggccaccgccaggttgtccgcgcgctgttctgcacaggcgagggcgactttgacatcaccaaagatggtgatgggaccggcaggacctggcatcttcatctgaaggtaggcgtaatgggtggcggccatgaacttcaccaacgcgggtctgcccaggaccgcgttgtagggcagattgagatccgccacatcgaagtcgatccgctcggtgcggaaattggtggagtcgccgaaggttactgggagctcgacgtgaccaagcggccacgtgtgtcccggagtaacgccaatgattggtagcgacggttggagcttcatccccggggccttgagcgcgtcaaaggcagccggggagatgatgttcagactggccccgccgtccaccagcacacgcttcatcttgacgttgtggatagtcggggagaccaccagggccaacttccctccccgagcgagtaccgtcggatggtcgctttggtcgaaggtgagctttacctccgaccagcgctccttccggatcgcctcgtgggtgggaacagcggccagaagttcccgcctcatggccttgaaccctcgacgagaagtgcgagcgcaagcccccccatcgaatgtggcgacggtcccttggggtatctggaaatccagatcctcactacctccgccatcgtcagcgggggccttgttcttggcctcctctcgctggtcactggttgaggtcacgggagccttgccctcccgtcgaccctgccgcttttcctcatcggcctttcggaaccgctcggccaagttcttgaccgagcggcaagtcggcgagactgtggcggtaagtgttgtgcaccgaacaccacttgtccgccgaccggccttcgtcagagggggcggtggagctcggcttatcgccggtaatctttcctttgcacggggcccgcgtgggcctgtctgccgcaaggacatggccctcgtcgtcggaacgggccggtttcctcttgcctctcctatctcgccgcttagagcggggggcagactcgggggcagccgcagccgccgcaccggtgccaggagagttccaggcccacgcctcctccttccgggccaccttgtccgccagctcgaagagctcggtggtggtcttgggctccttggaggcgatcttctccagcatgcggttatgccgcacgccgttccggaatgcatacactactgcgtgggctggaatgcacggtatggtgtggcggacctggcagaagcgctgtatatacgaacggagggactcgtcgtccttccgccgcacagcatgcaggtccgcttcttccctctggcggggatatgtcccctggaagttcgtgacgaactgctggcacagatcctcccaggagtgaatggagtcggggggctgggtcatcaaccagccacgcgcctgacccttaagggccatgggaaagtagttcgccataacccggtcgtcaccccctgccgccacgatgatcgtggtgtagatttggaggaactcggaggggctgatgctgccatcatacttctccgtgaggttggggcgaaacttcgggggccaccggacattccggagactcgcaacgaaagccctacaacctgccccccaggggtgagcgtggggggagggctgcctcatgaagaagagcgtggcactccagatgaggaggcgccttcccccatgcgggtagcccggcgctcgtcccggcgtcgctcgatgttgacccgagcgtcctgcgccccgtagaggtcatcgtgacacctccgccttcccgagacctccgatgtcgttggagccgtggatcgcctcgaggttgtggccgatcgacgggcccttctcctcgccgatgaggaaagggtagccggggcacttgatgtacctgtggcggtggattggccacccgtggccaactgccgctgggcagcggtgaccaagttggccacgtcgtccagccagcgccgtacgggggtctccggctccgggttcaccgggggatgccgcagaagggcgcccacctcctgaagcgcgccttggggcgaaccaccgtctccgtagtcgagacggcgacgtgctccgacgcgttgcgacctccctccgcctgggagcggggagagggccgtaccacttgaacgggttggatcctccctccgcgaaggctcgcggcggggaggacttcgagagccatggcgatggtgacgagggtcacggttttcgcgcgaatccccactcccagacggggtgggggtaaacgccaccgcttcctccgcttccaggacctcgaccatgacggtctagggcctggtccgacttggggcggaaggaagcgacttcttctcaaacgcgcaactgccccctacctggcgcgccagctgtcgagggaagacccccgatcgcggggagtcgcgtgacccccctttcgtgagttcggccgagggcagagactcgtctctccgaagtcgcgtgttcgtccctccgatccccggttagatagcacacaattcgattacccaggttcaggccgctctgaagcgtaataccctactcctgcgtttgggattatcagtgaactcttacaaggttccccttgctttcccctccaaaagcccccccccctttttgtgggcaaggtcctccttttatagcttaaggggttaccacatgcgccgtctctaccctcctcctccatcggagggaggggagggggccctgctcgctcctgccaccgggtttcaacccgtgccttttgctggaagctaagcatcggttggtccttgagtggggcccagcgccgtctcccgcctgacacggggggcagccctgtcattccctcataaatgagtgaggacttgcggcagccctgcatcgaacgacgtccggatgtgacggggcgtacctaccgccactccgcggagacagaggcgacgtgggggcacgaccacccgttcaatcggccgtgaccggcaatagactggtcacagaccggtcgcatccgactgacaggggtcgttcgggcacgccgcacgtctgccctcgccgcattgaatgcaacgagggacggttggggcgctgcacatttatggccgcccagcctggactcccctctcacacttccccccgagggagggactcgggctaaaacgacgggagagctcgaggggggtgacgtcacccccgaggctctccccatcgccttagtgactcacgaggtatgtgggtgggaccagctcacagagccacgtggctggaaagggaattagctctcctttactccacatacccccgggcccatatctccgacaattgtatatctaatgtaaattatgaagttatatatgatgataaaatctatatttttagcttgaatggcacgcaaaacttgaaaaatataactcaaaatatcatttcttttcaaagttgaattatagtgaaatttattcttcaactataaatttagtcataaacaatttaaaacatatcgaaaattagttatatgatttagatgttataaaacaaaacttttgcttttctaactaactagtaaccatgcacgtgcaaggcatgtttttcttcatattatattatacaaaatacatgattacattaaaaaatttagaactgcagcttctgaaacataatatgtttgatgaataaaatttgtatagaacatcctcatacacctataaaaaaggaataagcagcacatcatcactccaataatattcgtcaaaatacgatccagaacggaaatagcatgttcttgatttagataataacttcataatctgaaaactgtacttaaccataatatattagtgcacttcattggatagtttttatacaacaccaattcgattcatataggaatatcaaaagaatgataaaatcatttaatacagaattctaagtattgctaagcaaacattagtgaaaccatctggttattagaagtattacacctcttcatattatatatttagtaaaaggagtgcatcttgctgattcccattagccttatcatcatcgtaacataataaaagaggagtaaatcacattctgtcgatgctccaaccagagagttgtaaaaagtgacaacttgagtgtctaatcctttgtacaaattccaaatgttacttgaacaataatcttagaccacaagtcatgctagcatcaatacaaaatgagaaaaggttacatgtcagatctttaattgatagtcggaaaaggaccttgtaatatgtatcatggacatgatcctccttttttgctccactttcctcctgattcctgaagtcaaaagccccaaagtagattaacatttagattgctacctccaacgctacttgctgcaatttcaatgaagcactgcaccaagcttttcaaatgaaataaccacgatgcatcattcatggggatcagtggcacctgattattgcaatagctggtaaaaaaagggaagagattggttcatacatcaccattgccatctaattccttcaaggtagcaagatgatgtcccatagttccagacgatgctcagaaagcattgaaactgagaaaataatttgcattaaatcatgtctggcattccctgggtggaaaaatagagaaaaacaaaattcataccaagattacacataaaacagacaacatggaatctgtgttggtaagaaataaatcattgaaagaaaagaaatcaatgcctcagtttacttccatacataacgaaagttaaaataaattcaaaaatttactgagaacaaccaaagcacacctgtcgggctgatttatttttctttctccaatcacatagattctgttcatgaactgctacatatagcacataacaagctgtagagagaggagtgttaaattcaagtacatgtacatgattaatcttcagaataaaactggtattaatcctttcacccatggcgataaagtgctagctattttcagctgcttgatcagaatccaacaaagcatgcgtgcatcttgttctaacaaatattcttccacctcaatagcaataatctttacagaaataatgttgtattacaagttgactaacggaatgggtatgaaagggatcaaaaccaaaattcaggggcatacaagggagtagacaaaactcgggggcgtagaagggattggctaaactttctggggtatagaaggaattttctcaaaattgttatgaaaacggaacatgcaatggttggtccaattttaggaaagagtaagaactaattcttttacaaacttattagattttcttcaaatagtacaagttagtccttttatcattttacttatatcattatagatctaatttaattattgtgcaattatatacaacaataaaatctacataattctttttcataatttcataattgtttgaatgtcacgcaaaagtcataaaatataacctcaaagtgacatatgaaaagttaattatagtgaatatggctctttaaatgtaaaataatcataaacagatcaatagtagtcgtataatttagatgttatagttgcttactttatagcattacttttgttttctaactaatatattttaggaatattactcatagttatattcattttacttttgtgattgtagatctaatgtaaattatgaagttacatacgatgataaaatctatatttttagcttgaatggcacacaaaagtcaaaaaatataactcaaaatatcatttcttttcaaagttgaattatagtgaaatttattcttcaactataaatttagtcataaacaatttaaaacatatcaaaaattagttatatgatttagatgttataaagcaaaacttttgcttttctaactaataaatcataaaaataggagaaggaataggtgaaaaggaggatgtacttttttaaaaaaataataaggtacgtatgtacatacatgcttaggtgagcccgattttgggaggtaaactttttctttaagataaatctaatggttgtaaggcttaggtgggtccaattttgggaggtgataattattgggtgttacgttttcaagtataagatatatacctcatccataatactacatacttgcagtaaatttgagtttttaactataaatttattaatcataaataatctaacaacataagcatatcaataatacatgtatgatttaaatgctttagttgtttaactgttttaaattttgctttctaactaatagaaagaaaaatgagagatataatagaaggaaaagagaagaacattcctacatctaatctaatggttaaaaaaaatgagtccaccaatttaactgcaaattgaaggttagatttgttttatttaaacaacataagcatatcaaaaatgcacgtatgatttaaatgctatagttgtttaaatgttttaaattttgctttctaactaatagaaagaaaaatgatagatataatagaaggaaaagaaaagaacgttcctacatctaatctaatggtccaaaagaatgagtccaccaatttaaatgcaaattgaaggttagattttttttatttaccatgtagtagcttaggagtgtttatagattgccatttgacgatctaggagcatttgtagaaagtttaaaagacttttagtgtataatagatagatagaatcaaatgcttagtctcagggcggaaataatggtttcaggccctgtatatgtgcagtgtgcactttcaagtttcaatctggaaatttttaagaactaataatgtttcaatctggatataactgagtgttagaagaacctgattattagtccccactcttttcagatcaagggggataatacatcatcggattattcatataccatgctttcaaagctactacgatttctaaaaaaaaatcatcggcattggactaccccaccttttttttggggaatatgcaaaaggcttacatacaattgtattataagaagcggtagttcagatacaacattgccttactgcaagcagccggcaaaacatccacgagattacataggtttattctcacaaaacgcccgcattcaaaattcttattagtcggctctacatgttgagcgtgcgaacatacgcatagtaccttccttgtatttttgtgattgcttcatgtaaaagagttaactcaggtgtgtcatggttagagcacaaagccttagcaggccatcagcaacatcaaagttggtgttaaaccaccacctctatatcacattttattggcccacgtcggccaacatcgcaactactattggcttcaaatgggccaggggtgttacatgaattgcctagataatcttttccaagctatttattagctttattatttacaatgtttaattaatcaatgtatttggtccacctcttttcttgttcttgacacttcaaattttgataaaacttgttaataatcaaacgcaagtgatcctcttcaaatatttgtctcatatggggctccatggttcccggacaccatggtatcaaatatataaattcactcaattttttcaaattgtgagtatatacctagttataagaattcgattcatacctatacctatcaacaaaacaactcaaatttaactttagttcacaatccatgattacatacctaactaattatatgtttggatgctatatacctagaatcaaaatctaacaaaaacaagttcaaattcagttcaaacttgctttaaactagatttatgtatgtatatgtatatgtatatgtatatgtatacatatgcatatatatatatatatatatatatatatatatatatatatatataagtttatgtaaatacatatctatatatatacatatatagatatacatatacatatatagatatacatatatgtatatacatatgcatatatatagatatacatatatgtaaattttggattttttgcactcgagcacacatgcacccacaatctacaccctgtatataggttgcacgaatcttagggcaggtacattaggtactaacaaaatgtaacatacatgatgtttactttatacaatattttttacaagtgcaaaataccttgttgatgtaaaatttttttatatgtaaaacattgaggttatcaaattatcagatcaaatttaccttgtttatataaattatcttgtttatataattacctcgtttatgtagttttggatggtaggatatatcatttttgcaagatgataatttggtgatataatagatgtaacattttctcatattgactaaatatcatgttataaaaaaataagggttagagaaataccatgcacaaatttcatggacatgactacatagagaccgcactctatattgtcttaaccagagacttgtcttagacttggttgcatactaatacaccaaatgatatatgttttatagatttttcttttaaattatttatccgatttataatccgattaaaccattgtgcttgttgcaattaaatctttacaacaagatcttacatgactatattttgatagaagaaatatttatgttgtaaattacttttattatatatgtaagttacttttatcatatatatatatataagttacttttcgatttgattaaattacttattatacaatcataatgttgtgagttgtttattttattatcttttttagtcaatttcataatttgtgttgattaaatttaatttttagatagactcacgtttttacctccataacggatctaattttaatatcatgacaaaattacttcctttttatcttaagttacttctataatatatgtaaattacttttaagctttactgaaattacttttatatgtctaagaagtaatttagtgaaatctaaaagtaatttagatatactataaaagtaatttataatttatcatcaaaatataatcatatgagatcttgttataaagatttagttcttacgaacacaaccgtgtaatcgaatcatagatcggatgagtggtttaagaaaaaattttatttgaaacataggtgataggaatatatttcctacttacttgatggggactagtactacagttaaggcatctagatagggtctctcgttagcactaaaacgagaggcctcttgatttagattttgccaaatttcatatatgattgttactgaaaaaaaaaattggtaaaaaaatgcaacatacttattatatgtgttgtgtaccaacctaatcttttctttctatagtaagaattcatttgatcaaagttaggtacattttgcagcacgagtcttggaacacatcaatggtatagattgtgggtgcatgtgagctcgagtgcaaatatatatatatatatatatatgcatatgtatatgtatatgtatatgtatatgtatatgtatatgtatatgtatacatattgaagaatattgaagatactttacacacttccttgatagtaaaagagcctttgtgatactcagaactttccacaactcattcacggtttcaagatgagaaatccaatcaaaaccagaactagaaacaccactcaaaatcaaattttgagctatgcaattgttgtattaagaagcaactttgtttgcatcaataatacccaaaggaaacgtataagcagttgaaactttttgctaaatatcaaaaccttgagaaatgatgtaagatttcctccgagatttcaagaattaactataagtcaactccaagaaaaactatgagcttggttgaaaacctataggccataacaagagtacacgtggctctgatataacttttaggattgaaacacagctaaccgaacctactagaggtggggtgattggtagcggaaccaaaaacaaaacaaatctgggacaatcagtggaagatctaggaagagatagtagggggggtcttaataaactagataaagctacagccccattcccgcatacatatagcaaaaagatttagcgggggctttgtgggggctcccatagtactcgcgccagtagtggggactcgagaccctgccgtccccatgctggatccgcctctagagacaataaatattcacccaaaagaaaagaccaaaaggagaactcttaactattgttctagcacaattttattaagataaaataaaatcaagcaaaaaaaaccaaattaactagaacaatgtaaaagatagtgagattaccacgtattcctttgcatcaaagtgtttacaaagtgcatacaaaagcactcctctatgatctcacaaactgaactctagatgcaagagcaaacatgtgaaaccctttttttttctcttcatatttggtcactccctctagtgtttatatacccatatccttcataaccaagtacagttcctaatctaactaggactaggactccgtcatattccaactcttttctatctttccaaattaggattcagatccctccaaatttggacttcctccaagtttaacttctttatccatatacgcacacctcccatgtagttctctagcctgagcatcccatatgacatccttgactatatggaccttaaccctctgtgtttagtcccgatccagtcaccaaccacgcttctgcggcctcaaccaatgcatgcacatgagaataatatctaaaccatattccgagcacaagttcttgccaacttgatttaaattagtgcaacaatagagtattatatgtaacatgtaatgtacaaaaactacgaagaagtcatatacatgaaattcaacttgtctgaacatatttgcaaattgttcatcacaacataaccttcaccatgacattacaatttattcgaggacagaaatgaatgaattaattgttacaaatttgaaaaattgttcttaaaaaatattctaattaaaaaaatttattgaagatttgttttttaaaaaaaaaacttgagtttaggaagcttgtctgtgataatccggttcccttaatcaaaaaataatagtcccttaatcatcccaattatatatcacttcaaccattatttcagcaccttaatcattacaaccatcatttcatgccattgccaattgcccagcaactgctagcagttttccgccgaacctgatggcagaggtggaagtcactttgctggagggcgggatcgagtggctggcgcagaccatcctggagaacctggaaagcgacaagctggacgagtggattcgtcaggttgggctctctgatgccaccgagaagcttaagtcagagatggagagggtgaaagtggtggtcgctgccgttaggcagagagcaatcgggaacaagctgacccgatcgctcggtcgtctcagggagctgctctacgacgccgacaatgccgtcgatgagctcgactactacaggctccagcaccaggtccaaggaggtaatataagcgtccgtacgcatgtaagatgtacatcacctctttcgtctgttggttcaaatgcatggcaacaaggcgctcctgaaagcgcgcataaacatgcaccagtgcaagcagatcaggtagtgagaccaaaatttaccactgatatttcgagaagcagtggtgggcctggtggcaaaagatggtccaaggtatggaagtactttgttgccactgaatatgaaaatggaaaggctgtgaaggtcaaatgtagtttctgtaacactgagctcaagtgtcaaaatggggcgtcggctttgcatattcacctccgtagcaaccgttgtacggataagcgtaaagaccaatcgaggtaggatttatgttctcttcttttctttttctttaaaaatagttaaaaaagtaagtgctgaactaatatcgctcttactgttgaactctgtggttccggcagcagaaaaaggacgaggattattggtgagtcaacgcacaacaatgcagtagtcgacgacctcgactactacaggctccagcaccaggtccaaggaggtaatataaacgtaactagatgaaaccccgcgtgttgctgcgggacttcaatataataataataagtaaaaataatgtgtaagatagcttaacaacataaacttacataagtactaaaatcaattgatgtgataatagcttaattttttttagataatggattatattaaacccggcctctacaccttgcgagggtgtacacagccaaaataatagtttaattagagaagagaaaaataatattaaccaagtgaggatgaacttacataggtgcataagatattataataaataatgaagatatatattgaaatattatgtgaattatgtgtgataataatttaacatgcctttattttaaaagctctaaaatgtaataaattttaaattatagataatatagcatgtttgtatgatattaaatgtattgattgttaatagatgatgatgtgtcaacttgttagtgaatgatgatgtggcatctttgcatgttaagctttaggagttagtgggttataactttatagtaagataggatatgtaagatgtatccgttttagtttctgcccagggaggtgcattcattccgctttctcttggttcagatgcatggcaaggcgctcctgaaagcgcggataaacatgcaccactgcaagcagaggcagtgacaccgaaaagtaccactgatatttcgagtagcagtggtggcaaaacaggccggtccggggcatgggagaacttacgtatcactgaattggaaaatggaaagcctttgaaggcaagatgtgttcgtaacactgtgctcgagtgtggttctcacacagggacgtcggttttgcgtaatcacctccatagcatcagttgtaagaacaagcgtcgagcaagctacccaaacctatcgaggtaggatttatgttcttttcttcttcttgtttttacccggcactatttttaactaatgtaacatacttgcatggttcatcacgtcagcagcgattgtgtgatccttcttttttttttcctttttaattttgcagtacttctgacaccacagcaaatatcaccattactgatcaactcggtggttccggcagcagaaaaaggatgaagattaattccggcagaaaaaggatgaggaatattgatgagtcaacacacaacaatgcagtcgattcacacccttggaacaacaatgcagtcgattcacgcccttggaataaggctgaattttctagcaggatacagcaaataactggtgggttacaagttgccatcagggctgttagtgaggttctcacgctacatggagcggaccttgttggaaactcaaatcatcgtacaagtacaacaacgaccctgatccgaacgtcaagtcttagcccacacaaaatttatggaagagacgcggagaagaatgccatcctgaagattattacagatgacagttacgatggactaactgtagtgcccattgtaggcattgcaggagttgggaagacagctcttgctcaacttgtatacaatgatccaatggtgaaacataactttgatcaggtatgggtttgggtatctaaagactacgatggagtgatgctcacgatggagattctagattttgtctctcaagaaagacatgaagtatctcatcaaagaaaagaaagacacaatgcaataagtagttttgccaaacttcaggagatcttgaaaaggcatatggacatccgcacgaaaaaattcctgcttgttttggatgacgtatggaatagcatggatgataacacatggaacattctattagctccattgaaatcaaatcgtgcaaaaggcaatatgatacttgtgacaactagaattttgtctcttgcacaacgggtggggacagtcgaaccgattaagttagattctttgtcaaatgaggacttttggttattttttaaagcatgcgcatttgatgacgaaaattataaagcacatccaagtctaaacatcattgggcagaaaatagctgacaagttaaatggtaacccattggcagcagaaacagcagggcaaatattaagaaagcatcttacaattgatcactggagcaacactctgaggaatgaagattggaaatccctgaatataagtagaggaatcatgccttctttgaagcttagctatgatcagctgccttactatttacaacaatgtttcttgtattgttcaatattccctagtggttattgcttccttggacaggagttgatccatatttggatttctcaaggttttgtgcatcgtaactcttcaagtaagagactggaagagatcgggagggagtacctttctaatttggtaaactccggcttctttcagccaggtgcacagacaaatcagttcactcaagttgacaagacatcctatgttatgtgtgggcttattcatgattttgcaaggatggtttcgagaacagagtacgcaactatagataatccacagtgcaataaaatgttgccaactatacgtcacttgtcaatattaaccgattctacacaccaagaagatcctgattgtggcaaggttgaagaaagaattagaaatgcagttaaaccagtgaaacatttgagaactttggtgctaattgggcagcataaccatatattcctcaaatccttcaaagatatagtccagaaggaacatcatctacgtgtcttgcaaatctctgcaccatttactgatattgacccctcgttattgaatctggtgaatcctacccatattcgctatataaagtttgatgacggagctttgcctctatctgtcggcaagttttatcatctccaagtatttgatgctggttcagaatctgatcttattatacctaatgatatggataacctcgttagcctgaggcatcttgtagcagcaaagcaagtgttctcgtccatcactagcattggcaaaatgacctctcttcaggaattaaatgactttagtgttcataattctttgggcttgaaccaactacagtccttgaaccaacttgtacagctcggtgtgtctggacttgaaactattacgactagaatcgaagcttgtggtgcaagactaagagacaaacataatttagaaaagctacatttgtcctcgaaggatgcaaaggatggatatgacagtggcatgagctctgaaaatgagtatgacacagacatgagttctgaaaatgaaactgacagtgacatagaacctctgatggagggactgacgatggctgatacaaatatatccccagttttaaaaaatttgccaggcatagcacgtgaggtgcttgatggtcttgaaccacatcacagtctcaagcatctacggatatctgggtataatggagctacctccccaacttggtttccgtcttcacttacctgtctgcaaacacttcacctagaaaaatgtgaaaaatggcaaagacttcctttagaaaggctaacgttacttagaaagcttgtgttgatcaaaatgaggaatgcaacagaagtctcaattccatcagtggaggagcttgtattaactgaattgataagcttgaaggcatgctcctgcacttccatcaggaacttgaatgacaatttaaaggttttgaagattaagagttgccctgcactggaggtctttccattgtttgagaactacccgcaatttgagattgagcagtcgtcatggttttcctgtctaagggagattaccatctatggttgccctcatttgcgtgtgcacaaacctcttccgccttcacctaatgtcgagaaattatccatcacccgagtttcaacacttccatcaatcgaggggtcatctagtggaacattaagaatcggattttcatatgattggatggatgaatgggatgaatcttctgatcagatgataacgttggatgacaaagttatggcgttccgtaacttgaggttcttaagtggattggtaatatgtggttgtcaaaatcttacgactatatcattggacagtttaagacaactcaggtctttgaagagtttggaaatatccaactgcctagaacttttcgctccaaatgtcccatcagatacccgtgaagacatggcagctgcagatcacaacaccctcctatgtcttaaacttctcgatattagaaattgtggaataacagggaagtggctatctctgttgctacaacacatgaagaccctacaagagttgaggttacaagactgcgagcagataacaggtctatctataggagaggaagaatgtggtcaaccaaatcttatgtcagctacggagactccatcattgggattttccgctgaaaacaaacttccacgccttccgttaaatatcatatgctctctgaagaagatatatatcaaatggtgctctgatttaacatttcatgggagcaaggatggtttggctggatttacctcccttgagcagctagagatttcggtatgccccaagctcatcgattccttattgcataatgatggaaatgttgagcaggcgaatagaagatggctcctccctatatcccttgaagaacttattgtacaatatgatggtccctttgaaatgcttcaactctgctttccagggaacctcacccgcctgaaaaaactgtttctaatgagagaggtaagtttgacatctctgcagttacattcctgcacagcactccaagagctgacaattcgttgtgcgcgcttacaaattactagtaattctctggaaggcttgcaatcgcttaattctctggaaggcttgcaatcgctcggcaacctaaggctgctgcaggtaaagggccttagagctcacatgggttacctccttccacaatcacttgaggaactttgcatcaacaactgttttattgaaaccctgtgcccccgctttcaaatgaatctcacctgcctaaaaaaattagaggtgcaccactctgaatttttgatatctctagagctgcaatcatgcacggcactcgaagagttgacgattaaatattgtaaaacactcgccgtactggagggcttgcaattcctctatggcctcaggagtttgcaagtacacgaatgccccagattgcctccatgtttggataatttgtcatggcaaggccatctgcgactagaaaagttgtgcatcgatgatacctctatccttaccacgtcattctgcaagcacctcacttcccttgaatacctaaagatttacggtttcagaagcgaagagtggagactaacggatgagcaagagagagcgctccagctcctcacgaccctgcaagagctccgctttacgcattgctactctcttcaagatcttcctgcaggtctgcacagccttcactccctcaagaggttggacatcttgtgttgctatggcatcaggaagctgccagaaaagggtctcccaccttcgctggaagaactggatatctatggttgcagaaaagagctagatgaccagtgcagaacgctaggaagcaagctgaaggtcaaaatcgatgggagtcttgtgtcttcataattgaattattgggtggcttgacaggaacttcttttatttacccaactggtatcgatttattccttccaaaggtacaattctgcattccaccaagttatcttcacaatcaacacatgctcagagttattagttagtactaaactaggcagttggtcgtcatgttatcataggcactgacgttggcatgtgacatcaactaggcatgtgggggactgtttggttggagtcctgaactgaagtgttttcgcctgacctgtcgtttccctggcgcctgtctcgtagtacttgtttggttgatgtcctggatcgtttcatgggtggccggcctggcctggctcaggcctacaaaatccaacgccggactctgccctacatcatttatttactcttgtttattttcaagtttcgatgtagaaccatatagacaaaacgcaggagtatatttctatttgtttttcacgttaatgaagtctcttgatattgtttattgaggtaattatcaaagaatgatatgtactgttatcctcaggcatacaactgcaaccaaacaagcactcgctcaggctgcctcctgagcaggcaaaaatctagactgtatacatgaatgaccagaagcttaaatcctattatatgcttgcgttgatgcataataacattgatagcatttgtcctactatcttgtttcatctcatttccactgcatatatctccaagcacagagattaatatattggcaacaatacatgcaaatgaggtcttataaaaaaaatggatttgctctttcatacaactaggaaggtagcccgcgcattcgcgcgggcatgtctcatacgtaggataaatttgagaaacttataaaggcggtataccaacgaaataatatctgcaaagttttaaaattgtgttttttaatgaattttctttgttagaattgcatgacgaatttaattattatttataaaattacctattggagattatggttggtaacttacggtataaaaaatggtacctaagacatatgctcttgagattccataggaaaagagggaaaacttaagttataaaagacattacatatacaaccatatataattaatggctctacccacttttgtagaaaaattgataatcaatttattatttgatggcaaacatcgattggtcatagctatggataattcctcataggaaattatgagttcctatgatgaatgtttttttataaagataaatataatagttgaaaatagtgggtctatcgatttattaaacaaatagtgggcctatcgatttattaaacaaatagtgggtccaccggtttattagaacgccacgtaacggtttagtagcgttttgtagaatgccacatggtaatttaggagcgtttgtacgaagtttaatggacttttaatatataatcgaagatatctctttgtacctaataaagtataaaaacataagaatgaagtgttaatctaggaagagggtggtgtaatatgaatcaattattataattatgtaattacctaggagattaggacagagggattaagtagtttaatagatctataatggttaaaaataataggataagtgtaaatcgatggtgccatattttgcctgttactcacaactttttgaattttttaatcataaattacggaggagatcagcagtgagggattaattagtttaataaatctaatctaatcatcaaaataatatttttctcggtttaagtgtaaatcgataatatgatgttttgcattttacttagaacttttagattttattgatcttaaattaatttgtttaattataaattaaatcatgacaaattagctcatcatgtttaccttgaattgctagctataaaatagaaagtagagagaatggcagacacgtgacatatatgttacggtcaacataataataataataataataataataataataataataataataataataataataataataatatatgaggtatattattattattttcatatataataggtagttgtatatgagatataagtacgtacttactatttacaggatgggtggattaggtttgggtggttaactttttatttaagaaaaatttaatgttgtaaacaataggtccatcaaatataatgaaaatcaacggttatttgttagagtggcatgtggtggcttaggaacacttctatgatgccacgtggcgatttaggactatttgtagaaagtttcatggacttttagttaatacatatcaaataatgatgtaaaataatgtgtccatcgattttagtggaaaccaatgattcgatgtttctctaatttaaaagtgccacgcaacgattagaaacatttatatgatatttaatggactttaacatttgtgggggtattctttctataaagataagttcaattttaattgtgattgaaaaaacaaaagaaaggaacaaagggaaaaaaaaaggagaacggaacaggcgagcagtacgtgcgtactgcacggatgtggggccggtgagaggttttggtttttaagataggtctaacgatgtaaaataatgatttatctattttattagagtgccacgtggcaatatgagagcgttttgagggagccacatggcagcttgagaatgtttatagggagtttaatggacttttaggaaactatcttacattatttatttattgggtaaatatataatggtgtaaactttatttttaaatattaaatattacatagctaatttagtctcacataatctacgtgaatctatatataattacctatcacatgatcacagtgtatttatatttgcattaaccttgctcacctttacaaagtagctagccagaaataagacaaatgtacgcatttgtacgagtatttctgagaaagagaaaaagagaggggaaaaagaaaaaacggatgtctttcttcggtcttgtacgtacgtacgctcatgtacgtaccttacctacacggaaggagatgtgggaccatagttgtggacgttttggttttctaatatagatctaacgatgtaaaataatgggcccaccgatttaaatgaaaattaatggtcagatttctgtttcttattagagtgccacgtggcaacatgagagcgttttgaggtagccacatggcagcttgagagtgtttatagagagtttaatggacttttagtatataataataataataactagaaaggtagcccgcgcacatgcgcgtgcacctaatttaatatgtgtgaaattttattacgaatatctatatatgtgactttgtattaaaatattactttactatatataaattattatttaacttatttaatgtgctttcaacgttggttatgattttcttctaattacccttgtagcacataaattctaaactaaaattacttaaaatcgtacagtttttaatttatgagataatattatgtcgagacaatagatgtagttcctatgaaaaaattttgggtatttcaactacccatggggatatctttccccaaacatttgctcaaaaagtccaaatttttatcagattctattcgaaattttagatattttcgttctattagtcctgctaaaatatttgaatatttccatttagaacgggttttaaacccaacaattttttaaaacatttacataaattttgaattcttttcatgtccatttttaataaattggagaattccattctaccaagcatctttgaaatgatttgatatctgcaatttgggttataaatatatgtgggttaagcctacaacctgacatggggtaaaatcatctattctaattatctctctggaattgagtaagaatatgaaaatagaggtcatataataaatgcacatttctatattattctacagtaaatattatgaatttacgatgtagcatagcaagttttaccaaaaagaaataaatgaacatggtctttaattccatatggaattaactcactatattcactatattggtaatgccacctcagagttgtttcactgctaagatttcttggtgcatttcctcaaaaaaaaaagatttctcggtgcaatccacatgtttggtactatcagttggatgttcaactgagcatgttttttacttttaaaatgttgtctataaaaaattaacgctagatggagtacacattatgacataaaattgttatcatttaaagatttttttcctctaatttattagtgacttcatagattatatataattttatctcctaaaaatatcatttattgataaaaagcaccaaagtcaaattgtataggcaaattacccccatatacatgttgagagaaattaattaagttgatagaaagttgttacgtgtctaattggatacaggtaataggtctacaggctttattaaaaattaagatgtgatattttgatttataatgatgaattttcatttttttactaagtggatgcctcaagatggatgtgggtccggaatgtaatttttatgttagcatttaaatatactcaatatggatcttaaagttgatttcactaagaaaatacaatagttcaaacaaatcataagttgaatatatgatttaaaagattatattattttatttatattgttcccataaatgttagtaagatgagaaagagaagaaaagggatcatgggagagctagctagacctgttatgtggaggtccatttgtggttttttcccataaagataattttaatttcaattattataagaataaaaataagagaaaaggatgaacgatgcacaccacatagggataaggtgggcgagatgtttttcttaaataaattattgatatattttttttcaaaaaaataaaagattgatataatgactaaaataatgtgttggatgtttaatgtataaggcttcgacattgttgggtgggggagagaggaaaggaggtttgtttctttttttaaatacaaatctcataacaacctaaatggtatgcccaacgatttaagtcgaaaccaatgattatatgttttattcttaaatttttattatttactctatatacgaagtgttacagagtggtttacaaacatttataaggatggttaatgggttttaacgccatataggagcgcgaagggagtttgggatgttttacgtaagaaaaagatatatataataacctaaaataatgtaaatcgatgaacgaatgttttatatctttttagattttctctaatttaggtgtcaaatagatgtttggtaatatttatatgatgtttaatggacttattaggatgtgctagaggaagaatagatctagtagattgatggattttttaggcatgcatttttctaatttaattattatggaaatagaaaaatgaaaaaaaatggaaacggagggatcgtacgtacatacgttgctgatagattgatggatagtacgtacattgtggaggcgtcgtagggggattttattttaatgtaaaaataaatctaacggtcaaaattaataggtccaccaatttaaatgaaaatcgatggtcaaatttttctattttgttagagtgacatgtggcgtctcgagagcgtttataggatgccacatagcggcttaggagtgtttataggaagtttaatggacttttagtatataatagatatagatagatagatagataaatatcaaagtaaatgaaactatacaaaatgtacagacgcaccgcgaatgcaaaaaagctaactcattggaaatcaacacctgagctgcaaacagacgaggaaaaaaaaaactactacaaacggcacctgtcagccgcatctaaacacgaatctctggaacatgctgcatcttgtgatgcattccgttcttgtgccattgagttttgaagtccctgtgcaggtacagaagccagagcttcttcagggtccgaagggactcaatgccctcagggactatatccagctttgagagtgacacaacgtacaaaccttcaatggatggaagcgccccatccatgatcttaagctggttcacattgggcatgtgctttaagacaagtgtcttcaggtgggggaaagactctgttgaaagaaccaaaatgtttgcactatgcatgttgttcagtctcaaataagtgaggttcggcaaatttgaagcgagcatccctagtggatcttcgccaagatgacaccaacttagagctagatatttaagatttgtgccgttcccgtgaaatattgggcaatcaagtgtacccttagcccattgccctctgatgatcagtctgtggagttctgttgaccttggcctgagagcctcgaagcagagttcctcattctcatcttttgcagaaagaagcaagctggaaagaaatggcatgcttgacaatgaagcgaaaatatttgcacaatcagcagaacttatgttgtcaatccacacacttcttagttgcatcaatttcttcagctgctcggccaggtctttgctagactccacagtttctagagtctgcagctcttgcaagttggaaagttctttaggagcatgcattccaacaaagtaccggaaatctgactgcttctcatcaacgtatctatcggctaaaaggtgtcttagcttctttatcttaacaacacttcgtggtagcttctcaattttggtttgcttgatgtcaagcgtgtggaggttagataactttccaatagactccggaagtgatttgactttggtgcgtcgtaaaccaatgtagcgtaaattaaacatattcccaatagaagtcggcacttcagtaatctctgagtcttgcagctcaagaactgtaaggtagctagatccacacaaaattgaggataacatctcaagggacaatgaggttgtcgaaagtgagatcagggtccgaaggcgcatgaatttaactgttgatacagtatcatcactccatccacatgttgacaagcgacgaacttccttatccataagcaacattgtgccaagatcatttgctgaaccaaacctctcctctttagcaatagaaagagccaatacacgcacaatgtcatgcatcttacaggagtttaccctgccaatctcatcattgtccacaacttccagcatattccggtggatcagttccataaggtttccctctgcgacatcctctagcgtgttcttttctttgcttagcacaaagccttctgcaacccacaacctcacaaggctctcccgtgtcattgtgtagtcttcagggaacaagctacagtacaagaaacaatttctgaggtctcctgataggtcatggtagctcaaatttagaattgctcggacatgatcattgtttgctagctcagtccgaagctctttgtatattttattccaaacaaattctgctgctggtcttgaagacagaaggcttcctaccgttacaattgctagtggtaggccatgacacctatccactatagaattagcaaccttctctagctccttggggcatttgtggcccttgttatagaaagcccttctacagaagagttcaaatgcatcagcgccattcaaaggctggaggttgagacggcgtgctgaggtagcaagggctgcaacatcattctctcgagttgtgatgatgactcgagttgcttgaagattctggaatgcttcttgcatctgaaagtacacttttttgtcccagacatcatccagcacaatcaaacatttgctatcttcaatctttttcttaatttcttctttcaggtcatgtgcatccatattgttcaagctatcaagtgacagttcagtagacccaatctttctaaggagctttcttaatagagcctccacattgtaggtctgggagacaacaatccatgcatgagcagcaaagttgaccttttcacgctcataaatatttgtgactaatgtggtttttcccaacccacctataccagatactgttatcactgctctatctgactcatcagagtacaaccatccagccagcaatctcttgtggtcttcaatccccacaagatcttcatctttgacaagatatgggaagttgtcgtgagaccgtggtctgccactctcagcaagctggttgggattgagctgggaagggtgcaaccactgctctttaagctttataacttgctggatttgcttctctaacttgactacctcctcagcaatatcactaaataccatgacgtaatgagaacctttaacgaagtacttcttcaagaaaccttcttcctcaagttgaatagcatgatatgagtacttgtccattacgtcctcaacatggtaggctaactttctcacctctgcaatccaattctttacaactatatcagtgaggtaagaggtgcctatctgtagtataacactgttcatgattgtcagttgcttccttatttcttcgaccttctctggcagctccttcagattagtaaccttttcagacaccttggcaatgacagccttggcagcttcatctgctaacacgttgccaacctttttgacagcaaggagcactgcctcagccatttgttctgaaagaacacacaccatcatgttacaaaagagaacaacaagtggcttgcgatttctagtctccaatttaacttatatatcaaagaaaatttttatttattgatgcctttaattatcactattttcctggagaaatgtgtgctttcattgaacagaactccaatggtttctgcatgaaatgtgtggcactgttcagacttggataataatggtccactgtgagaaaagaaataatggtactcatgcaccgtgtccatagagtaattcttttaccacaagcatctatgatctcataaaacgtttaatgagaagttaatggcatctcataagatataactcagcaaataaatgacaagctttatttgatttgctcatgggcacagagaaggattccaaatcagaatggtaaacagtaccatatatgaaggccagtgctaagttatatttcattaatttacttggaacgattttttttggcaccttacgttggagttcggaaatcggctacgagattttccggacaaactatgctttcggaagagctctcctgcttggcggccttatcgtttggcttttttcgtaataagccaaaacggcttattagagaataaaaattaatttgtaggtaaaacttttatatatgtgttctcggtgacttaaaagccaatgctgtaaaagaaactacgttgaaaatatctcaaaatcaatgtcaaaattaagtttgaaaattcaaattttggctttttctttggctggataggccatccgatgggagcctgggtcagtaaaagaaacattgttatactgttatacatcatgaagtacagctattctgtatgtagttgtttactttaatttgagtatgcatttattacatttcaccaacatacatacagcttcagctgaagcaacttgtgggctgaacatagttcatacatattcaaagaaaataactggttcatcaacaagacagtgagcagaaagactatacattagataagcaagtttagcagcattacctggaacattcgactaacaactatgaagaacaaaaaaatgatccatagaacagaacacacaacaaatacacagccatactgagtagtgatttatatgcttgctcacctgctggaagaccagagaacagggctggagcgcaggtgctgtacggtcagtgtgctgaaccctgcaggatcaaacaagagaggatgagaacgcagtcagcaaagcagatcgtgatcgtgcacaaccggggacaagatttggccaaaaaaaaaaaaaacgctgccgcgcgcaggccgcggaaccacaccagacggaggcgtggcggcctcccttcgccggatgcgtcggcgcgcagcgtgcctagactcgcggtcgggaacgggcattccgacgaacacttggcgggcgccgcaaagggggggacactgggtgttcatcccaaagggggagataatcaatccttgcaaagtcaactcgcgtaccaatcggtcaacggaatgcggatcgaaatcgagagaaggaggagttcttcgcgatcttcgggtgatactaaagtcgccgagcacgagcaaatcgagagagacaacaaaaattctccgcggcgaaacggctctgcagcatgaactactactaatcgctcgcaagctccatggattctgcaagtgaaggaatgcacgcacctgatgaatcaatcgcaaggagctcgccgacgagagaggaggaggagactttgacccgctcggagctcttgaaccagctgcctgcagcttggttcttcttcttttcctttcttttttttttccttatttctttctgggtcaggtcgtcgctggcttttccgtctcctaataatttctggattttattggtaaggagacacggtgaagtggagtaccgtctccaggcgtccacaccgctactcagggggtcgtgttctgctccagggctacggctgcggcctgttcaagtgttcagttgtttgacataccgcgaaaaattaaatgaagccaaatgtaaatgctgaaaactactctctctatcctaaaatgtaagtatttttagctataaatctagacaactattgttacattttgggacagatgtagtatattacaattaaaaataacccaaaatcaccatgataagagtggcattcgcacatgcgcgggcactttatataatgtgagtgaaatgtttttatgaatatttatgtatgactaactttttattaaaatattttttactatattgtacactaaaattaagcttatttagtgtgctttgaacgctagttattttcttacaattacacttgcaacatataaaaataaattctaaaacaaaattacttgagatcatgcaattttgaaattacgagaaaacattatgtcaatggagtggttcctatgagaaaatttagggtattttgactccccagaatgatatctaacatcccaaaagaagttcaataatatcattttttgtttgattttattttaagttttatatattttcattctaacaatcctcataaaatgtttgattttttctatttagaatgggttttaaatctaccaacattttttttcagttttcaatttattttatgtcctatttttttgggagatttccattgtatgaaacatgcatgaaatgctttgatctttgcaatttggggcgaaaacatgaatgcatttatggcatagggtaaaggcatatgttctaaattttctctcatgaaatattgaagtaatggtcatagtatttttttgtgaactatcactaaactgaaatgtcttataacaaatacatgtttctagatgattctacatcaggactcaagatttgaaaatgttaacatgttttacaaaaaaaatatataaaaaagtgggcatgtaaaataaagtttccacagatttaaatcaaaaccgatggtcggattattttttttcaaaattctaagattttctccgatttaagaatgtcacatagtagcttataagcatttataagatgtttaatggatttgaatatcgcataggggtggggagaggagagaggtttggatttttctatttaaaaatactatcaatctaaaacaatgcatctgtcaatttaagtagaaattgatagctggatactttggttttttagattttatagtattttctctaatttaatagcactatgtagcggtttaaaagcgtttgtaggatgtttaaagtgatagaggaagagttagtagacctagtaggtgaaagtctatttgtggggctttattgcctgtgaaaataattttggttttaattaatttctcatgaagaaaaaaacatatagaaaagtaatgggagggaagtacgtacgtacattacacattcgattttgctagatatttgtcatcaaatttctccccctaatatttgtcagatgtaattacagtacaatcgtagtgtaattacactgtaacttacatgtaattatactgtaactataatgtaacttatatgtaacttttaagaatctctcggtaacatgttatttcagtaaaatagaggtcctgggaataaattctttcacatatgtatgtgctgtgatttttttcttcctcaccagaacaaatcttgtaatagatctaacgattcaaaattacgtgaaacttacatacaagttacactgtagttacatgcaagttacagtgtaattatatacaagttacaagtgtaattacactacgattgtactataattacatctgtcaaatttttagtagaaaatttgtcgacaaatgtataggtggttccttacattgatggttgatcattttttttagaaaaaaaagagagtaaattttataaaactataggtattttgcacaatttataacaaaactacagatttaatagctcgtttcataaaactacagatttagtgtctatgtttatcacagaacttatttcgtaaaactatagatttagtatatcgtttatcacagaactatacattaatatcttcatttattagaaaactacaggtttagtgtctccattatcacaaaactacaggttttagcattgttaaaacatgtagttttgtgataatggagaccctaaacttgtagttttgtgataaatgaagatattaaatctataatttttaaaacaagttcttaaatctatagttttgtgatatattgtgcaaagtatctatagttttgtgataaacgaagacactgaatctctaattttgtgaaataagttcttaattttgtagttttgtgatagatcgtgtaaagtatctgttgttcttttttaaaaaaagagagatggggcatataccgagcatgcaaaacttaaataaattcaaattgtaataaactatctccatcgtttaatacacaacaccattaatttttgattgaccatttatcttattaaaaaaaacttaaaattaattggtacagaactacaaattccatgtgatacaactatgattctgatcaaacaacagaaaacttgctaccaagtgagaagaaaatgattggttgctagattgtccaacaaaacaaatatcttaaagctacaagtcgtctcaatcatttcattaaaaaaaaaaagtggtctcgcatccagcagacaagattgggggaatcaaacattaggttaacactcacattcagctgtcaaataaatcatcaaaaattacccccatgtagtaattctagcataattaaactactttccgctgaaatccgaacatagttttctctttgctttattttgctgggaataattttctctttcttaattggtacaatattagttcaccaatgttgtgaatagaattgattgagcgagagagattgagtgagagagagaattgcacacaagaatgtagagaatgaatgagcgatcctttattgataacaatggttacaatatataggaggaagagaggcggcaacagaagacgcgatggtttgggaaccatcgcgtcccttgttagaatcgccgtgaggcgagaagaggtgtggggcacgaccaaccgtcgtgtcccagatttattgctgctctatccagatagatagatgagatcacccaaatatttcataacaaccaattaagttgcttcatacatgtactgtggagatcgccttggtactcaatactccctctgtattttaatgtatgacgttgttgactttttgttcaacgtttgaccattcgtcttattcaaaaaatttatgtaattatcatttattttattatgacttgattcgtcatcaaatattctttaagcataacataaatatttttatatttgcacaaaaattttgaataaaacgaatggtcaaatgttgatcgaaaagtcaacggcgtcatacgttaaaatacggagggagtacttactagcttgcattcagattgcaatatatactactccaaataaatagatgcaagaaaaagcaatagcagcaagtggttgtgctccagctagctgccggggtggtggtcaatgaacctgtccaagccggacctaccctcttgtggccggacatggcggatggtgccgacgaagccaccatcggaggcgctgggcagttgtcgttggagccggcaaagagcgaggaaagatcgccatggccactggagtcgttgcggcactcgcgcctcataatcgcggagggcggccacgcctggagatggtggtgcgtgtgaactgaagcactcgctcgccacggtgtagggtccgccgattaattgaactaggaaggtagcccgcgtattcacgcgggcatatattttagattgtgtttgaaaaacttatataaatggaatattaatatgcggtcatgtattttagattgtgttagaaaaacttatatagacataatatgaatatgacaaagctgaagaagttatcgaaacttggtttagtatcaaggttcttctcttccggccccgagtgctacagtcgttagatgtctaatgggatataggtgatgggtctaccagtttcataaaaagttatggtgggatattttgattttttttagtgagaaattttagcattttcttgtatttggtggaggccttgatatggatgtgggtccaaaatgtatttttttagcatgtaaatatagccaatattggtattaaattattgatttcagttaaaaaattatgatagttcaaacagatcataagggacagatggatagctggctatacatgattggtggaggttcgtttgtgttcttttctaatatgatagttcaaacagataatattaattttaattatcataaagatagaaaaacatagaaaagtaagaaatgtaacaccgcacactgcccggggatgtggggatggagggagggttgggatgtttcttttttttttgaatgaaatgctttatctaacgacctaaaataatctaaaataatgtgttgatttttaagtcaaaaccgatggccatatcttttataattttttttcatatttgttaggattttctctaatttaagtgagacaagcagtagcttagcatagcgtttatgtactttaatgcccacagggatggataggaagggaagatagttcgggatatttattttgaaaaaaaaataaaagatcaaataacgtgtaaaatagtgtgtatgtcaatttaagtggaaaccaatggtgggatgtttctcaaatttaagagggccacataatggtttagaaacatttatagaatatttaatggattttaatgcgtatgatatctatttgtgggtttttccctaaaagataattttaatttgggatgtgcttctatgcttctcactaaaaaaacctgcacggatattaaagctcatgaatgatttagattaaaagttgcttaacagagtaaaagtcattagggaggggcagttttgtcctgtgaagttatcacatactcgatcagccgatgaggagttacaaatcgatgactctcagcgatcgcgctcgcttcctccaccgtgcatcgcccgtatacttttgctgcgccacccccttcttcctcctctcgccacaccgcctgtcccggcgtgctccctgccttcgcccatttctgatttttcttgtcatgcttcttatctctacccccatctcattttttgatttggcaagaacatggatgccaatcaaatggtggattgaggtgctgcacatgtgtagccggaacagaggaggattcgctgttttggaatcatggaatgccttctttttttctcttgtacttgcgatcgaatgcggcggcaggcggggcggcgctgcatcaggattgaacgtttgggatatgggtaactacctcaaggacgaaattgcccttcctaataacgtttagcctgttaatcaccttttaatctagaccattcaccatctttaacattcgtgcttttttttttctgaggagcatagaagcacatcttgagatagatgtaatgatctaaaataatacggtaggaggttttcgatttttaagatggatctaacgatctaaaatagtaggtatatcgatttaaatgaaaattgatggtcagatttctctattttattagtgccacgtggtgccataaaagtatttatagcgtgccacgtggtagcttgggagtgtttgcagtaagtttaatgaatttttgaattaaattgatggacccaatattaattttaggcctttagattagatctaataatgaaaaaaaatagtgcctcccacctaacctccaaatgaataagaaaatttcgcggtatgtagaaaagaattaccgaaaacaatctttcgtgtacgtggactactcgctgatgaaaaaatacgttttcaccctgtgtacgtatatactactcaacgtagaaaaggaaaccgtgcgtacttgcaacctgaaagaaggattttaccatctaaaataataatgggtccacgatttaaatgaaaatcgattgttagatttctctattttattaaagtgccacgtggcaacttaagagtatttatagggtgccacatggcagcttgagagcgtttgtaggaagtttaatggacttttagtatataataactaggaaggtagcccgcgcaaatgcgcgggcatgcgtcatacgctaagtttgagacactgaaagcgatatattaaccagagaacatctaccccaatttccccgtaaattgtttttttagcattagttgctattataatttttaacctattagctttatatatatatatatatatatatatatatatgcaattatttataatcttcttcatcatttgaaaagaaggtagacttattttcttttgaaaatatttcaacgaaagtaaaagtaagtgtaaagcaaaagtagtgtgtttttattttttttattgcatatatattatcatgctccactaaattttatatagcctaatatgcatattgatatagtttgagagacaatccatgtataaacatttcatgtagagtgggtaggaggttctacgcaacctaacgttaatataattgcataataatatttttgaaatgtgatgtaccatgaatacacgcttgatttctttatgcaagatagatctaacgatttaaaataatggtttcactggtttaaaaggcataggagtattataaaagtgtactttaaaaattatggctgttttaatatccaacattattttgaattaaacataactcaactggttaggttccttgtggtggaaattgcccactcgttttcaagtcccagatttgacaaattcttttagtggtagacgacgtacccatcgacagcgaatcactcatggtgactttatcaatctcaaaatatgtcgtcccaatctctcgaaggtactcatagaggtagagtgtgcatgtgtgcgttcgtaggggtgagtgtgcgtgcgttgttagcgtctgcattgactttgtttctcaagaaaatatctaacattattgataagtttatttgggacgggtggacggtgcgtagtatgtacatgtccaggagattttttttctcaatccggtggatagtagtgcgtattgtacatgtgagaattatttttcagtttttttgtgaacagtacgtatatacatttctctcgtagcaattaggtgggaccgtggtagggttaacccatctttttttcaatggtaattactttttttctttttttgccgtgagtagtatagtatggaaatacgtatatatattattatatgagatgtatttttaataaatctaatctaataatttaaaaatagtggg

SEQ ID NO: 4 - DNA, 49600 bp, Magnaporthe grisea
  misc_feature (1)..(49600): Continuation of Sequence ID 1, representing bases 148,881 through 198,480
tccgtcgaattaaatgaaaatcgagggttggatgttttttcttttttctcaaaattcgtaaggttttctctaatttattagagtaccacgtgtcagcttaggagcattggtaggagtctcacatggcggcttgagagtattctaagaagtttaatggatttttcatatacaatatatagatacacagaacaaatacagatgcagcttttgcttgcttgttctgaagctgaacattaccgttatattaccatgtatacagaaaggaaatttagctttatctttacttccatataatttctattgccttatcatgtagaatttggtacagaataggtcatttctatcttaagctcggaatgatatataggtggacaaacagctaggatatgactcattactttttaagtaaatcttatttagttatgcatgttttgtgatgcatcaaactatggtataatctatattttgtttttctcataattcagaatttttagaactttttgaagttcataaatctgttttagctatgcatgccggtgaagtgatatatgatgaaatagtatataatatcactttcttttaataattgttcatatatgtttgaataacatgtaaagggtctcaaaaaaacttaaatgaatataattttacaagttaaaatatagtcaacatgtgtcttaaattctaattgtgattttaatcataaataacacaatgtgtaaatagatcaaaaattgaacgtacaatttaaaagttaaaaagttagtattatttaaaatttactgttgcaaaaaatatgtatttagaagtatgcttatttaaaatttattgttgcaaaagctttcataaagagagaatgggaggaaaaaaacatagaggatttcctaaaagaaaatggagggaaggatggacgtcttttttttttaaggatgagaaaaattaaatctaatccaatggcttgaagtgttatgtccactaattttaatattaaaattaaagggtagatatttatttttcaggatatatgatttagatgttacaaatgttgaaattttttaatcaaatttcccccgttagctaatataggaaaaatgaattatacttagaggggcgggagagagaggggagtacgtgcggagagggcgaacgtaacgacttaggggagaggtggggaaggtatgtggtctttctttccatctctttgtttatatgagattaaatccgacggttaaaataatgggcccatcaatttaagtgaaagtggatgttagatattcttttctcaggatttatagtttttttaatttattacagtgccacgtgacggcttaagagcgtttgtagatgccacatgacggttcaggagcgtttgcagaaagtctaatagacttttagtatataattattaatattaatttggtttttatgtcatatgaatcgaaaagaaaaaaaaagaaaaggagggatgtggggcccatcatactaaataatatctataatatataaaaatatataatatattactggtatgtgcatatatttttgcatatatatatataggctggagttaatatatatgggttggaataattaattcggtttttattgtcgtgagcgaaaaagaaaagaaaagagaaaagatggatgtggtgccgtgcatgaagtacgtaagtacgtacatgcagctacgtgggttatgtgggtcttttaagaaaaatcggattggttatttttattatagatcaaatagtaaaaaataatgggtccaccagattaaatgaaaactaatggttagatttattttagaattctaaatttattagagcgccacgtggcggtatagtagtgtctgtagggtgccacatgatagtatgagagcatttctagaaagtctaatcgatttttagtatataataactgatttaagagtgccacacaacagttagaaatatttatatgatatttaatggactttaacgcgtttaatgtctatttgtgggggtattcttttctataaagataagttcaattttaactgtgattggaaaaagaaaagaaaggagcagagggaaaaaagacaacggaacatagggagcagtacgtacgtgagtactgcacggaggtggggccggtgggagtttttgatttttaatatagatctaacggatttaaatgaaaatcgatggtcggatttcactattttattagagtgccacgtggcaatgtgagagtattttgaggaagccacgtggcagcttgagagcgtttatagggagtttaatggacttttagtatataatactaggaaggtagcccccgctgatgcgcgggtatcttatttagtggtgtttgaaattttgttatgaatgtttatgtatatgatttttttaaaaaaaatattttttctcttctaaatatgtgtatatcaataatttgtttgggattgttttaaaagttagtttcgatcactggacgtacagacgtgcaaagaggggacatgtttctaggcatttaatttatctatacttatgttggaccaatgggtacgtcaggtcttttgagggggtgtctgtgacatcctagttcgattagatatggcctatgttttatttaaaggatgtgatttgtgttgtgtgcactttagtagtaccatcttgctagttaagcaagagtgcaactaacttgtaaggcattgtccctctaaccatatcattttcgtgttgattattttacatgaagcgacaacgaaagtgtaagtggggtgatatctgtaagtgggagtgccagttaaataggctgtgatatgttgttctagagaacggattgttacaaattgtaaactaaaattacatgaatcatgtcatcttaaatttatgagaaaaaatgtcgcaacaatggatacggttcatatgaaactatcatttatactatatgtgtttcttttttaaaaaattagcatcttaatttaaacttttaagtaatactttttcttgtatactaaagatgcatttatagattttaggatatattaatttaagaaatttcctattaaaaatatggtaatatgacttctttatctaattaaagacataatcacatacttctatttgcttctactacctccatccataaattaaaggattcaaattttatccgattttaatactactcatcccatccaaccatccattattcattttttttttatctaacagtactctaacaatcttctaactctgcacataccacttatttaatgagccttatttagagaggggaatcttagtatatttttttcattcttccttaattgctataaaattttaggattcattataggacagacatagtaattaaaaaaagcttcattcatatatggatttacgaatttatttgtacgtttgtagattgatttagctttacatatttatataggtaaatagacctcgtatacatgttgagagatataattaagttagtagaagttgttagatgcctaatggtataaagtaatgagcccaccatttttattaaattctttttaggattttcttttatttagtggttaacttaattgccgtaaggaagaaaaaaaagaggcgaaagccagtgggttcgtctcatccgtgaggagggaagtggatatgtgcattatacatatggattttgacggcctgatcgatcggtaggttttatttttaagatatagatctaacattttaaataaggtccaccggtttgggtaagatgtttttttctcaaaattttttatataaagatatattagagcgccatgtggtgatttaggaatgtttatgggaagccacgtgatagcttgagagaatttgtaggatatctaatggatttttagtatataataatagataattcatgtcatattaattaacatatcactcagtaattaaataatttgtatgtgtagtgtccctgataatatccaagtgatgttgcgattttttttttctgagttcacccctgaaaattaagggtatatacatccaagtgacgtattataatttttatatagaaacatatgtaccaagagattatttagctatctaattaatttttatactacacgtatttggtgttagccaaaaaaaataggaaatatggaaggggagggttagagggggagagagggagttacgtacatacatgtgcacattttttttcgttttaaaacgtattatgtaaacttgtgatcggagcgggggaacgtacatatttttttttggaagataagatttagtggtgaaaattaataatctaatgatttaaaatgttgggtccaccggtttataggaaagtgccacgtggcagtataggagtgtttgtaggaatgccatgtaatggacttttagtatataatgatgctcaaatttctaagaatttttttctagttttaaaatgtgataagttcgatgaatctttattgcgtagaatgatttaagatgacactaacattttaatgtttcgaggttgtttggttgttgcccgtatgttcaaatgatgtatttaaatttttatagagcaaaatagtaatatagatgttgatttacttaattaattacaaataaaattactgtttaaataaattaaaaaccggatgctgttttgatgcgatatgatcaaataatatattttagtttttgtacatagtaaaatatgggtgatatggattttaaattattagttgtttttccattaaaaaaattagttaaaaaaagaattgggagaaggagtaaaacgtacgtccatactccgtactatatacgcacgtacggctggaggggtgggaggatagtttgtttcttttgatagactagcaaaatgcccgtgcttcgctacgggtataatgaaaggttaaatgctataaatacacggttaacatgtatgtaataatatttcaagatagataaattgtttattgggttaatgataatcaagactaataagaaacaccattgcatattgatataatactcatattgtcatagcacacgctctctctcaactcttgcatggcacgagtctgatataacttctaaaatccaacatggactcgcgtggagacggatcgtttaacggctcatgtgtggatgatagatggtgcggactctaatatgtcgtgttttgcagatgacgaaaagaaaaaaataattatgtttaatttttaaattaaaaactggttcattataagatttcagaatcactaaatatctgttgaagaaagaggtacaaaacttttgacttttcagccatcgaatgtgtatggtctgtgctaatggtggagagaaaaaaaaaaggatgtgcatggtccgtgctagtggcggagagaaaaaaattgcgcgcaaataggatttgagatctggagacaaagtagatttattccgagtaataataagtgtaaaagttttaggtagggtaggtcaatctggcctaggagaggcccatagacagtgaaagtaataatacgaaactcctaattagtgcatacgcgtcgcgaatttaagtggtgcgcgttttgagcccatccatcgcacagtcgcgcgcgacctattttcccttttttttattatcttttgtttcgagacttctttttcacgcaattggttgccacagtgtcttctctattttttcgcacctcttgcgcaaggagtcagatcaaaaattgaaaaaaaactcacgcagttggttgccaccatgtctcctcttctctgttttcgcacctctttttttccttttacgtaattagtttccggtattacaatctactttacttcttttcttttttactaaaaattaaaaattactttaataattgaaaagttataaaaaaataagttataccaaaattgaatactttcacttaagatttcgaaacttcaactcgaattttgaaaactttcaactacatatttgaaattttcaactattcttttaaaagttttaactaaattatttatgtgttttttctattgaactttgtttttagtgaattccctacctgttattatcctaacttaccgcataaaaaaggaaaaaaaaaagataaagcgtcgggaggaattttttcccaagaaaaaagcgaaaaaaacaaataaagaaaaaggaaacaatagaaacttaaatgggccaaagcctgttataataggcattaaagggagagggtggtgggaatagaaccctggtggccacaatgaagattttctccaataaccaagtaggcaagctactacttgtgatcacatgttatatcagttaatacatatcctatattaacctagttttgaggtagactgaaaaggatccatccagaccattgagcggattaaaacgattttagcaacgtgattcttacggaaaccttacgaattttttttattaggtatagatttaattagttttgtgttatctataacagatctaatatggcataaataatgggcccactgatttaagtgaaaatcaatggtgggttatttttagaaacttttgatattttttaatttattaaaataccacatgccacatggcggcttgagagcgtttataggatgtccatgtggtggcttgagaacgtttgtaggaagtttcatggactttagtatataatagatagaacagaatagatagatagataatagataatagataggggcaaattagtacttttcatgttgtttctctctcaatctgcaccacatataataaaatatggctgttgatgtccggtagctaattacgaggtttttatgatatcctctaactaatttatgtctttgcaatgccttatagctaattacacatttttaggtgtcacgtaacaattttttttttgccaaaacgtatgcacacaagttgcaaatttgactaaaacagaagatattttttctagcaaagaaagaatttcttttttcttatcaaagacgaacttaaagatttggatttttttcacaaaaaggggaaaaaattaatcaacatcggtacggaataaaacaacaaactcattcttgacattacccagcaatggagtagaatattcatttcaaggaaggagaaaaagcatgccggattagaatggacaatttcacaatttcatcgaattcgcactctgatgatacgaatgaacaattggtatataaatgaatagaagcaaccttcagctacaaacatcgattagcaaaacttgcaacacaaaacacagatctgagccttatcactccatgtaaatttgcttcaattttccaatcgaatttgtcggtctagctctcggctccgccgccggcgaggcatgggcagctgcagaaacaaaacagagaaatcagcaagagcaaagcatcaaacaactcataaaacaaagcacagagtgacgaatcgatatgaaatctaatacaatctaaataattgctgttcgattttcaaagaacaaattggagtaccatttggccaagaagctcggtctctcctgcgcccgctgaggttgcgacggcggctgctgcgggtttgccggacgcccgacgaagtagtcttctccccccaaaaacgaaaacgaagcaaaaaaaaaaatagcaaaccaaccagacaaaattgactcaatttttgtgtcaaaaaacgattgcataaaacgaaaacgaagcaaggttcagcaaaacatcgggcaaaactgattcttttgtgcccaaaatttgatcgaatcgagcttgcaacgttgcacgtaccaccagggatctgcgcgttggcagcaggccgctgctcgtcggcgccctgctgctgttctttcgccggctccgcgtggttcatcggccgccccacgaagtaccctgcagcaaacgaaaaattcttagactgatataaagaagcaagaattcctttaggctttagcatgattgattgatacaaacaaaaaaaaaaaagaagagaaacgaaagcttttgagtgatcagagtgacacgtaccaggaacatcgctgctcatggcggcggcggtcgacggatcgacggtcaccggcggcagctggttggtttcgtaggaggagaagctactgagcgcacacaacgagatggaaaagaagacgaagagggtaaggaggcacaggaagatgttccttcctctctcccaggctggcctacgccaatgattcgcgcagagattgatattttttttcggccgcgacgacctaaaacccaagtcgttgctcaagtggcatcaccaaccagcgaccactactactcgaagaccagctcagtaactagagtatcaggaatttgcaaacatggcatgagatagagaatatcaaagcatccactatttgttatttctttccacgaaattgcaaacatggcatgcaatgagaatatcaaatcatccactgtttgttttttttattttcaccgtactggcaaccgttttagttaggacagatactctggatcttgtaagttgtcatgacagggaaaacttcattgtcttacagatatttgggccgtgttagtttggctgcaagtgaaagagatcggcctcttctacattcaaaggcatgtgactttattactgatatataggtccgatgaatcataaacctacgtatcaatgataaatgtatgatgcttttgaagacaaagaatatcaatcaatgtgaaagatgcatgattttcgcaggccttaaattgtagacataatggctggccacttgtgtaaatcacttttgactgtttgcaaaaagctttttcttttaagtagcccctgaagattttacacatttgtgttctttttcaatacttattttaagaacaaacccttaaaaactgatcgcacaaatggaccctaaaacttctaacatctcagcacaaaaatccttggagttgaccaatggcaccatgctagaagcaagattaatatactacctccatcccaaaatatagcaatttttagctatgaatctggacacatacgtgtccagattcatagctaaaaaatcttacattttaggacggagggagtagtaactaagcgacctgcttgttggtgctaagatgttggagttcactcctaagcttgggcttggctgtgactgagattggggttcattcctacaattaagtttcataggattgtatttgtaaataagtattgcaaaaggaacatgatataaaatgtttcaatatgatagtccataaggttgatgcattggctgttagtcttggaaattagatgaaagtaaaggtgtctacttgattagtatagtatggactggtggtggcgaatataagattctaagtgtaatttggggtgtgtttgttcacgtcaaaattagaagtttgattgaaattggaacgatgtgatggaaaagttgaaagtgtgtgtgcgtagaaaagttttgatgtgatggaaaagttggaagtttgaagaaaaagttaggaattaaaccaggccttgatttgaatgagtataataattgattattgtccaagtttatttaccctgaaccaggattaattaatgaagaccacaagcatgccaaataccagaatacctatcaatccatgtcagcgaagagtcttagaacttttccagttaatttatagtcattttcatctcaaagcaaacgctgaaaaattaaaatccatgacagtttggttcaattgcttgaagacttgcagttgcaaataagtcggtcaattgtcaaagtcatctcattgatcgctgctctcgtttgcaaacattattattctttttcgttgaaaatgacaaatgattgttcgttgtaattaataggcgattgagtcaaaaaaaaatttctgaaccatccaagagaaagttcaaagttttcatattcatgtggaccttttcctgacaaagacgacttggataaaacaatccttttaatgagaccagtagcaatatagtaaattgatagatccaaacaggtaagcaaaatactaatggataacgcggaataatctcatttgcttgggatatgattgtacaattcacaacagtgtaactgcaggaaaagaaaaactttggaagaaacaaaacttgtatttcctaaaatgataacttctccatcaaaattccactgatttcttgatgaactgtcgattaacattgcgaatacagccgtgttgatctagttcacattttgaccggcagtgaagcacccgaagctgcagagattcaatgagccaatgcatcatgcaaagagaaaaacaaatcaatgggtaactacagacagaagcatctgatattctgtacacgaattgcagcttacagtttcttaaagaagcccggttcattcctctgtgcacccttagcatcatttggtctgaccggattgccattgtagtagccttagtagtagaaaaggaaaatcagaagataaagatttctcttaatctttataagtttgaaatttgaaaagttgaaaactatcataggacatagcaaacaaaacagcctgaggtaatagaagaatgcatagaagactgaaaattaagaacccttttcaaagcacttatcaacctcaagttataacaagatctgctacatcatatccggcaaattgatgtatgcagaaaactaagcaaattgatgtatgcagaaaactaattaatcctgtatacctaccagggaattgagcgttctgttcctcagccggccgtgaagcttgctgttcttcatggttcaatgggcgaccaacgaagtagcctgcatatgaacaacacccacaaagtaagaaaatttacagaaaatatgttattcaaacgtgaaatagttcgcctatggatgattggttaagcttgtagcatgcacatacctggaggattgtcactcatgttggatcaatcgatctccagccggttagctcctcttccggcgacggcgaggcgagaggtagacgaaggcgacgaccaggatgcagaggagactgacttgagaggtgtattctaatgaaacttttcaggtccacggccgttttattggcttttctctaaccccgttctaactttgattatacagattttaccttggttttcgttagcatattttttaaacagttaaatggtatatttcgtgtgaaaacttgttatatacaagttgttttaaaatatcaaataaatccatttttaagtttaaaataattaaaatttaattaatcaaaatggcaatgacttcctcgttttacgttccattagctaccagtagtgattagaataagcagacggattgtatggagcaatcggatctacagttgtgccaactagctaagctataggtcatcaaccaccgagtcacagtaaaaatgatcgcgtggtctggtgttactgaaagcgatacaattgcagctcagttttttttttccaaaatggcccacacataagataagatggtgtgaaatttcatattgataaagctgggaaaaagtacaaaagattataaccctacgaaattattatcaaggaaaaaaaaaaaaacctccaccacacgtgctagtaactcctgcacataccaacccgaccaacgcttggccaaatgccactagccctattagtgacagagacatgattttcgggatgggtattcaagatttccatagaaaaaaatattactcactctgtttcatattataagactttttagcattgcccacatttatatagatgttaataaatctaaacacacacacatatatatatatatatatatatatatatatatatatatatatatacatctacgtgcaaagtcgcatcttcaaattcattctacatagagaataacaaaaaagataaaattctgacaaaattgcaaccttaaaactgtcagattttttgtttttttgttacggctaaaatataatgaatttgacgttaagattttaaccctaggtgtaatacaattgaaagtatgtgtatgattttttctagatttattggtgacattttttagttggtgtgcaccgtgtgtacacgtgagggcctgtgtgcataggatatgttgccatatatatatatatatatatatatatatatatatatatatatatatgcgagcaatgctagaaagtcttataatatgaaatagagaaagtagtaacattaggagctacatagaggaaaaataaggtagaatcaactgataaaaaggcagggattgaaatggagaagagggagggttgaggtgcgccatgctcatcatcgtcgtttgtctgttcaaattaaagaaaattgctaagatttgggaaaaccgccggtttcctccatagggccggtctgaccgtcgtttgttggtcggtcagaccgctacttgagggccggtctgtccagcggcatgtggccggtcagaccggcagagtccaagcccgagtctgttttcgtcaggtctcaggattccttactcaggaggctatatttcgggtttcctttgaattctatcctgagttggacgtggaggaggacctgtaggaggcaagatcaacccctatataagggacaaggccggttcaattgagcatacaatctacacacaatccaacttagccaattaaattgctttttcaatatttcaatctagttctagtctagttcatccatctttgtcgatttgtgccataaatcgtctgccttcactacgagagtgcgaaatcctttgtaggtttgtcccgtaaaccttccgtttacccacgagacgggtagttatctattgttctatctaaatcggctccgctagccggtttagtttatcaaaacccatcttggtctagcttttgctagattgaggtggttggcgatactacaatcaccgcaaggcgtttaggtgtctcgatcgtgcttgtctacttgtcaaaaaagttgccaaacacgtgccattccatggtgtctcagactcttggtgctcgttgtacactcatgccatcccatgccatctcgggacaactcggtgtccgcaccaagaagcatggcgcctaccccctatcagttttatgggtaaggcatacccaatagtgttgggttaagcttagcgggacctaccatataccaagtcttatacggaatcaaatcttgcatacggaaggtattccagataaggaaggagaagcagagttctacatggaaactacaaggactattcggatcgtatccatatttatctttctagttctatttggataaggggacatctatgggtataaatacaagacccctaaaagaagagggggagacggaccaagacaagccgagtctaaatagacaggcaacagtcaaaatatacatcaatacatgccgccagacatcaacataagagataaacctaaacagatccaatctggtgccacagaggctgacaagagggatctagcgctatctctgatatcgcccaattcatattagaggaggaggactaccctgttgtcgacaacgtgtggtggttcatgccgccgtgttgacgatgattagataagttatcccaaatattgtacttgtgtgattctgatgaataagagcaacactggtttcaattagcaggagtagagctattacctggtagataaggagcctgaacctacatgaaaatccttgtctccgtttattttaccttagtctcgcttataccctgatacgaatgatccccaaactctgcaaataccgtagtcgtgatctacaacatcgacactctctccccctccccctctaactcgatgccaaggtctccagcgtgggtgcacgaaacctcgatgccaagctcaccggcaccgagtagagtccatatccataattagatttttggagtctatttattcataaataagttttacaaaaagagtcataatttttttaaaaaaatcgagcataccaaaaggtggacgaagacgacgaccaagaaatcgaggaaacttgacttgagagatttttatactaagacgacttggaggtctccggccgttttagttggatgaaaacataagaaatcagtttgttagctttctctaagctgtcattagcgagtagaataaggagactggaattatatttttgcagttgccgactagctaactaggtcctcaacaactgagtcataagtacaaggactgttaaggtgacacggtatgtttcttcctactccctccgtcctcaaatataacgatatgtttcttcctactccattcgtacttaaatataaaggattttaaacagatgtgacacttactagtacatgtccagattccttgtactaaaaaaatgtctcatccgtctaaaatctcttatattttgggcaaagggagtatatgtccaagatgcagcgaacagcctgaccagtgagctggctgcaggacagtaaactaatcaaaactgtatcttttctaatttgcatactggactacagatattctattcctttcgatatgaacaattctgatttactttttcttttcctttttactataggcaaaataatctattgagcaataatttctcgagtttctggtccatttaaaaagaatccagcatcccccactgcatgcggtttggagggtatactataaattttgagatcaggaaaaaggaagcttccacagcggactcccgtcctacacgcacaacttggccagttgataattggcactgcaaatgcttagccgcgtcgacttcgcgtcaagtcgtcggcggctagttcagagaaacagacaaggatctgattgagatttgaggctagtggctttgtgctttatattttcttcgttgcctatcttaaacctcacatgctctgatcgtctcgccagctctgctcttctagacgctctgatcgtctgcagctcaggttagattgatttccactggatcttgctttgtatcatctagctagtttgtagctgttatcagctttaacttttctgattatgtctgctggaaaattctgcctttttctttcgtgaatttcgccttgttgagaggagggatcactaattagtctctttgctagaaaatattgggaaatttgttctgaaatttgaccctgagcgagcatatctaaggccagcagcacattctcagttcagagatggggaaagaacctttgtgttgttgttttgttcagtgctgattcaactgcaacatttagcgattactgttgtttgcagtgcatcaccttaaatactgtgtgttaattacatgtttcccgtatttttatcatttattatcttattgttggccatctactgcaaggacatcttaagctaaaattgaatgcccgtggaaactattaccaatcatttcaccaagaacaaagcatgaaagatgagtagcccaacgaaattttctaatggccaatcaatttgcgatatagttgaaatgttgctttacaaaagatccagtgctactatagcggctgtacagaattaatatggattacaaaaggaagacaaaaacaactaggaccaaataaagaaacagtaacaagtcaaccagataaacgagatttgaagggcaaacatctattatattattaaaggaatagaagaagaagcctctatgtttgctctcatggtctataaattctcacattaattagggaaaaagaaaagacagagtccatatagaaatacaatttagaaatagctgaaattcagaattaaaaaatatggaatcttagaagaggagactagagtccatatagaaatacaatttagaaatagttgaaattcagaattaaaaaataaggaatattagaagatgagactagagtccatatatagaaatacaattaggaaataaataactgaaattcggaattaaaaataaggaatattaaaagtagagtatatagtccatatagaaatacaattaggaaataactgaaattcggaattaaaaataaggaatattaaaagtggagtatagagtccatatagaaatacaattatgaaataactgaaattcggaattcaaaataaggaatattagaagtagagtatacagtccatatagaaatacgattaggaaataactgaaattcggaattaaaaataaggaatattagaagtagagtatagagtccatatagaaattcaatgaagaaaaaaaatagaaattcgaaataaaaaaataaggaatattagaagttgagtatagagtccatataggaatttaaaactaactaaaatttggaataaacataataaaattaaaagtagagtttagagtcagtataaaaatacaatttataaataactaaaatttaaaattaagaaaaacatgggaagaagagtttaaagtcactataggaatacaatttaaaagtaactgaaattcgaaattaaaaattaaagaatattgaaagatgagtttagagtccagatagaaatacaattagaaataataaaaattcagaaataaaaataaataatattggaagaagagcatagagtctatatagaaatacaatttacagaaaattcggaattaaaaaaaagaaatattaaaacacgagtctagagtccatatagaaatatatataatttacaaataactaaaatttgatattaaaaataattaataactaacacgtatataaaatataatatgaatattatgtattagtagttttgtaaagttattgtaaaatttaaaattatgttgtcattttaatatatttgaataatataatgagaaaacatatatgcaattatatgggaaaaaacataatggtgctagccgcgcagtctgcgcgggccaccatgctagtttttattataagaataaattgtaagcgtgttgggattgaacatgggaccttgggattgaaaccacgcaccccttatcactacattatcaagtgcatctcataacaaatcaaccatgaccctgcctatacaacagatctacaccagtgctaaacggtactccctccgttcgtaatacgagggattatattttttttaatagaaattaaggaataggaaaaagtacagataaagggaagcactaattggattaggccgattggcacattgaataaactaaaaaaaagtaagaaaaagctaagggcccctttgaatcacatgaataaaaaaacggaggaatatgaaaaacatagaattctgacaggaatgtaagtgtaaaagaggattgcaaaacacaggaataacgtatgaatgaccatttgattggaccacagaaaaaacacaggaatttgatgagagataaagactcaaaacgattttttcatgaggttctacctcttgttaaaattcctccaaaacttgtatgggaagaggcattccataggaattttataggattccataggattcattcctttaattcaaaaggctttgtaagaaaaattcctataggaatgaaatcctctaaaatttctatcaatttcctttgaatcaaagggggcctaaaatgttgctgcatcccttctgttgtttgctcacctcaggatccacgatcccttgtatttgtggacaaatcgaagggttataatctcttatattcacagttagagggtagttcttttcagcaactcaaaacgacaacttttttctttttcgttaatgtgttttccgtattgctaaacggtgtgttttgtaaaaggatatgtaaaatgttgacctctacctgttataagcaaaagaatcgaaccacatgcgcttcagcacttccatgctgagaaagagaacactggaatatgtggtgtgcatctctacttcatattaagagcattgctggtctgctccttgtcacttaaaaagaaattgagtcacagtcatgttctttaagtacttatcagtaggttcagcagcttaatgccagaattcacaagtaggtttctactgacaagaaacctatcatctaagcatggtgtaacagactagaagagcagaatccaaaacagagttgtcattagaaatggctgcttaaaagaaaagtaagattagaaacaaactatgatcatgtttgatattatgttcagatatatctgggctgtttcatatatacttcattgcatttaagaacatggaagaagtgatggtgcatgaaatactcacacatattagatactggcctttgtatgaagcattaaaaagctgggcacatatgagttatactaaatgatcaatctatctgtggaaccattaaaaacctgaatagagacctacttcaagaaacaacacactcaactaaactatgagactatctgattatgagttactttgatctcctattaagggcctatttgggggagctttagattctgagaagcaactgtttggtagctagattctgagaatctggaaaagctctgaaacccagcttctccagcttctggcttcttagttcatttttcagattctgtaactacagattctcagaagctgtggactgtttggggtagcttctagcagaagcagcttttgggaaaagttacagctgggacaagctcccccaaacaggacctaagttacacagttggtcacattgcagagttatgaaacaattgccatagaacattacccttacaattaagattactgttgaggctggtacgtaatgaggagctcctttacctatatctgttggtacaccatgattaacattagaaatatatatatatatggcttgcaaatgcccacaagggcccaaaaaacaatcttagctgcaattgttttacaactgtaacaacaaccttttttatcttatcaattcgttatgtgactatctccatgattcatgattctgttctgcagcactaaggtaatggcagaagctgtgctgcttgccatctcaaagatcagtattgcattgggagatgaagccaccagagctgtcatagccaaactgtctgggaaggtaactaacttgagggaacttccagacaaagttgagtatatcagaagggaattgcgtgtcatgaaagatgtaatacaagatttagattccacaaataccaacatgaatgttgtaaagggttggatcgacgagctgcggaagctggcttaccgtgttgaagacataatggacaagtactcatactatgcttgtcaactgcagcaagaaggatcagtgatgaggttcgtaagagtacattatgcaggtgttttcagtgaagttgctagtgaggtaatgaagataaagggtgatattgaacaggttaaaagacagcaaacggaatggttacctacagttcagctcattccaagaactcccactgacatcgaaacaccgcaatctcaaggaagaaggaagttgcttgaatgtggcgatcctgtggggattgaatacaacaggaaaaggttgcttgaattactgtactctgaagaaccaggtcacaaagtaataacagtgtctggtatgggtggactgggaaaaaccaccctggccttagacgtatatgaacgtgaaaagatcaagttcccagttcatgcctggatcactgtgtcacagacttggacgatccttagtctttgtaggcagctagtcagtgagctcatacgcatggaacaagaatcatcagaatctaaggaagatctaatcaacaaaatgggcgttcaagatttgacagaagaattaaacagaaggactgaaaacagtacatcatgtttgattgtgctagatgatgtctgggaccagaacgtgtacttcgaaatacagggcaagcttaagaatccccaagcaagtcgcattatcatcacgacacggatggaacatgtggcagttcttgctccctctgaatgccatctcaagatacaggctttgggtgaaattgatgcattcaaccttttctgcagaagggcattttacaacagaaaggaccataggtgcccgctggaccttgagaatgtggctgcctccatagtaagtaagtgtaaggggctgcccctagcacttgttaccatgggtgtcctgatgtccacaaagctacaaactgagcatgcctggcaacaaatgtacaaccagcttcggagtgagctggccaaaaatgatgatgtcaaggcaattctgaaactaagctaccatgcattaccagctgaccaaaagaactgcttcttgtactgcagcctgttccctgaagattttcgcatatctcgtgagagccttgtgcggtactgggttgcagaaggttttgcggtgagaaccgaacacaatagaccagaagatgtggccgaaataaatctcatggaactgatccaccggaacatgcttgaagttgacgagtatgatgagcttggcagggtgagatcttgtaagatgcatgacattgtgcgcaacctggctctctcaattgctggacaggagaggttcggttgcgtaaatgattatggagctgtggaaaaggttgattgggaagtccgtcgtctgtcattattcttgaacaatggaaaaggttgtgcatcaacagtgaaatttccacatcttcgcacactacttgaaacaactacacaccctcctggattgttatcctcaattttgtctgaatccaaatacctcactgtcctagagctacaagattcagatatcactgaagtaccagcatgcataggtaaattgtttaatttacgttacattggcttaaggcggacaagactctgctcactaccagagtctattgagaagctctcaaacctgcaaactctggacatcaagcaaaccaaaatagagaagctaccacgtggaatcactaagatcaagaagctaaggcacctgctagctgatagatatgaagacgagaagcagtcagtgtttcgctatttcattggaatgcaagcacccaaagatctgtctaagttggaagaacttcagactcttgagactgtggaagccagcaaggacttggccgagcagctgaaggaactaatgcagataagaagtatttggattgacaacataagttctgctgattgtggaaatatttttgctacattatcaactatgccgcttctttctagcttgcttctttctgcaagagatgagaatgaacctctttgctttgaggctctccagcccatgtccaaagaactccacaggttaattatcagagggcaatgggccaagggcacattggactacccgatatttcgtagccacactacacatctcaagtacttagctctaagttggtgtaatcttggggaagatccactggggatgctcgcatcacacttgtcaaacctcacttatctaagactgaacaacgtgcacagttcgaaaactttggttcttgatgcagaggcgttcccccacctgaagactcttgtgttaatgcacatgcctgatgtcaaccagataaacatcacggatggcgcccttccatgcattgaaggtttatacattgtatcattgtggaagctggataaagtccctcaaggcattgaatcccttgcctccctgaagaagctctggttgaaggacctgcacaaagacttcaagactcaatggaaaggtgacgggatgcaccagaagatgctgcatgttgcagaggtccgtatatagatgttgcagattggttcttggcgcggcggctttcccatgcctgaagacacttgtgctgaagcacatgcctgatgtcaaccagctaaaaattataagtggtgcacttccagtcattgaaggtttgtacattgttgctctgtcagggctggagagtgtccctcctggcattgaaacccttcggaccctgaagaaactctggctggttggtctgcactgggactttgaagctcactggattgagagtgagatggaccaaaaaatggctgattgtgtaggggactaggagtgctagttaccttctccctacattagcaatcttgtttgatgcctctgcatttgtcaggtccattgaccattccaataagttcatcgttatggagttattgctactttgctagcactacctgaaacattcgctttcttatccatctggacctgaagtgttcttctgttattttgattgctttggagattgttgtaagaggcccttttactattgaactgcgtttagacaatgtttttcctttctgttcatttttgagatcgttgttctgcagaatgatctcatagcatttgtttattatctaatggtgggttcacttttatgagttagattgcattctcgaatttcagaaaaaataactaaagcaatcagatgatgcagcgtttgaactttgttgtaatctactactattgtttagctgaagaaattctgacaactcccatcgtaggccattgcaagcaagagcagatgcattgctgcacagatgtactatgcaagtgtgaaccctctgtgcatttcatgtgccagtattttaaccagattaaacatgcaagaaggatgaacacctagaaaacataattgaacgcactagaacacgaatctgccgcgcaattgacacgaaacccttaaaatcgacacctgggaatatcttcatggaatgaatcgaatggggcaaccccaccaaaatcgcagggaactgaatccgggcgttacctgaagaatccaggaagaggcgccgccgacggtcgtcgtccatggccgccgccggcgagcgcttgcttgggctcgtttactccgccgccgcagagtcgaggggattggtctccgatctgatctgggccgacgggttcatttcattatggtataagttcactttaggttcctcaatttgtcgccgagtctaccattcacccctgaaccgaaatacggtatagcacatcctcaactcataataccttgttaactttgatcactccacagtacagtgatcggttttggctgacacaactcacaatacctcgttaactttgatcactcgataatacagtgatcggttttggctgacatgatgagggggacccacgtggaccccatatgtctggagaaagctacccatcggacgtccgatgcctatcaaaaaatcggatggtcaaatgttgcagtttaccaataagtgaaacattaagttatatacggtaaaaaaaatcagcaactaaaacatttaaatattttatgaaacatggtgaagatacacgttgaaacatattgctatcacatatgaaacaacaagtagcatatgtttttcctttatcaaaatatagtcatgcgatatcttgttgtaaagatttaattaaaacgaatacaatggtgtaatcgggtcatatattggataagtaatttaagagaaaaatcattttgaagcttgctaaaataaatgcatgtgtgcatggatgagatggagagagaggagggagagggtagtagcagatagtatcaggaattttttgaaatacgctgaagacgcatgttgaaacatatcactatcacgtatgaaatatttagttttaacggtttgaaacacatgtttttttatataatataatcatatgatatcttattgtaaagatttaattgcaacaaatataacggtgtgatcggttcgtagatcgaataagtaatctagaagaaaaaatatttaaagagaagaggtaagaacaaaaggaaacaattggccatggatgttacagtgaaatagttcatcctatccatataccgatttacagtagatgacgcgtccgatatttcggttttgatcggacgtacgatataaagcattttccccacgtgtcagcggcccatctcttctccctcatctgcctctccctcttcactcttttttcccatctcttgagggcggcgcgcagtgaagcgatgggaggcgcgcgccacgcggcggccggcagaggcgatgacggagtcaccctgcgctccagtgcgtcagcgaacgtggagggaacccacggcttgccagacggccggcgtggtcacctgcgcgggcgacacggcacagtggcggatgtccgtgtccgtgcactgttcccgccacaccattttgaaatagttcactttagctgtcactaaagacaatctcaaatctatctacgttcttgtagtaaaaaaaaaagtcaaaatgcaagttcttcctaacgctacagaatgcaatatactcaacggagtttacaactatccttcgatgacatgttataatgtgtaaccttaacatcttaattaaatcattaattaatacatactacaaaattatcagcatttaaaatgcagaaactatattagtagtttttgcataaaaaaagatatttcatacagttatatttaaattatttttgctaatatatagtttgagagagaaatgatagcatggaccttgaggactagtatatcgatatccaaaacgtcatttcttttttggtaaagaagataccaagactatcataagtgtctacttattcgcttatcagccatagcaaaaatttaaatcttgaaatttgatttttctaaagttaattttgggtattgttattgcagttcattatttttcaatattagctttaaaccacaaacaatatcaatataaaacattgcttaatcatttttctactatttattgatcgtatcattctagcacaattcacacgtttgaaattaggtttttattaatgcctcactaaaactatggcgttacaatattatgtcattaactatttcttaccagagaaaatatttttctattacttacgcaacaacaatttatttatttatcataacaagcttataatagatgaaacattaccaccaattaaactatatcaatattcttctcacatacaacaagtaataggctagccatgttccttgcaaccggtaatcaccaaacaaagcacatttagttgcaagggagatcagcctcttccctcactatgggcacgccatattaaactagtgcatgttcattcaaatcaaattgagaagcatgtggtgcaactatttccacttgtggagccacaaccaccgaagaagaagcaccttgtgcgtggagctcgatgatgcggtttctaatggcctcccggagcttggccgcctcccgcttgacatttttgaccatctcgggagataaatcatttaagttcttgcggtgtccacctcctatctcagaaattatgttggtgatctcacgtttgtagttctcagccttcagattgtccagcttcttctggagcttcataatttgctgctggagtagggtcttggagtcttgcatccacttcttctggacagcctctggcatgtccatgagtttgctccacatgtttttggcctcttcaatagatggccataccactggatcacatgcttcatcgatgttgtacaccacgatactagcttggacactgcacaagattgatagctctgttacctttttaatcaatccagcacgacgcctcctaagagtcactctgcgggcagcgtttttggtatgccgtgccatagcacacctcacctacacgggcgcacggacacgaacctagtaatgaaaatgactcttgtgagagttgtttttctgtgtttgccgacagaggtagggtgatctttttatagtgaaaaacgtggtattctattttaggtgaatggcgcgtaaagagctctaaaatagtgaaaaagataagacaccataatagagagcgaaactttggtgtaatttaaattataatgagataagataaggtaaccgtaaagctagtgtagtttaaaatagcttcaggatagggtaaatcatatataacgtcggcatatattaggtgctatttacatttatgggatttgtccacacacaaatgcagatttggggcttgaggaatttggtccatggctcgaggccttagcctaaaagtttatggtgctagactttttttatagtgttaatttatatggggctcaatgtgggctatgctatcctaagattttattaatcagcacacggcttgtccgtgggcatagaacttgcctagttgtcgtacctatatggctatatatgaaatgttagcaacacttgtaacatcaatcacataataatacttgaaaagttattgtgatcatgtcattgtttattcagtgtgttgtgttgtttcaatagagtaccaactccacccagatggtgcggggccccgctcacccgtcgggctccctgcaggggcgggggaggttttccacgtggtgaccaatgcgggggcggggacgggggctggtgttgatgaaaaaatcgaacacaccggtctgggagatctgcttagctcctgtgcaggtacaaaggttgatgagatgcgagcgtgctagtcagtttgatcctgcaactgacaagatatgtaaatagtagatcaaatagccgatcgtctgacaagccgatggagtggttctagccgatagccgataatagccgatgccaataccagccgatagcgatagggtttaagcaatcggctatatgtccaatgtagataatgatataaagacaatcggctgatgatgatgtaataaaataacaatataatccagtataagccaatcggctagcattgatatgataaataagcatcgatccgaaggttaaagcacacatcggctggaggtccgatgtcatgaaatccacaagattagattaaacaatgaaacctttgttgtcatcggctaaatccaacttatatgtatatgcaatccttatgagccgatgcaacgtccagataactcaccggttgaaaccccgatgaaacccttattggcaatcaaaaagcaggctagagattatggttctaagcacgacttagtagatcaaacttaactgatgcagcactaagtatgaaaagaaacacaatatctagacaatcaagccgttgactgattttcagggtggtagatgtctaggctaatctaatctagcaaagcgatttagccgatactggcagaaaccctaaagtgagagacagccgatagagctaaattgagatgctaggactagattaacatagacatatgataaatagccaggcaaatatattatccaaaccagagtaatcctataggtcgaatgtactgatgcagccttgaacgacgccgacgtagacgatacaattgcctggaccggcggaacattggacttatcccttcgccggagatcgaagacgatgcagccccgcgtcgggtgccaggttccgccggaatgtaaaaacaaaagtagaagtaaaaagggtggcgatgcgccgaattgtattgatcgtagtgatagattacatagaccccgggtgtacatatttatacccatgggttgatacaagtccttgtcggacaagaaaaaaactttcctaaagataaaaggaaaagataaagtccttataggatactaaacacacttttctaaagataaaaggaaactaacaaactattcctaattaatagataacttgccatgccgcattctctttgaactcggtctcttctagataagcttcctttagtagatcaattttcttaaccgaatatagcaagaatccgacaactgacgacttgacaattctcatcggctaatcgcaggacttcaaagccgatgctgactctaagccgatgactactccgggcttaccaaatttcactgttaacagctgggggggagggaatgcggggagcgggggggttcctatccaacccggccccgccccgccccgctacccccctaggtgagggtggtgagaacggggacgaggccacactccggatagtgcgctgccgttgccgctagatcaggaggagtttgaggctggggagtagggagagagtaggctagcaggggcgacgaaaacggcgacgatgaattaggatggaaggcaaagtcaagtagtcaacaccagcatggggcagccctatcaaaccactcttttttttatgactatcgtgtccctgcgaattgaacggacatatttcaatcgtactggtggtacttagtacctcctgatacatcctcatggatggaaaaaactttcataatagtaattgatgaatttgtagggaaatttaaggattgcatcacctgagtttaggggttttttttctctggaattggagaatgcaatcttgctcatacaattggagtacgagagcacatactaatccattgttctaacctaaagaaagccagagaattcccacattatgccatggcaatgctagaacttgcacattgctgcactactgtgtgaactgtggagctgtgaaccctctttgcttacatgtggtgaatttgcagccacatgaaacatgctgcaagcctgcaagaatggagcaacaaagactgaaagccctagcccacgaacgtcgagaggaacccctaaaatcgagaccaacacatttgggttgggatatctctatggaacaaattgaatagaaacccccaataaaaccataggaaactgagtccgaatccttaccacgaaatccaagaaagagccgacgccgcatgggagcttgatggccgatgcataggagctcggcaccaaagcaggcgggcaggctgccgccgccccgtcagtcgctatagtcgtcgtcatcattgtcggcggcgagcgcttgctggggggctactgctccctccaccacccagactatgccataaggcgggctgggattagttgccactggggtgagccgacgcgacccaactctgctcgagatgaacaatttgagcattgtttatatatgtttgattattctagtttggtcttattcttctaggtccaatgtggaattgtgttctttgggcttcttctggcctagccacctagggtgttaatggatcacaaaattttcggttttgcgagatgttcagaagattattataatgagtaattctttttttttattacacaacaaactcacatgcacatatatcactgtacatgtaacaccacactcacacactcacgaatacgctccctaacacactctcaaatagatgaaaatcagcagcacgtatatgatttcactgattaaattcgtgttgaaagcccacttacatatgcataatatttgtgggtataactcatgacttcactaccgtatcactgatgcttttaagttgtctatgttgttgaactcaaaatgttatttctaacagtagagaaagcaatatactcgacgaagtttaccagcccgaaaacggtatcggatccatctgagttgaatcacacttaacactatttaacactatcgacctcgctttaacgcaaaaattccaaaatcagaattcctagcgaccgtgctgccttccttataagctgctccctctcttctcaaattggcgaggtgggactaaagttagctacagctgttgctgctgctcttgctctgctcgctgctgcatgctagctactgctgcttcatgttgctgctgtgggctgcatgcatgcatgggctggcctgctgctgcttgctacttgctgggctgcacgcatgggccaaggcccaaggcttagctgggctgctagtttgctcttttctctcttttctttttctctaattattgcggggcgttacagtgagtcatttactcatttgcccatctaattatgcactcgagcatacctttgggtcaataatgcatggagggatagcggcttggcaataaagaggagccctctccctctcttctctctcaccatagcatgaagaagcgctctcctcgccatctcccattgtcctcatgcagaagcagctgtgcggcgacagctcgacgatgaggaggaggattagtggatccgatggtggagcttgggctgcggcaagtcccgggcgatgaatccggcggcggtaagtcttccgtggccatgttggacttcaactgctgcgggtcaatgacagcaacgccacggcgaggatggtggctaggctacatcgacatggcctccttgctctctcccatccacaagccgcatgcatcagtggcgcggtagcatccgacggcatcaagaaggggaaggccacatccaacggtgtagggcattaccgtgtcttgagattagtcaaatttccatgaggtagtagtgtgacctcatgaaataaaaaatattactgcatcatgaaattagttaaaaattccatcacatattttctatacactgatgtacaaactggattgaaaaaacaagaaccactgaaaactaggatcaaatggacacaaaggggttttttcccttgaaatgagcaaacttgaaaaggcatggacaaattggtagttaggcttcaaaacaggattaaaagcgcaaatgccccattgtttattgattataactataaaagacggaatattgccaccaactaaactatcgaaatattctcctttcatgcaacaagtgatagactagctatgtgaggaccatgttccttgaaaccagtactcatgtaaaagtcttgccaaacaaagcacctttagttgcaggggagatcgacctcttccctcactatgggaggatcatcatcaaccgctgcaggttcattcaaatcaaattgaaaagcatgtggtgcaactatttcctcttgtggagccacaaccaccgaagaagaagcaccttgggcgcgaagctcgacaatgcggtttctaatagcctccctatgcttcgccacctccagcttgacacctttgaccatctcgggagatagatcatctaggttcttgcagagaccaactgatagctcagaaattatattggtgatctcacgtttgtagttctcagccttcagattgtccagcttcttctggagcttcgcaatttgctgctgaagtagggtcttggagtcttgcatccacttcttctggacagcctctggcatgtccatgagtttgctccacatgtctttggcctcctcaatggatggccatgccactggatcacctgtctcatttatgttgtacaccatgatactagccttgacactgcacaagattgatagctctgttacctttttaatcaatctagcactacgcttcctaagagtcactttgcgggcaatattgttggcctggtaagctttgccatggcacacctcacctatgcgcgcgcacagacacggacttggtaatggaaatgacttgtaatttgtttttgtgtgtgtttgcaaatagaggtaggtgacctttttatagtgaaaatcatggtattctattttaggtgaatagcgtataaagggctccaaaagagtgaaaaatataatacgccatatagggagaaaaactttgatgtaatttaaattctaatgagagaagataaggtaaacacaaagctagtatagtttaaatatcttcaggatagggtaaatcataacgtcggcatagattaggtgctatttatgtttgtgggatttgttgttcacacatatgcagatctgggccttgaggaacctggtccatgtctcgaggccttggactaaaagttcatggagctagactttttcataatgttagtttatatgggagctcaatgtgggctaggccatccctggacttattaatcaacacacggttgatccaccgacatagaacaagcctagttgtcaacttgtcatatatatatatggaatggttaggaacacttgtaacatcagtcatataatacatgaaaaattattttcatgtcatcgtcctatatctctaactctatgttatctgttttgtgctatgtaaattgctagagtgcgcggtaagtaaaggttgccacaccttgtaaatatttggtactttgatcttttcctgtccgaccacgcatatacaaacgtgacacacgcaagtaaatatcacgggtttaacttggtaagatacatttcataccccgcaagaacgcgcatctacataaatccccttgaggtgatcaaggatacatccgaactcgaattattttgcatctaccttatagccgtgcacataccttgaacagatgtacgcttcacgtggaattttgtaaatagtttcaattttgttacatatttatcaacaaaatttcttttaaaaagttgacaaatataattatagtacaatcgtacatagtgtaatgtacttttttagtcaaagttctttaggtttgagcaaatttatagaaaaattagtaacatctataacaccaaattagtttcattaaatccaacattgaatatattttaaaaaaatatttgttttgtatttgaaatatcactatatgtttctacaaacttatcaaactttaaaaaatttgactaagaaaaaaaatcaaatgacttatcatataaagcggagggagtaatttttatataattttgaactgttagatttgtttcaagatttgtgcaaacaaggaagggaaaaaattatgtgatttcagtgtcaacaaaaatttcacgaatttcggttttttcaaaacttttttacacgaaaatatttgaaatttaactatttttactaaatttgaattcataaaaattactaaaaaacctgaaaattgcgagggagattgttgcttgccagggggtccaaaattagtagcgcgaaaacatcgtctatatataggatatggattttcatccctctacaattatgcaagaccaaatttaacttctacaagttgcaacaaaaagaacaaaataagttgaaaatagttattcaacttctacgaaaactatcaatccaatcaatagctatcaccatgactaccatggaagacgccgggcggccgcggcatgacggcattcgcgctccgcctggcgaagcgtctcgccggcggtggcggcagcagcggcaacaacaacaagaagaacatcgtcttctcgccggtgtccctgtacgccgtgctggcactggcggtggctggggccaggggcaccaccctcgacgagctcctcgcactgcacggtgcggcgtcgctcgatgacctcacggagtccatccatcgcgtcatggaggtcgatctcgccgacgagtccgcgtctggcgagccgcccatctcctatgcctgcagcgcctggcacgatgagacgctcatgctgtcgtattcttccatggtcaccctaagcccatgttgccgccaccggcggtggtagggacgggcagaggaaggtgaaggggcggcggaggttggggttatgggagttggagaagggggaggcagaagtggtggtttggtggccgcagcagaggcaatggggacggaggaagcgatgcagagctgcggagggagtggtacgaaaaatctaagagtgtctgatattttttgtctttcatcaggcgtccgtgtaggagagtgtttcatattggggaaactattctaatccctcaaggggatgttccctcgtttgctcaaaaatcatctaaacggttatgaaaaattctggaaaaatttgacaacattcatacaacacatatatacaacttcccaaaatcttaagtctaaactcaactcacacgtcgagatataaaaaagacaaattcagcatatgaatagtagcgtaatgtttgtatctaaatttgtcttttttgtttctcgatgtgtagatcgaatttgaacatgaattttggtggactagtaggtatcattatactctacgttgttatttttttcagaatttttcacaactatttgcatcgaatttgaaaaaaaaaaggtatacgaggagatatcccctcgagggattagaatccactcccatcatattgtactacggagagagtagatatagctcttttttggaacacatactactaaatgggactatctatacaacattggactgaaaattagcctaaaaatctgtcaaatgtatctgtaacataattacactatgtttatactataattaaatatgtaattttcataattacattatatttgtactataattacatatgtaacttgcatgtaactcgcatgtaattacagtataagtaagatgtaagttcaaactagatcataaaaatataatagcccaatgaggtcacaggatcgatccctaaaaatggtttgctttttcttcctaaaagcacatatcttgatgcgtattaaactgttagcatgaatctagaggacacatccaacggtttagaaagtgaaagatttagccataaaaaatctgtcaacaaatatttagcaattccgctaaatttaaccgaattacaagtgaatggaaaatagagctaaatagcatcaacggcataaaaacgtaccattaaacgtaccctacgaggcatctagtggatgaccgcaaaacagctcaaatcctaccgtgtttaacacgacatatgtgtctcttatatgcatgcaggtgcgatacatataatccacggatagattacgtgtaatcctttcagcgtacgtaaaaaaaccgaattctcctccatggaagcatgggcacggttgttcgccggctccggcctggcggcgctgtccatgcgcctcaccaagcagctctccaccggcggcgaccacctcgccgcagccggcgtccagcagaacggcggcgccagcaaggccggccccagcaacctcgtcttctcgccgctgtccatctactccgcgctgtccgtggtcgccgccggcgcgcgcgggcgcacccagagcgagctcctcaaagccctcggcgccgggtcccgcgaggagctcgccgagaacgtcgccaacgatggcgcgcgccctccccgacggcaccccgcagcgcggcgggccgcgcgtcacgcacgcgtgcgcgatctggcatgagcgcgcgcgcacggtcaagccggcgttccgcgacgccgccgccgcctccttcaaggccgtgacgcgcgccgtcgacttcctccgcaatgtcagtaacacatacagaggacgcgatctatcaatctaatctccattaaattcttgcaactaatctctcttcttgatccgatcgatcagccagaggaagcttgtaaggagatcaacagatgggtgtcgacggcgacggagaacctaatcgacagcatcgtgtcaccggattcggtggacaagaacacgaggctcgtggtcaccagcgccgtctacttcaagggcagatgggcgaggccattcgacaaggagaagaccaagaaggacaagttccacctcctcgacggcggcggcgacgtcgacgccgatttcatgcgcagcggcgaggaccagtacatcgccgtgcaccgcgggttcaaggtgctcaggatgccgtacgcggcggcgcagcacgagcacaaccgggtctacaccaaggagcacctgcgccgccggttcgctgccgccgccgccgccgccgtcgtcccgaaggagatgcctgcgacgtcaccacggtactccatgtgcgtcttcctccccgacgagcgcgacgggctgtggaagctggaggacaggatggcggccggcggcgaggggttcctccgcaagcacatgccggagcggcgcgtcgaggtcggcgagttcaggatccccaggttcaagctctccttcggcgacagcgtcgtgcgcgcgctccggggtctcggtgtcagggccctgttcgacccggcgtgctggaggcggacaactccggcgaaccgccgctgttcgtgtcggacgtcgtgcaccgcgccgtgatcgaggtgaacgaggagggcaccgaggcggcggctgccacggcgatgattcttttaggtgcggcgccgaatgcggcgccgccgccgccacgggtggacttcgtcgccgaccatccgttcgcgttctttgtagtggaggaatcgtcgggtgcggtgctcttcgccgggcacgtcgttgaccccacaaagcagtagtaattcggtattcacgtattgaaatttgcacaaatcttaatggtagtggtaatctgagagcaaaagagagcattttgtttgtatcgatttgagaaatcgcgtggaagctagatacaaatcccttgatcgtttttaaactttttggtgggggaaatgtttgatctcaatcgttttccggactattcaataacgttgtgcatttttttttctgttttgggttagaattttcagtactaaaaaaaggattttcctgtataagaaactatttagattgcagacggatgtaactggtcgcgtctgtaaaaattgtcctcctatttttatatgcggacacttaagaggaccttttaaggatctgcagttccgcacacaaaaatagcatattttgcaggcgaacctcttaaagggccacacgtaaaaatcaattttcacatacatacctcttaaaaggaccgcacgtgaaaatacccctccctcctcatacatcttttccctctcaaccaaaaatttcatccttatcatctctctccatctctctcacctccccatccttatcctcattaacctctctcctcatcattatcctcgtttccctctccatctttacacctcacgggcgtggggcgggcgtgagcgggagcggtggcagcttggtgtcgcactccccggtggtggcgcccctcacccatccggatccggaggcagcgccggcgatgtggcagggagcggcggtgtgttcgctggagctagttcccaaccagcgcagtgcgcacacggaaaacggagcgatccattagcgcatgattaattaagtattagctaatttttttttaaaatggaacaatatgatttttttaagcaacttttgtatagaaactttgcaaaaaaacacatcatatagcagtttaggaagcgtgcgcgcggaaaacaaggcggagggagctgtgaaccagccctcccgaacacacgctgtgttcgccatttccagttcccacccaaaatagtactcacgaaaaatggagcggtccattagcgcgtgattaattaaatattcactaatttttttagaaaaatagattaatttgattttttaaatcaactttatagaaactttttgaaaaaaaaacacactggttaacagcttgaaaagcgtgcgcgcgaaaaacgagagagatgggtggaaaaaggggttgccgaacacacccttaggctgtgttcttcctcataggttcccaacccacctccctcgttttctgcatgcacgcttttcaaaccgctaaacagtacacatttgtaaaaagtttatataagaaagttgctttaaaaatcatattaatccatttttcaaatttttatagctaatacttaattaatcacgtgttaatctatcgctctgttttccgtacagaggggagaggttcccgacctttccaaaagaacgcagccttcgatgttgagtagataggtcaacagggtgtggccccaccgcataagggtcaggcttatagttggccaaaaggcctacaacttgacggcctgacccaaggcatgaagatttggtctgacacagacacagcatggcccgacccgaccgttagaccatgccttgggctcttcctcgtacgacgtgtcaacccggcccaacgctccgatatgccgtaatcaatcgacgaccagcccaaagagatgagggaaacaaaatagacttatttgagccattcaagacacgacccaaagagataagagagttaaaataaacttattccaaggaagaacacggtggactgatcccgccttcaaggggtggcacggtacaatcggagtcttttttagcattgcctaggccataggtacatcccatgaacgagcacgacatgacacggtgtgttggtcggaccataccggcccgactgatctcggaccgtgctttactcgtgcctgggccggggccgggccatgcccatttggccaatctagagtgctgcccatacctcacggaaacagcagtcaccaacctatccacgcgtttatcgccgtggacactgtccgtgctccaactgtcattaccttagccttgaaggctctcgttgcacgggacaggagagcattatacaccgtgtccacggtccacgccagcccaccctacccagctggctagccacacttcactcgccccttaattcctctactactactagtgtccttcaccgggaaactatcctgatccctcaagggaatatcctctcgtttacttaaaaatcatctaaatggttatgaaaaattctggaaaaatttgacaacattcatacaacacatgtatataactccacaaaatcataagtccaaactcaactcacatgtcgagatataaaaaagacaaattcagcgtataaatagtagtgtactgtttatatctaaatttgtcttttttgtttctcaatgtgtagatcgaatttgaacttgaattttggtagactagtaggtatcattatactctacattatcatttttttagaatttttcacaactatttacatcgaatttgaaagaaaaagatatacgagggatatctcctcgagggattagaaaccaatcccgtccttcacccatcgcgttggttcgcctggccgcctccgcgtctcctccgacctccgcgcggccgtccacccgcggctccgctccggctgtgcccgtcgcgcgcgcgcttcctctagggttagggttttccgtctccggccgccaggtttgctgccgcggccgtcgccgtccaatggggagcccgccgccgccgcgccgccggtgctgcgcgatccagggtttcgtggtgttgttcctcgtctacgtgctcgcggtgcttgtgctcgccggcggcgagctcttccgcgacgaccacccgctcgacctccgcttcccctgtacggcttttatcttttccccctttgtttctttcttctctctggaattggatcgatttgttcgttgttttcatggcgttaatctcctcgtttctgatctgtctagcctctcctggaattggatcctcctcctcctcctccgctcgattcctcctctcgcctcgctcgttgctgcttcgtctcggggagatcgcgagccgtcggggcaggtggtggcgaccggaatcggactctccgacgagcggcggacgaaaggacgggaacagttcgaccacggaggcgtgttcgcggcggtgcgccgcctccggcctgacggggatatcgctgcgcctcgccgagcagttctccgcggaggaggacggcggcggcgcgccgcggcaacctcgtcttctcgccgctgtccatctactccgcgctctccgtggtcaccgccggcgcgcggggcaccaccctgaccgagctgctcgccgccctcggcgcgccgtcccgcgacgcgctcgccaagaacgccgccgagatcgcgcgcgccctcgccggcggcacggcgacgggcgggccccgcgtcgcgcacgcgtgcggcctctggcacgagcgggcgaggtcgctcaagctggcgttccgggacgccgccgccgcctccttcaacgccgcaacccgcgccgtcgacttcctcgccaacgtaagccaataacattgtgacgcaatctatgtcaatctaatgtcactaacccacccgccattgatctcctctcgatcaatcctccatgcagccggaagaagcgaggaaggagatcaacagctgggttgcggccgcgacggagaacctcatcgacacgatcctcccgccggggtcggtgagcacggacacgggtctcgtggtcaccagcgccatctacttcaacggcacatggcagacgccattccgaaagcaggacaccaagaaggacaagttccacctcctcgacggccacggcaccgtcgacgcggacttcatgcgcaccggcgaggaccagtacatcgcggcgcacgacgggttcaaggtgctcaagatgccgtacgcccacgaccacgcggcgccacaaccctcgccgcggtactactccatgtacatcctcctccccgacgagagcgacggcctgtccagcctcgaggacaggatggcggcggccggtggcggcggcggcggcgagggcttcctcagcgagcacatgccggtgcgacgcgtcgaggtcggcgagttcaggatccccaggttcaagctctccttcagccgcagcgtcgtgcgcgcgctccggggtgtcggggtgaacgccgtgttcgatcgggccgagctgccggacatgatcgagggcgagccgctgcgcgtgtcggacgtcctgcacaaggccgtgatcgaggtgaacgaggagggcaccgaggcggcggcggccacggcagtgttaatggaaggtgcggcgcggtatgcgccgccgccgccgccaagggaggacttcgtcgccgaccatccgttcgccttctttgtggtggaggaatcgtcgggcgcggtactcttcgcggggcatgtcgttgacccgacgaaatcttagtcaacagaagtatagtatcattgtttttggaaattgctcgagtcttaacgctaatgtctatttctatccgtcccaaaaaaaaaaaaactgttttctcttttttattctcaaagataatttccaaaaaaaaaaaagaaaaactgttcttctaatgagaaactgcgtgggtatttaaatggactgcacgctaaattctttttttttctcaaacattagataattagaaaattcccaaaaaaagcaaagtgagaatttttctcttttttttcagttaattagagttttcgattgatttgttaagaaaaactgcgtgggtagttaaatggactgcataaggcggacaaagtaccgtaccttggcttgcgaagttgcgatgggaaatagatatttaagttctttcagaaaacatattgttcttcacgtgggcctgtatgataacttattcaaaataggttattcggttattcctcgtccatcagatccaacattagctattgatttgtagataaaaacttgatctgtactggtcacaacctaattaaacatgtctagccgcacatcatatctactgcttcctctatttcacaatgtaagtcattttagcatttcccacattcatattaatgttaataaagattcattaacatcaatataaatgtggaaaatgatagaatgacttacattgtgaaacggatggagtactacgtatacctgttgcataatatatgggattttggtgaagtactaggatatgtcacatccactcaaaatcattcatattaatatgtcgcatccacctaaaatcatttatagtactacgatatgtcacattcacccagaagctcttagggtgtgttcgattgggtggatgagcctgaatgggagatggccgcccgatttttcgtgttgtttgattggagggcatgatggatggggcgacctgcaatatgaaatattccctcgatatgctggatgagtgcattcgatcgatttggctggatgaagctactcagttttggtctgggtgatgtgtcatattctgattggttgtggttggtttaatttcttgttaagcaagctctaaatgattttttttctcctaaaccgtttattcaatttatgatttgattacaccattatatttgttataattaaatctttaaaacaagatctcgaatgattatattttgataaaaaaatatatatgacaaattggtctcactaacttttggcttttcccatccatgttatatccctccaaccaagaaattggatcgccatatctatacaaaccaaacaaaaaattagattggcatatccaacaaagtatagatggccatatctcatccatgtatgactgtgacccaaacacacccttatattatgggacatagggagtatcttttacgttggctccctattctggcgtgttagatctaatcatggtggcatctccgatgatcgaataggaaaaaaatgcattaaagttaaaataaaccgccaacatctctccggtttaggagaatttagagtatttaccatccaattcgcatgccccaacttttttactacccaattcatatgccctctataatatatccttaggactgctaattagtctatttggtaccactttatttgtttttcatgtatttctcttttcttaatcccatttgtattttttttaagttttgtggactgttttgcccctggacccacctgttagaacaaagggtgagaacaaagtctggatcgatcgatctctctgcccgagcatcgacatccactccaccgtagcgggccacgagcgccaccaccctgtcaccgtgcgccgccaaccccgccgacgtgtgccgctgtcggagagtgaactcctagagcaggggactaaaggggccccctttcagttcggccaggggcagcgggtgagaatcgagggttggtagaaagaggggtcgggcctctcgagaacaatgagagaaaatagatttatacaggttccggccgctgagaagcataacaccctactcctgtgtgtgtttgatttgtgaggaggcgcatgtacttgttggatctatccgagagtcatcctccacttcttcactaggcctggacctccttttatacctcaaggggttaccacagtggcccaaacgatctacctagggaagaaggaagacatcatttacttttattacacgtcttatttctctgacaaaggaaatcatgccttcggcgctccagcatggctgtatcaaatcacaacaggaaacacgggggacgagtgcgtcatttcccattaaccatctaaagactttgggtacccgttcccaccaaggaatggataccctaatttaaattagagtgggatattgtccccgacttgcacacggggcgcgatttctctcatcatgatgatgtgaccagtcggcatcggccacacgccgcctgacaaagggccagtgcgcaggcgcactcaccctatcacaccggtcactcaagcgtcacttagcctgtcaaccactcctgcacttcgcattaaatgcggcgtggcggggctgcagctcctgccttaagtgttgttagttgcacgctaggcgtgcccacctaacccgcgcatgttggcaggtgcatgcagggggtcatgacggcctgaccaccgactgggagggggcagctgtcactccaccccggtccgactcccctcgggggcaatgccacgtggtcctgacgacggcttcctccctctagtcatgatacccatgggcccatatcaccgagagccaccaaccccgcccccaacgccgaggagtgccaccgccaagcaccaccacttccgccgccgccgagcgccaccaccgatgcccccaccgccacagttctcagaatcaacaatgtccaactccagtgcttgtaattgttcctcctggcatgtaacccttgtcttctggaatgccttaagagaccacttgctggaactcggcaccgaaatctgctttgataggagatgcaaagttgattcgagctaggccggtgttggcgatcggtggcggtgctcggcggcggggtggcggtgctctcctagtcctggagctagggcgcgctcggcagtggaggcggtaggggcattgagctaggccgacggcggtgctcggcggcggggtggtggcgctcggcggtggggcatcgagctggggcagcggcggtgcttggtggcggcgctcagcagcggggttgtggcgctccggcagcggtgctcggtggcactcggcggtagggtggtggcactcagcgttggaggcggagtatgccagtcactggcggaggtgctctggagagatcgatcgatccagagaagagaaaacctctccttttgttctgacatgtggcccaggggcaaaacggtccacaaagcttgaaaaaaaatacaaatgggattaagaaatgagaaatatataaaacagagaaaatgttaccaaatagactaattagtagtcctaatgacatattatagagggcatgcgaattgggtagtaaatactctaaattctcccgatttaggtcatgtttagttcgtgaacaaaaaattttggcatgacacatcggatatacggacacacatttgaagtattaagtgtagaccaataacaaaacaaattacagattccgcctggaaactacgagacaaatttactaagcctaattaattcgtcattagcaaatatttactgtagcaccacattatcaaatcatggcgcgattagacttaaaagatttgtctctcaatttccgttagaactatgtaatttttttttgtccacatttaatactttatgtatttatctaaatattcgatgtgatgggtgaaaagtttttttttaactaaacaaggcctgatgcaggcaggcggcagccgttaaaaaaaaaatgcaagaggggtttggatggggctaaaacgttatagcccctgtcgactagggttgcagttaggtgaacctcaccactgcagcaaattggcatgtcatgttagcacacgaagtgggttgcaggggacatggtgcggagagcttgaagcgaggttcagcagcgaagcaatggcaggctcgagtgggagcacagcacggtaggcgtctcaaatatccaagtaagttaaaacctttgcatctcttattcaatgcagggaaaaaaactccacttaattactcgcctgctgttatttgtgattctggttcagaggccggtggatttcatggagcgagaacaatccatctcggaggttcctagctatcttgtgcaagggctcgggtgagtagtttgtgaatgtcatttggtcgattactttaatgtttgaaccattttgacttgggtcgagtttagttccaaccttttttcaaacttctaacttttccatcacatcaaaacttttctacacacacaaacttccaatttttccatcatatcgttccaattttaacaaaacttttaattttaacgtgaactaaacacacccttggttgtttcatttttgaatgagcaaaatgatggatgcaatatctggtagtgatatgaaccggtgacaacgccgtatttgtctacactgttcatgattttttttttgaggaaccgacgacaggagtttctcctgccggaatttatagaagaagagaatcggcccagttaataagagaaaccgggcccaaaaaccaaacaagcacggttaattaaatgggaaaccggcgaaaacctatacagaaaaggaaaagaaaacaactattgagacctcacaaaagtcatcgttgccgagcttgcccaaggcaagcagcttcgacaacataatgcacgaggtctccagaaccaccaaacatcctctagcggagcagaaaccttctgagcgaagccttcgacaagggaacaacatcaataatgctgtcaacgcccgttccaaaaggaacccagttttcacctgaagggagagagagccatcatgatgatacctccaaggaggaaacggcacccacaagcgttgatatcatcaacctagaaaaacactaggcaaggttttcacccgagactctctcaaattgctccaccaccaaaccctcatcgtgttgcctggacgtctgttgagggtcatcgttgccgagcttgcaagacaagcagctccgactctgcctcaacagtcaatcacattgcagctcgaccagagatcacctcacacacaggaacccaactgcagcaaacctacggcgcggccgacgaccagtgagacaggacgccggaaaccaactccacccgccacccctaccacacaaaggagctccccggcaatgactggaaacagcttagcctaagaagggaggaggacctaagaagagggcagcggacgccacaccgccggtgagcactggaagacgaacaactaaagaagaggggaaccggatctggagaaataggagtgagggagatggaggcgccgtccccgtcgccggcaaaatcgcagcaggtcggcagcccccagctaggtcagcggcagccggcgccggagcgcgccaccgagagccggactggagccgccgccgacgaatgcgagccgccgccgacccgatgccgtggtcgccgacgagcccacgccggagaagcccgtcaccggtccatccgcctacgcccgagcggcgtcggagtcgaccaagcccgtcaccggtcaccccggccagaggggcaaccggatctggcgactccgtcgcctgtcccgtcgccggagccgaccgccacggggagccccaccgccgcgccaccgtcgcggtggagagccccaccgccgcgcagtcccatcgccacgacctcgccgtcccgctgccatcgtcgccacgcccgcgccgggacgagacggagcggaggatgatggccccgccgccgccatcccagcgcgtcgcccggctttgccgtcaacgagctccggcggcggcgaagcacggaggagggaggaggaggggtggcggcggctagggtttcgcccccgagcagccgcgcgagggcgacgcgagggctgttttggatttcccttccactgttcatgatttgaagatagagaactctaaaataagagcatcccttgtcaatgctagagttgagattgatcaacttatcgttgcgcgcagtgtacacgaagcacagtgcagaaggggcgcgtctagtatacgaatgttcgcgtagcatgaacacaatagagggtaaattccgacaagtccagtccgtgtgcgtgggcaccttttactttcgaccgacgtgctagctggctcgttcgcttgcagcccaatagatacaacccacaatattttagcctaattcatttgtgtgtataaactttgtaggcatatactatatacctatagactttgcacatataaaatttataccatagaaaaaatagtgtagggattttacgcacataaacacatttagtatgcaaactttacaccatggaaaaatttgctgtagcaattttacacatagaaacaattagtatacaaatttatacacataattttctttgagagaaatacacggaaatatttggtatagaaattttatatttgcatggtccatgttttcttgctacagcgtagatgcccttcatttgaattaggtccaagcccatcataagagtaaaaaagaagaaagtctggcctcaatgagacaggcttggtccgaaaaacatatgggtccacatacttaaaaaatattaaagaactaaacacggcctgtgtagtgatgttatttgctctgttgcttagagcaggtataataagtcctaatcagcaggctgaagcagtggcgaagccaccgtaggggccagggagggccaacacccccctaacctctatgtgacctcccctcccctctcttctcccaaggatgattagcacttataatctagctattaattatatcattagttgcagatgagactaactatataggacaggacaactgcctagctatatatatatatgactcatgaaatgacttagctaaatgatttatcattttagaagtgattgcaagactccaattaatgaaacaaactgttcaatcatccatatattaaaaatattactgaattttaaattggcaaatcatgatatatgggttcttgttctctcttgtagctaatcgctgtaatatgtatgatatcgagttaccgatcatacatcaacgctataaaatcactgttcactaaaccttaagtgagttgccatgtctcgtagatggcaccctccatctctaaatcttggcttcgccactgggctgaagggtttgccatgtcatcaaaatcctaggtggaggagtgagaagagaagagagagaatgaagcgggcggctcctctatcgctcggttgaagcgcatcgtccgagaaaaatctctcgctttccagcccggtgcgagcgctaagggcacccacaatggttatctataggctctctacaagagatccatgtcagcatattttcctacatggaagatattaaatgaagagagagagcaaaactatctactaaattagagatggtctatagagaaaaacgaggcaatgcatgagagagatatagatacccatgtagacatactattgagatggtttactattaatctagtctattgctgagatgtacatgttttatagatagcaccttactttaccattgcgggtgctctaagggcacccacaatggttatctatgggctctctacaagagatccatgtcagcatattttcttacttggaagagattcaatgaagagagagagcaaagctatctactaatctggagatagtctatagagaaaaacgaggcaatgcattagagagctatagatacccatatagacataccattaaagtggtttactattaatctagtctattgctgagatgtacatattttatagatagcaccttactttactattgcgggtgctctaagccgctgctttgttcctccctccgatcccatgcatctctgccgccgccctcttccctagctcgcctcctctacgcggaaaattgccccagcctccctccaaatcgagtcccccctgctacaattcatagtataaaacactcaaatccatccccatgccaccaaattcgtcctcaaattgcctcctccaaaccatagacgccatttgtccccaaaccatagcaaccacagcaaggttgaacggcaaagcaagaggacgtttgtaccagcgtctctggaggtctcgccgccggcgactcagcccgtcacgccgccgccacctcagatcgtcctactgccatccttcctttcacatacagatctagagactggagccaggtttcccccagcaacaccacagtccatgagcatgtgtatcttgtttgatgaaatagccacaatagatcatatctcatgcatgcattaaatgcctaaatatttatcaaaggtagattaaccaccaacctatcagccggcctattatataagtgaaggctttaggtgatgtgtcactagtataacgcccactgctggcgttcttattatccttgctcttatctggttcaggctagatcgatatggatattctgtttgcctcgccttttgtgttatctgaaatttatatggcacaggaatgatcttttgttgtgtatgtatgtttgttcaaaatcttaaatgaatggaatgaattatcccttcagcattttgtggttagtattttaatttcgatgagatgaatgaccaactgtaatatttgtcatattgaatattttgtgagactgtcagttatgtcgtgaaaattgccatccataatatatgtcagattgacaactgtcactgtcaactcatagtaaaaaaaaaaatcagatcggaccgaccgttcaactggaaaaaaatcgaaaccaaagatcttcaaggttcggttcgaatgtaagaccggacatacatgtaaaactaccttgaaccggctattaaaccagcaccgattaaacctgatgaggccacaacagtcgtttaatggccttaatgtggcccaattagtctgccatgcacactcagttggcaaaaaatgaaaaaaatatatcctcacatgaatccggctgtcctagcagttgcgttatagctttaacatatattatatataaaaattgaaccgcggttcaaccacctggtgctcgattcatagtttgaccagcggaccccgtaaacccaggtgcccgattcatggtttttcgtactataccggcaagcaaacaattggctgtcttcggctgcccctcttcctaatccctcctctcgttttctatgcgcgagcttttcaaactgttaaacggtgtatttttttaaaatgtttatatacgaaagttgcttaaaaa

SEQ ID NO: 5 - DNA, 32548 bp, Magnaporthe grisea
  misc_feature (1)..(32548): Continuation of Sequence ID 1, representing bases 198,481 through 231,028
atcaaattaatctatttttttaaaaatattagctaatacttaattaatcacgtgctaataaaccgttccgttttccgtacgtacaccggtagggaagcaatggtgaacacagccctaaacaggtcaacctcaggtgttgactagagtactagacaggtcaaaagggtggccccaccgcaccacccaacggaatgcccaattgctcaccgaaacagcagcagtcacaacctgtccacgcgtttatcccccgtggacacggtccacgctctaaatgacaataccttagccttggagtctctcattgcacgtgagaggagtgcatacaccgcgtccacgcccacattcacacgccccccggtaattcctcgtccctactacagtactaccagtagtaaaactttcctgcgtctcctcccgtccaatggcgactccaccggcggccggggcgccgcgccggcggtggtgcgcgatccagggtctcgtcgtgttgttcctcgtctacgtgcttgcggtgcttgtgctcgccggcggcgagctcttccacgacgaccagctccagccccgcttcccatgtacgtgctcccggccggattcctggtttatcttttttttttcccccacgtttaattttttccccctttttttctctcttccgttctgcaagattggttcgtgatttccacgcgtttctgatctgtgtagcctctcctggaatcggatcctcttcctcctcctcctccgctcgaattctcctctcgcctcgctcgatgatccgccgtctcggggagatcgctcgccgtggtggaagcaggcggtggtggactggtggtgttcgaccggaatcgggctctccaagaagcgagggaggaaacagttcagccacggatcaggcgtgttcgcggcggtgcgccgcctccggcctggcggggatggcgctgcgcctcgccgagcgtctgtccttggaggaggacagcgtcggcggcggcaacctcgtgttctcgccgctgtccatctactccgcgctcaccgtggtcaccgccggcgcgcgggggaccaccctggccgagctgctcgccgccctcggcgcgccgtcctcccgcgacgcgctcgccgaggacgccggcgagatcgtgcgcgccctccccggcggctccggcacggcgacgggcgggccccgcgtcgcgcacgcgtgcggcctctggcacgatcggaggaggaacgtcaagccggccttccgcgacgccaccgccgcgtccttccaaggcacgacgcgcgccgtcgacttcctcgccaacgtaagtgatcgaatcatcgatctatatcaatctaatgtcacgccattaacgaatgacgatctccattgaaacggacatgcgcgacgcgtgtagccggaggaagcgaggaacgcgatcaacagctgggtcgcggcggcgacggagaacctcatcgacacgatcctcccgccggggtcggtgagcacggacacgcggctcgtggtcgccagcgccatctacttcaacgccacttggcagacgccattccgcaagcaggacaccaagaaggacaagttccacctcctcggcggcggcggcgacgtcgacgcggacttcatgcgcagcggcgacgaccagtacgtcgccgcgtacgacgggttcaaggtgctcaggatgccgtacaacacgcgtgcctcccgtacgcacacgcaaccgcagtactcgttgtgtgtcttcctccccgacgagcgcgacgggctgtggaccctcgccgacaggatggaggccggcggcggcgaggtcttcctccgggagcacatgccggagaagcgcgtcaaggtcggcgagttcaggatccccaggttcaagctctccttcgacggcagcatcaagaccgcgctccagggtgtcggggtcagggccgtgttcgatccggcagcggccgacctgtccgacgtgctggaggagggcaactccggcgatccgccgctgttcgtgtcggacgtcctgcatggggcagcgatcgaggtgaacgaggagggcacggaggtggcggcggccacggtggttattatgaaagggagggcgaggcggccatctccggcgccggcgccggtggacttcgtcgccgaccatccgttcgccttctttgtagtggaggagtcgtcgggtgcggtgctcttcgccggccacgtcgttgaccccactaatcctagtcaactgtaatgaaatttgcatggatcttaaatggtcgtggcaactagagagcaaattgagccttttattcgttttaaattggatatctaccctatagtatgaatttaaactggctagttctacgtttgctatattttagggtgaatgaaatagaaaattgctttgggtgtgttcggacctctagtttcccaacccaaatcccttgtttttcgcgcgcacgcttttcaaactgctaaacggtgcatttgttataaaaagtttctatacgaaagttgcttaaaaaaacaaattaatctattttttttaaaaaaaattagctaatacttaattaatcacgtgctaatagatcgttctgttttccgtgtggaggcactatgttcccatcatccacatgctaggcattcatatagacatgagaagacggctcactcagatctactggatcagcaagaaggcgctggcttcagttgagagtactggagctgtgtgcttttagtgagggaaaatctattcaggtttcagcgaaacgtttaagcagttttacattaacgccgtgtttagttgcaaattattcttcaaactttcaattttttcatcacatcaaaatttttctacacacacaaactttcaacttttccgttacatcgttccaatttcaaccaaaattccaattttggtgtgaactaaacacagtctaagatgctgctctttctagtgatatttgtactggtcatcgaagtcgttcacataggattggttgaggtgattgtaattgtcaaatgtgctgtcgtgcaacaaaaataaaggattacattcgagatggaaatttttgcttgtgtacataatagagcaagagtacagtagcatgctccgccgctggatctgggagggagggaggggccgggagccgccgccaccctcctccgccgccgctcgccgccaccctccgcctcccgccggtagccgccgccctccactgccgccgtacgcgtggccaccgccttccccccgcccccctcaggccagatctgggagagagtgagggaggggagtgccgccggccaccgccctccgccgccgcctccaccggtagccgccgccctccgctgccatcagcgccggggagaggaggagaggtgcggggaggggagaggtgaggcaaagaggacttagaaaaaaatctagggtatacagcctctatttatatttcctatgtattttcgcaggcggaggtccgcctgcaaaaatgaagggtatttttgcaggaagaccgtttaagtggtccacatgcaaaaatggattttcgcaggcgggcagcgggtttcaccccgatttacattttcgtagacggctatttgtatccgaaggttatgtccacctgcgaaaataagtatccaacatcggcaaaaatgccttttcttgtagtgtaatagagatgcgacatatcctaagactataaatttggagagatagttatctagatttatagttatataatatattacatttgtattaggttgcatttatattccagagggagtaatagctgccctatgtggcaagtggatcagtagatcaattggcatcaaacaatattggtgtgcaatctttcgatcattggttgtttttttagaaaacaaaaatattttcggactggcttgaatgctttcaaaacatgtaattgaacactcgccatcgagtcagaaaatcgttttcaatctactaattgatcggttaactaggtcctgtcatcgtgaagcaacataatgcaatctaataatctgtacacaagtcgaaggaagcatgtaactaagcaacaagaagctgcatatacatcatggagtagtacgcacgaatccaggtcaattggcaaactacacatttggcgcggcgaccctgaatctgaattgaacccatcataggcctgcctcatcaaactggttcgggcgggagagtctcagtagcacctgaggtcctgggtttgactccccgtgggagcgaatttttcaggatttaacggcattgtgctttcagtggtaggcgacgtacccgtcgacagcgaggcgcccgtggtgacttcgtcaatctctccaagatttgccagtccagtcttcgaagatgctcatagatatagggtttgcgtgcgcgtgttcataggggtcagtgcgcgtgcgttgtgagtatctgcgttgtactgtgtaatttttttttaaaaaaaaaactggttcgggcagggccccgtacgtggacctcgacgcctcgagcgccctctgcatctttccaaaaccctacaagtttgaggcggcgatcgatcggacaggcagcggggagacagatcgagcgagcggtaggcatatgggttttaggtcgttgtcgctgctgggctctctcgttcgcgggagtatccgctgttcctcctcctcgccgggatcgggaatcgctaatcgtctactatccctggtaaatcaaactcagaaacttttaactaataattatattaatcataatacatgatatatcgctagattcatattttcactctaatttcaaattcgttaataacgcaacatgaataaaccaatagtcaaaatatgccattgaaaatcgtgtaaaaaatatagatattataatttgagacggagggagtagtaatctgcttcttttgatcttttctcatctcagatctatacatcgtatgctttctccgctagcggctcataattattctcgcagaggaacgcgctgttcttctactccgatgcccgtcgtaagagtcttgtggcggccaacacctctgctttcgccgccagatcatcccgttgtctgcacagcatggtaaatttctttcaaatcttgctgtgtgtgtttccctcctttttcaatcccacagacacacacattcacgcgcgtgtttccatcttgctcccgttatttcgttcgttaattcatcgttgtgcaggggtctctggggtcgccggcggggaggagagcacgccggttccacgacttggtaaatttccttcatcgtttatctatctaagccttgttttacttccagataagctcacgttgaatcctcgtggtgcagagggatgtggtcttggaacatgccgatgctgctaccgctacgttgtctgttgttgttttctctgcggttgctggaattttgcatttgatgcaagatacaggtgagtttttaggtactccatgtgaaactagctgcctttgactgcatgtttcatatatgagctttgttctggtccagcagttggtacctccaggtactggtaccacaggtaccaaacgaaatccggccgttcaataaactggggtacgattggaaatgaagtaaagcagccagctagcgtgcgacgacgaaatctcctcatcttcacaggcatatccgagtcgccgctgccgtcgcattctgctctccttgacgcatgacatcgttcctcagctcgctccgtcgcgcacgtctctgtcaccgccgtcgctgctgcctgtcctccccccagccccagcgtgtccttgctctgcggcaacgacagtgggaccgatggccgacgctgccgcaacatgtatcctgacgcgccggactttgtcgccaccgccgctgtagtctgcaccctataattgttccggaatggaggttgtgttggagcttttcgcatgagtattgattttgtgtttgtctgacaaatcaacctgaagccatttaggcaaatcaacttcatgcttctctttttttttgcggggaacttcctgcttctcaatttattcctgtatgtatctttcttgctcaaaaactgttcaaccaaatcaaccatcgtcgtcgctagcaatgacccgtggagtgatgccgtcgtcgccgaagacaattatcggagcgctgccaccgcagttgcagatgatccctggagcaccgccgtcactgccgataactcctggaatgctgccgttgtcgccgctgacaacaactcctggagcaccgttgtggcctcagaagactggtggagcgcaccgtaattcctgaagcgtcgccggctcgccgccactgccgtagattttcctctgcaaagccttatccaaaacacgaatcaaatcgacgaaattcctttccaatgtgcaaatacaattttacccctccacctcattcccctttggcaggtaccacgtagggagtaaatggtacttgaaggtaccaattgatgtcagccatccgatcatacctatccagcggtccacatttggtaccgggcttcactgctggacagtaaaaaaactcttcatatataaccatcaatggttatgggaaattatttcgatcgcataagaatgtaccctcgtttgcttaaaagtcatccaaattgttatgaaaaattctggaaaattttgacaacactcatacaaaatatctatacaactttataaaatcttggctccaaactcaagtcacacgtcgacgtataagaaagacaaattcagtgtatgaatagcaatgtactgttcacgtctaattttgtcttttcgatgtgtagattgaatttgaacttgatttttaatggaccgatacatatcactgtactctacattgtcgatatttttttcagattttttcacaactatttgtatcgaattttgaaaaaaaaagcacacacgaagggataacccctcaagagattagaacccactcctcatactacatgttaaacacccttcagatttctcgtctttagctttccttttatgtttttcgccgggaagctttccttttatgttacattgccctcaaccctcacgagttcatactgtctgtacgtagacgaagctcggaaggaggacacacccactaagacaaatccttacttccgttccaaccagttgatggaagacttgatacgagagagggcgtcttacaacgaaacaacgcctgacgagaagaccgtggttcgagagtacatggaggatgatcaagacataagggcaaggttcaaggattggatgaaggagcatggcaggacatacaagcaggacgaggtggaggaggctaggcgattcaaaatattcaagtcagttgcaagattttctgatgcagccaacgatgattctgccaacgcagggcactcgacccgttttggcctgaatgagttttcagactggaaccaggaagaacttgctcggatgtgttgttgtatgccagctaggagtgatggtgggtacttcgaaatggtcctgtcttatcttgttccgcaggggatctggcatcaggtatagctgacatggagtacaaggtatgtattcccaatttcatctatacaatgtatattttaatttgtttttctgatagacatggatcgagtgcattaactggagaagcccttagcttgatctgtcccaaaatgtaacgtgttttggaacggagggagtagtagtaacctatcttgtacttcctgtgactcttttttggaatcggcaaattaagtgttaattatttctcagttggcttctcactcgtgaagcagttagtgtactggcttgaccttgaattccatgttgtgataggatcagggttgttttaaatctccgactatagctgccgctatgtccggttatatctgtttgagaagagttttagctcttttctcttccgtataatttagccgctatagatgcccctataactgttgagagggccaaccgctaaattagcctgctataaaacaatggttaggatgaataagaaatgcatttttgaactacctaattattatctgcgctagttttacttaaagagaaatatttgtgtactgattagtaaataccccgatcatatatatcttggatactggcagtttagttttgttatttctatcgtgtggcggcttcacaatcatgagttctcaattgttgattgcttgctgtattgatcgcttgctaagcacttgtaaagtagtttgattttgacacagaaatggcctttattcaggctagctaacgtcaatcatgtttgttgacaaatcaaatactccctataaatgcaatttcataaatatggtatcaactttgcactttgatgcttacggttctgtgtatttttttcttgaagcaaagtagttggagaattgttttgttttccttttcagccaaaaacggttttctattcaagtctctgtttttctgtaatcaatggatcttattaagctcttcattgagaaactctagggtggtggaccttaagaagtgaattttctctcacaggctaacagctacagcgcataaaccagctatagtttctgtaagttgtcatgatttatgttcatgtaatggtacagtagcagtgtgaatgttgatttacagcagcctgaccgtatcgaaaagtaacataaattgggtgcaatgtagagttttaaccattttcctgggacaaaaaaaaatttatacaaaattgccttgctcaaatctgatactttatttttgctttaatttgtatgctcgctggttgcacctttgattcttcgtctgtgtacttctctttcattatggggcttcttcagagctatagcctgcattctaattaatttcctttctcaaaagcaggagttttaagattgagaagggtcgagacgctatatatatatatatatatcatacgaagaccaacaagctgtcatgtgaaaaccagccaaaatgaatctgtttcagatgtcaaatgtccatatcttccgtttttgtaccctctaatgctacctgtctgaatagcataatgctagagttgttatggttatatgagctatcttgtaagggtggtagatgttgaagttaatataaatgggaatttctgaagctaattaagagtttgttttcccattggacaattgcattcttgtcctaactttgatgatcaaactctatatcacacaatactgaaagttacagtcgcgtgtactgtcacaccgagtcgttccctcgctggtggattaatatccccaacaccaacgatctaggctccagaatacgttcccatacgttatgccaagttctttgatcggtagttggaatatatctcttgttcaactaattctaaactctatcaaacatttgttgtcctagtatatttcgggtattctcatgtgattcccacaagctcaaatttattccattgtcatgtcatgcctagttttccatttgttcttcaaaataatgaaagaaaataaccgacctacaaaactcatgctcaatccaatcaagttactcgtcgtgtcgtcatgctttttttggccaaactcccttcgaagaagaatctgtcaccagagagactaaaacaataaaataagttgatccccgtgatgggcctatctgaggcatcattccttcgtcttggggccctcacattctaggcctcatcaaattaactagtttggtgtaccgcgcaaatgtccaacctttttaaggccgctcgtaatatagacgctatccttgccctcgaacccccgcatccttccaaaccctagcggcatgtttgccaagagaagaggattgggggtttgaaaggaggaagttgatggagggattggtcttgggagttccatttttactccccgtacattatttgggtagtagagatgacaattatggcgagtttgggtataaattaaattttacgtgatgatgtatggccgagatctatctcttacagttagaaggaaataggtgtcggttttaaataaactgacacctataggatattaggtgttttctagtagtgcacccttcttagcggtgccactcctggttgtagctttctttgcacgtcttccgacatcagcaaaatggaccacaattcaatgaaaaacatactttatgtattagctcagtagggtcatgagatttcttgtttttaaaacatgctgcatttttggctttccatatggtctagatcacagctgcaatctccacttccatagagggaatatgagctagatgattacggcctgttttggaagtgatttcgtcatcgtcgatcgtctgcattttttttccttcgatggtgacaacattgagtttagatcagggggctagagcgtcgagcttcctacaacaatcatttgtgtctatttcactctcttcgtcgtcgctggcaatagcgttctaagcttgggctacttcgttgttgtcacgccccaccctatcgcacagccaccaccaaggagggtgtgccgcaggagacagtatgccattctcacgcataaccatgataatgttacccactataacaacataaatttttatagtaccaataaaaaattataaattaattattggactgctaagagcaggtacaatagatgaattttgcccgcgatactacatgcatgcagcctaatatggtgagttggcagagaaatttggggagaaagagggtgagccggcgactaattagtcaccgcctccacgcgtgtgtcgaggcaagaaaagcaacatcatacgttttcctactggaccatacagttgtggggtccacgtgattagtagtatgtggatgagaagatgacatgtgtagacatgacactataaaaaaaagaaaagaaatgttttgtcattgagtggaggcagcttgtagactattgttgtacaaattagctatttggtgaaagagtattttattatacttgtcatgatttggagctgacagcttactcctctattaaacttgatctaataggatacatttatacttttaggttgcaattggttgcaaattcaggtagatcgggaagtatagcttttattctttggattgcaggatttgaggcctgccccttttttctactccatcagtttcatattataagtcattttgatttttttcctaataaaacttttttaaagtttaaccaaatttatagaaaaatatagtagcattttcaacacaaaatatatatattatcaaaatctattaaatctttcatctgatgaaactaatttggttttatacatgttactaaaattttctataaacttgattaaatctaaaaaaatttgattaaaaaaagtaaaaacaacatataattaaataatgggggtactaataatagataggttttaaaccatggcgatttcatgctatatatacgaaattgctaaaaaaactgtcgcaagtttaactataatgtaattacagtataatgtaattacagtatagttacattgtaaattacgctgtaactatatagttacattgtatctgcactgtaagtatattgtaatatctatataagttagatataaaactgtatatattattttaatacttatataaaagttatataatagttatagtggaatcatattacaattatactataattaaagtttttttcttaaagaacttagggcagttttttaatagtccctaacaatatacgatatactatgggcctgttcactttgatgccatttttaatcttaccaaattttggtaaagttgtcaaaaaagtggctacatttagtttactgccaaattttgaagatctatctatataacacccgacagatttttgttataaatcacacagatttttctaaccaccaaacggccaacttgctagccaaattaaaaagcaacgcctatagtgtagattaaggcgtgttaatccaagccacaaaactaactacttgtcagcacatatactaaaggctttttttaaaaaaaattacaccctaattacactatacttacatttgtaactatagtgtaattatagtataactaatatgtaagtactgtgtaactaatatataattatagtgtaactacactaaaaagtttttttaaaaaattacaccctagttacactatacttacacttgtaactatagtgtaattatagtgtaactacaccaaaatctttcttttaaaaaaattacaccctaattatactgtacttacgaatgtaactatagtgtaattatagtgtaactacaccagaatctttcttttaaaaaaattacaccctagttatactgtacttacgaatgtaactacagtgtaattgtagtgtaactaatatgaaattaatacactagtggagaaaccctttgtagtcccggttcgtaaccccctttagtcccggtttccaaaccgggagtaccaatccgggactaaagatcgctatctttagtcccgggtgaaataaccgggactaaagatcgagctgaccttttttttttgcatggccctgttgctgcatggtttatatatatatatatatatatatatatatatatatatatatatatatatatatatatatataaaaaccatgcatatatatgtttcaatacatatatatgcatacatatatatatgtatatatatatatatattatattatatgtatatacatgcataatatatatgtatttatacatatatatgttatgcaaatgcatatgtatatagatatatatatgaccaaaatatatttacattatagaaaatgtgtacacatgcatatagaaacatatgtagtcatatattaattacaatccattttatgtcctagctagctacgatttcgacgtagtagtagtcgctgctagctcagaagctaaggaccggtgaattgtgtttccgtcgtagtagaattcacccttgggatcaaggatatcttcgttgatgaatcccatgagttgttcttgaaccgctgcgataaattccttgtgtgtggtcaggttatccctcatgcgaataaactatatgaatgtatgaaattgattagtaattaaacaagaaatcgttacgtaatgaaattttgaatttatttgtacgtacatcgagctctcttgtggtgatgatttggtctgtaaggcagtggcaatactcacacacgtagtagccgcacaagttagttccctgcttttgctttgcgcactacaaataaatgacaaattaacggcgtgagatctaattaatatacagttgtatgtattaatttgaatcggagaaatataaatgtagagcatgtgtactcacaggaaatttgaacttccgcctaagtctttctctccatttgctgcggaccaaatgacggaaccgataccaagccctacatggagtttaaagctatgattcgtagtagcaattaacataacaagattttcttaattaacaaaggaacttatgacggtacctgtctataagttcgaaaaccttgtcaaacgtagactcttttttatccattgagtcatatacgttgacggtgcaggcctccaggtcgaagagtaaaagcacccagtggaatctgcaatgtgcgtgggatgcattacatatgaaacacttagcaagttcgtacgggaatgaaatttggtatgtaggaagacagtaaaattaaactaactctgtgttgtacggtaacagtatgaacgtcttgtaatgctgcgccttcaggagatggacgagattgtcctctgtttcctgcggatattggtcgagcattgcaacgtttactctccgagggtcgatgaatccagtatcgaaaaccctccgccgtcgggccctttgaatctccattctacaacgataaaagggagtattttattttatatagtctacttatatacaaggacctaattaattaaaggagacgtagtaattaagaattataattgaacgatatacttacaaaatccagcaactcataatagagacgtcgagggcgtccagctggtatagttcgtagattcccctgaaattgatccagagaatgtcatctccttgcaagaagtccgtgtccctgatcgtcgctccgatcatctctctaccggtggcgctcatctccatgtacagctgatggaacttgtacatttgtgtgggtagggactgcagcagctcaggcttgacaagcggtttaccgagttcgtacatgtatttcactttcgccttttcgattggtgcgactcctagcaattgatccgtagttagaccggtgtcagtaataaattgttctatcgtcatttctttactggtcaccaacggctcgatctcctggtttggttgctctccaagctgagggactggtttggactttccagaagatgctttcttcagcgttcgctcatagtccgatagcttgatggcctccttgacagatgcagacatacccctgaagaagttcctgacactggggtctataggaatcttcttttctggacttcgaggcttgaattgtctcttgacttctgatgcaacgtaagcgtcaagctcctcttgcgtgcgatcgtaccccgggtccttgttcttggcggcgtcaactttcgccttcttcactgcccttgtgtgggcaggcggtggagctgggggggcccttgactttgaaggtgccggaagaggagcttgcgggggagtaggtgtaggagcacgaggaggagtcggtgcaggatcctgaggaggagtcgatgctggagcctgaggtggagacggtgctatcggagcatgaggaggagacggtgcaggatcctgaggaggagatggtgcacgagacgccgcttgtcgcccagggaggatgatgtaccgcctgcgtcatagaataatggcatggcttgtttctcgtagatgcgtctcaccgtctcctccagggtaatccagctcgaggtcctcgtacgcgccttcgaccaactcaacttcgaccttcgagtatcctgctggaatcggcctgcagtggtaagtacctgaagggtccgttgggatggccatgcccgacgccaccttcatgttgtgagagattatttattagtaatcaacatatataagcagcaactagcggaatggctaaatcatacctttattgataagttcttgaaaggaatatgcagctcacatggtgtccgttgagtgatgtcatcaacgggacaggtcgattcgtcctgtgtttgcatggcgtccatgctctgtgatcctacctgccccgttgaggcgcagctgctacggtttcctgacaggcttaccattgcaggaggaatggtcagctggggatcatgggaccgatgtgcggccatgcgttcatcaaccttccttgccacctcctcttgcatgttgagttcgtagctcgatacctggaactctaggtctgcaatcttcgcctcggtatctctcttgctcctcatccaactcctgtacgtgtggatgtcctccttgaaaccaatcttccaaggaatcacccctttccctcgtgttcgtcctggatgctctggagtctgcagggcgagtgtcagctcgtccctatctctgtctggtcggaacgtgccctgagaggaggcttccactgcatctgttagtcgtcgcgcagcctctcgtatctgatcgccgaagaccagtgagccatcagctgggttgagcgttccaccgtgagcatagtaccagaacttcgatcgttccggccaattggctgttgccggttcgatacccctctcaatcaagctagcctccatctgctcccacttcggcatcgcgacgctatagcctcctgaccccaagtggtgatggtacttcttcttggcggcattttctttgtttctttccatcatcgcctgcccttgttcacctgtcttatatgcaacaaactcgtcccagtgatcccttagctttgggaatgtgtcgaagttcggtgtctgccccttcagtatatacttttggtacagatctcccttgaagctctgaaactgttctgccattttcttcagagtccaccttttcactttgtcctctgtacccgcaggaagggtgaatgtctcgagcattgtggtccacagcatctctttctctgaatctgggacaaagctctcatgatctccgcgtgcccttgttcttcgccagtacaccgtactgacaggcacgttatcccgcacaacccaaccgctgtgacgtacatagttcttggcggcttcggccggggcactaggacgtccatcttcttgcacttccgttatgatgtgccgaccctcaagcttcttcgctgcacctcgttgcccgcgtgccctcttctgtccaacggagggttgactaccactagcctcctcctcctggttcccctccacatccctttccacgtgcccctgctggttcccctccgcatccctctccacgttcccttcctcgttcaagtactggtttggatcctcgttcccctcttcttcattccagtactggctgcttccctccgcgattgtatcgtacaatatctgttcctcatcgcggtcagccatctgttgatacaagaaacatctgctaagtcacgagacatttatgttttaacaatctaagtcatgtatgctaagtgtaaatgtcgtacattagaaccctacacaaccttacgtgctaagtctaattacaaatcgtgatataagctaagtctaggtgacgtatattatataaccctagctagtaaataattatatactaagtctaattacaaataaaataatcttatattaaaactcaaataatattacatgctaagactacaatagtcatgtatatgctaagtgtttcgtgcgtatatgctaagtctaattaagactacaatagtcaattgctacttgtcgcaccaattccgtagtcaataattacatactaagtctaattacaaataaagtaatcttatattaaaactcaaataatattacatgctaagactaaaatagtcatgtatatgctaagtgtttcgtgcgtatatgctaagtctaattaagactacaatagtcaattgctacttgtcgcaccaattccgtagtcaataattacatactaagtataattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattagaacactacacaatcttatatgataagtccaattacaggtctaataatcttaattaattgcattacaaatataacaatcatttatattattacgtcacttgcctgctccaattccttctagggctagctcttccactcaggcttgacggacgactccgacaacacgtcttttctgcaagatacaaaaagatgtattaggataaatgcgaagtaacatatatatatatatatatatatatatatatattgcatttcacgttaacgttcgtcctcgttcgttcgctcgttcacgttccctagctcgttcacgttcgttcgctcattctcattcacgttcgttcgcttgttctcattcacgttcgttccctcgttctcgcttgtcttcgatcgatcgttctcgttctcgctctcattcgttcgatcgttctcgttctcgttcacattctcgcggcaacgtacgttgacgtgttcgttctcgctctcgttcgttcgatcgttctcgttctcgttcgttaacggcggtgtggcggcatcggggcggcggttggcgcggcggcgtggcggcggcggcggcggcgtggcgacggcggcggcggcgtggcgttgcggggccgtggcggcggcggcgtggcgtggcggcggcggcattgcgcgcgcggcggcgaaggcggcggcggcgtggcgacggcggcggcggcgtggcgttggcatgcggcggcggtgatggcagcggcgtggcgcgtcgtcgtcgtggtgcgttgtcgtcgtggcgcggcgtctcgtggcgggcggcgcgacggagagagatcgatatcggagagatcgagatgcatgaagggagatctggcggcggcggctctcaccccagagatgcggcggcagcggaggaggcggaggcggcggcgaggacgacggcgagatacggcggcgtcggcgcggggatttgccctaggcagtggcggcgaaggagagaggccgagagagatcgatcggccgaaaacgtctaagtgttggtgaagaagacgagggcgcaccgggaatatatatacccccggatctttactcccggttgttgagaacaaccgggactaaagatatctttagtcccggttgttgagaacaaccgggagtaaagaaaagatatttactcccggttgttgagaacaaccgggactaaagatgatctttagttccggttggtattaccaaccgggagtaaatatcttttcccgctatttcgaaattctttttaaacccggttagggttaagaaccgggactaaagattatagcactacatatgattttcgcttacatatgtgtttataaaatagaagttaatgcattaacattatttaaagtacaaagattaatgacaattacttcgaaaaattatttttgtctgtaataaattaagtggataaatcgtaataagcaaatatatgacttataattaataaaaattccaatatatatatatatatacttagcatatatatgaatgatattattatattggtaaaaactctaattaagatatgtatgtacatactgcatgatttaaaattaataaaaattgtaatcatcatatatacttagcgtaggaattatattaaattaaatgctaaaaactctaattaacatatgtaaatgccacatgcatgatttaaaccttttaaaaattcgaatagtcatatataagtagcatatgaaagatagtaaattaaattgtgaaaaactctaatatatgaatgatagttttaaaaatatattaaatttaatacttttaaaaattctccagtcaattataattagcatatgaatgatagtaaattaaatgttaaaaactctaattaacatatgaaattgccacttgcgtgatttaaaacttttaaaaatcctctagtcaattataattagcatatgaatgctagtaaattaaatgttaaaaactctaattaacatacatatgaaattgccacctgtgtgatttaaaacttttaaaaattgtaagtatcacatataactagcatatacatgatttaaacttgttgaaacctctaataatcgtgcgtaattaacatatgccatacatcaattgatttatatggataatcaatacatccaaaatagtttacatcatgtacacaataattacggcattacatcggcggtgacggttgaccgcacgtactttctcctcactattgttccctccttgtgatcgctgcgtgagtaaggggtgtcttcatttgataggaggatgctagggtcaatcgtcaccgtgaaagggggttgcccatccaactgatcgtaatcctcgtcagtcttgtcctcaactccgacgatttttcttttgcctgggagaaccacttgacgcttaggctcatcaggccctctgcccttctttcctttgctagacatgtccttcacgaaaaagacttgcgttacatcattggcaaggacaaaaggttcgtcggagtatccaaccttgttaaggtcaacagttgtcatcccactgtcatcaatcattacgcctccaccagtcaacctaacccattggcaccggaacagaggaaccttgagaggaccatagtcaagttcccatatgtcctcgatggtaccataatacgtggcagttgttccatcgtgtcccatggcatcgacacgaacagcgctgttttggttcgtgctcttcatgtcttgggctctcgtgtagaatgtgtacccattgatctcatatccctggaatgtcgcgatcgacccagacggtcccctcgccaggaaggcaagttgttggttgatcgactcgttacccatgagatgttgtcgtagccacgcggggaaagtatcaatgtgatgccgtgtaatccatgcatcggacttaccgatgttcctggcgcgaactagagccaagtgctcctcgatgtaaggagctaccaatgaagagtgttgcagaacagtgaaatgggctttacggaataaattgttgtctaccgtcattattgctttccttccgagagttccctttccccgtagtctcccttcatggcgtgattcaggtaccccgattgggcgaaggtcttcgataaattctacgcaaaattcgatgacctcctctgttccataacccttggcgatgcttgcctctggacgagcacggttacgaacatacttcttcagaacgcccatgtacctctcgaaaggaaacatgttgtgtaggtacataggcccgagaatacggatctctttcacaaggtgacaaagcagatgcgtcattatattgaaaaatgaaggtggaaatatcaactcaaaactgacgagacattgcaccacttcattctgaagggcttctaatctatccggatcgatgaccttctgcgaaattgcgttcatgaatgcacatagctttgttattgttgcccggacattgtctggaaggataccccttattacaactggtagcagttatgtcatcaacacgtgacagtcatgagactttaggtttgtgaacttcttctccttcgtgcttattattcgcttgatattcgtggagtatccagacggtacctttatgctctccaagcattcaaacatactttccttctctgccttgctaagagtgtagctggctggactcaagtaatggcttcctttctcctttggttccgggtgaaggtcgccgcgttgttccatatgcttcagatcattacgtgcttccagtgtatctttcgactttccgtatacacctaggaagccaagaaggtttacgcaaaggttcttagtgaggtgcatcacgtcgattgcgtggcgtacgtccaagaattcccaatatggtaactcccaaaatatagagttctttttccacatcgccgcgtgaccatcttcgctctctatatgctggcttccaggcccctttccgaacactactttaagatctttaaccatagcaaacactgttttcccgctgcgatgtttaggcttcgtacggtagtcggccttatgtttgaagtgcttgcctttcttccgtaccgggtggtttgctgcaaggaatcgacgatgacccatgtatacaaccttcctacagtgcttaagatacgtactttctgtttcatccatacagtgagtgcaagccttgtaccccttgttggactgtccggataggttgctaagtgcaggccaatcgttgatggttacgaacagcagcgctcgtaggttaaactcctcctgtttgtcctcgtcccacacggggacaccttccttcttccacaactgtttaagatcttctaccagtggtcttaggtacacgtcgatgtcgttaccaggttgcttggggccttgaataataatcggcatcattatgtacttcctcttcatgcatagccagggggggaggttgtagatacacatcgtaacgggccaagtgctatggccgctgctcatctctccaaaaggattcatgccatccgtactcaaaccaaaccgtatgtttcgtgcgtcctttccaaattctttaaattttctgtcgatgtttcgccattgcgaaccatcggcggggtgtctcagcatcccgtcctgttgacgctcttcagcgtgccatcgcaacattctagcattccccttgttcctgaacaaacgccttagccgtggtattatagggaaataccacatcaccttagcaggaattctcttctttgttagctgcccgtcaacttctcctggatcgtcccgtctaatcttgtatcgtagtgctttgcaaacagggcatgcttctaggttctcatactcctcaccgcgatataggatacaatcgttcggacatgcgtgaatcttatgaacttccagtcctaacgggcagactatcttcttagcctcgtacgttgtctcgggcaatttgtttccccccggaagaatgttcttgacgagtttcaataaatcgccaaatgccttgtcactaaccctattttttgccttccattgcaacaactccagagtggtatccaactttttgtgcccctgctcgcaacctgggtacaacgaagttctgtggtcctccaacatcttgtccaatttatgggcctccttttcactttcgcagtcctccctggcgtcctgcaacatctgaccaagatcatccgcaacgtcgttaccatcagcagctatttcctcctcgcccgtttgatttccttcaaatccaacatactgagcaaagtccggaatattgtcgtcttccacttcatcttcttccatttcaacaccttgctctccgtgggatgtccaacaattatagcttggcatgaaccccgactcaaacaagtggaaatgaatagtcctggatgcagaatactccttctgattcttacacttattgcatggacaacaaataaaacccctttgcctgttagcttcggccactctcaaaaaatagtgcacgccgtcaataaactctttggaccaccggtcagcgtacatccattgccgatccatctacatgagtcaaaaaaactgtacacaaataattattcttacaataatgacagtcatacaataattataagatatctttattcatacaaaaatcataaaatattacaccaaataattcttaaaaactatttgtaaagttttaaactaattttaatgtgttttacttcttttaattttcattttagtttgttttctattatttaagattaactttcactagagttttccttctcaattattttataaaaccttgtttagcaaatgaactaaatttctatatattctttcttccccacacacattttctctctcatcaacttacaactaaattttggagacaaaaaaagtgtgcaaaacataggtgcaaatgtagtatgacaaaaaaaacattggaggatgaagttgcaaaccttttaggcacctccgatttgtatgtaatcaccaaaataaattcaatagaaattttggcatgacctcccctcttttttgaagaaattttgaagcttgctcgggcagctggaggaggaagaagacatatatatagggatgggactttagtcccggttggtagcaccaaccggggctaaagatcaccgggatctttagtcccggttggtattaccaaccgggactaaagttacgatctgttaccaaccgggactaaagatcccgggggggcctgacaggccctgacagcattcaaaccgggactaaagatgatctttagtcccagttggtaacacaaaccgggactaaagatcaaatatgcccgttacccttttgaaccgggactaaagatcatctttagccccggtttttattgcatccgggactattgtggaaatcggccgaccgacgaaagatggtttctccaccagtgatatgtaagtacaatgtaactaatatatataattatagtgcaattataatataattaatatgtacatacagtataactaatacagtataagtaagtattgttttggtgaaacaattagttgctccttcttgctcagctagttatttcacgttcaaagcatcagatcttgggatcgactcccacggaggttacagtagtccttttttttggttgtaacggcagctagcacaaatcataatgcaatttagacggtggaaaatctgggagatttctaaaaaaaaaaatttgtcgacaaatttttagcaaaaccaaatttggataactatataagaaatcctgctaaaattttagtaagttgtcaaaattttgacaactatatcaaagttttggtaatgccaaattttggtaaggtcttattttgcatcaaagtgaacatgccctatgttattattacttcagcattcatggattataggttctataaaagccttgggccgtacgtcttgtgatcgagttgtgcaaatagatctctgagacaggcgtcgaacacctttttccaccatggaatcgtgtgcacggcggtgcgccgtctccggcctgatggcgctgtccatgcgcctcaccaagcagctctccgccgccgccgccgccagcaaggctggcgctgccggcaacctcgtcttctcgccgctgtccatctactccgcgctctccgtggtcaccgccggcgcgcgcgggcgcaccctgacagagctcctcggcgccctcggcgcagagtcccgcgagaagctcgccgcgaacgccggcgagatggcgcgtgctctccccgcccccggcggcggcgcggcacagccgggcggcggcccgcgcgtcgcgcacgcgtgcggagtctggcacgagcggacgcggacggttaggccggcgttccgcgacgccgccgccgcgtcgttcaacgccgcggccctcgccgtcgacttcctcaacaacgtaagcgaactatatatatatacacatgacaagatctaccaatctaatctgtcatgtatgcgtgtagccggaggaagcgaggaaggagatcaacagctgggttgcggcggcgacggagaacctcatcgacacgatcctcccgccggggtcggtgagcacggacacgggcctcgtggtcaccagcgccatctacttcaacggccaatggcggactcctttctgtaaggagataaccgagaagcgggcgttccaccgcctcgacggcggcgacgtcgaggcggacttcatgcgcagcggcgaggaccagtacatcgccgtgcacgacgggttcaaggtgctcaagatgccgtacgcggcttgtgtgagcgcgaggacgacgacgacgccgaggtactcgatgtacgtcttcctccccgacgagcgcgacggcctgtggagcctggaggacaggatggcggccggcggcgagggcttcctccgcgagcacacgccggagcggcgcgtcgaggtcggcgagttcaggatccccaggttcaagctctccttcgacgacagcgtcgtgggcgccctccagcgtctcggggtcagggacgtgttcaagccgttcgtggcggacctggccgacgtgctggaagcggagaactctggcgatgatccgccgctgtttgtgtcggacgtcaagcacaaggccgtcatcgaggtgaacgaggagggcaccgaggcggccgccgccaccgcggtttgtcttaccttcgcatcggcggcgccatcgtcacggcggccggcgagggtggatttcgtcgccgaccatccgttcgcgttcttggttttggaggagtcgtcgggtgcggtgctcttcgccggccacgttgttgacccgacagatgagtaattccatcttcacgtcatgaaatttgttttagctgcatttttgattcggttatgaaatttgtggacttccaattaagaacgatttgagttcctcgactccgaggagtggaattcaggttgtaagggtaggactaagccatgtttcgtcttgttcattggcatagagagacgcttctttcgtctcattatattgacacaatgtcacaatacacagtgaagagcggctccgataggactaagctatgtagtagatttttcttcgcaatccatttggtcagaaaaaaaaaaagaacagggcgttttactcatggaaacacaatgaacatatgccacaaactataattgcacagaacaattctcctttcttgagatgccacaaactaggatgagacgaaatttaggatgtaggggtttgtcttccgttttgctgtcatgacgctacttgtaaggacagccttttggtttctttctgcttattttgttgcttttagagctggtagcaagtttgggagtctttcatgttttccttttggagggctttgaacctccctatgctataaactggtgtctgggtgttcccagcacaatcctctggctttcaatagaagccgggccgtacggcctttctcttaaaaaaaataatggtaacatggagaaggcgtgcataagaacaattaagaagaaagctagttgttgtgggaaccctaatcaattagaataattccatcttctcctaataaaattcacacaaatggtcgaaccctaattgttgtgggaattacagagagcaaaacgaagtttttactaagaaattgcatggaggtgccttcccattgatttagacttgatgcaaattaaatttgttgaattccttttgaatgatcgattcaagttctattttcctttctcaaacacgcaagagagcgtcattatattattaattgagattttctcccattcttggaggggtatatacctaattacaatttgagagccataacccaacaaccaaacaaactttatgaagaaaaactaatagagaaactgcacccaacaaccaaacaaaatttatgaagaaaaactaatagagaaactgcttgtttagtacaaactttatgggaaaaaaaactaatagagcaaatttctgagaactacaagttcaatgactaaactatgaacttgctgcaactttagcaacttatttcagtttgcacggtaacacctttaaaaaaaaagtcctagcattatagttgcaaaccaactatagataggactgaaaatcaaattcccctttaagatatgcacgtagcgaggaggtgttgcacattttcttcctcttgatcagaaagctgccattaccccacgtgatcacgtctatggcgagccaaccgaaaggcaggccaacatctatcttttttttttaggccaatcaaacaaagagaacgcactagtacagaaaacgctattggtgccggttggtaactggcaataggtgccggtttcccaatcggcaccaattggtcggcaccaatacatccttccggcacctataggtaaaacagaaccggcacctataggtccacacgagccaaaaaaaaaacctcgagccgatccagaaccgaaatatccccacatcccgaatccagaagaacccctccacattctgtaatcccctccatccacaaatccagaataatatccccacatccaaaatcaaaatccaaagaacacatcaataacaataacaattcctacattcatttaattataattttcatccaatccaaatcttcatgttcttggtacacacatgaacaagagaagagaaattaaacaagagaagaaattaaaaactcgaaccacggcggcggtgacggggcccttggccgcctcctccttccccttaagcgtcgtcgggatgctgggaaggggagcaccgccgcccacgggaggggagggcgcggcggtgctgccgcctccgtgggagcccgccgcctcccctctcgccggatctggcgaaggggagggcacggccgccgccggcgtagcgggagctcgccgcctccctactggctctcatgtcgccggatctggaggagggagcccaccacctcccctcctgccggatctagcggaggggaggatgcggccggcgccgctgccacctctgccaagcagtcaccgatcgcgcgagcccgccgccacccacaagtggtcgccacctccgccaggcaaccgccggccgcgggagcccgccgcctccaaatcgagcgagagagagaggggaggtagacgcgagtgagagagagaggggagggagagagagggatgcgagagtagataaggacggtgggctcggttagtcggctaggctcggtttttttcaaaggtgccggttctattaaaaaaccggcacctctactatatgtgtcggtttttttaagaactgacacctatacttatattataggtgtcggttctaaacaaaaaaccgacacctatagtatatgtgttggttttttaatagaaccgctatagtgtttaaaggtgtcgattctacttttttttcatcgtgtggaggtgggaaaagggtcataggtgtcggttttaaatgaaccgacacctataaggccggtccctatgtgggtttttgtagtagtgatgacaacgtggtggaacctatgtacaacaaattacctaccaagggttatactccctctgtcccaaaatatttgacgtcgttgactttttaaaatatacttaaccgttcgttttatttaaaaacttttttgaaatatgtaaaactatatatatatatacataaaagtatattgataaatcaaatgataggaaaagaattaataattacttaatttttttgaataagacgaacgatcaaacatatttaaaaaagttaacggcgtcaaatatttagggacggaggagtactcaacagccctctacgaagtaacgaagctatgacgattttgttgagaaaactttcaatggggacgacgtctaaaaatgctgatcctctgttctaggggtgatattgcctccaagcgtgttgtataattagagatattccagtaatgcctttgccctcgagtcgtcccttatatgataaagcctcctatggtgatttttggggcagttcagaagttgtttcattgttccctttcttgaattgtttcattgccgacaacgctgaatttctcgggcggctgcagcgtcaagcttactacaatggtgatatgtgcctatttcatcactttggtggctcaacgtcttcccatagttgttcaactcctctttgtggatgttcaatgttcaatttgtaaataatgtttgactaggatgtcagttgttcattgaatcctcttgtatcttatcctaaggactcgttagtttgcttttatttttatttctagttcgattttggcgcacatcttctcttcttccgcatgtcgcatgcgcaaccagggtttaccttaccgccccggtaactacacggttaccgcggttaccaagcttactgcgatgtacggtaatataaataccatggtaacctccttaaattcaaataaatttaaaaaataatttgaatttttaataaattttgcacagtttttcacggttaccacgtttaccgtgtggtaatcgtgcttacctctaggacgcggtaaccctggccccggcggtttgaggaaccctgtcacaatgtgcgttggcgcgtggatacgatgaccgtacagcgcagcgtctggcggccactttgcctgggagcacgagccatcggatcgctcgtcgtcgtctcacgacctcgcctggattcctgttcgtgaggggggcttcttgcaaatataccaagatgcagacggttccaaaaaataattaaacagtaacggctggtaccaaggtaactgccatagtgaaattagatgtttggaaatgataataacttattaataatctattgtgcaaaaaaaagcatctatcagaggcacacaacgataataatatattaatttattacattacataaaaacatctatcagagacacacaacgataatataataacttattacagtgcagaaaaaacatatatcagagacaacgataatataactagatagatataaaagcagtcgggaaaccaggcggcagtaacccatcatggtgcagtgacctatttatgcctgggataatacgagtagaaaattatgcataagtcagtgattgtaccaactgaaatataagagataatctgagataacttgttttctctttgctagttacaccaggaacaaaaatataatgcaaggtaattaaagtcatttatgactgaaatagaggcgtcactacaataatgaaacgacactaaacttcaactgggtctaattgtggttgccatgctcggcgttctgaatgcattgtgctttagggtggattggtggctgacccgattgtgttgtgctctcgtatggcaaacacaacacaaacggcaaacttttatttagtgaaatatggcatccccagcagtaaaacctaaatatgcataaataaaatacagcataatcattgcactataggtgagactcaccccgaaacgaaattttttcttcaccctccatgtgggcaactccttacatgatatcttccctttgttcctacagcagagaggaaagggggagggagggagagtgagctttgcaatagcaacataaataaaatgttcaagtgaaagcagggttttaaatctcccgctaaactgccgctatctcccgctatagctgtttgagaaggattctagttatttgttctcatgtacaatttagccgctatatatagctccatttagcccgctatagctgctgctatagctgtttgagaagctaaccgctaaatgccttagctagctatttaaaacattgagtgaaaggaacaatcgcagtttaccagaacaaacattgaaaactgggcacctaccagtatgcaggagagcaacaggttatgatatggcacatgatgctataaggacctggaatatctttttcttctgcaattactccatctgttgcttgtgataatgtgccattactgaggtggtattgactaagatagattttggtgtctggcatgacaatggtgtcaggaacaatggtagggaacgccttagagctgacacagagtgacctcctaccaatgaagagtgcattgccatctatggatgtcacatggacagtccttcccagcataagatcagctagcctgtaaactgagtagtaaaccgcatcccattttgtgccaatcaccaaaatctcagagttgcattctaccagacggtaggaatacaatcgaaagctttcatcgcttgttggccatctaaattttgcaatcaactttggtgatggctccgataaacagtgctggggtgggtcgatctgtatgacctcatgctcacctctcaccgaatgaggtcgcagcagcacataaagcttcccatggaatggcaatgtagaagaacggtggttgatgcgatcccaggttgtagccaccctccattgctgatcaccggaggtagcaaaacatatgttccacacgccgatcggccgcatcattaccctgacgagtccatctcctacacccacgctgatgcaggcgccacagatgtttcgaagggagcaccaccgagacgcctcggagcggcagcgcacccgcggcaggagggtatccagcggcgggagctccgcggtgtcgccggtgaaggggtggaggaggcggatggctgtgtcgtggtccctctgcagcaggaggacgccgtcggcgtcgtagaggacgcagtggtccctgaaatggggcagcctgacgcggacgacggcgccggtcgacaggtggaaggagcggacgtggccgcggagcttgccgtggcccgggtggaggccgcggccctcgggcagcagcatccacccgcgcgggtggaagcgcgggtcgaggatgccgcggcggcgcgggcaggtggtggcggagcgccactggggacacacggcccggaagcggatgtagtcgcggaagtcgccggctgccactcgccatccgattaggctcaccaagtcctcgtgcagagacgcccacgacgaggatggatcgccggcgttaaccttccgcctactgctgcacgcaactaaggccatatgacatgccgctgcaacaaatattagcagcacatctagttcaccaaactatattcgcccaaggaacaaaaatagtacaatttttattagggaatcacctgcaggcaaaggaccagcggcagatgcaaaccttggcgttcgcagagggcggcgtatcagatgggggaggaagccctagggctaagccgctgcgccctacaaggccgacgagcctaaggctcagaagcccattaacgggacagagagcccaaaacatacggtctatagatagactcttgggactctaatctaaaccaccgccgggccagcacgcacgtccgcacggcctcctgcgccgggaggagggagaggatgcggtgcagtattgcgtccgggagtagggatggcaacggggtgggttgggctggaacggccccgtccccgacccctgacctctccacccggcccggcctcggcccctgccccgagtgaagatgcggggccaaaccatcccctgcccccggcccctgcagggatcc

SEQ ID NO: 6 - DNA, 358 bp, Magnaporthe grisea
agacgacactggtcacaaatgtgtatgaacgtgaaaagatcaacttctctgctcatgcatggatggttgtgtctcaaacctacactgtggatgctctattaaggaagctgcttaggaaagttggttacacagaaccaccactgtcaagtaacattgacaaaatggatgtgtatgatttgaaagaggaaataaagcgatgctcaaagttagaaaatgcttgatcgtacttgatgatgtctgggatcaagaagcatactttcaaatacgtgatgcattccagaatgaccaaggaagtcgcgtaataatcacaacacggaagaatcatgtggcagctcttgcttcctcaacatgtccttaa

SEQ ID NO: 7 - DNA, 505 bp, Magnaporthe grisea
  variation (365): A or G or C or T, unknown, or other
ggcttggaggccatgacacctatccactatagaattagcaaccttctctagctccttggggcatttgtggcccttgttatagaaagcccttctacagaagagttcaaatgcatcagcgccattcaaaggctggaggttgagacggcgtgctgaggtagcaagggctgcaacatcattctctcgagtttgatgacgactcgagttgcttgaagattctggaatgcttcttgcatctgaaagtcacttttttgtcccagacatcatccagcacaatcaaacatttgctatcttcaatctttttcttaatttcttctttcaggtcatgtgcatccatattgttcaagctatcaagtgacagttcagtngacccaatctttctaaggagctttcttaatagagcctccacattgtaggtntgggagacaacaatccatgcatgagagcaaagttgaccttttcacgctcataaatatttgtgactaatgtngtcttncccaccaagccc

SEQ ID NO: 8 - protein, 6 aa, Magnaporthe grisea
GVGKTT

SEQ ID NO: 9 - protein, 8 aa, Magnaporthe grisea
GMGGLGKT

SEQ ID NO: 10 - protein, 7 aa, Magnaporthe grisea
GGLGKTT

SEQ ID NO: 11 - protein, 7 aa, Magnaporthe grisea
GGLGKTT

SEQ ID NO: 12 - protein, 9 aa, Magnaporthe grisea
GNGGIGKTT

SEQ ID NO: 13 - protein, 8 aa, Magnaporthe grisea
GIAGVGKT

SEQ ID NO: 14 - protein, 9 aa, Magnaporthe grisea
GSGGVGKTT

SEQ ID NO: 15 - protein, 8 aa, Magnaporthe grisea
GAEGIGKT

SEQ ID NO: 16 - protein, 9 aa, Magnaporthe grisea
GMGGLGKTT

SEQ ID NO: 17 - protein, 9 aa, Magnaporthe grisea
GMGGMGKTT

SEQ ID NO: 18 - protein, 9 aa, Magnaporthe grisea
GFCGCGKTA

SEQ ID NO: 19 - protein, 9 aa, Magnaporthe grisea
GPAGIGKTA

SEQ ID NO: 20 - protein, 9 aa, Magnaporthe grisea
GMGGLGKTT

SEQ ID NO: 21 - protein, 8 aa, Magnaporthe grisea
LVVLDDWW

SEQ ID NO: 22 - protein, 10 aa, Magnaporthe grisea
ENFLIVLDDV

SEQ ID NO: 23 - protein, 11 aa, Magnaporthe grisea
SCLIVLDDVWD

SEQ ID NO: 24 - protein, 11 aa, Magnaporthe grisea
KCLIVLDDVWD

SEQ ID NO: 25 - protein, 10 aa, Magnaporthe grisea
KKFLIVLDDV

SEQ ID NO: 26 - protein, 11 aa, Magnaporthe grisea
KKFLLVLDDVW

SEQ ID NO: 27 - protein, 10 aa, Magnaporthe grisea
RYFIIIEDLW

SEQ ID NO: 28 - protein, 11 aa, Magnaporthe grisea
RYFIVIDDLWA

SEQ ID NO: 29 - protein, 9 aa, Magnaporthe grisea
KSCLIVLDD

SEQ ID NO: 30 - protein, 10 aa, Magnaporthe grisea
KRYLVVFDDV

SEQ ID NO: 31 - protein, 11 aa, Magnaporthe grisea
NRYFIVIDDIQ

SEQ ID NO: 32 - protein, 11 aa, Magnaporthe grisea
RRYLIIIDGLW

SEQ ID NO: 33 - protein, 11 aa, Magnaporthe grisea
RKCLIVLDDVW

SEQ ID NO: 34 - protein, 10 aa, Magnaporthe grisea
GSRIIITTRD

SEQ ID NO: 35 - protein, 12 aa, Magnaporthe grisea
NFQASRIIITTR

SEQ ID NO: 36 - protein, 11 aa, Magnaporthe grisea
PQASRIIITTR

SEQ ID NO: 37 - protein, 12 aa, Magnaporthe grisea
NFQATRVIITTR

SEQ ID NO: 38 - protein, 12 aa, Magnaporthe grisea
GNMIILTTRIQS

SEQ ID NO: 39 - protein, 9 aa, Magnaporthe grisea
GNMILVTTR

SEQ ID NO: 40 - protein, 13 aa, Magnaporthe grisea
NNSCSRILTTEIE

SEQ ID NO: 41 - protein, 12 aa, Magnaporthe grisea
GSRIITTTKVDE

SEQ ID NO: 42 - protein, 13 aa, Magnaporthe grisea
TSRIIVTTRKENI

SEQ ID NO: 43 - protein, 12 aa, Magnaporthe grisea
CRNGSRIVITTR

SEQ ID NO: 44 - protein, 13 aa, Magnaporthe grisea
DKDIGSRIVVTTT

SEQ ID NO: 45 - protein, 11 aa, Magnaporthe grisea
VNSFSRILITA

SEQ ID NO: 46 - protein, 12 aa, Magnaporthe grisea
DQGSRVIITTRK

SEQ ID NO: 47 - protein, 7 aa, Magnaporthe grisea
CGGLPLA

SEQ ID NO: 48 - protein, 8 aa, Magnaporthe grisea
CQGLPLAI

SEQ ID NO: 49 - protein, 7 aa, Magnaporthe grisea
CKGLPLA

SEQ ID NO: 50 - protein, 7 aa, Magnaporthe grisea
CHGLPLA

SEQ ID NO: 51 - protein, 7 aa, Magnaporthe grisea
LKGNPLA

SEQ ID NO: 52 - protein, 6 aa, Magnaporthe grisea
NGNPLA

SEQ ID NO: 53 - protein, 8 aa, Magnaporthe grisea
KCGGLPLA

SEQ ID NO: 54 - protein, 8 aa, Magnaporthe grisea
KCGGSPLA

SEQ ID NO: 55 - protein, 7 aa, Magnaporthe grisea
CDGLPLA

SEQ ID NO: 56 - protein, 7 aa, Magnaporthe grisea
CGGLPLA

SEQ ID NO: 57 - protein, 7 aa, Magnaporthe grisea
CDGQPLA

SEQ ID NO: 58 - protein, 7 aa, Magnaporthe grisea
CGGLPLA

SEQ ID NO: 59 - protein, 7 aa, Magnaporthe grisea
CQGLPLA

SEQ ID NO: 60 - protein, 11 aa, Magnaporthe grisea
CFLYCALFPED

SEQ ID NO: 61 - protein, 10 aa, Magnaporthe grisea
LRNCFLYCSL

SEQ ID NO: 62 - protein, 10 aa, Magnaporthe grisea
QKNCFLYCSL

SEQ ID NO: 63 - protein, 9 aa, Magnaporthe grisea
RNCFLYCSL

SEQ ID NO: 64 - protein, 11 aa, Magnaporthe grisea
LQQCVSYCSLF

SEQ ID NO: 65 - protein, 9 aa, Magnaporthe grisea
LQQCFLYCS

SEQ ID NO: 66 - protein, 10 aa, Magnaporthe grisea
CLKACLLYLS

SEQ ID NO: 67 - protein, 10 aa, Magnaporthe grisea
CLKTCLLYLS

SEQ ID NO: 68 - protein, 8 aa, Magnaporthe grisea
LKSCFLYL

SEQ ID NO: 69 - protein, 9 aa, Magnaporthe grisea
LRNCFLYCS

SEQ ID NO: 70 - protein, 9 aa, Magnaporthe grisea
LKACLLYFG

SEQ ID NO: 71 - protein, 8 aa, Magnaporthe grisea
FKTCLLYL

SEQ ID NO: 72 - protein, 9 aa, Magnaporthe grisea
LRNCFLYCS